(19) United States
(12) Patent Application Publication
(10) Pub. No.: US 2025/0186568 A1
(43) Pub. Date: Jun. 12, 2025

(54) CANCER VACCINES AND METHODS OF USE THEREOF

(71) Applicant: BriaCell Therapeutics Corp., Philadelphia, PA (US)

(72) Inventors: William V. Williams, Philadelphia, PA (US); Miguel Lopez-Lago, Philadelphia, PA (US); Markus Daniel Lacher, Redwood City, CA (US)

(73) Assignee: BriaCell Therapeutics Corp., Philadelphia, PA (US)

(21) Appl. No.: 18/840,333

(22) PCT Filed: Mar. 2, 2023
(86) PCT No.: PCT/US2023/014337

§ 371 Date: Aug. 21, 2024

Related U.S. Application Data
(60) Provisional application No. 63/316,251, filed on Mar. 3, 2022.
(60) Provisional application No. 63/415,475, filed on Oct. 12, 2022.

(51) Int. Cl. A61K 39/00; A61P 37/04; C07K 14/535; C07K 14/74

(52) U.S. Cl. CPC A61K 39/0011; A61P 37/04; C07K 14/535; C07K 14/70539; A61K 2039/5152; A61K 2039/5156; A61K 2039/55522; A61K 2039/812; A61K 2039/86; A61K 2039/876; A61K 2039/884

(57) ABSTRACT

The present disclosure provides modified human cancer cells that express exogenous human leukocyte antigen (HLA) alleles. The present disclosure also provides expression vectors for simultaneous expression of one or more HLA alleles. Methods for using the modified human cancer cells of the present disclosure as a whole-cell cancer vaccine for treating a cancer in a subject are provided.

### CROSS-REFERENCE TO RELATED APPLICATIONS

This application claims priority to U.S. Provisional Application No. 63/316,251, filed Mar. 3, 2022, and U.S. Provisional Application No. 63/415,475, filed Oct. 12, 2022, the disclosures of which are herein incorporated by reference in their entirety for all purposes.

### BACKGROUND

Developing effective immune responses is essential in developing vaccines as well as in cancer immunotherapy. Adaptive immune responses start with antigen presentation by a professional antigen-presenting cell (APC). These cells typically express Class II major histocompatibility complex (MHC) proteins along with co-stimulatory molecules such as CD80 and CD86. It is generally assumed that to be effective, a whole-cell immunotherapy needs to express immunogenic antigens which are the target of the immune response, and following uptake of immunogenic proteins, APCs such as dendritic cells (DCs) need to present such antigens to T cells. Thus, there is also a need for improved active immunotherapies for cancer and related diseases. The present disclosure satisfies these needs and provides related advantages as well.

### SUMMARY

In one aspect, the present disclosure provides a modified human cancer cell comprising: (a) one or more recombinant polynucleotides each encoding an allele of a human leukocyte antigen (HLA) class I gene; and (b) one or more recombinant polynucleotides each encoding an allele of an HLA class II gene, wherein one or more HLA alleles endogenous to the cell have been inactivated.

In a related aspect, the present disclosure provides a modified human cancer cell that expresses class I and II MHC proteins, wherein the modified human cancer cell comprises: (a) one or more recombinant polynucleotides each encoding an allele of an HLA class I gene (e.g., for expression of recombinant class I MHC proteins in the cell); and (b) one or more recombinant polynucleotides each encoding an allele of an HLA class II gene (e.g., for expression of recombinant class II MHC proteins in the cell), wherein one or more HLA alleles endogenous to the cell have been inactivated.

In some embodiments, the modified human cancer cell comprises (a) a first recombinant polynucleotide encoding a first allele of an HLA class I gene and a second recombinant polynucleotide encoding a second allele of the HLA class I gene, or (a) a first recombinant polynucleotide encoding a first allele of an HLA class I gene and a second recombinant polynucleotide encoding an allele of a second HLA class I gene. In some embodiments, the modified human cancer cell further comprises (b) a first recombinant polynucleotide encoding an allele of a first HLA class II gene and a second recombinant polynucleotide encoding an allele of a second HLA class II gene, or (b) a first recombinant polynucleotide encoding a first allele of an HLA class II gene and a second recombinant polynucleotide encoding a second allele of the HLA class II gene.

In some embodiments, the HLA class I gene comprises an HLA-A gene, an HLA-B gene, an HLA-C gene, an HLA-E gene, an HLA-F gene, an HLA-G gene, a beta-2-microglobulin (B2M) gene, or a combination thereof. In some embodiments, the allele of the HLA-A gene comprises an HLA-A*01:01 allele, an HLA-A*02:01 allele, an HLA-A*02:06 allele, an HLA-A*03:01 allele, an HLA-A*11:01 allele, an HLA-A*23:01 allele, an HLA-A*24:02 allele, an HLA-A*26:01 allele, an HLA-A*29:02 allele, an HLA-A*31:01 allele, an HLA-A*32:01 allele, an HLA-A*33:03 allele, an HLA-A*68:01 allele, or a combination thereof.

In some embodiments, the allele of the HLA-A gene comprises: (i) an HLA-A*01:01 allele and an HLA-A*68:01 allele; (ii) an HLA-A*02:01 allele and an HLA-A*11:01 allele; (iii) an HLA-A*03:01 allele and an HLA-A*23:01 allele; or (iv) an HLA-A*33:03 allele and optionally an HLA-A*24:02 allele.

In various embodiments, the HLA class II gene comprises an HLA class II alpha subunit gene, an HLA class II beta subunit gene, or a combination thereof. In some embodiments, the HLA class II gene comprises an HLA-DP gene, an HLA-DM gene, an HLA-DO gene, an HLA-DQ gene, an HLA-DR gene, or a combination thereof. In some embodiments, the HLA-DM gene comprises an HLA-DMA gene, an HLA-DMB gene, or a combination thereof. In some embodiments, the HLA-DR gene comprises an HLA-DRA gene, an HLA-DRB1 gene, an HLA-DRB3 gene, an HLA-DRB4 gene, an HLA-DRB5 gene, or a combination thereof.

In some embodiments, the HLA-DR gene is an HLA-DRB3 gene comprising an HLA-DRB3*01:01 allele, an HLA-DRB3*02:02 allele, an HLA-DRB3*03:01 allele, or a combination thereof. In some embodiments, the HLA-DR gene is an HLA-DRB4 gene comprising an HLA-DRB4*01:01 allele, an HLA-DRB4*01:03 allele, or a combination thereof. In some embodiments, the HLA-DR gene is an HLA-DRB5 gene comprising an HLA-DRB5*01:01 allele, an HLA-DRB5*01:02 allele, an HLA-DRB5*02:02 allele, or a combination thereof.

In certain embodiments, the allele of the HLA-DR gene comprises: (i) an HLA-DRB3*02:02 allele and an HLA-DRB5*01:01 allele; (ii) an HLA-DRB4*01:01 allele and an HLA-DRB3*01:01 allele; (iii) an HLA-DRB3*03:01 allele and an HLA-DRB5*01:02 allele; or (iv) an HLA-DRB5*02:02 allele and an HLA-DRB3*01:01 allele.

In certain other embodiments, the allele of the HLA-DR gene comprises: (i) an HLA-DRB3*02:02 allele and an HLA-DRB4*01:01 allele; (ii) an HLA-DRB4*01:01 allele and an HLA-DRB3*03:01 allele; (iii) an HLA-DRB3*01:01 allele and an HLA-DRB5*01:02 allele; or (iv) an HLA-DRB5*01:01 allele and an HLA-DRB5*02:02 allele.

In particular embodiments, the modified human cancer cell comprises one or more recombinant polynucleotides each encoding an allele of a human leukocyte antigen (HLA) class I gene having at least 85%, at least 90%, at least 95%, at least 96%, at least 97%, at least 98%, or at least 99% identity to a polynucleotide sequence of any one of SEQ ID NOS:22-29. In particular embodiments, the modified human cancer cell comprises one or more recombinant polynucleotides each encoding an allele of a human leukocyte antigen (HLA) class II gene having at least 85%, at least 90%, at least 95%, at least 96%, at least 97%, at least 98%, or at least 99% identity to a polynucleotide sequence of any one of SEQ ID NOS:30-35. In particular embodiments, the modified human cancer cell comprises (a) one or more recombinant polynucleotides each encoding an allele of a human leukocyte antigen (HLA) class I gene having at least 85%, at least 90%, at least 95%, at least 96%, at least 97%, at least 98%, or at least 99% identity to a polynucleotide sequence of any one of SEQ ID NOS:22-29, and (b) one or more recombinant polynucleotides each encoding an allele of a human leukocyte antigen (HLA) class II gene having at least 85%, at least 90%, at least 95%, at least 96%, at least 97%, at least 98%, or at least 99% identity to a polynucleotide sequence of any one any of SEQ ID NOS:30-35.

In particular embodiments, the modified human cancer cell comprises one or more recombinant polynucleotides each encoding an allele of a human leukocyte antigen (HLA) class I gene having a polynucleotide sequence of any one of SEQ ID NOS:22-29. In particular embodiments, the modified human cancer cell comprises one or more recombinant polynucleotides each encoding an allele of a human leukocyte antigen (HLA) class II gene having a polynucleotide sequence of any one of SEQ ID NOS:30-35. In particular embodiments, the modified human cancer cell comprises (a) one or more recombinant polynucleotides each encoding an allele of a human leukocyte antigen (HLA) class I gene having a polynucleotide sequence of any one of SEQ ID NOS:22-29, and (b) one or more recombinant polynucleotides each encoding an allele of a human leukocyte antigen (HLA) class II gene having a polynucleotide sequence of any one any of SEQ ID NOS:30-35.

In another aspect, the present disclosure provides a modified human cancer cell comprising: (a) a recombinant polynucleotide encoding at least one of an HLA-A*01:01 allele, an HLA-A*68:01 allele, an HLA-DRB3*02:02 allele, and an HLA-DRB5*01:01 allele; (b) a recombinant polynucleotide encoding at least one of encoding an HLA-A*02:01 allele, an HLA-A*11:01 allele, an HLA-DRB4*01:01 allele, and an HLA-DRB3*01:01 allele; (c) a recombinant polynucleotide encoding at least one of an HLA-A*03:01 allele, an HLA-A*23:01 allele, an HLA-DRB3*03:01 allele, and an HLA-DRB5*01:02 allele; and/or (d) a recombinant polynucleotide encoding at least one of an HLA-A*24:02 allele, an HLA-A*33:03 allele, an HLA-DRB5*02:02 allele, and an HLA-DRB3*01:01 allele, wherein one or more HLA alleles endogenous to the cell have been inactivated. In some embodiments, the modified human cancer cell comprises: (a) a recombinant polynucleotide encoding an HLA-A*01:01 allele, an HLA-A*68:01 allele, an HLA-DRB3*02:02 allele, and an HLA-DRB5*01:01 allele; (b) a recombinant polynucleotide encoding an HLA-A*02:01 allele, an HLA-A*11:01 allele, an HLA-DRB4*01:01 allele, and an HLA-DRB3*01:01 allele; (c) a recombinant polynucleotide encoding an HLA-A*03:01 allele, an HLA-A*23:01 allele, an HLA-DRB3*03:01 allele, and an HLA-DRB5*01:02 allele; and/or (d) a recombinant polynucleotide encoding an HLA-A*24:02 allele, an HLA-A*33:03 allele, an HLA-DRB5*02:02 allele, and an HLA-DRB3*01:01 allele, wherein one or more HLA alleles endogenous to the cell have been inactivated. In particular embodiments, the recombinant polynucleotide comprises one or more codon optimized sequences encoding the alleles described herein (e.g., SEQ ID NOS:22-35).

In yet another aspect, the present disclosure provides a modified human cancer cell comprising: (a) a recombinant polynucleotide encoding at least one of an HLA-A*01:01 allele, an HLA-A*68:01 allele, an HLA-DRB3*02:02 allele, and an HLA-DRB4*01:01 allele; (b) a recombinant polynucleotide encoding at least one of an HLA-A*02:01 allele, an HLA-A*11:01 allele, an HLA-DRB4*01:01 allele, and an HLA-DRB3*03:01 allele; (c) a recombinant polynucleotide encoding at least one of an HLA-A*03:01 allele, an HLA-A*23:01 allele, an HLA-DRB3*01:01 allele, and an HLA-DRB5*01:02 allele; and/or (d) a recombinant polynucleotide encoding at least one of an HLA-A*33:03 allele, an HLA-DRB5*01:01 allele, and an HLA-DRB5*02:02 allele, wherein one or more HLA alleles endogenous to the cell have been inactivated. In some embodiments, the modified human cancer cell comprises: (a) a recombinant polynucleotide encoding an HLA-A*01:01 allele, an HLA-A*68:01 allele, an HLA-DRB3*02:02 allele, and an HLA-DRB4*01:01 allele; (b) a recombinant polynucleotide encoding an HLA-A*02:01 allele, an HLA-A*11:01 allele, an HLA-DRB4*01:01 allele, and an HLA-DRB3*03:01 allele; (c) a recombinant polynucleotide encoding an HLA-A*03:01 allele, an HLA-A*23:01 allele, an HLA-DRB3*01:01 allele, and an HLA-DRB5*01:02 allele; and/or (d) a recombinant polynucleotide encoding an HLA-A*33:03 allele, an HLA-DRB5*01:01 allele, and an HLA-DRB5*02:02 allele, wherein one or more HLA alleles endogenous to the cell have been inactivated. The modified human cancer cell of (d) may further comprise an HLA-A*24:02 allele. In particular embodiments, the recombinant polynucleotide comprises one or more codon optimized sequences encoding the alleles described herein (e.g., SEQ ID NOS:22-35).

In various embodiments, the modified human cancer cell further comprises a recombinant polynucleotide encoding a cytokine. In some embodiments, the cytokine comprises a chemokine, an interferon, an interleukin, a tumor necrosis factor, or a combination thereof. In some embodiments, the recombinant polynucleotide encoding the cytokine comprises a recombinant polynucleotide encoding an early T cell activation antigen-1 (ETA-1), a lymphocyte-activating factor (LAF), an interleukin-1 family member (IL-1α, IL-β, IL-1Ra, IL-18, IL-33, IL-36Ra, IL-36α, IL-36β, IL-36γ, IL-37, IL-38), an interleukin-2 (IL-2), an interleukin-3 (IL-3), an interleukin-4 (IL-4), an interleukin-5 (IL-5), an interleukin-6 (IL-6), an interleukin-7 (IL-7), an interleukin-8 (IL-8), an interleukin-9 (IL-9), an interleukin-10 (IL-10), an interleukin-12 (IL-12), an interleukin-13 (IL-13), an interleukin-15 (IL-15), an interleukin-17 (IL-17), an interleukin-18 (IL-18), an interleukin-21 (IL-21), an interleukin-23 (IL-23), an interleukin-25 (IL-25), an interleukin-33 (IL-33), an interferon alpha (IFN-α), an interferon lambda 1 (IFNλ1 (IL-29)), an interferon lambda 2 (IFNλ2 (IL-28A)), an interferon lambda 3 (IFNλ3 (IL-28B)), an interferon lambda 4 (IFNλ4), a granulocyte-macrophage colony-stimulating factor (GM-CSF), a macrophage CSF (CSF-1), a macrophage migration inhibitory factor (MIF), a CD40L molecule (CD40L), a RANTES molecule (RANTES), a monocyte chemoattractant protein (MCP-1), a monocyte inflammatory protein (MIP-1α, MIP-1β), a lymphotactin, a fractalkine, or a combination thereof. In some embodiments, the cytokine comprises a granulocyte-macrophage colony-stimulating factor (GM-CSF). In some embodiments, the cytokine comprises an interferon alpha (IFN-α), such as e.g., IFN-α2a or IFN-α2b. In some embodiments, the cytokine comprises a GM-CSF and an (IFN-α), preferably IFN-α2a or IFN-α2b. In some embodiments, the IFN-α is an exogenous protein comprising a pegylated IFN-α2a.

In some embodiments, the cytokine comprises a granulocyte-macrophage colony-stimulating factor (GM-CSF) encoded by a polynucleotide having at least 85%, at least 90%, at least 95%, at least 96%, at least 97%, at least 98%, or at least 99% identity to SEQ ID NO:39. In some embodiments, the cytokine comprises an interferon alpha (IFN-α) encoded by a polynucleotide having at least 85%, at least 90%, at least 95%, at least 96%, at least 97%, at least 98%, or at least 99% identity to SEQ ID NO:40. In some embodiments, the cytokine comprises a GM-CSF and an IFN-α, such as e.g., IFN-α2a or IFN-α2b. In some embodiments, the recombinant polynucleotide encoding the CM-CSF comprises SEQ ID NO:39. In some embodiments, the recombinant polynucleotide encoding the IFN-α comprises SEQ ID NO:40. In some embodiments, the cytokine comprises an interleukin-7 (IL-7). In some embodiments, the cytokine comprises an IL-7 encoded by a polynucleotide having at least 65%, at least 70%, at least 75%, at least 80%, at least 90%, at least 95%, or at least 99% identity to SEQ ID NO:41. In some embodiments, the recombinant polynucleotide encoding the IL-7 comprises SEQ ID NO:41. In some embodiments, the cytokine comprises an IL-7 encoded by a polynucleotide having at least 65%, at least 70%, at least 75%, at least 80%, at least 90%, at least 95%, or at least 99% identity to SEQ ID NO:42. In some embodiments, the recombinant polynucleotide encoding the IL-7 comprises SEQ ID NO:42. In some embodiments, the cytokine comprises an interleukin-12 (IL-12). In some embodiments, the cytokine comprises a bioactive form of IL-12 containing subunits p40 and p35. In some embodiments, the cytokine comprises an IL-12 encoded by a polynucleotide having at least 65%, at least 70%, at least 75%, at least 80%, at least 90%, at least 95%, or at least 99% identity to SEQ ID NO:44. In some embodiments, the recombinant polynucleotide encoding the IL-12 comprises SEQ ID NO:44.

In various embodiments, the modified human cancer cell further comprises a recombinant polynucleotide encoding a co-stimulatory molecule. In some embodiments, the recombinant polynucleotide encoding the co-stimulatory molecule comprises a recombinant polynucleotide encoding a CD86 molecule (CD86), CD80 molecule (CD80), 4-1BB ligand molecule (4-1BBL, also known as TNFSF9 or CD137L), ICOS ligand molecule (ICOS-L), CD70 molecule (CD70 a.k.a. CD27L), CD40 molecule (CD40), OX40 ligand molecule (OX40L), GITR ligand molecule (GITRL), TIM-4 molecule (TIM-4), LIGHT molecule (LIGHT), ICAM1 molecule (ICAM1), LFA3 molecule (LFA3), a CD30 molecule (CD30), or a combination thereof.

In some embodiments, the modified human cancer cell further comprises a recombinant polynucleotide encoding an antigen of a pathogen, a tumor-associated antigen, a neo-antigen, an allergen, an antigen that is the target of an autoimmune response, a fragment thereof, or a combination thereof.

In some embodiments, one or more HLA-A and/or HLA-DR alleles endogenous to the modified human cancer cell have been inactivated. In some embodiments, the one or more HLA-A alleles comprises an HLA-A*24:02 allele and/or an HLA-A*11:01 allele. In some embodiments, the one or more HLA-DR alleles comprises one or more HLA-DRB3 alleles. In some embodiments, the one or more HLA-DRB3 alleles comprises an HLA-DRB3*01:01 allele and/or an HLA-DRB3*02:02 allele.

In any of the above embodiments, one or more of the recombinant polynucleotides present in the modified human cancer cell is present on a vector in the cell. In some embodiments, the one or more recombinant polynucleotides each encoding an allele of an HLA class I gene and the one or more recombinant polynucleotides each encoding an allele of an HLA class II gene are present on the same vector in the cell. In some embodiments, the vector comprises two recombinant polynucleotides each encoding an allele of an HLA class I gene. In other embodiments, the vector comprises two recombinant polynucleotides each encoding an allele of an HLA class II gene. In other embodiments, the vector comprises two recombinant polynucleotides each encoding an allele of an HLA class I gene and two recombinant polynucleotides each encoding an allele of an HLA class II gene. In yet other embodiments, the one or more recombinant polynucleotides each encoding an allele of an HLA class I gene and the one or more recombinant polynucleotides each encoding an allele of an HLA class II gene are present on separate vectors in the cell. In yet other embodiments, the one or more recombinant polynucleotides each encoding two alleles of HLA class I genes and the one or more recombinant polynucleotides each encoding two alleles of HLA class II genes are present on separate vectors in the cell.

In some embodiments, the one or more of the recombinant polynucleotides present in the modified human cancer cell comprises a sequence having at least 85% identity to any one of SEQ ID NOS:22-35. In some embodiments, the one or more of the recombinant polynucleotides present in the modified human cancer cell comprises a sequence encoding a polypeptide having at least 85% identity to a polypeptide encoded by any one of SEQ ID NOS:22-35. In some embodiments, the one or more of the recombinant polynucleotides present in the modified human cancer cell comprises the sequence of any one of SEQ ID NOS:22-35.

In some embodiments, the human cancer cell is a human cancer cell line. In certain embodiments, the human cancer cell line is a breast cancer (e.g., SV-BR-1), prostate cancer (e.g., PC-3, LNCaP), melanoma (e.g., SK-MEL-24), or lung cancer (e.g., NCI-H2228) cell line.

In various embodiments, the one or more recombinant polynucleotides present in the modified human cancer cell each comprises a heterologous polynucleotide sequence between the polynucleotide sequence encoding a first HLA allele and a second HLA allele. In some embodiments, the heterologous polynucleotide sequence encodes one or more self-cleaving peptides. In some embodiments, the self-cleaving peptide is positioned at the 3′ end of the first HLA allele and at the 5′ end of the second HLA allele. In some embodiments, the self-cleaving peptide comprises T2A, P2A, E2A, and/or a combination thereof. In some embodiments, the heterologous polynucleotide sequence is a non-coding sequence.

In another aspect, the present disclosure provides a modified human cancer cell comprising: (a) one or more vectors each comprising a recombinant polynucleotide encoding at least one gene selected from GM-CSF, IFN-α, CD86, IL-12, CD80, HLA-DRA, IL-7, and 4-1BBL; (b) one or more vectors each comprising a recombinant polynucleotide encoding at least one HLA-A allele selected from an HLA-A*01:01 allele, an HLA-A*68:01 allele, an HLA-A*02:01 allele, an HLA-A*11:01 allele, an HLA-A*03:01 allele, an HLA-A*23:01 allele, an HLA-A*24:02, and an HLA-A*33:03 allele; and/or (c) one or more vectors each comprising a recombinant polynucleotide encoding at least one HLA-DR allele selected from an HLA-DRB3*02:02, HLA-DRB5*01:01, HLA-DRB4*01:01, HLA-DRB3*01:01, HLA-DRB3*03:01, HLA-DRB5*01:02, and/or HLA-DRB5*02:02, wherein one or more HLA alleles endogenous to the cell have been inactivated.

In certain embodiments, the modified human cancer cell comprises: (a) four vectors each comprising a recombinant polynucleotide encoding two different genes selected from GM-CSF, IFN-α, CD86, IL-12, CD80, HLA-DRA, IL-7, and 4-1BBL. In certain other embodiments, the modified human cancer cell comprises: (b) one vector comprising a recombinant polynucleotide encoding an HLA-A*01:01 allele and an HLA-A*68:01 allele, or an HLA-A*02:01 allele and an HLA-A*11:01 allele, or an HLA-A*03:01 allele and an HLA-A*23:01 allele, or an HLA-A*24:02 allele and an HLA-A*33:03 allele; and/or (c) one vector comprising a recombinant polynucleotide encoding an HLA-DRB3*02:02 allele and an HLA-DRB5*01:01 allele, or an HLA-DRB4*01:01 allele and an HLA-DRB3*01:01 allele, or an HLA-DRB3*03:01 allele and an HLA-DRB5*01:02 allele, or an HLA-DRB5*02:02 allele and an HLA-DRB3*01:01 allele.

In particular embodiments, the modified human cancer cell comprises: (a) four vectors each comprising a recombinant polynucleotide encoding two different genes selected from GM-CSF, IFN-α, CD86, IL-12, CD80, HLA-DRA, IL-7, and 4-1BBL; (b) one vector comprising a recombinant polynucleotide encoding an HLA-A*01:01 allele and an HLA-A*68:01 allele; and (c) one vector comprising a recombinant polynucleotide encoding an HLA-DRB3*02:02 allele and an HLA-DRB5*01:01 allele.

In particular embodiments, the modified human cancer cell comprises: (a) four vectors each comprising a recombinant polynucleotide encoding two different genes selected from GM-CSF, IFN-α, CD86, IL-12, CD80, HLA-DRA, IL-7, and 4-1BBL; (b) one vector comprising a recombinant polynucleotide encoding an HLA-A*02:01 allele and an HLA-A*11:01 allele; and (c) one vector comprising a recombinant polynucleotide encoding an HLA-DRB4*01:01 allele and an HLA-DRB3*01:01 allele.

In particular embodiments, the modified human cancer cell comprises: (a) four vectors each comprising a recombinant polynucleotide encoding two different genes selected from GM-CSF, IFN-α, CD86, IL-12, CD80, HLA-DRA, IL-7, and 4-1BBL; (b) one vector comprising a recombinant polynucleotide encoding an HLA-A*03:01 allele and an HLA-A*23:01 allele; and (c) one vector comprising a recombinant polynucleotide encoding an HLA-DRB3*03:01 allele and an HLA-DRB5*01:02 allele.

In particular embodiments, the modified human cancer cell comprises: (a) four vectors each comprising a recombinant polynucleotide encoding two different genes selected from GM-CSF, IFN-α, CD86, IL-12, CD80, HLA-DRA, IL-7, and 4-1BBL; (b) one vector comprising a recombinant polynucleotide encoding an HLA-A*24:02 allele and an HLA-A*33:03 allele; and (c) one vector comprising a recombinant polynucleotide encoding an HLA-DRB5*02:02 allele and an HLA-DRB3*01:01 allele.

In various embodiments, one or more HLA-A and/or HLA-DR alleles endogenous to the cell have been inactivated. In some embodiments, the one or more inactivated endogenous HLA-A alleles in the modified human cancer cell comprises an HLA-A*24:02 allele and/or an HLA-A*11:01 allele. In some embodiments, the one or more inactivated endogenous HLA-DR alleles in the modified human cancer cell comprises one or more HLA-DRB3 alleles. In some embodiments, the one or more inactivated endogenous HLA-DRB3 alleles comprises an HLA-DRB3*01:01 allele and/or an HLA-DRB3*02:02 allele.

In some embodiments, one or more of the recombinant polynucleotides comprises a sequence having at least 85% identity to any one of SEQ ID NOS:22-44. In some embodiments, one or more of the recombinant polynucleotides comprises the sequence of any one of SEQ ID NOS:22-44.

In some embodiments, the human cancer cell is a human cancer cell line. In certain embodiments, the human cancer cell line is a breast cancer (e.g., SV-BR-1), prostate cancer (e.g., PC-3, LNCaP), melanoma (e.g., SK-MEL-24), or lung cancer (e.g., NCI-H2228) cell line.

In some embodiments, a modified human cancer cell described herein is a replication-incompetent modified human cancer cell. In some embodiments, the modified human cancer cell is rendered replication incompetent by irradiation, freeze-thawing, and/or mitomycin C treatment. In some embodiments, the modified human cancer cell is a human cancer cell line. In some embodiments, the modified human cancer cell further comprises an inactivated CD47 molecule. In some embodiments, the modified human cancer cell further comprises an inactivated Ii(CD74) molecule.

In another aspect, the present disclosure provides a composition comprising a modified human cancer cell described herein. In some embodiments, the composition comprises at least 1,000,000 cells. In some embodiments, the composition comprising the modified human cancer cell described herein is formulated as a pharmaceutical composition and further comprising a pharmaceutically acceptable carrier. In some embodiments, the pharmaceutical composition further comprises a cryoprotectant.

In another aspect, the present disclosure provides a method for treating cancer in a subject, the method comprising administering to the subject a therapeutically effective amount of a pharmaceutical composition described herein. In some embodiments, the pharmaceutical composition is administered every week. In some embodiments, the pharmaceutical composition is administered at a dose of at least 1,000,000 cells. In some embodiments, the pharmaceutical composition is administered intradermally in the upper back or thighs of the subject. In some embodiments, the method further comprises administering to the subject a therapeutically effective amount of cyclophosphamide at least 2 days prior to the administering of the pharmaceutical composition. In some embodiments, the method further comprises administering to the subject a therapeutically effective amount of IFN-α2a, pegylated IFN-α2a, or IFN-α2b at least 1 hour after and at least 1 day following the administering of the pharmaceutical composition.

In another aspect, the present disclosure provides a kit for treating a subject in need thereof with cancer comprising a pharmaceutical composition described herein. In some embodiments, the kit further comprises a therapeutically effective amount of IFN-α2b. The kit may comprise instructions for treating the subject using any of the methods described herein.

In another aspect, the present disclosure provides a vector comprising: (a) a recombinant polynucleotide encoding an HLA-A*02:01 allele, an HLA-A*11:01 allele, an HLA-DRB4*01:01 allele, and an HLA-DRB3*03:01 allele; (b) a recombinant polynucleotide encoding an HLA-A*01:01 allele, an HLA-A*68:01 allele, an HLA-DRB3*02:02 allele, and an HLA-DRB4*01:01 allele; (c) a recombinant polynucleotide encoding an HLA-A*03:01 allele, an HLA-A*23:01 allele, an HLA-DRB3*01:01 allele, and an HLA-DRB5*01:02 allele; and/or (d) a recombinant polynucleotide encoding an HLA-A*33:03 allele, an HLA-DRB5*01:01 allele, and an HLA-DRB5*02:02 allele, wherein each recombinant polynucleotide in (a)-(d) further comprises a polynucleotide encoding a self-cleaving peptide between two HLA alleles. In some embodiments, the vector in (d) further comprises a recombinant polynucleotide encoding an HLA-A*24:02 allele. In some embodiments, the polynucleotide encoding the self-cleaving peptide is positioned at the 3′ end of a first HLA allele and at the 5′ end of a second HLA allele. In some embodiments, the self-cleaving peptide comprises T2A, P2A, E2A, and/or a combination thereof.

In various embodiments, the recombinant polynucleotide present in any of (a)-(d) in the vector described herein comprises at least one of a sequence having at least 85%, at least 90%, at least 95%, at least 96%, at least 97%, at least 98%, or at least 99% identity to any one of SEQ ID NOS:22-35. In some embodiments, the recombinant polynucleotide present in any of (a)-(d) in the vector comprises a sequence encoding a polypeptide having at least 85%, at least 90%, at least 95%, at least 96%, at least 97%, at least 98%, or at least 99% identity to a polypeptide encoded by any one of SEQ ID NOS:22-35. In some embodiments, the recombinant polynucleotide comprises at least one of any one of SEQ ID NOS:22-35. In some embodiments, the recombinant polynucleotide comprises a MNDU3 promoter.

In some embodiments, the recombinant polynucleotide present in any of (a)-(d) in the vector described herein comprises a HLA class I gene having at least 85%, at least 90%, at least 95%, at least 96%, at least 97%, at least 98%, or at least 99% identity to any one of SEQ ID NOS:22-29. In some embodiments, the recombinant polynucleotide present in any of (a)-(d) in the vector described herein comprises a HLA class II gene having at least 85%, at least 90%, at least 95%, at least 96%, at least 97%, at least 98%, or at least 99% identity to any one of SEQ ID NOS:30-35. In some embodiments, the recombinant polynucleotide present in any of (a)-(d) in the vector described herein comprises a HLA class I gene having at least 85%, at least 90%, at least 95%, at least 96%, at least 97%, at least 98%, or at least 99% identity to any one of SEQ ID NOS:22-29, and a HLA class II gene having at least 85%, at least 90%, at least 95%, at least 96%, at least 97%, at least 98%, or at least 99% identity to any one of SEQ ID NOS:30-35.

In some embodiments, the recombinant polynucleotide present in any of (a)-(d) in the vector described herein comprises a HLA class I gene having a polynucleotide sequence of any one of SEQ ID NOS:22-29. In some embodiments, the recombinant polynucleotide present in any of (a)-(d) in the vector described herein comprises a HLA class II gene having a polynucleotide sequence of any one of SEQ ID NOS:30-35. In some embodiments, the recombinant polynucleotide present in any of (a)-(d) in the vector described herein comprises a HLA class I gene having a polynucleotide sequence of any one of SEQ ID NOS:22-29, and a HLA class II gene having a polynucleotide sequence of any one of SEQ ID NOS:30-35.

In various embodiments, the vector further comprises a recombinant polynucleotide encoding a cytokine. In some embodiments, the cytokine comprises a chemokine, an interferon, an interleukin, a tumor necrosis factor, or a combination thereof. In some embodiments, the recombinant polynucleotide encoding the cytokine comprises a recombinant polynucleotide encoding an early T cell activation antigen-1 (ETA-1), a lymphocyte-activating factor (LAF), an interleukin-1 family member (IL-1α, IL-β, IL-1Ra, IL-18, IL-33, IL-36Ra, IL-36α, IL-36β, IL-36γ, IL-37, IL-38), an interleukin-2 (IL-2), an interleukin-3 (IL-3), an interleukin-4 (IL-4), an interleukin-5 (IL-5), an interleukin-6 (IL-6), an interleukin-7 (IL-7), an interleukin-8 (IL-8), an interleukin-9 (IL-9), an interleukin-10 (IL-10), an interleukin-12 (IL-12), an interleukin-13 (IL-13), an interleukin-15 (IL-15), an interleukin-17 (IL-17), an interleukin-18 (IL-18), an interleukin-21 (IL-21), an interleukin-23 (IL-23), an interleukin-25 (IL-25), an interleukin-33 (IL-33), an interferon alpha (IFN-α), an interferon lambda 1 (IFNλ1 (IL-29)), an interferon lambda 2 (IFNλ2 (IL-28A)), an interferon lambda 3 (IFNλ3 (IL-28B)), an interferon lambda 4 (IFNλ4), a granulocyte-macrophage colony-stimulating factor (GM-CSF), a macrophage CSF (CSF-1), a macrophage migration inhibitory factor (MIF), a CD40L molecule (CD40L), a RANTES molecule (RANTES), a monocyte chemoattractant protein (MCP-1), a monocyte inflammatory protein (MIP-1α, MIP-1β), a lymphotactin, a fractalkine, or a combination thereof. In some embodiments, the cytokine comprises a granulocyte-macrophage colony-stimulating factor (GM-CSF). In some embodiments, the cytokine comprises an interferon alpha (IFN-α), such as e.g., IFN-α2a or IFN-α2b. In some embodiments, the cytokine comprises a GM-CSF and an IFN-α, preferably IFN-α2a or IFN-α2b.

In some embodiments, the vector further comprises a recombinant polynucleotide encoding a co-stimulatory molecule. In some embodiments, the recombinant polynucleotide encoding the co-stimulatory molecule comprises a recombinant polynucleotide encoding a CD86 molecule (CD86), CD80 molecule (CD80), 4-1BB ligand molecule (4-1BBL a.k.a CD137L), ICOS ligand molecule (ICOS-L), CD70 molecule (CD70 a.k.a. CD27L), CD40 molecule (CD40), OX40 ligand molecule (OX40L), GITR ligand molecule (GITRL), TIM-4 molecule (TIM-4), LIGHT molecule (LIGHT), ICAM1 molecule (ICAM1), LFA3 molecule (LFA3), a CD30 molecule (CD30), or a combination thereof.

In some embodiments, the vector further comprises a recombinant polynucleotide encoding an antigen of a pathogen, a tumor-associated antigen, a neo-antigen, an allergen, an antigen that is the target of an autoimmune response, a fragment thereof, or a combination thereof.

In another aspect, the present disclosure provides a recombinant polynucleotide comprising a sequence encoding an allele of an HLA class I gene having at least 85%, at least 90%, at least 95%, at least 96%, at least 97%, at least 98%, or at least 99% identity to any one of SEQ ID NOS:22-29 and optionally at least one heterologous polynucleotide sequence. In some embodiments, the recombinant polynucleotide comprises the sequence of any one of SEQ ID NOS:22-29. In some embodiments, the recombinant polynucleotide comprises a sequence encoding a polypeptide having at least 85%, at least 90%, at least 95%, at least 96%, at least 97%, at least 98%, or at least 99% identity to a polypeptide encoded by any one of SEQ ID NOS:22-29. In some embodiments, the recombinant polynucleotide encoding the allele of the HLA class I gene comprises the sequence of any one of SEQ ID NOS:22-29. In some embodiments, the recombinant polynucleotide further comprises a recombinant polynucleotide encoding a second allele of the HLA class I gene or an allele of a second HLA class I gene.

In some embodiments, the recombinant polynucleotide further comprises a recombinant polynucleotide encoding an allele of an HLA class II gene. In some embodiments, the recombinant polynucleotide encoding the allele of the HLA class II gene has at least 85%, at least 90%, at least 95%, at least 96%, at least 97%, at least 98%, or at least 99% identity to any one of SEQ ID NOS:30-35. In some embodiments, the recombinant polynucleotide comprises the sequence of any one of SEQ ID NOS:30-35. In some embodiments, the recombinant polynucleotide comprises a sequence encoding a polypeptide having at least 85%, at least 90%, at least 95%, at least 96%, at least 97%, at least 98%, or at least 99% identity to a polypeptide encoded by any one of SEQ ID NOS:30-35. In some embodiments, the recombinant polynucleotide encoding the allele of the HLA class II gene comprises the sequence of any one of SEQ ID NOS:30-35.

In various embodiments, the recombinant polynucleotide comprises a heterologous polynucleotide sequence encoding an amino acid sequence of a cytokine. In some embodiments, the cytokine comprises a chemokine, an interferon, an interleukin, a tumor necrosis factor, or a combination thereof. In some embodiments, the heterologous polynucleotide sequence encoding the cytokine comprises a heterologous polynucleotide sequence encoding an early T cell activation antigen-1 (ETA-1), a lymphocyte-activating factor (LAF), an interleukin-1 family member (IL-1α, IL-β, IL-1Ra, IL-18, IL-33, IL-36Ra, IL-36α, IL-36β, IL-36γ, IL-37, IL-38), an interleukin-2 (IL-2), an interleukin-3 (IL-3), an interleukin-4 (IL-4), an interleukin-5 (IL-5), an interleukin-6 (IL-6), an interleukin-7 (IL-7), an interleukin-8 (IL-8), an interleukin-9 (IL-9), an interleukin-10 (IL-10), an interleukin-12 (IL-12), an interleukin-13 (IL-13), an interleukin-15 (IL-15), an interleukin-17 (IL-17), an interleukin-18 (IL-18), an interleukin-21 (IL-21), an interleukin-23 (IL-23), an interleukin-25 (IL-25), an interleukin-33 (IL-33), an interferon alpha (IFN-α), an interferon lambda 1 (IFNλ1 (IL-29)), an interferon lambda 2 (IFNλ2 (IL-28A)), an interferon lambda 3 (IFNλ3 (IL-28B)), an interferon lambda 4 (IFNλ4), a granulocyte-macrophage colony-stimulating factor (GM-CSF), a macrophage CSF (CSF-1), a macrophage migration inhibitory factor (MIF), a CD40L molecule (CD40L), a RANTES molecule (RANTES), a monocyte chemoattractant protein (MCP-1), a monocyte inflammatory protein (MIP-1α, MIP-1β), a lymphotactin, a fractalkine, or a combination thereof. In some embodiments, the cytokine comprises a granulocyte-macrophage colony-stimulating factor (GM-CSF). In some embodiments, the cytokine comprises an interferon alpha (IFN-α), such as e.g., IFN-α2a or IFN-α2b. In some embodiments, the cytokine comprises a GM-CSF and an IFN-α, preferably IFN-α2a or IFN-α2b. In some embodiments, the IFN-α is an exogenous protein comprising a pegylated IFN-α2a.

In various embodiments, the recombinant polynucleotide comprises a heterologous polynucleotide sequence encoding one or more self-cleaving peptides between two HLA alleles. In some embodiments, the recombinant polynucleotide comprises a heterologous polynucleotide sequence encoding one or more self-cleaving peptides between an HLA allele and another HLA allele, a cytokine, a co-stimulatory molecule, or a combination thereof. In some embodiments, the self-cleaving peptide is positioned at the 3′ end of a first HLA allele and at the 5′ end of a second HLA allele. In some embodiments, the self-cleaving peptide comprises T2A, P2A, E2A, or a combination thereof.

In some embodiments, the recombinant polynucleotide comprises a heterologous polynucleotide sequence comprising a promoter. In some embodiments, the promoter comprises an MNDU3 promoter or an EF1α promoter.

In various embodiments, the recombinant polynucleotide comprises a heterologous polynucleotide sequence encoding an amino acid sequence of a co-stimulatory molecule. In some embodiments, the co-stimulatory molecule comprises a CD86 molecule (CD86), CD80 molecule (CD80), 4-1BB ligand molecule (4-1BBL a.k.a CD137L), ICOS ligand molecule (ICOS-L), CD70 molecule (CD70 a.k.a. CD27L), CD40 molecule (CD40), OX40 ligand molecule (OX40L), GITR ligand molecule (GITRL), TIM-4 molecule (TIM-4), LIGHT molecule (LIGHT), ICAM1 molecule (ICAM1), LFA3 molecule (LFA3), a CD30 molecule (CD30), or a combination thereof.

In some embodiments, the recombinant polynucleotide comprises a heterologous polynucleotide sequence comprising a sequence having at least 85%, at least 90%, at least 95%, at least 96%, at least 97%, at least 98%, or at least 99% identity to any one of SEQ ID NOS:36-44. In some embodiments, the heterologous polynucleotide comprises a sequence encoding a polypeptide having at least 85%, at least 90%, at least 95%, at least 96%, at least 97%, at least 98%, or at least 99% identity to a polypeptide encoded by any one of SEQ ID NOS:36-44. In some embodiments, the heterologous polynucleotide sequence comprises the sequence of any one of SEQ ID NOS:36-44.

In some embodiments, the heterologous polynucleotide sequence encodes an amino acid sequence of an antigen of a pathogen, a tumor-associated antigen, a neo-antigen, an allergen, an antigen that is the target of an autoimmune response, a fragment thereof, or a combination thereof.

### BRIEF DESCRIPTION OF THE DRAWINGS

FIG. 1 depicts a schematic drawing of BC1.68.3.4 (BC1). Shown is the plasmid backbone, pCC1-c with inserts of four HLA alleles including HLA-A*01:01, HLA-A*68:01, HLA-DRB3*02:02 and HLA-DRB4*01:01. The plasmid contains a cytomegalovirus (CMV) enhanced promoter on the 5′ end (CMV enhanced 5′ LTR). AMP: Ampicillin resistance gene. PSI: packaging signal. RRE: Rev responsive element. cPPT: central polypurine tract. EF1a: EF1 alpha promoter. MNDU3: MNDU3 promoter. T2A, P2A, and E2A: 2A self-splicing peptides.

FIG. 2 depicts a schematic drawing of BC2.11.4.3 (BC2). Shown is the plasmid backbone, pCC1-c with inserts of four HLA alleles including HLA-A*02:01, HLA-A*11:01, HLA-DRB4*01:01 and HLA-DRB3*03:01. The plasmid contains a cytomegalovirus (CMV) enhanced promoter on the 5′ end (CMV enhanced 5′ LTR). AMP: Ampicillin resistance gene. PSI: packaging signal. RRE: Rev responsive element. cPPT: central polypurine tract. EF1a: EF1 alpha promoter. MNDU3: MNDU3 promoter. T2A, P2A, and E2A: 2A self-splicing peptides.

FIG. 3 depicts a schematic drawing of BC3.23.3.5 (BC3). Shown is the plasmid backbone, pCC1-c with inserts of four HLA alleles including HLA-A*03:01, HLA-A*23:01, HLA-DRB3*01:01 and HLA-DRB5*01:02. The plasmid contains a cytomegalovirus (CMV) enhanced promoter on the 5′ end (CMV enhanced 5′ LTR). AMP: Ampicillin resistance gene. PSI: packaging signal. RRE: Rev responsive element. cPPT: central polypurine tract. EF1a: EF1 alpha promoter. MNDU3: MNDU3 promoter. T2A, P2A, and E2A: 2A self-splicing peptides.

FIG. 4 depicts a schematic drawing of BC24.33.5.5 (BC4). Shown is the plasmid backbone, pCC1-c with inserts of four HLA alleles including HLA-A*24:02, HLA-A*33:03, HLA-DRB5*01:01 and HLA-DRB5*02:02. The plasmid contains a cytomegalovirus (CMV) enhanced promoter on the 5′ end (CMV enhanced 5′ LTR). AMP: Ampicillin resistance gene. PSI: packaging signal. RRE: Rev responsive element. cPPT: central polypurine tract. EF1a: EF1 alpha promoter. MNDU3: MNDU3 promoter. T2A, P2A, and E2A: 2A self-splicing peptides.

FIG. 5 depicts common co-stimulatory molecules and their ligands presented on T cells and dendritic cells (DC), respectively.

FIG. 6 depicts a dual mechanism of action of Bria-IMT and Bria-OTS therapeutics. SV-BR-1-GM cells secrete GM-CSF that supports antigen presentation by DCs. Cancer cell antigens, following degradation of cells, are taken up by DCs and presented to CD4⁺ and CD8⁺ T cells, which induce a tumor-directed immune response. SV-BR-1-GM cells can also directly activate T cells to stimulate cancer fighting, as an additional boost to the immune response.

FIG. 7 depicts an experimental strategy to develop Bria-OTS cell lines. (A) Endogenous HLA-A/DRB genes are first KO by CRISPR/Cas9 to generate Bria-KO cells. GM-CSF and other cytokines and co-stimulatory molecules are then overexpressed by sequential lentiviral transduction of 4 unique constructs to generate Bria-APTC. Lastly, two HLA-A and two HLA-DRB alleles are overexpressed by lentiviral transduction, to generate a collection of 4 cell lines (Bria-OTS-1/2/3/4) for each parental tumor cell. (B) Lentiviral vectors used to express two different genes from independent promoters. Cytokines and co-stimulatory molecules (Left Table) used to generate Bria-APTC. HLA-A and HLA-DRB alleles (Right Table) used to generate final Bria-OTS products.

FIG. 8 depicts the expression of GM-CSF and cytokines/co-stimulatory molecules in SV-BR-1-APTC. SV-BR-1-KO Clone 17 was sequentially transduced with lentiviruses expressing 8 different cytokines and co-stimulatory molecules and cloned. (A) Levels of secreted GM-CSF, IFN-α, IL-12, and IL-7 were measured by ELISA. (B) Expression of surface receptors CD86 and CD80 in SV-BR-1-APTC as measured by flow cytometry.

FIG. 9 depicts validation of SV-BR-1-APTC using a modified MLR assay. (A) Bria-APTC or SV-BR-1KO cells are incubated with PMBCs at different ratios. At days 1, 2, 3, and 7, T cell proliferation and secretion of IFNγ, IL-2, and IL-4 are measured to assess T cell activation. (B) 10,000 SV-BR-1-KO or APTC per well were co-cultured in a 96 well plate with PBMCs from healthy donors at the indicated ratios. After 24 hours of incubation, secreted IFNγ was measured by ELISA. N=3.

FIG. 10 depicts the functional characterization of prostate-derived Bria-APTC. PC3-APTC or PC3KO cells (10,000/well in a 96 well plate) were incubated alone or with PBMCs at the indicated ratios for 24 hours. IFNγ levels were measured in the supernatant by ELISA.

FIG. 11 depicts expression of GM-CSF and cytokines/co-stimulatory molecules in PC3-APTC and expression of HLA-A alleles in PC3-Bria-OTS. PC3-KO Clone A4 was sequentially transduced with lentiviruses expressing 8 different cytokines and co-stimulatory molecules and cloned. (A) Expression of surface receptors CD86 and CD80 in PC3-APTC as measured by flow cytometry. (B) Expression of HLA-A alleles in PC3-Bria-OTS cell lines as measured by flow cytometry. (C) Levels of secreted IL-7, GM-CSF, and IL-12 in PC3-APTC were measured by ELISA.

FIG. 12 depicts KO of HLA alleles in H2228 and SK-MEL-24 cells. Expression of HLA-DR was measured in single clones of H2228-KO and SK-MEL-24-KO cells by flow cytometry, in comparison with wild type (WT) cells.

FIG. 13 depicts validation of HLA functionality with TCR transgenic Jurkat cells.

FIG. 14 depicts validation of HLA functionality in PC3-Bria-OTS using a T Cell Activation Bioassay (NFAT). The assay consists of a genetically engineered Jurkat T cell line with endogenous TCR alpha and beta chains knocked out using clustered regularly interspaced short palindromic repeats (CRISPR)-Cas9. These cells also express a luciferase reporter driven by a TCR pathway-dependent promoter. (A) Jurkat cells were transduced with a TCR specific for WT1 peptide (HLA-A*24 restricted) and incubated with the indicated cell lines and appropriate WT1 peptides. After 6 hours, the bioluminescent signal was quantified using the Bio-Glo-NL™ Luciferase Assay System and a standard luminometer such as the GloMax® Discover System. Transgenic HLA induces T-cell activation at rates comparable to endogenous HLAs. (B) Flow cytometry analysis showing HLA-A*24 expression in the indicated cell lines. (C) Jurkat cells were transduced with a TCR specific for MAGE-A3 peptide (HLA-A*01 restricted) and incubated with the indicated cell lines and appropriate WT1 peptides. After 6 hours, the bioluminescent signal was quantified using the Bio-Glo-NL™ Luciferase Assay System and a standard luminometer such as the GloMax® Discover System. Transgenic HLA induces cell activation at higher rates than endogenous HLAs. (D) Flow cytometry analysis showing HLA-A*01 expression in the indicated cell lines.

FIG. 15 depicts validation of HLA functionality in SV-BR-1-Bria-OTS using a T Cell Activation Bioassay (NFAT). The assay consists of a genetically engineered Jurkat T cell line with endogenous TCR alpha and beta chains knocked out using CRISPR-Cas9. These cells also express a luciferase reporter driven by a TCR pathway-dependent promoter. Jurkat cells were transduced with a TCR specific for WT1 peptide (HLA-A*24 restricted) and incubated with the indicated cell lines and appropriate WT1 peptides. After 6 hours, the bioluminescent signal was quantified using the Bio-Glo-NL™ Luciferase Assay System and a standard luminometer such as the GloMax® Discover System.

### DETAILED DESCRIPTION

### I. Introduction

Induction of effective immune responses with whole-cell immunotherapies is an effective approach to treat and prevent diseases such as cancer. It is generally assumed that to be effective, a cancer vaccine needs to express immunogenic antigens co-expressed in patient tumor cells, and that antigen-presenting cells (APC) such as dendritic cells (DCs) need to cross-present such antigens following uptake of vaccine cell fragments.

The transformational technology central to the present disclosure is the development of a therapeutic strategy that addresses the current gap in safe and reliable treatments for patients suffering from cancer. The whole-cell immunotherapies described herein significantly improve upon previous attempts to develop whole-cell therapeutic vaccines, as the disclosed approach works through two discrete, but complementary mechanisms, to stimulate a patient's immune system. In particular, the modified human cancer cells described herein have features of both cancer cells (which express tumor associated antigens) and dendritic cells (which present tumor-associated antigens to T cells). In certain aspects, the modified human cancer cells described herein (e.g., Bria-OTS cell lines) express one or more immunomodulatory cytokines, one or more co-stimulatory molecules, one or more human major histocompatibility complex-I (MHC-I) (e.g., HLA-A, HLA-B, HLA-C) molecules, and one or more MHC-II (e.g., HLA-DRB3, HLA-DRB4, HLA-DRB5, HLA-DRA) molecules. The modified human cancer cells described herein can directly activate CD4⁺ T-cells in an antigen-specific HLA-restricted manner and function as APC. In certain aspects, the modified human cancer cells described herein advantageously provide the following features: (a) dual mechanisms of action to achieve strong clinical activity by both producing and presenting tumor-associated antigens for eliciting an immune response in the subject; (b) precision therapy by matching therapies to patients based upon HLA antigens; and (c) improved safety by reducing or eliminating the use of current chemotherapeutic and hormone-based therapies that are associated with severe adverse events that can be life threatening. Thus, the modified human cancer cells described herein can provide rapid and cost-effective treatments of cancer. In some instances, the modified human cancer cells can be used as “off-the-shelf” cell lines that do not require personalized manufacturing and can be administered immediately either after patient HLA genotyping or as a mixture of cells to cover about 70% to over 99% of the cancer patient population.

In certain aspects, the modified human cancer cells described herein are HER-2/neu positive, allogeneic, whole cell breast cancer (BC) cell lines that secrete granulocyte-macrophage colony stimulating factor (GM-CSF) in situ and augment dendritic cell activity for breast cancer immunotherapy. In addition, the modified human cancer cells described herein can be engineered to express multiple HLA alleles to match, for example, at least 90% or more than 99% of the breast cancer patient population. For instance, HLA-A and HLA-DRB3/4/5 alleles are the least polymorphic HLA loci and can match the maximum number of patients with the fewest number of alleles.

Described herein are compositions of modified human cancer cells or cell lines for targeted immunotherapy of cancers. Additionally, kits containing the “off-the-shelf” cell lines and methods for preventing or treating cancer in a subject in need thereof are provided.

### II. Definitions

Unless specifically indicated otherwise, all technical and scientific terms used herein have the same meaning as commonly understood by those of ordinary skill in the art to which this disclosure belongs. In addition, any method or material similar or equivalent to a method or material described herein can be used in the practice of the present disclosure. For purposes of the present disclosure, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The terms “subject,” “individual,” and “patient” are used interchangeably herein to refer to a vertebrate, preferably a mammal, more preferably a human. Mammals include, but are not limited to, murines, rats, simians, humans, farm animals, sport animals, and pets. Tissues, cells and their progeny of a biological entity obtained in vivo or cultured in vitro are also encompassed.

As used herein, the term “administering” includes oral administration, topical contact, administration as a suppository, intravenous, intraperitoneal, intramuscular, intralesional, intratumoral, intradermal, intralymphatic, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The term “treating” refers to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. Therapeutic benefit can also mean to effect a cure of one or more diseases, conditions, or symptoms under treatment.

The term “effective amount” or “sufficient amount” refers to the amount of a modified cancer cell or other composition that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific amount may vary depending on one or more of: the particular agent chosen, the target cell type, the location of the target cell in the subject, the dosing regimen to be followed, whether it is administered in combination with other compounds, timing of administration, and the physical delivery system in which it is carried.

For the purposes herein, an effective amount is determined by such considerations as may be known in the art. The amount must be effective to achieve the desired therapeutic effect in a subject suffering from cancer. The desired therapeutic effect may include, for example, amelioration of undesired symptoms associated with cancer, prevention of the manifestation of such symptoms before they occur, slowing down the progression of symptoms associated with cancer, slowing down or limiting any irreversible damage caused by cancer, lessening the severity of or curing a cancer, or improving the survival rate or providing more rapid recovery from a cancer.

The effective amount depends, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the distribution profile of a therapeutic agent (e.g., a whole-cell cancer vaccine) or composition within the body, the relationship between a variety of pharmacological parameters (e.g., half-life in the body) and undesired side effects, and other factors such as age and gender, etc.

The term “pharmaceutically acceptable carrier” refers to a substance that aids the administration of an active agent to a cell, an organism, or a subject. “Pharmaceutically acceptable carrier” refers to a carrier or excipient that can be included in the compositions of the disclosure and that causes no significant adverse toxicological effect on the subject. Non-limiting examples of pharmaceutically acceptable carriers include water, sodium chloride, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g. antimicrobial preservatives, antioxidants, chelating agents and buffers), for preventing the action of microorganisms (e.g. antimicrobial and antifungal agents, such as parabens, chlorobutanol, sorbic acid and the like) or for providing the formulation with an edible flavor etc. In some instances, the carrier is an agent that facilitates the delivery of a modified cancer cell to a target cell or tissue. One of skill in the art will recognize that other pharmaceutical carriers are useful in the present disclosure.

The term “nucleic acid” or “nucleotide” as used herein refers to a polymer containing at least two deoxyribonucleotides or ribonucleotides in either single- or double-stranded form and includes DNA, RNA, and hybrids thereof. DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. RNA may be in the form of small interfering RNA (siRNA), Dicer-substrate dsRNA, small hairpin RNA (shRNA), asymmetrical interfering RNA (aiRNA), microRNA (miRNA), mRNA, tRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J Biol. Chem., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “gene” means the segment of DNA involved in producing a polypeptide chain. The DNA segment may include regions preceding and following the coding region (leader and trailer) involved in the transcription/translation of the gene product and the regulation of the transcription/translation, as well as intervening sequences (introns) between individual coding segments (exons).

The terms “vector” and “expression vector” refer to a nucleic acid construct, generated recombinantly or synthetically, with a series of specified nucleic acid elements that permit transcription of a particular polynucleotide sequence in a host cell. An expression vector may be part of a plasmid, viral genome, or nucleic acid fragment. Typically, an expression vector includes a polynucleotide to be transcribed, operably linked to a promoter. The term “promoter” is used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Other elements that may be present in an expression vector include those that enhance transcription (e.g., enhancers) and terminate transcription (e.g., terminators). In the context of the present disclosure, co-expression of multiple genes (e.g., polynucleotides ending an HLA class I allele and/or an HLA class II allele) may be achieved by co-transfection of two or more vectors, the use of multiple or bidirectional promoters, or the creation of bicistronic or multicistronic vectors. Gene co-expression may be driven by using a plasmid with multiple, individual expression cassettes. Generally, each promoter creates unique mRNA transcripts for each gene that is expressed. Bicistronic or multicistronic vectors simultaneously express two or more separate proteins from the same mRNA. Bicistronic vectors may contain an Internal Ribosome Entry Site (IRES) to allow for initiation of translation from an internal region of the mRNA. Multicistronic vectors containing one or more self-cleaving 2A peptides are advantageous as they allow gene co-expression from the same cassette. In some instances, multicistronic vectors are preferred when only a portion of the plasmid is packaged for viral delivery, or the relative expression levels between two or more genes is important.

The terms “self-cleaving peptide” and “self-cleaving 2A peptide” refer to a short peptide that can produce equimolar levels of multiple genes from the same mRNA. These peptides were first discovered in picornaviruses. Self-cleaving peptides are thought to function by making the ribosome skip the synthesis of a peptide bond at the C-terminus of a 2A element, leading to separation between the end of the 2A sequence and the next peptide downstream. The “cleavage” occurs between the glycine and proline residues found at the C-terminus, meaning the upstream cistron typically has a few additional residues added to the end, while the downstream cistron typically starts with the proline. Non-limiting examples of self-cleaving peptides include T2A, P2A, E2A, and F2A.

“Recombinant” refers to a genetically modified polynucleotide, polypeptide, cell, tissue, or organism. For example, a recombinant polynucleotide (or a copy or complement of a recombinant polynucleotide) is one that has been manipulated using well known methods. A recombinant expression cassette comprising a promoter operably linked to a second polynucleotide (e.g., a coding sequence) can include a promoter that is heterologous to the second polynucleotide as the result of human manipulation (e.g., by methods described in Sambrook et al., Molecular Cloning—A Laboratory Manual, Cold Spring Harbor Laboratory, Cold Spring Harbor, New York, (1989) or Current Protocols in Molecular Biology Volumes 1-3, John Wiley & Sons, Inc. (1994-1998)). A recombinant expression cassette (or expression vector) typically comprises polynucleotides in combinations that are not found in nature. For instance, human manipulated restriction sites or plasmid vector sequences can flank or separate the promoter from other sequences. A recombinant protein is one that is expressed from a recombinant polynucleotide, and recombinant cells, tissues, and organisms are those that comprise recombinant sequences (polynucleotide and/or polypeptide). A recombinant cell is one that has been modified (e.g., transfected or transformed), with a recombinant nucleotide, expression vector or cassette, or the like.

The term “amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and their combinations. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and their combinations.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid. Amino acids may be referred to by either the commonly known three letter symbols or by the one-letter symbols recommended by the IUPAC-IUB Biochemical Nomenclature Commission.

The terms “identity,” “substantial identity,” “similarity,” “substantial similarity,” “homology” and the related terms and expressions used in the context of describing amino acid sequences refer to a sequence that has at least 60% sequence identity to a reference sequence. Examples include at least: 60%, 65%, 70%, 75%, 80%, 85%, 86%, 87%, 88%, 89%, 90%, 91%, 92%, 93%, 94%, 95%, 96%, 97%, 98%, or 99%, sequence identity, as compared to a reference sequence using the programs for comparison of amino acid sequences, such as BLAST using standard parameters. For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default (standard) program parameters can be used, or alternative parameters can be designated. The sequence comparison algorithm then calculates the percent sequence identities for the test sequences relative to the reference sequence, based on the program parameters. A “comparison window” includes reference to a segment of any one of the number of contiguous positions (from 20 to 600, usually about 50 to about 200, more commonly about 100 to about 150), in which a sequence may be compared to a reference sequence of the same number of contiguous positions after the two sequences are optimally aligned. Methods of alignment of sequences for comparison are well-known. Optimal alignment of sequences for comparison may be conducted, for example, by the local homology algorithm of Smith and Waterman, 1981, by the homology alignment algorithm of Needleman and Wunsch, 1970, by the search for similarity method of Pearson and Lipman, 1988, by computerized implementations of these algorithms (for example, BLAST), or by manual alignment and visual inspection.

Algorithms that are suitable for determining percent sequence identity and sequence similarity include BLAST and BLAST 2.0 algorithms, which are described in Altschul et al., 1990, and Altschul et al., 1977, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (Henikoff and Henikoff, 1989). The BLAST algorithm also performs a statistical analysis of the similarity between two sequences (Karlin and Altschul, 1993).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., alleles), wherein the amino acid residues are linked by covalent peptide bonds. As used herein, the amino acid sequence of a polypeptide is presented from the N-terminus to the C-terminus. In other words, when describing an amino acid sequence of a polypeptide, the first amino acid at the N-terminus is referred to as the “first amino acid.”

When used in the context of describing partners of a recombinant polypeptide, the term “heterologous” refers to the relationship of one polynucleotide fusion partner to the other polynucleotide fusion partner: the manner in which the fusion partners are present in the recombinant polynucleotide is not one that can be found in a polynucleotide naturally occurs or encoding a naturally occurring protein. For instance, a “heterologous polynucleotide” fused with an HLA class I allele and/or an HLA class II allele to form a fusion polynucleotide may be one that is originated from a protein other than an HLA class I protein or an HLA class II protein, such as a granulocyte-macrophage colony-stimulating factor (GM-CSF), a co-stimulatory molecule (e.g., CD80), or a 2A self-splicing peptide (e.g., T2A, P2A, E2A). On the other hand, a “heterologous polynucleotide” may be one derived from another portion of the HLA class I or HLA class II protein that is not immediately contiguous to the HLA class I or HLA class II allele. A “heterologous polynucleotide” may encode a peptide containing modifications of a naturally occurring protein sequence or a portion thereof, such as deletions, additions, or substitutions of one or more amino acid residues.

The terms “gene editing”, “genome editing”, “genome engineering”, and “genome manipulation” are used interchangeably. These terms refer to a type of genetic engineering in which DNA is inserted, deleted, modified or replaced in the genome of a living organism. Genome editing can be site-specific. Non-limiting examples of genome editing techniques include the use of nucleases such as clustered regularly interspaced short palindromic repeats/Cas9 (CRISPR/Cas9) nucleases, meganucleases, transcription activator-like effector nucleases (TALENs), and zinc-finger nucleases (ZFNs). Viral vectors such as integrase-defective lentiviral vectors (IDLVs), adenoviruses and adeno-associated viruses (AAVs) are typically used to deliver DNA for genome editing. Delivery technologies for genome editing are known in the art and any approach may be used for inactivating specific HLA alleles in the modified human cancer cells described herein. (See e.g., review by Yin et al., 2017. “Delivery technologies for genome editing.” Nature Review Drug Discovery. 16, 387-399).

The term “cancer” is intended to include any member of a class of diseases characterized by the uncontrolled growth of aberrant cells. The term includes all known cancers and neoplastic conditions, whether characterized as malignant, benign, recurrent, soft tissue, or solid, and cancers of all stages and grades including advanced, pre- and post-metastatic cancers. Examples of different types of cancer include, but are not limited to, gynecological cancers (e.g., ovarian, cervical, uterine, vaginal, and vulvar cancers); lung cancers (e.g., non-small cell lung cancer, small cell lung cancer, mesothelioma, carcinoid tumors, lung adenocarcinoma); breast cancers (e.g., triple-negative breast cancer, ductal carcinoma in situ, invasive ductal carcinoma, tubular carcinoma, medullary carcinoma, mucinous carcinoma, papillary carcinoma, cribriform carcinoma, invasive lobular carcinoma, inflammatory breast cancer, lobular carcinoma in situ, Paget's disease, Phyllodes tumors); digestive and gastrointestinal cancers such as gastric cancer (e.g., stomach cancer), colorectal cancer, gastrointestinal stromal tumors (GIST), gastrointestinal carcinoid tumors, colon cancer, rectal cancer, anal cancer, bile duct cancer, small intestine cancer, and esophageal cancer; thyroid cancer; gallbladder cancer; liver cancer; pancreatic cancer; appendix cancer; prostate cancer (e.g., prostate adenocarcinoma); renal cancer (e.g., renal cell carcinoma); cancer of the central nervous system (e.g., glioblastoma, neuroblastoma, medulloblastoma); skin cancer (e.g., melanoma); bone and soft tissue sarcomas (e.g., Ewing's sarcoma); lymphomas; choriocarcinomas; urinary cancers (e.g., urothelial bladder cancer); head and neck cancers; and bone marrow and blood cancers (e.g., acute leukemia, chronic leukemia (e.g., chronic lymphocytic leukemia), lymphoma, multiple myeloma). As used herein, a “tumor” comprises one or more cancerous cells.

The term “allele” refers to a particular form or variant of a gene. Alleles can result from, for example, nucleotide substitutions, additions, or deletions, or can represent a variable number of short nucleotide repeats. In the context of human leukocyte antigen (HLA) genes, HLA alleles are named by the World Health Organization Naming Committee for Factors of the HLA system. Under this system, an HLA gene name is followed by a series of numerical fields. At a minimum, two numerical fields are included. As a non-limiting example, HLA-A*02:101 denotes a specific allele of the HLA-A gene. The first field, separated from the gene name by an asterisk, denotes an allele group. The second field, separated from the first field by a colon, denotes the specific HLA protein that is produced. In some instances, a longer name is used (e.g., HLA-A*02:101:01:02N). In this example, the third numerical field denotes whether a synonymous DNA substitution is present within the coding region, and the fourth numerical field denotes differences between alleles that exist in the non-coding region. In some other instances, an HLA allele name is contains a letter at the end. Under the HLA allele naming system, “N” denotes that the allele is a null allele (i.e., the allele produces a non-functional protein), “L” denotes that the allele results in lower than normal cell surface expression of the particular HLA protein, “S” denotes that the allele produces a soluble protein not found on the cell surface, “Q” denotes a questionable allele (i.e., an allele that nay not affect normal expression), “C” denotes that the allele produces a protein that is present in cell cytoplasm but is not present at the cell surface, and “A” denotes an allele that results in aberrant expression (i.e., it is uncertain whether the particular HLA protein is expressed). One of skill in the art will be familiar with the various gene alleles and their naming conventions.

The term “allele profile” refers to a collection of alleles of one or more genes in a particular sample. The sample may be obtained from a subject, a particular cell or cell type (e.g., a breast cell or breast cancer cell), or from an engineered cell (e.g., a cancer cell that has been engineered to express one or more proteins). In some instances, an allele profile describes the alleles of a single gene that are present in a sample (e.g., in a cell obtained from a subject or a cancer vaccine cell), or may describe the alleles that are present for two or more genes in a sample. As a non-limiting example, an allele profile may list the alleles that are present for the HLA-A gene in a particular sample. For a diploid cell, only one allele may be present (e.g., if both chromosomes contain the same allele, such as HLA-A*02:01). Alternatively, two different alleles may be present (e.g., the allele profile contains HLA-A*02:01 and HLA-A*24:02, or HLA-A*02:01 and HLA-A*03:01). In other instances, the allele profile enumerates the alleles that are present for two or more genes. As a non-limiting example, an allele profile may describe the alleles of the HLA-A and HLA-DRB3 genes that are present in a patient sample.

For purposes of illustration only, an allele profile of a subject may indicate that the HLA-A*02:01 and HLA-A*24:02 alleles of the HLA-A gene are present, and that the HLA-DRB3*03:01 allele of the HLA-DRB3 gene is present. Furthermore, allele profiles can be compared. As a non-limiting example, a subject can have an allele profile containing the HLA-A*02:01 and HLA-A*24:02 alleles, while a cancer vaccine cell can have a profile containing the HLA-A*02:01 and HLA-A*03:01 alleles. In this example, if the two allele profiles are compared, then there is a partial match between the profiles (i.e., the HLA-A*02:01 allele is present in both profiles). As another non-limiting example, if the vaccine cell has an allele profile containing HLA-A*02:01 and HLA-A*24:02, then the subject and vaccine cell profiles are a complete match with respect to this particular gene.

The term “human leukocyte antigen (HLA)” refers to a gene complex that encodes human major histocompatibility complex (MHC) proteins, which are a set of cell surface proteins that are essential for recognition of foreign molecules by the adaptive immune system. The HLA complex is found within a 3 Mbp stretch of chromosome 6p21. Class I MHC proteins, which present peptides from inside the cell, are encoded by the HLA-A, HLA-B, HLA-C, HLA-E, HLA-F, and HLA-G genes. HLA-A, HLA-B, and HLA-C genes are more polymorphic, while HLA-E, HLA-F, and HLA-G genes are less polymorphic. HLA-K and HLA-L are also known to exist as pseudogenes. In addition, beta-2-microglobulin is an MHC class I protein, encoded by the (B2M) gene. Non-limiting examples of HLA-A nucleotide sequences are set forth under GenBank reference numbers NM_001242758 and NM_002116. A non-limiting example of an HLA-B nucleotide sequence is set forth under GenBank reference number NM_005514. Non-limiting examples of HLA-C nucleotide sequences are set forth under GenBank reference numbers NM_001243042 and NM_002117. Anon-limiting example of an HLA-E nucleotide sequence is set forth under GenBank reference number NM_005516. A non-limiting example of an HLA-F nucleotide sequence is set forth under GenBank reference number NM_018950. A non-limiting example of an HLA-G nucleotide sequence is set forth under GenBank reference number NM_002127. A non-limiting example of a B2M nucleotide sequence is set forth under GenBank reference number NM_004048.

Class II MHC proteins, which present antigens from the outside of the cell to T lymphocytes, are encoded by the HLA-DP, HLA-DM, HLA-DO, HLA-DQ, and HLA-DR genes. HLA-DM genes include HLA-DMA and HLA-DMB. HLA-DO genes include HLA-DOA and HLA-DOB. HLA-DP genes include HLA-DPA1 and HLA-DPB1. HLA-DQ genes include HLA-DQA1, HLA-DQA2, HLA-DQB1, and HLA-DQB2. HLA-DR genes include HLA-DRA, HLA-DRB1, HLA-DRB3, HLA-DRB4, and HLA-DRB5. Non-limiting examples of HLA-DMA and HLA-DMB nucleotide sequences are set forth under GenBank reference numbers NM_006120 and NM_002118, respectively. Non-limiting examples of HLA-DRA, HLA-DRB1, HLA-DRB3, HLA-DRB4, and HLA-DRB5 nucleotide sequences are set forth in GenBank reference numbers NM_01911, NM_002124, NM_022555, NM_021983, NM_002125, respectively.

The term “vaccine” refers to a biological composition that, when administered to a subject, has the ability to produce an acquired immunity to a particular pathogen or disease in the subject. Typically, one or more antigens, or fragments of antigens, that are associated with the pathogen or disease of interest are administered to the subject. Vaccines can comprise, for example, inactivated or attenuated organisms (e.g., bacteria or viruses), cells, proteins that are expressed from or on cells (e.g., cell surface proteins), proteins that are produced by organisms (e.g., toxins), or portions of organisms (e.g., viral envelope proteins). In some instances, cells are engineered to express proteins such that, when administered as a vaccine, they enhance the ability of a subject to acquire immunity to that particular cell type (e.g., enhance the ability of a subject to acquire immunity to a cancer cell). As used herein, the term “vaccine” or “whole-cell cancer vaccine” includes but is not limited to the modified cancer cell(s) of the present disclosure.

The term “cytokine” refers to small proteins released by cells that have a specific effect on the interactions and communications between cells. Cytokines are generally known as lymphokines (e.g., cytokines made by lymphocytes), monokines (e.g., cytokines made by monocytes), or chemokines (e.g., cytokines made by one leukocyte and acting on other leukocytes). Cytokines may act on the cells that secrete them (e.g., autocrine action), on nearby cells (e.g., paracrine action), or on distant cells (e.g., endocrine action). In the context of the present disclosure, cytokines may comprise a chemokine, an interferon, an interleukin, and/or a tumor necrosis factor (TNF). As an example, cytokines may comprise an early T cell activation antigen-1 (ETA-1), a lymphocyte-activating factor (LAF), an interleukin-1 family member (IL-1α, IL-β, IL-1Ra, IL-18, IL-33, IL-36Ra, IL-36α, IL-36β, IL-36γ, IL-37, IL-38), an interleukin-2 (IL-2), an interleukin-3 (IL-3), an interleukin-4 (IL-4), an interleukin-5 (IL-5), an interleukin-6 (IL-6), an interleukin-7 (IL-7), an interleukin-8 (IL-8), an interleukin-9 (IL-9), an interleukin-10 (IL-10), an interleukin-12 (IL-12), an interleukin-13 (IL-13), an interleukin-15 (IL-15), an interleukin-17 (IL-17), an interleukin-18 (IL-18), an interleukin-23 (IL-21), an interleukin-23 (IL-23), an interleukin-25 (IL-25), an interleukin-33 (IL-33), a type I interferon family member (IFN-α, IFNβ, IFNε, IFNκ, IFNω), a type II interferon family member (IFNγ), a type III interferon family member (IFNλ1 (IL-29), IFNλ2 (IL-28A), IFNλ3 (IL-28B), IFNλ4), a granulocyte-macrophage colony-stimulating factor (GM-CSF), a macrophage CSF (CSF-1), a macrophage migration inhibitory factor (MIF), a CD40L molecule (CD40L), a RANTES molecule (RANTES), a monocyte chemoattractant protein (MCP-1), a monocyte inflammatory protein (MIP-1α, MIP-1β), a lymphotactin, and/or a fractalkine.

The term “granulocyte macrophage colony-stimulating factor (GM-CSF)” refers to a monomeric glycoprotein also known as “colony stimulating factor (CSF2)” that is secreted by cells such as macrophages, T cells, mast cells, natural killer (NK) cells, endothelial cells, and fibroblasts. GM-CSF functions as a cytokine that affects a number of cell types, in particular macrophages and eosinophils. As part of the immune/inflammatory cascade, GM-CSF stimulates stem cells to produce granulocytes (i.e., neutrophils, eosinophils, and basophils) and monocytes. The monocytes subsequently mature into macrophages and dendritic cells after tissue infiltration. A non-limiting example of a CSF2 nucleotide sequence (the gene that encodes GM-CSF) in humans is set forth under GenBank reference number NM_000758.

The term “interferon” refers to a cytokine that is produced in response to infection or other inflammatory stimuli. Interferons are signaling proteins that are synthesized and released by host cells in response to a pathogen (e.g., viruses, bacteria, parasites, tumor cells). Interferons are classified into three subgroups type I interferons, type II interferon (IFNγ), and type III interferons. Functionally, these cytokines modulate immune cell function. Although type III interferons are structurally distinct from type I interferons, they have overlapping functions, and both signal through the Janus kinase (JAK)-signal transducer and activator of transcription (STAT) pathway to induce transcription of interferon-stimulated genes (ISGs) and promote immune responses. (See e.g., Goel et al. (2021). Interferon lambda in inflammation and autoimmune rheumatic diseases. Nat Rev Rheumatol 17, 349-362). Type I interferon proteins include IFN-α, IFN-β, IFN-ε, IFN-κ, IFN-τ, IFN-δ, IFN-ζ, IFN-ω, and IFN-ν. Interferon alpha proteins are produced by leukocytes and are mainly involved in the innate immune response. Genes that encode IFN-α proteins include IFNA1, IFNA2, IFNA4, IFNA5, IFNA6, IFNA7, IFNA8, IFNA10, IFNA13, IFNA14, IFNA16, IFNA17, and IFNA21. Non-limiting examples of IFNA1, IFNA2, IFNA4, IFNA5, IFNA6, IFNA7, IFNA8, IFNA10, IFNA13, IFNA14, IFNA16, IFNA17, and IFNA21 human nucleotide sequences are set forth in Gene Bank reference numbers NM 024013, NM_000605, NM 021068, NM_002169, NM_021002, NM_021057, NM_002170, NM_002171, NM_006900, NM_002172, NM_002173, NM_021268, and NM_002175, respectively. As an example, the gene IFNA2 encodes IFN-α2a, IFN-α2b, and IFN-α2c variants. As used herein, the terms “IFN-α” and “IFN-α2” are used interchangeably, and they refer to interferon proteins IFN-α2a or IFN-α2b. Type III interferon proteins include interferon lambda 1 (IFNλ1 (IL-29), interferon lambda 2 (IFNλ2 (IL-28A)), interferon lambda 1 (IFNλ3 (IL-28B)), and interferon lambda 4 (IFNλ4). Interferon lambda family members signal through the common IL-10 receptor subunit 2 (IL-10R2). Human interferon lambda proteins are encoded by four IFNL genes, IFNL1 (1129), IFNL2 (IL28A), IFNL3 (IL28B), and IFNL4.

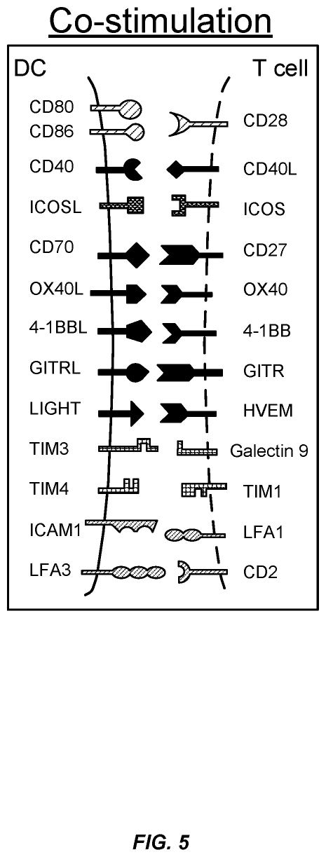

The term “co-stimulatory molecule” refers to a cell surface molecule that amplifies or counteracts the initial activating signals provided to T cells from the T cell receptor (TCR) following its interaction with an antigen/major histocompatibility complex (MHC). Co-stimulatory molecules generally may influence T cell differentiation and fate. Co-stimulatory molecules belong to three major families, namely the immunoglobulin (Ig) superfamily, the tumor necrosis factor (TNF)-TNF receptor (TNFR) superfamily, and the T cell Ig and mucin (TIM) domain family. (See e.g., Rodriguez-Manzanet, Roselynn et al. “The costimulatory role of TIM molecules.” Immunological reviews vol. 229, 1 (2009): 259-70.) Exemplary co-stimulatory molecules and ligands include, but are not limited to, CD28 and ligands B7-1 (CD80), CTLA-4, PDL-1, or B7-2 (CD86), CTLA-4 and ligands B7-1 (CD80) or B7-2 (CD86), ICOS and ligand ICOS-L, CD27 and ligand CD70, CD30 and ligand CD30L, CD40 and ligand CD40L (a.k.a. CD154), OX40 and ligand OX40L, GITR and ligand GITRL, TIM-1 and ligands TIM-1, TIM-4, IgA, or phosphatidylserine (PtdSer), TIM-2 and ligands H-ferritin or semaphorin 4A (Sem4A), and TIM-4 and ligand phosphatidylserine (PtdSer). In the context of the present disclosure, co-stimulatory molecules may comprise a CD86 molecule (CD86), CD80 molecule (CD80), 4-1BB ligand molecule (4-1BBL a.k.a CD137L), ICOS ligand molecule (ICOS-L), CD70 molecule (CD70 a.k.a. CD27L), CD40 molecule (CD40), OX40 ligand molecule (OX40L), GITR ligand molecule (GITRL), TIM-4 molecule (TIM-4), LIGHT molecule (LIGHT), ICAM1 molecule (ICAM1), LFA3 molecule (LFA3), CD30 molecule (CD30), and combinations thereof (See e.g., FIG. 5).

The term “tumor antigen” refers to an antigenic substance produced in tumor cells that may trigger an immune response in the host. Tumor antigens generally refer to tumor-associated antigen (TAAs) or tumor-specific antigens (TSAs). Typically, TSAs are found in cancer cells only and are not in healthy (e.g., non-cancerous) cells. TSAs may arise from oncogenic driver mutations that generate novel peptide sequences (e.g., neoantigens). A non-limiting example of a TSA is alphafetoprotein (AFP) expressed in germ cell tumors and hepatocellular carcinoma. TAAs have elevated levels in tumor cells and may express at lower levels in healthy cells. A non-limiting example of a TAA is melanoma-associated antigen (MAGE) expressed in the testis along with malignant melanoma.

The terms “CD47 molecule”, “integrin-associated protein (IAP)”, “MER6” and “OA3” are used interchangeably herein. CD47 generally refers to a variably glycosylated transmembrane protein of the immunoglobulin superfamily (IgSF). It contains an extracellular domain (ECD) with a single Ig-like domain, five transmembrane domains with short intervening loops and a 34 amino acid cytoplasmic tail at the C-terminus. CD47 is overexpressed on the surface of many types of cancer cells. CD47 forms a signaling complex with signal-regulatory protein α (SIRPα) on macrophages, neutrophils, and T lymphocytes, enabling the escape of these cancer cells from macrophage-mediated phagocytosis. SIRPα is expressed on myeloid cells, including macrophages and dendritic cells (DCs). Without being bound by any particular theory, CD47 typically provides a “do not eat me” signal to macrophages through binding to SIRPα to prevent phagocytosis. (See e.g., Li et al., “Vaccination with CD47 deficient tumor cells elicits an antitumor immune response in mice.” Nat Commun 11, 581 (2020)). Cells that lack CD47 are rapidly cleared from the bloodstream by splenic red pulp macrophages. (See e.g., Oldenborg et al., “Role of CD47 as a marker of self on red blood cells.” Science 288, 2051 (2000)). As an example in the context of a cancer prophylactic or therapeutic, vaccination with CD47-deficient tumor cells may induce antitumor immunity. (See Li et al., supra)

The terms “Ii(CD74) molecule”, “CD74 molecule”, “Cluster of Differentiation 74”, “major histocompatibility complex (MHC) class II-associated Invariant chain (Ii)”, “HLA class II histocompatibility antigen gamma chain”, and “HLA-DR antigen-associated invariant chain (Ii)” refer to a protein that is encoded by the CD74 gene in humans. The Invariant chain (Ii) is a polypeptide present in professional antigen presenting cells where it regulates peptide loading onto MHC class II molecules and the peptidome presented to CD4⁺ T lymphocytes. Generally, activation of CD4⁺ T cells requires delivery of a costimulatory signal plus an antigen-specific signal consisting of peptide bound to an MHC II molecule. CD4⁺ T cells are typically activated by professional antigen presenting cells (APC), which endocytose exogenously synthesized antigen and process and present it in the context of their own MHC II molecules. This processing and presentation process requires Invariant chain (Ii), a molecule that is coordinately synthesized with MHC II molecules and prevents the binding and presentation of APC-encoded endogenous peptides. Tumor-reactive CD4⁺ T cells are typically activated to tumor peptides generated by the antigen processing machinery of professional APC, rather than peptides generated by the tumor cells. Without being bound by any particular theory, tumor cells lacking Ii(CD74) may present a novel repertoire of MHC II-restricted tumor peptides that are not presented by professional APC, and therefore may be highly immunogenic. Once activated, CD4⁺ T cells produce IFNγ and provide help to CD8⁺ T cells and do not need to react with native tumor cells. Therefore, the MHC II vaccines have the potential to activate CD4⁺ Th1 cells that facilitate antitumor immunity. For instance, Ii(CD74)-negative breast cancer cells are capable of presenting unique peptides that activate tumor specific T cells from breast cancer patients. (See e.g., Chornoguz et al., 2012. “Major histocompatibility complex class II+ invariant chain negative breast cancer cells present unique peptides that activate tumor-specific T cells from breast cancer patients.” Molecular & Cellular Proteomics (11)11:1457-67 (2012)). As an example in the context of a cancer prophylactic or therapeutic, vaccination with Ii(CD74)-deficient tumor cells may induce antitumor immunity. (See Chornoguz et al., supra)

The term “survival” refers to a length of time following the diagnosis of a disease and/or beginning or completing a particular course of therapy for a disease (e.g., cancer). The term “overall survival” includes the clinical endpoint describing patients who are alive for a defined period of time after being diagnosed with or treated for a disease, such as cancer. The term “disease-free survival” includes the length of time after treatment for a specific disease (e.g., cancer) during which a patient survives with no sign of the disease (e.g., without known recurrence). In certain embodiments, disease-free survival is a clinical parameter used to evaluate the efficacy of a particular therapy, which is usually measured in units of 1 or 5 years. The term “progression-free survival” includes the length of time during and after treatment for a specific disease (e.g., cancer) in which a patient is living with the disease without additional symptoms of the disease. In some embodiments, survival is expressed as a median or mean value.

### III. Detailed Description of the Embodiments

The present disclosure is based, in part, on the inventors' discovery that modified human cancer cells (e.g., modified human cancer cell lines) containing selected HLA alleles can elicit a robust immune response in the subject. Here, the inventors engineered targeting constructs containing selected HLA-A and HLA-DRB3/4/5 alleles. To eliminate the possibility of competition between endogenous HLA alleles and exogenous HLA alleles introduced as part of the delivery construct in the modified cancer cells, the inventors inactivated an endogenous HLA class I and/or HLA class II allele (e.g., a HLA-A and/or a HLA-DRB3 allele) by genome editing. The engineered delivery construct containing the selected HLA alleles is transfected/electroporated or transduced with HLA knock-in constructs into a human cancer cell to generate a clonal cell line.

In one aspect, the present disclosure provides a modified human cancer cell comprising: (a) one or more recombinant polynucleotides each encoding an allele of a human leukocyte antigen (HLA) class I gene; and (b) one or more recombinant polynucleotides each encoding an allele of an HLA class II gene, wherein one or more HLA alleles endogenous to the cell have been inactivated. In some embodiments, the modified human cancer cell expresses at least one HLA class I allele and at least one HLA class II allele. Without being bound to any theory, the presence of both an HLA class I allele and an HLA class II allele advantageously allows the CD4+ T cells be activated by the class II molecules to provide help and boost the activity of the CD8+ T cells being activated by the class I HLA molecules. It has also been noted that CD8+ T cells can make cytokines which may provide help to CD4+ T cells, and CD4+ T cells can be cytotoxic. The expression of both class I and class II molecules on the same modified cancer cell provides a self-sustaining feedback loop eliciting an immune response in a subject receiving the modified cancer cell.

In some embodiments, the recombinant polynucleotides are integrated into the genome of the cell. In other embodiments, the recombinant polynucleotides are present on one or more vectors in the cell. In some instances, all of the recombinant polynucleotides can be present on the same vector. In other instances, each recombinant polynucleotide can be present on a separate vector. In yet other instances, two, three, four, five, six, or more recombinant polynucleotides can be present on the same vector. Any number of combinations of recombinant polynucleotides on a single vector and any number of vectors in a cell is permitted. In certain instances, all of the recombinant polynucleotides encoding HLA class I alleles can be present on one vector, and all of the recombinant polynucleotides encoding HLA class II alleles can be present on another vector. In particular embodiments, each vector in the cell comprises two recombinant polynucleotides. As a non-limiting example, recombinant polynucleotides encoding two unique HLA class I alleles (e.g., HLA-A alleles) can be present on the same vector. As another non-limiting example, recombinant polynucleotides encoding two unique HLA class II alleles (e.g., HLA-DRB3, HLA-DRB4, and/or HLA-DRB5 alleles) can be present on the same vector. As a further non-limiting example, recombinant polynucleotides encoding two unique cytokines and co-stimulatory molecules (e.g., pairwise combinations of GM-CSF, IFN-α, CD80, CD86, IL-12, IL-7, HLA-DRA, and 4-1BBL) can be present on the same vector. In particular embodiments, the cell comprises: (a) a vector comprising recombinant polynucleotides encoding two unique HLA class I alleles (e.g., HLA-A alleles); (b) a vector comprising recombinant polynucleotides encoding two unique HLA class II alleles (e.g., HLA-DRB3, HLA-DRB4, and/or HLA-DRB5 alleles); and (c) one or more vectors (e.g., one, two, three, four, or more vectors) each comprising recombinant polynucleotides encoding two unique cytokines and co-stimulatory molecules (e.g., pairwise combinations of GM-CSF, IFN-α, CD80, CD86, IL-12, IL-7, HLA-DRA, and 4-1BBL).

The recombinant polynucleotide in the context of the present disclosure may comprise polynucleotides encoding at least one HLA-A class I allele and at least one HLA-A class II allele. Generally, the HLA alleles are selected based on their profile. Without being bound by any particular theory, HLA alleles are highly polymorphic. A less polymorphic HLA allele may be present in a larger proportion of the population. In some embodiments, the HLA (e.g., HLA-A and/or HLA-DRB) alleles selected for the modified human cancer cell described herein have low polymorphism and have at least a single match in at least 50%, at least 55%, at least 60%, at least 65%, at least 70%, at least 75%, at least 80%, at least 85%, at least 90%, at least 95%, or at least 99% of the population. As a non-limiting example, the HLA-A alleles selected for the modified human cancer cell described herein match the HLA-A alleles in about 92% of the population. As another non-limiting example, the HLA-DRB (e.g., HLA-DRB3, HLA-DRB4, and/or HLA-DRB5) alleles selected for the modified human cancer cell described herein match the HLA-DRB alleles in about 98% of the population. In some embodiments, the population is a cancer patient population.

In some embodiments, the HLA class I gene is an HLA-A gene, an HLA-B gene, an HLA-C gene, an HLA-E gene, an HLA-F gene, an HLA-G gene, or a B2M gene. In other embodiments, the HLA class I gene is a combination of an HLA-A gene, an HLA-B gene, an HLA-C gene, an HLA-E gene, an HLA-F gene, an HLA-G gene, and/or a B2M gene. In some embodiments, the modified human cancer cell comprises recombinant polynucleotide(s) encoding alleles of one or more (e.g., 1, 2, 3, 4, 5, 6, 7, 8, 9, 10, or more) HLA class I genes.

Examples of suitable HLA-A alleles include but are not limited to HLA-A*11:01, HLA-A*01:01, HLA-A*02:01, HLA-A*03:01, HLA-A*26:01, HLA-A*29:02, HLA-A*32:01, HLA-A*24:02, HLA-A*33:03, HLA-A*68:01, HLA-A*31:01, and HLA-A*02:06. Modified human cancer cells of the present invention can comprise one or more (e.g., 1, 2, 3, 4, 5, 6, 7, 8, 9, 10, 11, 12, or more) recombinant polynucleotides encoding HLA-A alleles. In some embodiments, the one or more HLA-A alleles are each present at a median frequency of at least about 2% in a population. In other embodiments, the one or more HLA-A alleles are each present at a maximum frequency of at least about 5% in a population. In still other embodiments, the one or more HLA-A alleles are each present at a median frequency of at least about 2% and a maximum frequency of at least about 5% in a population.

Examples of suitable HLA-B alleles include but are not limited to HLA-B*13:02, HLA-B*41:01, HLA-B*18:03, HLA-B*44:02, HLA-B*07:02, HLA-B*35:01, HLA-B*40:01, HLA-B*35:08, HLA-B*55:01, HLA-B*51:01, HLA-B*44:03, HLA-B*58:01, HLA-B*08:01, HLA-B*18:01, HLA-B*15:01, and HLA-B*52:01. Modified human cancer cells of the present disclosure can comprise one or more (e.g., 1, 2, 3, 4, 5, 6, 7, 8, 9, 10, 11, 12, 13, 14, 15, 16, or more) recombinant polynucleotide(s) encoding HLA-B alleles. In some embodiments, the one or more HLA-B alleles are each present at a median frequency of at least about 2% in a population. In other embodiments, the one or more HLA-B alleles are each present at a maximum frequency of at least about 5% in a population. In still other embodiments, the one or more HLA-B alleles are each present at a median frequency of at least about 2% and a maximum frequency of at least about 5% in a population.

Examples of suitable HLA-C alleles include but are not limited to HLA-C*04:01, HLA-C*07:02, HLA-C*07:01, HLA-C*06:02, HLA-C*03:04, HLA-C*01:02, HLA-C*02:02, HLA-C*08:02, HLA-C*15:02, HLA-C*03:03, HLA-C*05:01, HLA-C*08:01, HLA-C*16:01, HLA-C*12:03, and HLA-C*14:02. Modified human cancer cells of the present disclosure can comprise one or more (e.g., 1, 2, 3, 4, 5, 6, 7, 8, 9, 10, 11, 12, 13, 14, 15, or more) recombinant polynucleotide(s) encoding HLA-C alleles. In some embodiments, the one or more HLA-C alleles are each present at a median frequency of at least about 2% in a population. In other embodiments, the one or more HLA-C alleles are each present at a maximum frequency of at least about 5% in a population. In still other embodiments, the one or more HLA-C alleles are each present at a median frequency of at least about 2% and a maximum frequency of at least about 5% in a population.

In some embodiments, the HLA class II gene is an HLA class II alpha subunit gene. In other embodiments, the HLA class II gene is an HLA class II beta subunit gene. In particular embodiments, the HLA class II gene is a combination of HLA class II alpha subunit and HLA class II beta subunit genes.

In other embodiments, the HLA class II gene is an HLA-DP gene, an HLA-DM gene, an HLA-DO gene, an HLA-DQ gene, and/or an HLA-DR gene. In some instances, the HLA-DO gene is an HLA-DOA gene. In other instances, the HLA-DO gene is an HLA-DOB gene. In particular instances, the modified human cancer cell comprises recombinant nucleotides encoding both HLA-DOA and HLA-DOB gene alleles. In some instances, the HLA-DM gene is an HLA-DMA gene. In other instances, the HLA-DM gene is an HLA-DMB gene. In particular instances, the modified human cancer cell comprises recombinant nucleotides encoding both HLA-DMA and HLA-DMB gene alleles.

In some embodiments, the HLA-DR gene is an HLA-DRA gene, an HLA-DRB1 gene, an HLA-DRB3 gene, an HLA-DRB4 gene, and/or an HLA-DRB5 gene. In particular embodiments, the modified human cancer cell comprises recombinant polynucleotides encoding alleles of one or more (e.g., 1, 2, 3, 4, 5, or more) HLA-DR gene(s).

Examples of suitable HLA-DRB3 alleles include but are not limited to HLA-DRB3*02:02, HLA-DRB3*01:01, and HLA-DRB3*03:01. Examples of suitable HLA-DRB4 alleles include but are not limited to HLA-DRB4*01:01 and HLA-DRB4*01:03. Examples of suitable HLA-DRB5 alleles include but are not limited to HLA-DRB5*01:02, HLA-DRB5*01:01, and HLA-DRB5*02:02. Modified human cancer cells of the present disclosure can comprise one or more (e.g., 1, 2, 3, 4, 5, or more) recombinant polynucleotide(s) encoding HLA-DRB3/4/5 alleles. In some embodiments, the one or more HLA-DRB3/4/5 alleles are each present at a median frequency of at least about 2% in a population. In other embodiments, the one or more HLA-DRB3/4/5 alleles are each present at a maximum frequency of at least about 5% in a population. In still other embodiments, the one or more HLA-DRB3/4/5 alleles are each present at a median frequency of at least about 2% and a maximum frequency of at least about 5% in a population.

In some embodiments, the modified human cancer cell comprises at least one recombinant polynucleotide encoding one or more HLA class I genes selected from any one class I gene, a codon optimized version, a variant thereof, or a fragment thereof listed in Table 1 (SEQ ID NOS:1-13, 22-29). For instance, the modified human cancer cell may comprise a recombinant polynucleotide encoding 1, 2, 3, 4 or more HLA class I genes driven by one or more promoters. In some embodiments, the modified human cancer cell further comprises at least one recombinant polynucleotide encoding one or more HLA class II genes selected from any one class II gene, a codon optimized version, a variant, or a fragment thereof listed in Table 1 (SEQ ID NOS:14-21, 30-35). For instance, the modified human cancer cell may comprise a recombinant polynucleotide encoding 1, 2, 3, 4 or more HLA class II genes driven by one or more promoters. 101231 In some embodiments, the modified human cancer cell comprises at least two recombinant polynucleotides each encoding a HLA class I gene selected from any one class I gene, a codon optimized version, a variant, or a fragment thereof listed in Table 1 (SEQ ID NOS:1-13, 22-29). For instance, the modified human cancer cell may comprise a first recombinant polynucleotide encoding a first HLA class I gene and a second recombinant polynucleotide encoding a second HLA class I gene. In some embodiments, the modified human cancer cell comprises at least two recombinant polynucleotides each encoding a HLA class II gene selected from any class II gene, a codon optimized version, a variant thereof, or a fragment thereof listed in Table 1 (SEQ ID NOS:14-21, 30-35). For instance, the modified human cancer cell may comprise a recombinant polynucleotide encoding a first HLA class II gene and a second recombinant polynucleotide encoding a second LA class II gene.

TABLE 1
Listing of SEQ ID NOS

| POLYNUCLEOTIDES | SEQ ID NO |
|---|---|
| HLA-A*01:01 | SEQ ID NO: 1 |
| HLA-A*68:01 | SEQ ID NO: 2 |
| HLA-A*02:01 | SEQ ID NO: 3 |
| HLA-A*11:01 | SEQ ID NO: 4 |
| HLA-A*03:01 | SEQ ID NO: 5 |
| HLA-A*23:01 | SEQ ID NO: 6 |
| HLA-A*33:03 | SEQ ID NO: 7 |
| HLA-A*24:02 | SEQ ID NO: 8 |
| HLA-A*02:06 | SEQ ID NO: 9 |
| HLA-A*26:01 | SEQ ID NO: 10 |
| HLA-A*29:02 | SEQ ID NO: 11 |
| HLA-A*31:01 | SEQ ID NO: 12 |
| HLA-A*32:01 | SEQ ID NO: 13 |
| HLA-DRB3*02:02 | SEQ ID NO: 14 |
| HLA-DRB4*01:01 | SEQ ID NO: 15 |
| HLA-DRB3*03:01 | SEQ ID NO: 16 |
| HLA-DRB3*01:01 | SEQ ID NO: 17 |
| HLA-DRB5*01:02 | SEQ ID NO: 18 |
| HLA-DRB5*01:01 | SEQ ID NO: 19 |
| HLA-DRB5*02:02 | SEQ ID NO: 20 |
| HLA-DRB4*01:03 | SEQ ID NO: 21 |
| HLA-A*01:01 (codon optimized) | SEQ ID NO: 22 |
| HLA-A*68:01 (codon optimized) | SEQ ID NO: 23 |
| HLA-A*02:01 (codon optimized) | SEQ ID NO: 24 |
| HLA-A*11:01 (codon optimized) | SEQ ID NO: 25 |
| HLA-A*03:01 (codon optimized) | SEQ ID NO: 26 |
| HLA-A*23:01-79.6% (codon optimized) | SEQ ID NO: 27 |
| HLA-A*23:01-80.05% (codon optimized) | SEQ ID NO: 28 |
| HLA-A*33:03 (codon optimized) | SEQ ID NO: 29 |
| HLA-DRB3*02:01 (codon optimized) | SEQ ID NO: 30 |
| HLA-DRB5*01:01 (codon optimized) | SEQ ID NO: 31 |
| HLA-DRB3*03:01 (codon optimized) | SEQ ID NO: 32 |
| HLA-DRB5*01:02 (codon optimized) | SEQ ID NO: 33 |
| HLA-DRB4*01:01-79.78% (codon optimized) | SEQ ID NO: 34 |
| HLA-DRB4*01:01-80.27% (codon optimized) | SEQ ID NO: 35 |
| HLA-DRA (codon optimized) | SEQ ID NO: 36 |
| 4-1BBL -77.85% (codon optimized) | SEQ ID NO: 37 |
| 4-1BBL -75.91% (codon optimized) | SEQ ID NO: 38 |
| CSF2 (codon optimized) | SEQ ID NO: 39 |
| IFNA2 (codon optimized) | SEQ ID NO: 40 |
| IL7-72.64% (codon optimized) | SEQ ID NO: 41 |
| IL7-77.32% (codon optimized) | SEQ ID NO: 42 |
| CD80 (codon optimized) | SEQ ID NO: 43 |
| IL-12 (bioactive form) | SEQ ID NO: 44 |

In some embodiments, the modified human cancer cell comprises one or more recombinant polynucleotides encoding a combination of at least a cytokine, a co-stimulatory molecule, a heterologous antigen (e.g., an antigen of a pathogen, a tumor-associated antigen, a neo-antigen, an allergen, an antigen that is the target of an immune response), a variant thereof, or a fragment thereof. The cytokine can be a chemokine, an interferon, an interleukin, or a tumor necrosis factor. The cytokine can be selected from an early T cell activation antigen-1 (ETA-1), a lymphocyte-activating factor (LAF), an interleukin-1 family member (IL-1α, IL-β, IL-1Ra, IL-18, IL-33, IL-36Ra, IL-36α, IL-36β, IL-36γ, IL-37, IL-38), an interleukin-2 (IL-2), an interleukin-3 (IL-3), an interleukin-4 (IL-4), an interleukin-5 (IL-5), an interleukin-6 (IL-6), an interleukin-7 (IL-7), an interleukin-8 (IL-8), an interleukin-9 (IL-9), an interleukin-10 (IL-10), an interleukin-12 (IL-12), an interleukin-13 (IL-13), an interleukin-15 (IL-15), an interleukin-17 (IL-17), an interleukin-18 (IL-18), an interleukin-21 (IL-21), an interleukin-23 (IL-23), an interleukin-25 (IL-25), an interleukin-33 (IL-33), an interferon alpha (IFN-α), an interferon lambda 1 (IFNλ1 (IL-29)), an interferon lambda 2 (IFNλ2 (IL-28A)), an interferon lambda 3 (IFNλ3 (IL-28B)), an interferon lambda 4 (IFNλ4), a granulocyte-macrophage colony-stimulating factor (GM-CSF), a macrophage CSF (CSF-1), a macrophage migration inhibitory factor (MIF), a CD40L molecule (CD40L), a RANTES molecule (RANTES), a monocyte chemoattractant protein (MCP-1), a monocyte inflammatory protein (MIP-1α, MIP-1β), a lymphotactin, or a fractalkine. The co-stimulatory molecule can be selected from at least one of a CD86 molecule (CD86), CD80 molecule (CD80), 4-1BB ligand molecule (4-1BBL, also known as TNFSF9 or CD137L), ICOS ligand molecule (ICOS-L), CD70 molecule (CD70 a.k.a. CD27L), CD40 molecule (CD40), OX40 ligand molecule (OX40L), GITR ligand molecule (GITRL), TIM-4 molecule (TIM-4), LIGHT molecule (LIGHT), ICAM1 molecule (ICAM1), LFA3 molecule (LFA3), a CD30 molecule (CD30), and a combination thereof.

In some embodiments, the expression of the HLA alleles, heterologous antigens, cytokines (e.g., GM-CSF, and/or IFN-α2), and co-stimulatory molecules are under the control of two or more different promoters. In some instances, the expression of each HLA alleles, heterologous antigens, cytokines (e.g., GM-CSF, and/or IFN-α2), and co-stimulatory molecules is under the control of a separate promoter. In some embodiments, the expression of the HLA alleles, heterologous antigens, cytokines (e.g., GM-CSF, and/or IFN-α2), and co-stimulatory molecules are under the control of a single promoter. In some instances, the HLA alleles, heterologous antigens, cytokines (e.g., GM-CSF, and/or IFN-α2), and co-stimulatory molecules are expressed as a polycistronic mRNA in a multicistronic vector. In particular instances, one or more cistrons are separated by internal ribosomal entry sites. In other instances, one or more cistrons are separated by a self-cleaving peptide (e.g., a T2A, P2A, E2A, F2A). The one or more recombinant polynucleotides may be introduced into an expression vector for synthesizing the corresponding HLA alleles. Exemplary vector construction is discussed in detail in Section B, Example 1 and Example 5.

In some embodiments, the modified human cancer cell comprises (a) one or more vectors each comprising a recombinant polynucleotide encoding at least one gene selected from CSF2, IFN-α2, CD86, IL-12, CD80, HLA-DRA, IL-7, and/or 4-1BBL (e.g., four vectors each comprising a recombinant polynucleotide encoding two different genes); (b) one or more vectors each comprising a recombinant polynucleotide encoding at least one gene selected from HLA-A*01:01, HLA-A*68:01, HLA-A*02:01, HLA-A*11:01, HLA-A*03:01, HLA-A*23:01, HLA-A*24:02, and/or HLA-A*33:03; and (c) one or more vectors comprising a recombinant polynucleotide encoding at least one gene selected from HLA-DRB3*02:02, HLA-DRB5*01:01, HLA-DRB4*01:01, HLA-DRB3*01:01, HLA-DRB3*03:01, HLA-DRB5*01:02, and/or HLA-DRB5*02:02.

In some embodiments, the modified human cancer cell comprises (a) one or more vectors each comprising a recombinant polynucleotide encoding at least one gene selected from CSF2, IFN-α2, CD86, IL-12, CD80, HLA-DRA, IL-7, and/or 4-1BBL (e.g., four vectors each comprising a recombinant polynucleotide encoding two different genes); (b) a vector comprising a recombinant polynucleotide encoding an HLA-A*01:01 allele and an HLA-A*68:01 allele, or an HLA-A*02:01 allele and an HLA-A*11:01 allele, or an HLA-A*03:01 allele and an HLA-A*23:01 allele, or an HLA-A*24:02 allele and an HLA-A*33:03 allele; and (c) a vector comprising a recombinant polynucleotide encoding an HLA-DRB3*02:02 allele and an HLA-DRB5*01:01 allele, or an HLA-DRB4*01:01 allele and an HLA-DRB3*01:01 allele, or an HLA-DRB3*03:01 allele and an HLA-DRB5*01:02 allele, or an HLA-DRB5*02:02 allele and an HLA-DRB3*01:01 allele.

In various embodiments, each vector comprises a recombinant polynucleotide encoding one or more 2A splicing peptides selected from T2A, P2A, or E2A located between polynucleotides encoding two of the genes, two of the HLA alleles, and/or a gene and an HLA allele.

In some embodiments, the modified human cancer cell comprises one or more HLA-A and/or HLA-DR alleles endogenous to the cell that have been inactivated. In some embodiments, the human cancer cell further comprises an inactivated HLA-DRB3 allele, HLA-DRB4 allele, and/or HLA-DRB5 allele. In some embodiments, the one or more HLA alleles that have been inactivated have a sequence of at least 85% identity to any one of SEQ ID NOS: 1-36 (Table 1), a variant thereof, or a fragment thereof. In some embodiments, the modified human cancer cell comprises an inactivated HLA-A*24:02 allele and/or an HLA-A*11:01 allele, a variant thereof, or a fragment thereof. In some embodiments, the modified human cancer cell further comprises an inactivated HLA-DRB3*01:01 allele and/or an HLA-DRB3*02:02 allele, a variant thereof, or a fragment thereof. As a non-limiting example, the modified human cancer cell may comprise at least one inactivated HLA-A allele and at least one inactivated HLA-DRB3/3/5 allele. In some embodiments, the modified human cancer cell comprises an inactivated HLA-A*24:02 allele, HLA-A*11:01 allele, DRB3*01:01 allele, HLA-DRB3*02:02 allele, or a combination thereof. In some embodiments, the modified human cancer cell further comprises an inactivated CD47 molecule, a variant thereof, or a fragment thereof, such that cell surface expression of CD47 is reduced or eliminated. In certain instances, the modified cancer cells described herein deficient in CD47 expression have increased immunogenicity. In some embodiments, the modified human cancer cell further comprises an inactivated Ii(CD74) molecule, a variant thereof, or a fragment thereof. In some embodiments, the modified cancer cells described herein (e.g., Bria-OTS cell lines) comprise inactivated Ii (CD74), a variant, or a fragment thereof, such that cell surface expression of Ii(CD74) is reduced or eliminated. In certain instances, the modified cancer cells described herein (e.g., Bria-OTS cell lines) that are deficient in Ii (CD74) expression have increased immunogenicity. In some embodiments, the modified cancer cells described herein (e.g., Bria-OTS cell lines) that overexpress HLA-DRA overcome the inhibitory effect of Ii on tumor cells. In some embodiments, overexpression of HLA-DRA and deficiency in Ii(CD74) expression in the modified cancer cells described herein (e.g., Bria-OTS cell lines) have a synergistic effect on inducing antitumor immunity. As a non-limiting example, the modified human cancer cell may comprise an inactivated HLA-A*24:02 allele, HLA-A*11:01 allele, DRB3*01:01 allele, HLA-DRB3*02:02 allele, or a combination thereof, and an inactivated CD47 molecule, a variant thereof, or a fragment thereof. As another non-limiting example, the modified human cancer cell may comprise an inactivated HLA-A*24:02 allele, HLA-A*11:01 allele, DRB3*01:01 allele, HLA-DRB3*02:02 allele, or a combination thereof, and an inactivated Ii (CD74) molecule, a variant thereof, or a fragment thereof. As yet another non-limiting example, the modified human cancer cell may comprise an inactivated HLA-A*24:02 allele, HLA-A*11:01 allele, DRB3*01:01 allele, HLA-DRB3*02:02 allele, or a combination thereof, an inactivated CD47 molecule, a variant thereof, or a fragment thereof, and an inactivated Ii (CD74) molecule, a variant thereof, or a fragment thereof.

In some embodiments, the modified human cancer cell is derived from a human cancer cell line. Any number of human cancer cells or cancer cell lines are suitable for use in the compositions and methods described herein, including, for example, clonal or non-clonal human cancer cells or cancer cell lines. Non-limiting examples of human cancer cell lines include the following cell lines and subclones thereof: the SV-BR-1, SVCT, MDA-MB-231, MDA-MB-157, ZR-75-30, ZR-75-1, Hs 578T, MCF7, T47D, MTSV1-7 CE1, 1-711B2, VP303, VP267, and VP229 breast cancer cell lines; the PC-3, LNCaP (e.g., clone FGC), Shmac 5, P4E6, and VCaP prostate cancer cell lines; the NCI-H2228, SHP-77, COR-L23/R, COR-L23/5010, MOR/0.2R, NCI-H69/LX20, ChaGo-K-1, and Meta 7 lung cancer cell lines; the SK-MEL-24 melanoma cell line; the UM-UC-3, T24/83, ECV304, RT4, and HT 1197 bladder cancer cell lines; the MDST8, C170, GP5d, GP2d, and LS 123 colon cancer cell lines; the MFE-280 and MFE-296 endometrial cancer cell lines; the CAKI 2, A.704, G-402, ACHN, G-401, UM-RC-7, and RCC4plusVHL renal cancer cell lines; the SK-HEP-1, Hep 3B, PLC/PRF/5, Hep G2, and Huh-7D12 liver cancer cell lines; the HL60, Eos-HL-60, JVM-13, Sci-1, and Ri-1 leukemia cell lines; the BHL-89, COR-L24, U937(CD59+), My-La CD8+, and HGC-27 lymphoma cell lines; the A375-C6, GR-M, VA-ES-BJ, MEWO, and COLO 818 skin cancer cell lines; the AsPC-1, HuP-T4, HuP-T3, BxPC-3, and CFPAC-1 pancreatic cancer cell lines; the 8505C, 8305C, FTC-238, TT, R082-W-1, and K1 thyroid cancer cell lines; the HeLa DH, HR5-CL11, HtTA-1, HR5, X1/5, HeLa, C-4I, C-4 II, HeLa S3, Ca Ski, HeLa229, Hep2 (HeLa derivative), HeLa B, Bu25 TK-HeLa Ohio, and HeLa (AC-free) cervical cancer cell lines; the NB69, BE(2)-C, BE(2)-M17, SK-N-BE(2), and SK-N-DZ brain cancer cell lines; the OV7, OV17R, OV58, OV56, A2780ADR, A2780, COLO 720 E, SW 626, SK-OV-3, PA-1, 59M, OAW28, TO14, PE023, and COV362 ovarian cancer cell lines; the IMR 32 abdominal cancer cell line; the SW 13 adrenal cortex cancer cell line; the TR146 buccal mucosa cancer cell line; the SK-GT-4 esophageal cancer cell line; the TE 671 embryonic cancer cell line; the FLYRD18 fibrosarcoma cell line; the 1411H germ cell tumor cell line; the MFM-223 mammary gland cancer cell line; the H-EMC-SS muscle cancer cell line; the Detroit 562 pharyngeal cancer cell line; the BeWo placental cancer cell line; the Mero-95 pleural cavity cancer cell line, the SW 837, SW 1463, CMT 93, HRT-18, and HRA-19 rectal cancer cell lines; the Y79, WERI, and RB247C retinal cancer cell line; the CHP-100 spinal cancer cell line; the KARPAS 1718 splenic lymphoma cell line; the AGS and KATO-III stomach cancer cell lines; the NTERA-2 clone D1 testicular cancer cell line; the SCC-9, H357, H103, BICR 56, and PE/CA-PJ49 tongue cancer cell lines; the MES-SA/Dx-5, MES-SA, COLO 685, and COLO 684 uterine cancer cell lines; and the HMVII vaginal cancer cell line. In particular embodiments, the human cancer cell line is a breast cancer (e.g., SV-BR-1), prostate cancer (e.g., PC-3, LNCaP), melanoma (e.g., SK-MEL-24), or lung cancer (e.g., NCI-H2228) cell line. The cell lines described herein and others are available, for example, from Sigma-Aldrich (www.sigmaaldrich.com). In other embodiments, the modified human cancer cell is derived from a cancer cell obtained from a tumor biopsy, for example, from a subject who is to be treated for cancer prior to modification of the cancer cell.

### A. Selected HLA Alleles

In one aspect, the modified human cancer cell described herein (e.g., Bria-OTS cell line) may express combinations of HLA genes having a sequence of any one of SEQ ID NOS: 1-35 (Table 1), a variant thereof, or a fragment thereof. As an illustration, a modified human cancer cell described herein (e.g., Bria-OTS cell line) may comprise: a combination of HLA-A*01:01, HLA-A*68:01, HLA-DRB3*02:02 and/or HLA-DRB4*01:01 alleles; a combination of HLA-A*02:01, HLA-A*11:01, HLA-DRB4*01:01 and/or HLA-DRB3*03:01 alleles; a combination of HLA-A*03:01, HLA-A*23:01, HLA-DRB3*01:01 and/or HLA-DRB5*01:02 alleles; or a combination of HLA-A*33:03, HLA-DRB5*01:01 and/or HLA-DRB5*02:02 alleles, and optionally an HLA-A*24:02 allele. As another illustration, a modified human cancer cell described herein (e.g., Bria-OTS cell line) may comprise: a combination of HLA-A*01:01, HLA-A*68:01, HLA-DRB3*02:02 and/or HLA-DRB5*01:01 alleles; a combination of HLA-A*02:01, HLA-A*11:01, HLA-DRB4*01:01 and/or HLA-DRB3*01:01 alleles; a combination of HLA-A*03:01, HLA-A*23:01, HLA-DRB3*03:01 and/or HLA-DRB5*01:02 alleles; or a combination of HLA-A*24:02, HLA-A*33:03, HLA-DRB5*02:02 and/or HLA-DRB3*01:01 alleles.

In another aspect, the modified human cancer cell described herein (e.g., Bria-OTS cell line) may express: (a) at least one HLA gene having a sequence of any one of SEQ ID NOS: 1-21 (Table 1), a variant thereof, or a fragment thereof; (b) at least one codon optimized HLA gene (e.g., polynucleotides ending an HLA class I allele and/or an HLA class II allele) having a sequence of any one of SEQ ID NOS: 22-35 (Table 1), a variant thereof, or a fragment thereof; and/or (c) at least one codon optimized co-stimulatory molecule having a sequence of any one of SEQ ID NOS: 36-44 (Table 1), a variant, thereof or a fragment thereof. For instance, a modified human cancer cell described herein (e.g., Bria-OTS cell line) may comprise an HLA-A*68:01 allele, a variant thereof, or a fragment thereof, a codon optimized HLA-A*01:01 allele, a variant thereof, or a fragment thereof; and a co-stimulatory molecule, a variant thereof, or a fragment thereof.

In some aspects, any one of the HLA genes, variants thereof, or fragments thereof may have a polynucleotide sequence having at least 70%, 75%, 80%, 85%, 90%, 95%, 99%, or higher identity or similarity with its counterpart wild-type sequence. In some embodiments, any one of the HLA genes, variants thereof, or fragments thereof may have a polynucleotide sequence having at most 70%, 75%, 80%, 85%, 90%, 95%, or 99% identity or similarity with its counterpart wild-type sequence. In some embodiments, a polynucleotide encoding the modified HLA allele or codon-optimized HLA allele, a variant thereof, or a fragment thereof has at least 70%, 75%, 80%, 85%, 90%, 95%, 99%, or higher identity or similarity with its counterpart wild-type sequence. In some embodiments, a polynucleotide encoding the modified HLA allele or codon-optimized HLA allele, a variant thereof, or a fragment thereof has at most 70%, 75%, 80%, 85%, 90%, 95%, or 99% identity or similarity with its counterpart wild-type sequence. In some embodiments, a polynucleotide encoding the modified HLA allele or codon-optimized HLA allele, a variant thereof, or a fragment thereof has about 10% to 99%, about 30% to 80%, about 40% to 95%, about 60% to 85% identity or similarity with its counterpart wild-type sequence.

### B. Expression Vectors for Selected HLA Alleles

In some embodiments, the modified human cancer cell described herein contains one or more expression vectors for expressing the recombinant HLA alleles. A wide variety of expression vectors may be used, such as retroviral vectors, lentiviral vectors, adenoviral vectors, adeno-associated viral vectors, plasmids or transposons. Viral vectors that may be used, for example, include vectors based on HIV, SV40, EBV, HSV or BPV. The expression vectors may be replication-defective by design such that the viral vector is defective for one or more functions that are essential for viral genome replication or synthesis and assembly of viral particles. Many of the currently existing replication-defective viruses can carry large therapeutic genes, effectively transduce various types of cells, and provide long-term and stable expression of genes of interest.

Lentiviruses are a subset of retroviruses commonly used in research. Lentiviruses can transduce both dividing and non-dividing cells without a significant immune response. These viruses also integrate stably into the host genome, enabling long term transgene expression. A common lentivirus is the human immunodeficiency virus (HIV), which uses the envelope glycoproteins of other viruses to target a broad range of cell types.

One safety feature of lentiviruses is that the components necessary to produce an infectious viral particle (a virion) are generally divided among multiple plasmids. For instance, an infectious viral particle may comprise plasmids that components of the viral capsid and envelope (typically called the packaging and envelope plasmids), and plasmid that encodes the viral genome (typically called the transfer plasmid). Common lentiviral packaging and envelope plasmids that can be used herein include, but are not limited to, pRSV-Rev, pMDLg/pRRE, psPAX2, pCMV delta R8.2, pMD2.G, pCMV-VSV-G, pCMV-dR8.2 dvpr, pCI-VSVG, pCPRDEnv, pLTR-RD114A, pLTR-G, pCD/NL-BH*DDD, psPAX2-D64V, pCEP4-tat, pHEF-VSVG, pNHP, pCAG-Eco, and pCAG-VSVG. Common lentiviral transfer plasmids that can be used herein include, but are not limited to, pLKO.1 puro, pLKO.1-TRC cloning plasmid, pLKO.3G, Tet-pLKO-puro, pSico, pLJM1-EGFP, FUGW, pLVTHM, pLVUT-tTR-KRAB, pLL3.7, pLB, pWPXL, pWPI, EF.CMV.RFP, pLenti CMV Puro DEST, pLenti-puro, pLOVE, pULTRA, pLX301, plnducer20, pHIV-EGFP, Tet-pLKO-neo, pLV-mCherry, pCW57.1, pLionII, pSLIK-Hygro, and pInducer10-mir-RUP-PheS.

There are multiple approaches to produce lentiviral vectors. (See Logan et al. “Factors influencing the titer and infectivity of lentiviral vectors.” Hum Gene Ther. 2004 October; 15(10):976-88. doi: 10.1089/hum.2004.15.976. PMID: 15585113; Dull, T et al. “A third-generation lentivirus vector with a conditional packaging system.” Journal of virology vol. 72, 11 (1998): 8463-71. doi:10.1128/JVI.72.11.8463-8471.1998). Alternatively, lentiviral vectors may be purchased from commercial providers. In general, production of lentiviral vectors involves multiple steps including plasmid development and production, cell expansion, plasmid transfection, viral vector production, purification, fill and finish. (See e.g., www.addgene.org/viral-vectors/lentivirus/; www.thermofisher.com/us/en/home/clinical/cell-gene-therapy/gene-therapy/lv-production-workflow.html).

The lentiviral vector may be designed to express one or more genes of interest simultaneously. Various molecular strategies are available, including the use of multiple promoters, signals of splicing, fusion of genes, cleavage factors and multicistronic vectors. (See e.g., review by Shaimardanova et al., “Production and application of multicistronic constructs for various human disease therapies.” Pharmaceutics 2019, 11, 580.)

Multicistronic vectors generally contain sequences encoding the nucleotide sequences of internal ribosome entry site (IRES) and self-cleaving 2A peptides. The use of IRES and self-cleaving 2A peptides allows simultaneous expression of two or more separate proteins from the same mRNA.

Self-cleaving 2A peptides are used for the production of multicistronic vectors due to their small size and self-cleavage ability. 2A peptides are composed of 16-20 amino acids and originate from viral RNA. Common 2A peptides used to produce multicistronic vectors are F2A (2A peptide derived from the foot-and-mouth disease virus), E2A (2A peptide derived from the equine rhinitis virus), P2A (2A peptide derived from the porcine teschovirus-1), and T2A (2A peptide derived from the Thosea asigna virus).

In constructs with 2A peptide sequences, translation is initiated once and synthesis along mRNA occurs continuously. During translation, the first peptide breaks from the second peptide in the 2A region. 2A peptide cleavage site is located between glycine and proline. The cleavage process occurs inside the ribosome during protein synthesis. The formation of a normal peptide bond between the amino acids is inhibited only at the cleavage site, thus the cleavage does not affect the translation of the subsequent protein and synthesis continues without the dissociation of ribosome. Thus, translation of multiple genes are dependent of each other. The use of different 2A peptides may influence the expression levels of downstream protein. Combinations of the order of 2A peptide sequence may prevent gradual decrease in the gene expression from the first to the last in multicistronic constructs. For instance, the combination of 2A peptide sequences in the following order, namely T2A, P2A, and E2A, is optimal when creating multicistronic vectors containing four genes.

In some embodiments, the modified human cancer cells described herein are expressed using non-viral approaches. Exemplary methods include, but are not limited to, cationic lipids such as liposomes and lipoplexes, polymers or polyplexes and dendrimers, naked plasmids for direct delivery, electroporation, ultrasound and micro bubbles, magnetofections, inorganic molecules.

In one aspect, the present disclosure provides an expression vector for the modified human cancer cell described herein. The vector may comprise one or more recombinant polynucleotides encoding at least one HLA-A class I allele and at least one HLA-A class II allele (e.g., at least one HLA-DRB3 allele, at least one HLA-DRB4 allele, and/or at least one HLA-DRB5 allele) selected from any one of SEQ ID NOS: 1-35 (Table 1), a variant thereof, or a fragment thereof. The recombinant polynucleotide may further comprise at least one co-stimulatory molecule, and/or at least one HLA allele selected from any one of SEQ ID NOS: 36-44 (Table 1). In some embodiments, the HLA allele is a codon-optimized HLA allele (e.g., HLA-DRA). In some embodiments, the recombinant polynucleotide further comprises a sequence encoding one or more cytokines (e.g., GM-CSF, IFN-α2 including IFN-α2a and IFN-α2b). In some embodiments, the recombinant polynucleotide encoding the HLA-A class I alleles and HLA-A class II alleles (e.g., HLA-DRB3 alleles, HLA-DRB4 alleles, and/or HLA-DRB5 alleles), co-stimulatory molecules, heterologous antigens, and/or cytokines, each has a sequence having at least 70%, 75%, 80%, 85%, 90%, 95%, 99%, or higher identity or similarity with their counterpart wild-type sequence; each has a sequence having at most 70%, 75%, 80%, 85%, 90%, 95%, or 99% identity or similarity with their counterpart wild-type sequence; or each has a sequence having about 10% to 99%, about 30% to 80%, about 40% to 95%, about 60% to 85% identity or similarity with their counterpart wild-type sequence. In various embodiments, the expression vector comprises one or more recombinant polynucleotides encoding one or more promoters for driving expression of the selected HLA alleles, codon-optimized HLA alleles, co-stimulatory molecules, or adjuvant. The expression vector may contain recombinant polynucleotides encoding a MNDU3 promoter, an EF1α promoter, or both. For example, the expression vector may contain a first recombinant polynucleotide encoding a MNDU3 promoter and a second recombinant polynucleotide encoding an EF1α promoter. In particular embodiments, the expression vector contains a recombinant polynucleotide encoding a MNDU3 promoter and an EF1α promoter.

Table 2 illustrates the construction of exemplary expression vectors using a multicistronic lentiviral vector. Each lentiviral vector carries two cytokine genes and four HLA alleles. GM-CSF is encoded by the CSF2 gene, while IFN-α2a and IFN-α2b are variants encoded by IFNA2.

TABLE 2
Constructs for generation of exemplary modified human cancer cell lines

| | | | MNDU3 Promoter | | | |
|---|---|---|---|---|---|---|
| | EF1α Promoter | | | 2A# | 2A# HLA- | 2A# HLA- |
| | | 2A# | HLA-A | HLA-A | DRB3/4/5 | DRB3/4/5 |
| Cell Line ID | Cytokine 1 | Cytokine 2 | Allele 1 | Allele 2 | Allele 1 | Allele 2 |
| Bria-OTS01/Lenti-1 | CSF2 | T2A# | A*01:01 | T2A# | P2A# | E2A# |
| BC1.68.3.4 (BC1)) | | IFNA2 | | A*68:01 | DRB3*02:02 | DRB4*01:01 |
| Bria-OTS02/Lenti-2 | CSF2 | T2A# | A*02:01 | T2A# | P2A# | E2A# |
| (BC2.11.4.3 (BC2)) | | IFNA2 | | A*11:01 | DRB4*01:01 | DRB3*03:01 |
| Bria-OTS03/Lenti-3 | CSF2 | T2A# | A*03:01 | T2A# | P2A# | E2A# |
| (BC3.23.3.5 (BC3)) | | IFNA2 | | A*23:01 | DRB3*01:01 | DRB5*01:02 |
| Bria-OTS04/Lenti-4 | CSF2 | T2A# | A*24:02 | T2A# | P2A# | E2A# |
| (BC24.33.5.5 (BC4)) | | IFNA2 | | A*33:03 | DRB5*01:01 | DRB5*02:02 |

#Self-splicing 2A peptides

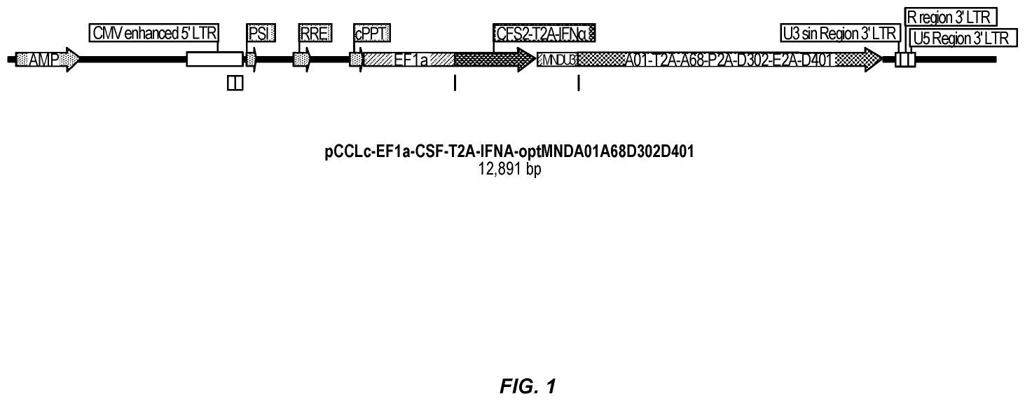

In some illustrations, FIGS. 1-4 depict components of multicistronic lentiviral vectors for expressing the selected HLA alleles in the human modified cancer cell. Construction of the expression vector is described in Example 1.

Referring to FIG. 1, a recombinant polynucleotide encoding, from 5′ to 3′, an EF1α promoter, a GM-CSF (encoded by the CSF2 gene), a T2A splicing peptide, an IFN-α2b (encoded by the IFNA2 gene), a MNDU3 promoter, an HLA-A*01:01 allele, a T2A splicing peptide, an HLA-A*68:01 allele, a P2A splicing peptide, an HLA-DRB3*02:02, an E2A splicing peptide, and an HLA-DRB4*01:01 allele is inserted into the pCCLc backbone. As shown in FIG. 1, the EF1α promoter is located at the 5′ end of a sequence encoding the GM-CSF and IFN-α (e.g., IFN-α2a, or IFN-α2b). Thus, synthesis of both GM-CSF and IFN-α is driven by the EF1α promoter. The MNDU3 promoter is located at the 3′ end of the sequence encoding the GM-CSF and IFN-α and at the 5′ end of the sequence encoding the four HLA alleles and three 2A splicing peptides. Each of the three 2A splicing peptides is located between polynucleotide sequences encoding two HLA alleles. In particular, three 2A splicing peptides are present in the order of T2A, P2A, and E2A, for maximizing expression of all four HLA genes.

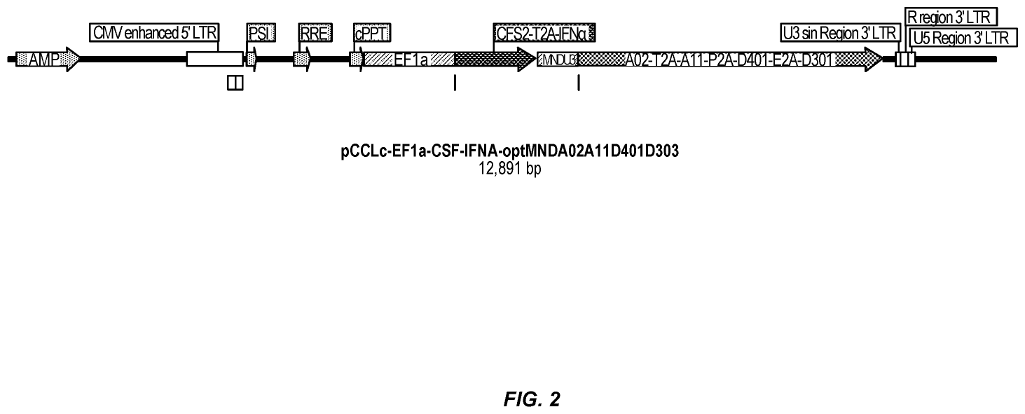

Referring to FIG. 2, a recombinant polynucleotide encoding, from 5′ to 3′, an EF1α promoter, a GM-CSF (encoded by the CSF2 gene), a T2A splicing peptide, an IFN-α (such as IFN-α2a or IFN-α2b encoded by the IFNA2 gene), a MNDU3 promoter, an HLA-A02:01 allele, a T2A splicing peptide, an HLA-A*11:01 allele, a P2A splicing peptide, a HLA-DRB4*01:01 allele, an E2A peptide, and an HLA-DRB3*03:01 allele is inserted into the pCCLc backbone. As shown in FIG. 2, the EF1α promoter is located at the 5′ end of a sequence encoding the GM-CSF, T2A splicing peptide and IFN-α. Thus, synthesis of both GM-CSF and IFN-α is driven by the EF1α promoter. The MNDU3 promoter is located at the 3′ end of the sequence encoding the GM-CSF and IFN-α and at the 5′ end of the sequence encoding the four HLA alleles and three 2A splicing peptides. Each of the three 2A splicing peptides is located between the polynucleotide sequences encoding two HLA alleles. In particular, three 2A splicing peptides are present in the order of T2A, P2A, and E2A, for maximizing expression of all four HLA genes.

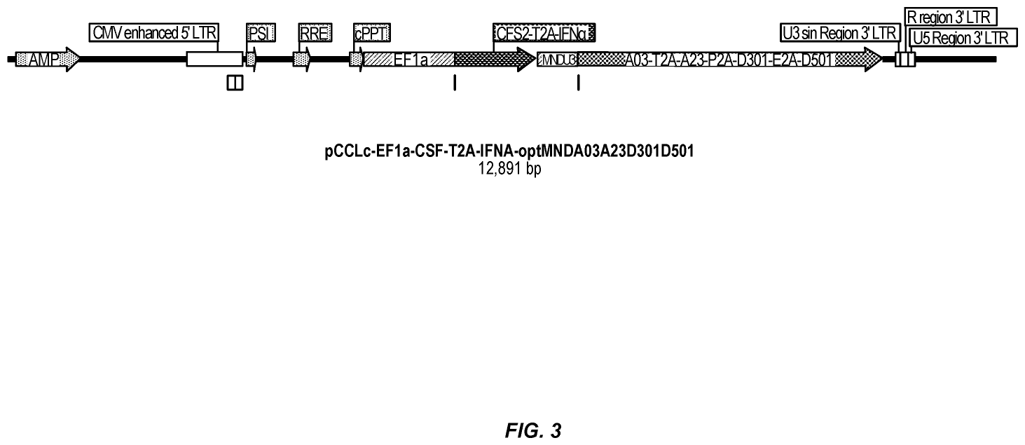

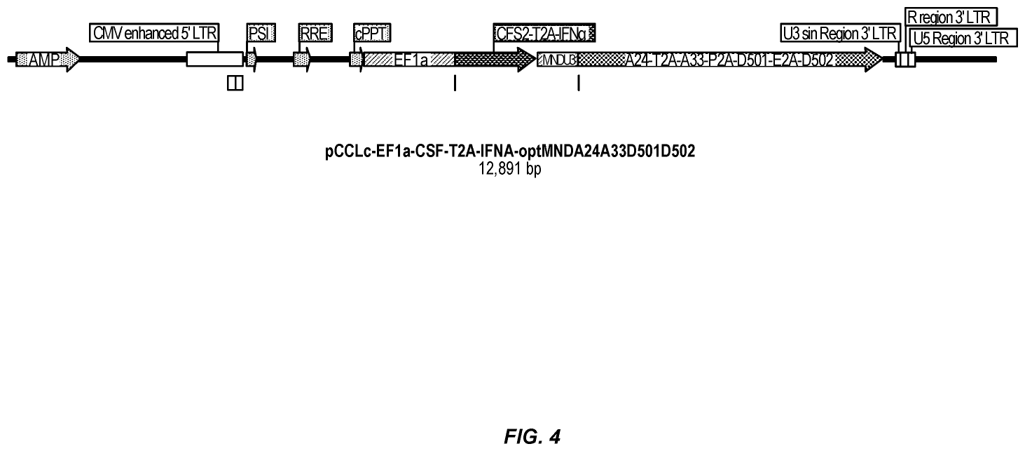

FIG. 3 and FIG. 4 show similar construction of the expression vector in FIG. 2. In particular, the expression vector in FIG. 3 is designed to express the HLA alleles (from 5′ to 3′): HLA-A*03:01, HLA-A*23:01, HLA-DRB3*01:01, and HLA-DRB5*01:02. The expression vector in FIG. 4 is designed to express the HLA alleles (from 5′ to 3′): HLA-A*24:02, HLA-A*33:03, HLA-DRB5*01:01, and HLA-DRB5*02:02. As shown in FIG. 3 and FIG. 4, a 2A splicing peptide (in the order of T2A, P2A, and E2A) is located between polynucleotide sequences encoding each two HLA alleles, two promoters, or an HLA allele and a promoter.

In another embodiment, the expression vector is a multicistronic lentiviral vector designed to express a combination of any one of the cytokines or HLA genes under the control of an EF1α promoter and/or a MNDU3 promoter. An exemplary construction of multicistronic lentiviral vectors is illustrated in Table 3. As an illustration, the expression vector comprises: (a) one or more recombinant polynucleotides each encoding at least one gene selected from CSF2, IFNA2, CD86, IL-12, CD80, HLA-DRA, IL-7, and/or 4-1BBL (also known as TNFSF9 or CD137L); (b) one or more recombinant polynucleotides each encoding at least one gene selected from HLA-A*01:01, HLA-A*68:01, HLA-A*02:01, HLA-A*11:01, HLA-A*03:01, HLA-A*23:01, HLA-A*24:02, and/or HLA-A*33:03; or (c) one or more recombinant polynucleotides each encoding at least one gene selected from an HLA-DRB3*02:02 allele, LA-DRB5*01:01 allele, LA-DRB3*03:01 allele, HLA-DRB4*01:01 allele, LA-DRB3*01:01 allele, HLA-DRB5*01:02 allele, and/or HLA-DRB5*02:02 allele. As an example, the expression vector may comprise a recombinant polypeptide encoding a CD86 gene driven under the control of an EF1α promoter and an IL-12 gene driven under the control of a MDNU3 promoter. As another example, the expression vector may comprise a recombinant polypeptide encoding a CD86 gene driven under the control of a MDNU3 promoter and an IL-12 gene driven under the control of an EF1α promoter.

TABLE 3
Lentiviral constructs for generation of exemplary modified human
cell lines comprising a co-stimulatory molecule and an HLA allele

| Lentiviral ID No. | EF1α Promoter | MNDU3 Promoter |
|---|---|---|
| GM-IFN | CSF2 | IFNA2 |
| GM | CSF2 | — |
| CD86-IL-12 | CD86 | IL-12 |
| CD80-HLA-DRA | CD80 | HLA-DRA |
| IL-7-4-1BBL | IL-7 | 4-1BBL (TNFSF9; CD137L) |
| A2-A11 | HLA-A*02:01 | HLA-A*11:01 |
| A1-A68 | HLA-A*01:01 | HLA-A*68:01 |
| A3-A23 | HLA-A*03:01 | HLA-A*23:01 |
| A24-A33 | HLA-A*24:02 | HLA-A*33:03 |
| DR401-301 | HLA-DRB4*01:01 | HLA-DRB3*01:01 |
| DR302-501 | HLA-DRB3*02:02 | HLA-DRB5*01:01 |
| DR303-502 | HLA-DRB3*03:01 | HLA-DRB5*01:02 |
| DR301-522 | HLA-DRB5*02:02 | HLA-DRB3*01:01 |

In some embodiments, the expression vector comprises one or more recombinant polynucleotides each encoding at least one immunomodulatory molecule, co-stimulatory molecule, or cytokine (e.g., two or more immunomodulatory molecules, co-stimulatory molecules, or cytokines) including, but not limited to, CSF2, IFNA2, CD86, IL-12, CD80, HLA-DRA, IL-7, and/or 4-1BBL (also known as TNFSF9 or CD137L). In some embodiments, the expression vector comprises one or more recombinant polynucleotides each encoding at least one immunomodulatory molecule, co-stimulatory molecule, or cytokine (e.g., two or more immunomodulatory molecules, co-stimulatory molecules, or cytokines) selected from CSF2 and IFNA2; CSF2; CD86 and IL-12; CD80 and an HLA-DRA allele; or IL-7 and 4-1BBL.

In some embodiments, the expression vector comprises one or more recombinant polynucleotides each encoding at least one HLA class I allele (e.g., two or more HLA class I alleles) including, but not limited to, an HLA-A*01:01 allele, HLA-A*68:01 allele, HLA-A*02:01 allele, HLA-A*11:01 allele, HLA-A*03:01 allele, HLA-A*23:01 allele, HLA-A*24:02 allele, and/or HLA-A*33:03 allele. In some embodiments, the expression vector comprises one or more recombinant polynucleotides each encoding at least one HLA class I allele (e.g., two or more HLA-A alleles) selected from an HLA-A*01:01 allele and an HLA-A*68:01 allele, an HLA-A*02:01 allele and an HLA-A*11:01 allele, an HLA-A*03:01 allele and an HLA-A*23:01 allele, and/or an HLA-A*24:02 allele and an HLA-A*33:03 allele.

In some embodiments, the expression vector comprises one or more recombinant polynucleotides each encoding at least one HLA class II allele (e.g., two or more HLA class II alleles) including, but not limited to, an HLA-DRB3*02:02 allele, HLA-DRB5*01:01 allele, HLA-DRB4*01:01 allele, HLA-DRB3*01:01 allele, HLA-DRB3*03:01 allele, HLA-DRB5*01:02 allele, and/or HLA-DRB5*02:02 allele. In some embodiments, the expression vector comprises one or more recombinant polynucleotides each encoding at least one HLA class II allele (e.g., two or more HLA-DRB alleles) selected from an HLA-DRB3*02:02 allele and an HLA-DRB5*01:01 allele, an HLA-DRB4*01:01 allele and an HLA-DRB3*01:01 allele, an HLA-DRB3*03:01 allele and an HLA-DRB5*01:02 allele, and/or an HLA-DRB5*02:02 allele and an HLA-DRB3*01:01 allele.

In some embodiments, the expression vector is capable of expressing the at least one immunomodulatory molecule, co-stimulatory molecule, or cytokine, the at least one HLA class I allele (e.g., an HLA-A allele), and/or the at least one HLA class II allele (e.g., an HLA-DRB allele) in a cancer cell line (e.g., a modified human cancer cell line). In some embodiments, the expression vector is capable of expressing one or more (e.g., at least two) immunomodulatory molecules, co-stimulatory molecules, or cytokines in a cancer cell line (e.g., a modified human cancer cell line); or the expression vector is capable of expressing one or more (e.g., at least two) HLA class I allele in a cancer cell line (e.g., a modified human cancer cell line); or the expression vector is capable of expressing one or more (e.g., at least two) HLA class II allele in a cancer cell line (e.g., a modified human cancer cell line). In various embodiments, one or more endogenous HLA-A and/or HLA-DR3 alleles in the cancer cell line have been inactivated. In some embodiments, at least one of an endogenous HLA-A*24:02 allele, an HLA-A*11:01 allele, an HLA-DRB3*01:01 allele, and/or an HLA-DRB3*02:02 allele in the cancer cell line has been inactivated.

In various embodiments, each of the one or more recombinant polynucleotides comprising a gene encoding an immunomodulatory molecule, co-stimulatory molecule, or cytokine selected from CSF2, IFNA2, CD86, IL-12, CD80, HLA-DRA, IL-7, and/or 4-1BBL, or any one of the HLA-A alleles, or any one of the HLA-DRB alleles (e.g., HLA-DRB3 alleles, HLA-DRB4 alleles, and HLA-DRB5 alleles) further comprises a 2A splicing peptide located between any of the two genes, any of the two HLA alleles, or a gene and an HLA allele.

In some embodiments, the expression vector comprises a recombinant polynucleotide encoding a cytokine, a chemokine, an interferon, an interleukin, and/or a tumor necrosis factor. In some embodiments, the recombinant polynucleotide encodes one of the cytokines selected from at least one of an early T cell activation antigen-1 (ETA-1), a lymphocyte-activating factor (LAF), an interleukin-1 family member (IL-1α, IL-β, IL-1Ra, IL-18, IL-33, IL-36Ra, IL-36α, IL-36β, IL-36γ, IL-37, IL-38), an interleukin-2 (IL-2), an interleukin-3 (IL-3), an interleukin-4 (IL-4), an interleukin-5 (IL-5), an interleukin-6 (IL-6), an interleukin-7 (IL-7), an interleukin-8 (IL-8), an interleukin-9 (IL-9), an interleukin-10 (IL-10), an interleukin-12 (IL-12), an interleukin-13 (IL-13), an interleukin-15 (IL-15), an interleukin-17 (IL-17), an interleukin-18 (IL-18), an interleukin-21 (IL-21), an interleukin-23 (IL-23), an interleukin-25 (IL-25), an interleukin-33 (IL-33), an interferon alpha (IFN-α), an interferon lambda 1 (IFNλ1 (IL-29)), an interferon lambda 2 (IFNλ2 (IL-28A)), an interferon lambda 3 (IFNλ3 (IL-28B)), an interferon lambda 4 (IFNλ4), a granulocyte-macrophage colony-stimulating factor (GM-CSF), a macrophage CSF (CSF-1), a macrophage migration inhibitory factor (MIF), a CD40L molecule (CD40L), a RANTES molecule (RANTES), a monocyte chemoattractant protein (MCP-1), a monocyte inflammatory protein (MIP-1α, MIP-1β), a lymphotactin, and/or a fractalkine. In some embodiments, the cytokine comprises a GM-CSF. In some embodiments, the cytokine comprises an IFN-α, such as e.g., IFN-α2a or IFN-α2b.

In some embodiments, the expression vector further comprises a recombinant polynucleotide encoding a co-stimulatory molecule selected from at least one of CD86 molecule (CD86), CD80 molecule (CD80), 4-1BB ligand molecule (4-1BBL a.k.a CD137L), ICOS ligand molecule (ICOS-L), CD70 molecule (CD70 a.k.a. CD27L), CD40 molecule (CD40), OX40 ligand molecule (OX40L), GITR ligand molecule (GITRL), TIM-4 molecule (TIM-4), LIGHT molecule (LIGHT), ICAM1 molecule (ICAM1), LFA3 molecule (LFA3), a CD30 molecule (CD30), and/or a combination thereof.

In some embodiments, the expression vector further comprises a recombinant polynucleotide encoding a heterologous antigen (e.g., an antigen of a pathogen, a tumor-associated antigen, a neo-antigen, an allergen, an antigen that is the target of an autoimmune response, or a fragment thereof).

In one aspect, the present disclosure provides an expression vector comprising a recombinant polynucleotide comprising a sequence encoding at least one allele of an HLA class I gene having at least 85%, at least 90%, at least 95%, at least 96%, at least 97%, at least 98%, or at least 99% identity to any one of SEQ ID NOS: 22-29 (Table 1) and optionally at least one heterologous polynucleotide sequence. In some embodiments, the present disclosure provides an expression vector comprising a recombinant polynucleotide comprising a sequence encoding at least one allele of an HLA class I gene having a polynucleotide of any one of SEQ ID NOS: 22-29 (Table 1) and optionally at least one heterologous polynucleotide sequence. In some embodiments, the recombinant polynucleotide further comprises a sequence encoding a second allele of the HLA class I gene or an allele of a second HLA class I gene. In some embodiments, the recombinant polynucleotide further comprises a sequence encoding an allele of an HLA class II gene. The allele of the HLA class II gene has at least 85%, at least 90%, at least 95%, at least 96%, at least 97%, at least 98%, or at least 99% identity to any one of SEQ ID NOS: 30-35 (Table 1). In some embodiments, the allele of the HLA class II gene has a polynucleotide of any one of SEQ ID NOS: 30-35 (Table 1).

In various embodiments, the heterologous polynucleotide sequence encodes an amino acid sequence of a cytokine, a chemokine, an interferon, an interleukin, or a tumor necrosis factor. In some embodiments, the heterologous polynucleotide sequence encodes one of the cytokines selected from at least one of an early T cell activation antigen-1 (ETA-1), a lymphocyte-activating factor (LAF), an interleukin-1 family member (IL-1α, IL-β, IL-1Ra, IL-18, IL-33, IL-36Ra, IL-36α, IL-36β, IL-36γ, IL-37, IL-38), an interleukin-2 (IL-2), an interleukin-3 (IL-3), an interleukin-4 (IL-4), an interleukin-5 (IL-5), an interleukin-6 (IL-6), an interleukin-7 (IL-7), an interleukin-8 (IL-8), an interleukin-9 (IL-9), an interleukin-10 (IL-10), an interleukin-12 (IL-12), an interleukin-13 (IL-13), an interleukin-15 (IL-15), an interleukin-17 (IL-17), an interleukin-18 (IL-18), an interleukin-21 (IL-21), an interleukin-23 (IL-23), an interleukin-25 (IL-25), an interleukin-33 (IL-33), an interferon alpha (IFN-α), an interferon lambda 1 (IFNλ1 (IL-29)), an interferon lambda 2 (IFNλ2 (IL-28A)), an interferon lambda 3 (IFNλ3 (IL-28B)), an interferon lambda 4 (IFNλ4), a granulocyte-macrophage colony-stimulating factor (GM-CSF), a macrophage CSF (CSF-1), a macrophage migration inhibitory factor (MIF), a CD40L molecule (CD40L), a RANTES molecule (RANTES), a monocyte chemoattractant protein (MCP-1), a monocyte inflammatory protein (MIP-1α, MIP-1β), a lymphotactin, and/or a fractalkine. In some embodiments, the cytokine comprises a GM-CSF. In some embodiments, the cytokine comprises an IFN-α, preferably IFN-α2a or IFN-α2b.

In some embodiments, the heterologous polynucleotide sequence encodes one of the co-stimulatory molecules selected from at least one of a CD86 molecule (CD86), CD80 molecule (CD80), 4-1BB ligand molecule (4-1BBL a.k.a CD137L), ICOS ligand molecule (ICOS-L), CD70 molecule (CD70 a.k.a. CD27L), CD40 molecule (CD40), OX40 ligand molecule (OX40L), GITR ligand molecule (GITRL), TIM-4 molecule (TIM-4), LIGHT molecule (LIGHT), ICAM1 molecule (ICAM1), LFA3 molecule (LFA3), a CD30 molecule (CD30), and/or a combination thereof.

In various embodiments, the heterologous polynucleotide sequence is located between sequences encoding two HLA class I genes, for example, the heterologous polynucleotide sequence is located at the 3′ end of the sequencing encoding a first HLA class I gene and at the 5′ end of the sequence encoding a second HLA class I gene. Similarly, the heterologous polynucleotide sequence may be located between sequences encoding two cytokines, two co-stimulatory molecules, or two heterologous antigens. In some embodiments, the heterologous polynucleotide sequence may be located between sequences encoding an HLA allele, a cytokine, a co-stimulatory molecule, and/or a heterologous antigen. In various embodiments, the heterologous polynucleotide sequence encodes one of the 2A splicing peptides selected from T2A, P2A, or E2A.

### C. Compositions

In one aspect, the present disclosure provides a composition comprising a modified human cancer cell as described herein, the modified human cancer comprising (a) one or more recombinant polynucleotides each encoding an allele of a human leukocyte antigen (HLA) class I gene; and/or (b) one or more recombinant polynucleotides each encoding an allele of an HLA class II gene, wherein one or more HLA alleles endogenous to the cell have been inactivated. In some embodiments, the HLA class I gene(s) and HLA class II genes(s) are selected from any of SEQ ID NOS:1-35 listed in Table 1.

In one aspect, the present disclosure provides a composition comprising a modified human cancer cell as described herein, the modified human cancer cell comprising (A) one or more vectors each comprising a recombinant polynucleotide encoding at least one gene selected from CSF2, IFN-α2, CD86, IL-12, CD80, HLA-DRA, IL-7, and/or 4-1BBL (also known as TNFSF9 or CD137L); (B) (a) one or more vectors each comprising a recombinant polynucleotide encoding at least one gene selected from HLA-A*01:01, HLA-A*68:01, HLA-A*02:01, HLA-A*11:01, HLA-A*03:01, HLA-A*23:01, HLA-A*24:02, and/or HLA-A*33:03, or (b) a vector comprising a recombinant polynucleotide encoding an HLA-A*01:01 allele and an HLA-A*68:01 allele, or an HLA-A*02:01 allele and an HLA-A*11:01 allele, or an HLA-A*03:01 allele and an HLA-A*23:01 allele, or an HLA-A*24:02 allele and an HLA-A*33:03 allele; and (C) (a) one or more vectors comprising a recombinant polynucleotide encoding at least one gene selected from HLA-DRB3*02:02, HLA-DRB5*01:01, HLA-DRB4*01:01, HLA-DRB3*01:01, HLA-DRB3*03:01, HLA-DRB5*01:02, and/or HLA-DRB5*02:02, or (b) a vector comprising a recombinant polynucleotide encoding an HLA-DRB3*02:02 allele and an HLA-DRB5*01:01 allele, or an HLA-DRB4*01:01 allele and an HLA-DRB3*01:01 allele, or an HLA-DRB3*03:01 allele and an HLA-DRB5*01:02 allele, or an HLA-DRB5*02:02 allele and an HLA-DRB3*01:01 allele. In some embodiments, one or more HLA-A and/or HLA-DR3 (e.g., HLA-DRB3) alleles endogenous to the human cancer cell have been inactivated. In some embodiments, at least one of an endogenous HLA-A*24:02 allele, an HLA-A*11:01 allele, an HLA-DRB3*01:01 allele, and/or an HLA-DRB3*02:02 allele endogenous to the human cancer cell has been inactivated.

In some embodiments, the composition comprises at least 10,000 cells, at least 100,000 cells, at least 1,000,000 cells, at least 1,250,000 cells, at least 1,500,000 cells, at least 2,000,000 cells, at least 2,500,000 cells, at least 3,000,000 cells, at least 3,500,000 cells, at least 4,000,000 cells, at least 4,500,000 cells, at least 5,000,000 cells, at least 10,000,000 cells, at least 12,500,000 cells, at least 15,000,000 cells, at least 20,000,000 cells, at least 25,000,000 cells, at least 30,000,000 cells, at least 35,000,000 cells, at least 40,000,000 cells, at least 45,000,000 cells, or at least 50,000,000 cells. In some embodiments, the composition comprises at least 1,000,000 cells. In some embodiments, the composition comprises at least 20,000,000 cells.

In some embodiments, the composition comprises at most 10,000 cells, at most 100,000 cells, at most 1,000,000 cells, at most 1,250,000 cells, at most 1,500,000 cells, at most 2,000,000 cells, at most 2,500,000 cells, at most 3,000,000 cells, at most 3,500,000 cells, at most 4,000,000 cells, at most 4,500,000 cells, at most 5,000,000 cells, at most 10,000,000 cells, at most 12,500,000 cells, at most 15,000,000 cells, at most 20,000,000 cells, at most 25,000,000 cells, at most 30,000,000 cells, at most 35,000,000 cells, at most 40,000,000 cells, at most 45,000,000 cells, or at most 50,000,000 cells. In some embodiments, the composition comprises at most 20,000,000 cells. In some embodiments, the composition comprises at most 40,000,000 cells.

In some embodiments, the composition comprises about 1,000,000 to about 50,000,000 cells, about 5,000,000 to about 35,000,000 cells, about 10,000,000 to about 25,000,000 cells, about 15,000,000 to about 20,000,000 cells, or about 35,000,000 to about 40,000,000 cells. In some embodiments, the composition comprises about 1,000,000 cells. In some embodiments, the composition comprises about 20,000,000 cells. In some embodiments, the composition comprises about 40,000,000 cells.

In another aspect, the present disclosure provides a pharmaceutical composition. In some embodiments, the pharmaceutical composition comprises any of the compositions described herein and a pharmaceutically acceptable carrier. For example, the pharmaceutical composition may comprise a modified human cancer cell or cell line comprising at least 1, 2, 3, 4, 5, or more recombinant polynucleotides encoding at least one HLA class I allele having SEQ ID NOS:1-13, 22-29 in Table 1 and at least one HLA class II allele having SEQ ID NOS: 14-21, 30-35 in Table 1, and one or more endogenous HLA alleles have been inactivated. Additionally, the CD47 molecule and/or Ii(CD74) molecule endogenous to the modified human cancer cell or cell line may be inactivated. Further, the modified human cancer cell or cell line may comprise one or more co-stimulatory molecules, heterologous antigens, and/or cytokine described herein (e.g., Table 3, FIG. 5). The at least 1, 2, 3, 4, 5, or more recombinant polynucleotides may comprise a heterologous sequence encoding, e.g., a co-stimulatory molecule, a heterologous antigen, a cytokine, or a 2A splicing peptide. Thus, the recombinant polynucleotide encoding the one or more HLA alleles, co-stimulatory molecule, heterologous antigen, and/or cytokine may be separated by a sequencing encoding a 2A splicing peptide (e.g., T2A, P2A, E2A). Generally, at least 1, 2, 3, 4, 5, or more recombinant polynucleotides are cloned into an expression vector (e.g., a replication defective lentiviral vector) for synthesis of the HLA alleles, co-stimulatory molecule, heterologous antigen, and/or cytokine, and introduced into the modified human cancer cell or cell line. Accordingly, the modified human cancer cell or cell line provided in the pharmaceutical composition may have at least 1, 2, 3, 4, 5, or more expression vectors, each comprising at least 1, 2, 3, 4, 5 or more recombinant polynucleotides encoding the HLA alleles, co-stimulatory molecule, heterologous antigen, and/or cytokine.

In some embodiments, the pharmaceutical composition further comprises a cryoprotectant, an interferon alpha (e.g., IFN-α2a or IFN-α2b), and/or an interferon lambda family member (e.g., an interferon lambda 1 (IFNλ1 (IL-29)), an interferon lambda 2 (IFNλ2 (IL-28A)), an interferon lambda 3 (IFNλ1 (IL-28B)), an interferon lambda 4 (IFNλ4)). In some embodiments, the interferon alpha (e.g., IFN-α2a or IFN-α2b) is expressed by a vector comprising a polynucleotide sequence of the IFNA2 gene in the modified cancer cell as described herein. In some embodiments, the interferon alpha is a pegylated IFN-α2a provided exogenously. In some embodiments, the pharmaceutical composition further comprises one or more excipients. In some embodiments, the pharmaceutical composition further comprises CryoStor CS10, CryoStor CS2, or CryoStor CS5 cryopreservation media. In particular embodiments, the pharmaceutical composition comprises cells cryopreserved in CryoStor CS10, CryoStor CS2, or CryoStor CS5 cryopreservation media.

In some embodiments, the pharmaceutical composition is formulated in a dosage form comprising a total number of modified cancer cell per dose for administration to a subject in need therefor. In some embodiments, the pharmaceutical composition is formulated as an “off-the-shelf” product for self-administration to a subject in need thereof. In some embodiments, the pharmaceutical composition may have at least at least 10,000 cells, at least 100,000 cells, at least 1,000,000 cells, at least 1,250,000 cells, at least 1,500,000 cells, at least 2,000,000 cells, at least 2,500,000 cells, at least 3,000,000 cells, at least 3,500,000 cells, at least 4,000,000 cells, at least 4,500,000 cells, at least 5,000,000 cells, at least 10,000,000 cells, at least 12,500,000 cells, at least 15,000,000 cells, at least 20,000,000 cells, at least 25,000,000 cells, at least 30,000,000 cells, at least 35,000,000 cells, at least 40,000,000 cells, at least 45,000,000 cells, or at least 50,000,000 cells. In some embodiments, the pharmaceutical composition comprises at least 1,000,000 cells. In some embodiments, the pharmaceutical composition comprises at least 20,000,000 cells.

In some embodiments, the pharmaceutical composition comprises at most 10,000 cells, at most 100,000 cells, at most 1,000,000 cells, at most 1,250,000 cells, at most 1,500,000 cells, at most 2,000,000 cells, at most 2,500,000 cells, at most 3,000,000 cells, at most 3,500,000 cells, at most 4,000,000 cells, at most 4,500,000 cells, at most 5,000,000 cells, at most 10,000,000 cells, at most 12,500,000 cells, at most 15,000,000 cells, at most 20,000,000 cells, at most 25,000,000 cells, at most 30,000,000 cells, at most 35,000,000 cells, at most 40,000,000 cells, at most 45,000,000 cells, or at most 50,000,000 cells. In some embodiments, the pharmaceutical composition comprises at most 20,000,000 cells. In some embodiments, the pharmaceutical composition comprises at most 40,000,000 cells.

In some embodiments, the pharmaceutical composition comprises about 1,000,000 to about 50,000,000 cells, about 5,000,000 to about 35,000,000 cells, about 10,000,000 to about 25,000,000 cells, about 15,000,000 to about 20,000,000 cells, or about 35,000,000 to about 40,000,000 cells. In some embodiments, the pharmaceutical composition comprises about 1,000,000 cells. In some embodiments, the pharmaceutical composition comprises about 20,000,000 cells. In some embodiments, the pharmaceutical composition comprises about 40,000,000 cells.

In some embodiments, the pharmaceutical composition is formulated in the form of a suspension. The formulation of pharmaceutical compositions is generally known in the art (see, e.g., REMINGTON'S PHARMACEUTICAL SCIENCES, 18TH ED., Mack Publishing Co., Easton, PA (1990)). Prevention against microorganism contamination can be achieved through the addition of one or more of various antibacterial and antifungal agents. In particular embodiments, the pharmaceutical composition is a liquid formulation comprising cells resuspended in Lactated Ringer's solution.

Pharmaceutical forms suitable for administration include sterile aqueous solutions or dispersions and sterile powders for the extemporaneous preparation of sterile injectable solutions or dispersions. Typical carriers include a solvent or dispersion medium containing, for example, water-buffered aqueous solutions (i.e., biocompatible buffers, non-limiting examples of which include Lactated Ringer's solution and CryoStor cryopreservation media (e.g., CS2, CS5, and CS10, containing 2%, 5%, and 10%, respectively of DMSO; available from BioLife Solutions, Bothell, WA)), ethanol, polyols such as glycerol, propylene glycol, polyethylene glycol, suitable mixtures thereof, surfactants, or vegetable oils.

Sterilization can be accomplished by an art-recognized technique, including but not limited to addition of antibacterial or antifungal agents, for example, paraben, chlorobutanol, sorbic acid or thimerosal. Further, isotonic agents such as sugars or sodium chloride may be incorporated in the subject compositions.

Production of sterile injectable solutions containing modified cancer cell(s), and/or other composition(s) of the present disclosure can be accomplished by incorporating the compound(s) in the required amount(s) in the appropriate solvent with various ingredients enumerated above, as required, followed by sterilization. To obtain a sterile powder, the above sterile solutions can be vacuum-dried or freeze-dried as necessary.

In some embodiments, the modified cancer cell(s), and/or other composition(s) provided herein are formulated for administration, e.g., intradermal injection, intralymphatic injection, oral, nasal, topical, or parental administration in unit dosage form for ease of administration and uniformity of dosage. Unit dosage forms, as used herein, refers to physically discrete units suited as unitary dosages for the subjects, e.g., humans or other mammals to be treated, each unit containing a predetermined quantity of active material calculated to produce the desired therapeutic effect in association with the required pharmaceutical carrier. In some instances, more concentrated dosage forms may be prepared, from which the more dilute unit dosage forms may then be produced. The more concentrated dosage forms thus will contain substantially more than, e.g., at least 1, 2, 3, 4, 5, 6, 7, 8, 9, 10, or more times the amount of the modified cancer cell(s), and/or other composition(s).

In some embodiments, the modified cancer cell(s), and/or other composition(s) provided herein are formulated for administration e.g., one or more doses over a period of time. In some embodiments, the modified cancer cell(s), and/or other composition(s) are formulated for administration every week, every 2 weeks, every 3 weeks, every 4 weeks, every 5 weeks, or every 6 weeks. In some embodiments, the modified cancer cell(s), and/or other composition(s) are formulated for administration every month, every 2 months, every 3 months, every 4 months, every 5 months, every 6 months, every 12 months, every 18 months, or every 24 months.

A dose may include, for example, about 50,000 to 50,000,000 (e.g., about 50,000, 60,000, 70,000, 80,000, 90,000, 100,000, 110,000, 120,000, 130,000, 140,000, 150,000, 160,000, 170,000, 180,000, 190,000, 200,000, 250,000, 300,000, 350,000, 400,000, 450,000, 500,000, 550,000, 600,000, 650,000, 700,000, 750,000, 800,000, 850,000, 900,000, 950,000, 1,000,000, 1,500,000, 2,000,000, 2,500,000, 3,000,000, 3,500,000, 4,000,000, 4,500,000, 5,000,000, 5,500,000, 6,000,000, 6,500,000, 7,000,000, 7,500,000, 8,000,000, 8,500,000, 9,000,000, 9,500,000, 10,000,000, 11,000,000, 12,000,000, 13,000,000, 14,000,000, 15,000,000, 16,000,000, 17,000,000, 18,000,000, 19,000,000, 20,000,000, 25,000,000, 30,000,000, 35,000,000, 40,000,000, 45,000,000, 50,000,000, or more) modified human cancer cells. In some embodiments, a dose may contain about 1,000,000 modified human cancer cells. In some embodiments, a dose may contain about 5,000,000 modified human cancer cells. In some embodiments, a dose may contain about 10,000,000 modified human cancer cells. In some embodiments, a dose may contain about 20,000,000 modified human cancer cells.

A dose may also include, for example, at least about 5,000,000 to 100,000,000 (e.g., about 5,000,000, 6,000,000, 7,000,000, 8,000,000, 9,000,000, 10,000,000, 15,000,000, 20,000,000, 25,000,000, 30,000,000, 35,000,000, 40,000,000, 45,000,000, 50,000,000, 55,000,000, 60,000,000, 65,000,000, 70,000,000, 75,000,000, 80,000,000, 85,000,000, 90,000,000, 95,000,000, 100,000,000, or more) modified human cancer cells. In some embodiments, a dose may include at least about 1,000,000 modified human cancer cells. In some embodiments, a dose may include at least about 5,000,000 modified human cancer cells. In some embodiments, a dose may include at least about 10,000,000 modified human cancer cells. In some embodiments, a dose may include at least about 20,000,000 modified human cancer cells.

A dose may alternatively include, for example, at least about 100,000,000 to 1,000,000,000 (e.g., about 100,000,000, 150,000,000, 200,000,000, 250,000,000, 300,000,000, 350,000,000, 400,000,000, 450,000,000, 500,000,000, 550,000,000, 600,000,000, 650,000,000, 700,000,000, 750,000,000, 800,000,000, 850,000,000, 900,000,000, 950,000,000, 1,000,000,000, or more) modified human cancer cells.

In some embodiments, the modified human cancer cells are irradiated. The irradiation dose may be, for example, between about 2 and 2,000 Gy (e.g., about, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, 1,000, 1,100, 1,200, 1,300, 1,400, 1,500, 1,600, 1,700, 1,800, 1,900, or 2,000 Gy). In some embodiments, the modified human cancer cells are irradiated with a dose of about 200 Gy. In some embodiments, the modified human cancer cells are irradiated with a dose of about 100 Gy.

Methods for preparing such dosage forms are known to those skilled in the art (see, e.g., REMINGTON'S PHARMACEUTICAL SCIENCES, supra). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., REMINGTON'S PHARMACEUTICAL SCIENCES, supra).

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates (i.e., the parabens); pH adjusting agents such as inorganic and organic acids and bases; sweetening agents; and flavoring agents. The dosage forms may also comprise biodegradable polymer beads, dextran, and cyclodextrin inclusion complexes.

In some embodiments, the pharmaceutical composition for administration may be an oral delivery vehicle such as a capsule, cachet or tablet, each of which contains a predetermined amount of the composition to provide the correct incremental dose to the patient. Oral delivery vehicles may be useful, for example, in avoiding contact between the composition and the mouth and upper gastrointestinal tract. For oral administration, the therapeutically effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

In some embodiments, the therapeutically effective dose takes the form of a pill, tablet, or capsule, and thus, the dosage form can contain, along with the modified cancer cell(s), and/or other composition(s) described herein, any of the following: a diluent such as lactose, sucrose, dicalcium phosphate, and the like; a disintegrant such as starch or derivatives thereof; a lubricant such as magnesium stearate and the like; and a binder such a starch, gum acacia, polyvinylpyrrolidone, gelatin, cellulose and derivatives thereof.

In some embodiments, a suitable carrier masks the composition, e.g., the modified cancer cell(s), and/or other composition(s) from the mouth and upper gastrointestinal (GI) tract and reduces or prevents local itching/swelling reactions in these regions during administration. For example, a carrier may contain one or more lipid, polysaccharide or protein constituents. In some cases, the carrier is a food product.

For topical administration, the therapeutically effective dose can be in the form of emulsions, lotions, gels, foams, creams, jellies, solutions, suspensions, ointments, and transdermal patches. For administration by inhalation, the modified cancer cell(s), and/or other composition(s) described herein can be delivered as a dry powder or in liquid form via a nebulizer. Aerosol formulations can be placed into pressurized acceptable propellants such as dichlorodifluoromethane. For parenteral administration, the therapeutically effective dose can be in the form of sterile injectable solutions and sterile packaged powders. Preferably, injectable solutions are formulated at a pH of from about 4.5 to about 7.5.

The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water. The lyophilized dosage form may further comprise a suitable vasoconstrictor, e.g., epinephrine. The lyophilized dosage form can be provided in a syringe, optionally packaged in combination with the buffer for reconstitution, such that the reconstituted dosage form can be immediately administered to an individual.

In some embodiments, the therapeutically effective dose may further comprise other components, for example, anti-allergy drugs, such as antihistamines, steroids, bronchodilators, leukotriene stabilizers and mast cell stabilizers. Suitable anti-allergy drugs are well known in the art.

### D. Methods for Treating Cancer

In another aspect, the present disclosure provides a method for treating cancer in a subject. In some embodiments, the method comprises administering to the subject a therapeutically effective amount of a pharmaceutical composition of the present disclosure (e.g., a pharmaceutical composition comprising modified cancer cells of the present disclosure) described herein.

In some embodiments, the method comprising administering to the subject an effective amount of the pharmaceutical composition intradermally in the upper back or thighs. Without being bound by any theories, the upper back and thighs are chosen for patient acceptability as these areas have less nerves in the skin and are thus less sensitive. Additionally, the draining lymph nodes in the proximity may convey antigens from breast tumors in the upper and lower torso, which are common sites for breast cancer metastases. The method may further comprise administering to the subject the pharmaceutical composition in an interval of every week, every 2 weeks, every 3 weeks, every 4 weeks, every 5 weeks, or every 6 weeks. In some embodiments, the method comprises administering to be subject the pharmaceutical composition in an interval of every month, every 2 months, every 3 months, every 4 months, every 5 months, every 6 months, every 12 months, every 18 months, or every 24 months. In some embodiments, the method comprises administering to the subject the pharmaceutical composition for at least 6 weeks, 12 weeks, 24 weeks, 36 weeks, 48 weeks, 52 weeks or longer. In some embodiments, the method comprises administering to the subject the pharmaceutical composition for at least 1 month, 2 months, 3 months, 4 months, 5 months, 6 months, 8 months, 10 months, 12 months, or longer. In some embodiments, the method comprises administering to the subject the pharmaceutical composition for not more than 6 weeks, 12 weeks, 24 weeks, 36 weeks, 48 weeks, or 52 weeks. In some embodiments, the method comprises administering to the subject the pharmaceutical composition for not more than 1 month, 2 months, 3 months, 4 months, 5 months, 6 months, 8 months, 10 months, or 12 months.

In some embodiments, the method comprising administering to the subject an effective amount of the pharmaceutical composition through oral administration, topical contact, administration as a suppository, intravenous, intraperitoneal, intramuscular, intralesional, intratumoral, intradermal, intralymphatic, intrathecal, intranasal, or subcutaneous administration. In some embodiments, administration of the effective amount of the pharmaceutical composition is performed by parenteral administration (e.g., intravenous, intramuscular, intra-arteriole, intradermal, subcutaneous, intraperitoneal, intraventricular, and intracranial) or transmucosal administration (e.g., buccal, sublingual, palatal, gingival, nasal, vaginal, rectal, or transdermal). In some embodiments, the method comprising the use of liposomal formulations, intravenous infusion, or transdermal patches.

In some embodiments, the method further comprising administering to the subject one or more doses of cyclophosphamide intravenously at least 1 day, at least 2 days, at least 3 days, at least 4 days, at least 5 days, or longer, prior to administering to the subject the pharmaceutical composition described herein. In some embodiments, the cyclophosphamide is administered at least about 2-3 days prior to administering to the subject the pharmaceutical composition described herein. In some embodiments, a low-dose of cyclophosphamide at about 100, 150, 200, 250, 300, or 450 mg/m² is administered to the subject.

In some embodiments, the method further comprising administering to the subject one or more doses of an interferon-alpha-2b (IFN-α2b), IFN-α2a, or a pegylated IFN-α2a intradermally at the inoculation site of the pharmaceutical composition described herein. In some embodiments, the method further comprising administering to the subject one or more doses of IFN-α2b, IFN-α2a or pegylated IFN-α2a intradermally not more than 1, 2, 3, 4, 5, 6, 7, 8, 9, 10, 12, 24, 48, 60, 72, or 84 hours following administering to the subject the pharmaceutical composition described herein. In some embodiments, the method further comprising administering to the subject one or more doses of IFN-α2b, IFN-α2a or pegylated IFN-α2a intradermally at about 1-4 hours, about 2-6 hours, about 8-12 hours, about 10-24 hours, about 20-48 hours, or about 60-72 hours following administering to the subject the pharmaceutical composition described herein. In some embodiments, the method further comprising administering to the subject one or more doses of IFN-α2b, IFN-α2a or pegylated IFN-α2a intradermally no later than 5, 10, 15, 20, 25, 30, 45, 50, 60, 72, or 84 hours after administering to the subject the pharmaceutical composition. In some embodiments, the method further comprising administering to the subject one or more doses of IFN-α2b, IFN-α2a or pegylated IFN-α2a intradermally no later than 1, 2, 3, 4, 5, or 6 days following administering to the subject the pharmaceutical composition. In some embodiments, the method further comprising administering to the subject one or more doses of IFN-α2b, IFN-α2a or pegylated IFN-α2a intradermally no later than about 1-6 days, 2-3 days, or 3-5 days following administering to the subject the pharmaceutical composition. In some embodiments, the method further comprising administering to the subject a first doses of IFN-α2bIFN-α2b, IFN-α2a or pegylated IFN-α2a intradermally between 1 to 4 hours and a second dose of IFN-α2b, IFN-α2a or pegylated IFN-α2a intradermally between 1-3 days following administering to the subject the pharmaceutical composition. In some embodiments, the IFN-α2b, administered is at a low-dose between about 1-20,000 IU, 100-15,000 IU, 5000-12,000 IU, or 9,000-11,000 IU. In some embodiments, the IFN-α2b administered is at dose of about 10,000 IU. In some embodiments, the IFN-α2a or pegylated IFN-α2a, administered is at a low-dose between about 0.01-0.1 micrograms (mcg), 0.05-0.15 mcg, 0.06-0.12 mcg, or 0.09-0.11 mcg. In some embodiments, the IFN-α2b administered is at dose of about 0.1 mcg.

In some embodiments, the method further comprises administering to the subject one or more additional therapies. Examples of suitable additional types include, but are not limited to, chemotherapy, immunotherapy, radiotherapy, hormone therapy, a differentiating agent, and a small-molecule drug. One of skill in the art will readily be able to select an appropriate additional therapy.

Chemotherapeutic agents that can be used in the present disclosure include but are not limited to alkylating agents (e.g., nitrogen mustards (e.g., mechlorethamine, chlorambucil, cyclophosphamide, ifosfamide, melphalan), nitrosoureas (e.g., streptozocin, carmustine (BCNU), lomustine), alkyl sulfonates (e.g., busulfan), triazines (e.g., dacarbazine (DTIC), temozlomide), ethylenimines (e.g., thiotepa, altretamine (hexamethylmelamine)), platinum drugs (e.g., cisplatin, carboplatin, oxalaplatin), antimetabolites (e.g., 5-fluorouracil (5-FU), 6-mercaptopurine (6-MP), capecitabine, cytarabine, floxuridine, fludarabine, gemcitabine, hydroxyurea, methotrexate, pemetrexed), anthracycline anti-tumor antibiotics (e.g., daunorubicin, doxorubicin, epirubicin, idarubicin), non-anthracycline anti-tumor antibiotics (e.g., actinomycin-D, bleomycin, mitomycin-C, mitoxantrone), mitotic inhibitors (e.g., taxanes (e.g., paclitaxel, docetaxel), epothilones (e.g., ixabepilone), vinca alkaloids (e.g., vinblastine, vincristine, vinorelbine), estramustine, corticosteroids (e.g., prednisone, methylprednisolone, dexamethasone), L-asparaginase, bortezomib, and topoisomerase inhibitors. Combinations of chemotherapeutic agents can be used.

Topoisomerase inhibitors are compounds that inhibit the activity of topoisomerases, which are enzymes that facilitate changes in DNA structure by catalyzing the breaking and rejoining of phosphodiester bonds in the backbones of DNA strands. Such changes in DNA structure are necessary for DNA replication during the normal cell cycle. Topoisomerase inhibitors inhibit DNA ligation during the cell cycle, leading to an increased number of single- and double-stranded breaks and thus a degradation of genomic stability. Such a degradation of genomic stability leads to apoptosis and cell death.

Topoisomerases are often divided into type I and type II topoisomerases. Type I topoisomerases are essential for the relaxation of DNA supercoiling during DNA replication and transcription. Type I topoisomerases generate DNA single-strand breaks and also religate said breaks to re-establish an intact duplex DNA molecule. Examples of inhibitors of topoisomerase type I include irinotecan, topotecan, camptothecin, and lamellarin D, which all target type IB topoisomerases.

Type II topoisomerase inhibitors are broadly classified as topoisomerase poisons and topoisomerase inhibitors. Topoisomerase poisons target topoisomerase-DNA complexes, while topoisomerase inhibitors disrupt enzyme catalytic turnover. Examples of type II topoisomerase inhibitors include amsacrine, etoposide, etoposide phosphate, teniposide, doxorubicin, and fluoroquinolones.

In some embodiments, the chemotherapeutic agent is a topoisomerase inhibitor. In some instances, the topoisomerase inhibitor is a topoisomerase I inhibitor, a topoisomerase II inhibitor, or a combination thereof. In particular embodiments, the topoisomerase inhibitor is selected from the group consisting of doxorubicin, etoposide, teniposide, daunorubicin, mitoxantrone, amsacrine, an ellipticine, aurintricarboxylic acid, HU-331, irinotecan, topotecan, camptothecin, lamellarin D, resveratrol, genistein, quercetin, epigallocatechin gallate (EGCG), and a combination thereof. EGCG is one example of a plant-derived natural phenol that serves as a suitable topoisomerase inhibitor. In some instances, the topoisomerase inhibitor is doxorubicin.

Immunotherapy refers to any treatment that uses the subject's immune system to fight a disease (e.g., cancer). Immunotherapy methods can be directed to either enhancing or suppressing immune function. In the context of cancer therapies, immunotherapy methods are typically directed to enhancing or activating immune function. In some instances, an immunotherapeutic agent comprises a monoclonal antibody that targets a particular type or part of a cancer cell. In some cases, the antibody is conjugated to a moiety such as a drug molecule or a radioactive substance. Antibodies can be derived from mouse, chimeric, or humanized, as non-limiting examples. Non-limiting examples of therapeutic monoclonal antibodies include alemtuzumab, bevacizumab, cetuximab, daratumumab, ipilimumab (MDX-101), nivolumab, ofatumumab, panitumumab, pembrolizumab, retifanlimab, rituximab, tositumomab, and trastuzumab.

Immunotherapeutic agents can also comprise an immune checkpoint inhibitor, which modulates the ability of the immune system to distinguish between normal and “foreign” cells. Programmed cell death protein 1 (PD-1) and protein death ligand 1 (PD-L1) are common targets of immune checkpoint inhibitors, as disruption of the interaction between PD1 and PD-L1 enhance the activity of immune cells against foreign cells such as cancer cells. Examples of PD-1 inhibitors include pembrolizumab, retifanlimab and nivolumab. An example of a PD-L1 inhibitor is atezolizumab.

Another immune checkpoint target for the treatment of cancer is cytotoxic T lymphocyte-associated protein 4 (CTLA-4), which is a receptor that downregulates immune cell responses. Therefore, drugs that inhibit CTLA-4 can increase immune function. An example of such a drug is ipilimumab, which is a monoclonal antibody that binds to and inhibits CTLA-4.

The term “radiotherapy” refers to the delivery of high-energy radiation to a subject for the treatment of a disease (e.g., cancer). Radiotherapy can comprise the delivery of X-rays, gamma rays, and/or charged particles. Radiotherapy can be delivered locally (e.g. to the site or region of a tumor), or systemically (e.g., a radioactive substance such as radioactive iodine is administered systemically and travels to the site of the tumor).

The term “hormone therapy” can refer to an inhibitor of hormone synthesis, a hormone receptor antagonist, or a hormone supplement agent. Inhibitors of hormone synthesis include but are not limited to aromatase inhibitors and gonadotropin releasing hormone (GnRH) analogs. Hormone receptor antagonists include but are not limited to selective receptor antagonists and antiandrogen drugs. Hormone supplement agents include but are not limited to progestogens, androgens, estrogens, and somatostatin analogs. Aromatase inhibitors are used, for example, to treat breast cancer. Non-limiting examples include letrozole, anastrozole, and aminoglutethimide. GnRH analogs can be used, for example, to induce chemical castration. Selective estrogen receptor antagonists, which are commonly used for the treatment of breast cancer, include tamoxifen, raloxifene, toremifene, and fulvestrant. Antiandrogen drugs, which bind to and inhibit the androgen receptor, are commonly used to inhibit the growth and survival effects of testosterone on prostate cancer. Non-limiting examples include flutamide, apalutamide, and bicalutamide.

The term “differentiating agent” refers to any substance that promotes cell differentiation, which in the context of cancer can promote malignant cells to assume a less stem cell-like state. A non-limiting example of an anti-cancer differentiating agent is retinoic acid.

Small molecule drugs generally are pharmacological agents that have a low molecular weight (i.e., less than about 900 daltons). Non-limiting examples of small molecule drugs used to treat cancer include bortezomib (a proteasome inhibitor), imatinib (a tyrosine kinase inhibitor), and seliciclib (a cyclin-dependent kinase inhibitor), and epacadostat (an indoleamine 2,3-dioxygenase (IDO1) inhibitor).

In some embodiments, the method of treating cancer of the present disclosure further comprises selecting a whole-cell cancer vaccine for the subject according to a method of the present disclosure described herein. In particular embodiments, the subject has stage I, stage II, stage III, and/or stage IV cancer. In other embodiments, the cancer is transitioning between stages. In some embodiments, the subject has a pre-cancerous lesion. In some embodiments, the subject does not have cancer.

In some embodiments, treating the subject comprises inhibiting cancer cell growth, inhibiting cancer cell proliferation, inhibiting cancer cell migration, inhibiting cancer cell invasion, ameliorating or eliminating the symptoms of cancer, reducing the size (e.g., volume) of a cancer tumor, reducing the number of cancer tumors, reducing the number of cancer cells, inducing cancer cell necrosis, pyroptosis, oncosis, apoptosis, autophagy, or other cell death, or enhancing the therapeutic effects of a composition or pharmaceutical composition. In some embodiments, treating the subject results in an increased survival time. In some instances, overall survival is increased. In other instances, disease-free survival is increased. In some instances, progression-free survival is increased. In particular embodiments, treating the subject results in a reduction in tumor volume and/or increased survival time.

In particular embodiments, treating the subject enhances the therapeutic effects of an anti-cancer therapy such as a chemotherapeutic agent, an immunotherapeutic agent, radiotherapy, hormone therapy, a differentiating agent, and/or a small-molecule drug.

Therapy such as modified cancer cell(s), composition(s), and pharmaceutical composition(s) of the present disclosure can be administered using routes, dosages, and protocols that will readily be known to one of skill in the art. Administration can be conducted once per day, once every two days, once every three days, once every four days, once every five days, once every six days, or once per week. Therapy can be administered 1, 2, 3, 4, 5, 6, 7, 8, 9, 10, 11, 12, 13, 14, or more times per week. In some cases, modified cancer cell(s), composition(s), and/or pharmaceutical composition(s) of the present disclosure are administered as a single dose, co-administered (e.g., administered in separate doses or by different routes, but close together in time), or administered separately (e.g., administered in different doses, including the same or different route, but separated by about 1, 2, 3, 4, 5, 6, 7, 8, 9, 10, 11, 12, or more hours). In cases where multiple doses are to be administered in the same day, or where a single dose comprises one or more components (e.g., the modified cancer cell(s) and IFNa are administered separately), administration can occur, for example, 1, 2, 3, 4, 5, 6, 7, 8, 9, 10, 11, 12, or more times in a day.

In some cases, therapeutic administration can occur about once per week, about every two weeks, about every three weeks, or about once per month. In other cases, therapeutic administration can occur about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more times per month. Treatment can continue for about 1, 2, 3, 4, 5, 6, 7, 8, 9, 10, 11, 12, or more weeks; about 1, 2, 3, 4, 5, 6, 7, 8, 9, 10, 11, 12 or more months; or longer. At any time during treatment, the therapeutic plan can be adjusted as necessary. For example, depending on the response to modified cancer cell(s), compositions, or pharmaceutical composition(s) of the present disclosure, a different vaccine may be selected, one or more additional therapeutic agents or drugs may be chosen, or any aspect of the therapeutic plan can be discontinued. One of skill in the art will readily be able to make such decisions, which can be informed by, for example, the results of allele profile comparison, changes in the activity and/or number of an immune cell, and/or changes in the the presence or level of one or more biomarkers.

The modified cancer cell(s), composition(s), and pharmaceutical composition(s) of the present disclosure can be administered by any suitable route, including those described herein. In some embodiments, the administration is by intradermal or intralymphatic injection. In some embodiments, the whole-cell cancer vaccine (e.g., comprising modified cancer cells of the present disclosure) is given separately from interferon alpha (IFNa). In some instances, the IFNa is injected locally. IFNa can be given before and/or after the vaccine is administered. Timing of the separate injections can be any suitable interval, including those described herein.

One of skill in the art will readily be able to administer the number of appropriate modified cancer cells to include in a particular dose. A dose may include, for example, about 50,000 to 50,000,000 (e.g., about 50,000, 60,000, 70,000, 80,000, 90,000, 100,000, 110,000, 120,000, 130,000, 140,000, 150,000, 160,000, 170,000, 180,000, 190,000, 200,000, 250,000, 300,000, 350,000, 400,000, 450,000, 500,000, 550,000, 600,000, 650,000, 700,000, 750,000, 800,000, 850,000, 900,000, 950,000, 1,000,000, 1,500,000, 2,000,000, 2,500,000, 3,000,000, 3,500,000, 4,000,000, 4,500,000, 5,000,000, 5,500,000, 6,000,000, 6,500,000, 7,000,000, 7,500,000, 8,000,000, 8,500,000, 9,000,000, 9,500,000, 10,000,000, 11,000,000, 12,000,000, 13,000,000, 14,000,000, 15,000,000, 16,000,000, 17,000,000, 18,000,000, 19,000,000, 20,000,000, 25,000,000, 30,000,000, 35,000,000, 40,000,000, 45,000,000, 50,000,000, or more) modified cancer cells. In some embodiments a dose may contain about 1,000,000 modified cancer cells. In some embodiments a dose may contain about 5,000,000 modified cancer cells. In some embodiments a dose may contain about 10,000,000 modified cancer cells. In some embodiments a dose may contain about 20,000,000 modified cancer cells.

A dose may also include, for example, at least about 5,000,000 to 100,000,000 (e.g., about 5,000,000, 6,000,000, 7,000,000, 8,000,000, 9,000,000, 10,000,000, 15,000,000, 20,000,000, 25,000,000, 30,000,000, 35,000,000, 40,000,000, 45,000,000, 50,000,000, 55,000,000, 60,000,000, 65,000,000, 70,000,000, 75,000,000, 80,000,000, 85,000,000, 90,000,000, 95,000,000, 100,000,000, or more) modified cancer cells.

A dose may alternatively include, for example, at least about 100,000,000 to 1,000,000,000 (e.g., about 100,000,000, 150,000,000, 200,000,000, 250,000,000, 300,000,000, 350,000,000, 400,000,000, 450,000,000, 500,000,000, 550,000,000, 600,000,000, 650,000,000, 700,000,000, 750,000,000, 800,000,000, 850,000,000, 900,000,000, 950,000,000, 1,000,000,000, or more) modified cancer cells.

In some embodiments, the modified cancer cells are irradiated. The irradiation dose may be, for example, between about 2 and 2,000 Gy (e.g., about, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, 1,000, 1,100, 1,200, 1,300, 1,400, 1,500, 1,600, 1,700, 1,800, 1,900, or 2,000 Gy). In particular embodiments, the modified cancer cells are irradiated with a dose of about 100 Gy.

In some embodiments, treating the subject results in a decrease in the presence or level of one or more heterologous antigens measured or detected in a sample obtained from the subject. In some embodiments, treating the subject results in an increase in the presence or level of one or more biomarkers measured or detected in a sample obtained from the subject. In particular embodiments, treating the subject results in no change the presence or level of the one or more biomarkers.

In some embodiments, treating the subject results in an increase in the activity and/or number of one or more immune cells. In some instances, the increase is produced in one cell type. In other instances, the increase is produced in multiple cell types. In some embodiments, the cell in which the level of activity and/or number is increased is selected from the group consisting of a peripheral blood mononuclear cell (PBMC), a lymphocyte (e.g. T lymphocyte, B lymphocyte, NK cell), a monocyte, a dendritic cell, a macrophage, a myeloid-derived suppressor cell (MDSC), and a combination thereof. In particular embodiments, the level of activity and/or number of immune cell(s) is measured using methods of the present disclosure described herein.

In some embodiments, an increase in immune cell activity and/or number indicates that the subject should be administered one or more additional doses of the pharmaceutical composition (e.g., comprising modified cancer cells of the present disclosure). In some instances, a different vaccine is administered. One of skill in the art will recognize that an increase in immune cell activity and/or number will occur, in some instances, after 1, 2, 3, 4, 5, 6, 7, 8, 9, 10, 11, 12, 13, 14, or more doses of the vaccine have been administered.

In some embodiments, a sample is obtained from the subject. In other embodiments, a sample is obtained from a different subject or a population of subjects. Samples can be used for the purposes of selecting an appropriate cancer vaccine of the present disclosure, monitoring the response to vaccine therapy, and/or predicting how the subject will respond to vaccine therapy. Samples obtained from a different subject and/or a population of subjects can be used, for example, to establish reference ranges to facilitate comparisons that are part of the methods of the present disclosure. Samples can be obtained at any time, including before and/or after administration of the modified cancer cell(s), pharmaceutical composition(s), and/or other composition(s) of the present disclosure. In some embodiments, the sample comprises whole blood, plasma, serum, cerebrospinal fluid, tissue, saliva, buccal cells, tumor tissue, urine, fluid obtained from a pleural effusion, hair, skin, or a combination thereof. In general, the sample can comprise any biofluid. For HLA typing, any cell, tissue, or biofluid type is suitable, as long as it contains a sufficient amount of DNA or RNA to allow typing. In some instances, the sample comprises circulating tumor cells (CTCs). The sample can also be made up of a combination of normal and cancer cells. In particular embodiments, the sample comprises circulating tumor cells (CTCs). The sample can be obtained, for example, from a biopsy, from a surgical resection, and/or as a fine needle aspirate (FNA). Samples can be used to determine, measure, or detect HLA allele(s), immune cell activity and/or number, and/or biomarker(s), as described herein.

In some embodiments, the results of the HLA typing (e.g., the alleles present in an allele profile, the results of a comparison of allele profiles), immune cell activity and/or number measurement, and/or biomarker presence or level determinations are recorded in a tangible medium. For example, the results of assays (e.g., the alleles present in an allele profile, the results of a comparison of allele profiles, the activity level and/or number of immune cells, the presence or level (e.g., expression) of one or more biomarkers and/or a prognosis or diagnosis (e.g., of whether or not there is the presence of cancer, the prediction of whether the subject will respond to a vaccine, or whether the subject is responding to a vaccine) can be recorded, e.g., on paper or on electronic media (e.g., audio tape, a computer disk, a CD, a flash drive, etc.).

In other embodiments, the methods further comprise the step of providing the results of assays, prognosis, and/or diagnosis to the patient (i.e., the subject) and/or the results of treatment.

### E. Kits

In another aspect, the present disclosure provides a kit for treating a subject with a cancer. In some embodiments, the kit comprises a modified cancer cell line, a composition, and/or a pharmaceutical composition of the present disclosure described herein. The kits are useful for treating any cancer, some non-limiting examples of which include breast cancer, ovarian cancer, cervical cancer, prostate cancer, pancreatic cancer, colorectal cancer, gastric cancer, lung cancer, skin cancer, liver cancer, brain cancer, eye cancer, soft tissue cancer, renal cancer, bladder cancer, head and neck cancer, mesothelioma, acute leukemia, chronic leukemia, medulloblastoma, multiple myeloma, sarcoma, and any other cancer described herein, including a combination thereof.

Materials and reagents to carry out the various methods of the present disclosure can be provided in kits to facilitate execution of the methods. As used herein, the term “kit” includes a combination of articles that facilitates a process, assay, analysis, or manipulation. In particular, kits of the present disclosure find utility in a wide range of applications including, for example, diagnostics, prognostics, therapy, and the like.

Kits can contain chemical reagents as well as other components. In addition, the kits of the present disclosure can include, without limitation, instructions to the kit user, apparatus and reagents for sample collection and/or purification, apparatus and reagents for product collection and/or purification, apparatus and reagents for administering modified cancer cell(s) or other composition(s) of the present disclosure, apparatus and reagents for determining the level(s) of biomarker(s) and/or the activity and/or number of immune cells, apparatus and reagents for detecting HLA alleles, sample tubes, holders, trays, racks, dishes, plates, solutions, buffers or other chemical reagents, suitable samples to be used for standardization, normalization, and/or control samples. Kits of the present disclosure can also be packaged for convenient storage and safe shipping, for example, in a box having a lid. For instance, the kits may be stored and shipped at room temperature, on wet ice or with cold packs, or frozen in the vapor phase of liquid nitrogen or in dry ice.

In some embodiments, the kits also contain negative and positive control samples for detection of HLA alleles, immune cell activity and/or number, and/or the presence or level of biomarkers. In some embodiments, the negative control samples are non-cancer cells, tissue, or biofluid obtained from the subject who is to be treated or is already undergoing treatment. In other embodiments, the negative control samples are obtained from individuals or groups of individuals who do not have cancer. In other embodiments, the positive control samples are obtained from the subject, or other individuals or groups of individuals, who have cancer. In some embodiments, the kits contain samples for the preparation of a titrated curve of one or more biomarkers in a sample, to assist in the evaluation of quantified levels of the activity and/or number of one or more immune cells and/or biomarkers in a biological sample.

### IV. Examples

The present disclosure will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1: Generation of Bria-OTS Breast Cancer Immunotherapy Cell Lines

This example illustrates one embodiment of generating the cell lines described herein.

Bria-OTS are experimental, HER-2/neu positive, allogeneic, whole cell breast cancer (BC) cell lines designed to secrete granulocyte-macrophage colony stimulating factor (GM-CSF) in situ and augment dendritic cell activity. In addition, the Bria-OTS cell lines are engineered to express multiple human leukocyte antigen (HLA) alleles to match >99% of the breast cancer patient population. Bria-OTS consist of 4 separate BC cell lines: BC1, BC2, BC3, and BC4 as detailed below. Each of the four Bria-OTS cell lines expresses the CSF2 gene encoding GM-CSF.

Parental cell line. The Bria-OTS cell lines were derived from the breast cancer parent cell line, SV-BR-1, which expresses multiple cancer-associated antigens and immune stimulating factors including Class II HLA molecules that directly activate CD4+ T cells to enhance the immune response. Generation of the initial SV-BR-1 cell line is described in WO 2017/147600, which is incorporated herein in its entirety.

The parent cell line, SV-BR-1, contains HLA-A*24:02, HLA-B*35:08, HLA-B*55:01, HLA-C*01:02, HLA-C*04-01, and HLA-DRB3*01:01, endogenous HLA alleles, which are expressed; in addition HLA-DRB3*02:02, HLA-DRB1*11:04, HLA-DRB1*13:03 are expressed at lower levels but enhanced by exposure to interferon-gamma (IFNγ); and HLA-A*11:01, which is not detected. In the Bria-OTS cell lines, expression of HLA-A*24:02 and DRB3*01:01 have been functionally knocked out using CRISPR technology as described below. Bria-OTS cell lines instead express the following combinations of HLA genes: BC1.68.3.4 (BC1): HLA-A*01:01, HLA-A*68:01, HLA-DRB3*02:02, HLA-DRB4*01:01; BC2.11.4.3 (BC2): HLA-A*02:01, HLA-A*11:01, HLA-DRB4*01:01, HLA-DRB3*03:01; BC3.23.3.5 (BC3): HLA-A*03:01, HLA-A*23:01, HLA-DRB3*01:01, HLA-DRB5*01:02; and BC33.5.5 (BC4): HLA-A*33:03, HLA-DRB5*01:01, HLA-DRB5*02:02.

To prepare the therapeutic composition described herein, a vial from the master cell bank (MCB) can be thawed, grown in culture, harvested, and cryopreserved.

Inactivation of endogenous HLA antigen expression in cell lines. SV-BR-1 endogenous HLA alleles including: HLA-A*24:02, HLA-A*11:01, HLA-B*35:08, HLA-B*55:01, HLA-DRB3*01:01, and HLA-DRB3*02:02, of which all except HLA-A*11:01 are expressed under standard culture conditions. To eliminate the possibility of competition between endogenous SV-BR-1 HLA alleles and exogenous HLA alleles introduced as part of Bria-OTS-1 construction, Bria-KO (SV-BR-1-KO) was engineered by inactivating endogenous HLA-A and HLA-DRB3 alleles. Using clustered regularly interspaced short palindromic repeats/CRISPR-associated protein-9 (CRISPR/Cas9) technology, synthetic guide RNAs (sgRNAs) were designed to introduce frameshift mutations into the endogenous HLA alleles, rendering the HLA proteins nonfunctional as described below. sgRNAs Guide RNAs were transfected into Bria-SV-BR-1 cells using Lipofectamine CRISPRMAX Cas9 Transfection Reagent. Knock-out (KO) cells were evaluated by flow cytometry using anti-HLA-A or anti-HLA-DR antibodies. For example, HLA-A null cells were sorted out via negative isolation with Dynabeads (Invitrogen) and HLA-A24 antibody. Two rounds of negative selection were performed to enrich for HLA-A*24:02 negative cells. Additional rounds of negative selection using Dynabeads coupled to anti-HLA-DR (recognizing HLA-DR03, encoded by the HLA-DRB3 gene, in complex with HLA-DRα) antibody were similarly performed and clonal derivatives were obtained. Subsequently, genes were introduced, either by transfection or transduction, for immunostimulators (e.g., GM-CSF) and other exogenous HLA alleles. It was confirmed that Clone 17 contains HLA-A*24:02 and HLA-DRB3*01:01 frameshift mutations, resulting in mutant HLA antigens that are not recognized by antibodies in flow cytometry assays. Thus, two of the four target HLA alleles in the parent cell line, Bria-SV-BR-1, were successfully inactivated, resulting in the generation of Clone 17.

Clone 17 was subsequently transduced separately with 4 lentiviral vectors (V1, V2, V3, and V4 respectively) to induce expression of GM-CSF, interferon-alpha (IFN-α), and 4 exogenous HLA genes—2 HLA-A genes and 2 HLA-DRB3 or HLA-DRB4 or HLA-DRB5 genes. The lentiviral constructs used to develop the corresponding Bria-OTS cell lines are shown in Table 2.

The result was 4 cell lines all expressing GM-CSF and IFN-α, and BC1, BC2 and BC3 each expressing 4 unique HLA genes—2 Class I HLA genes and 2 Class II HLA genes, and BC4 expressing 3 unique HLA genes—2 Class I HLA genes and 1 Class II HLA gene, as noted in Table 2 and FIGS. 1-4. The cell lines were checked using flow cytometry on the cell lines developed after lentiviral transduction and by measuring GM-CSF in the culture supernatants.

CRISPR inactivation of endogenous HLA-A and HLA-DRB3 expression. Removal of HLA-A (*11:01 and *24:02) and HLA-DRB3 (*01:01 and *02:02) was done using CRISPR/Cas9 technology. Synthetic sgRNA with chemical modification were purchased from Synthego (HLA-A: A*C*A*GCGACGCCGCGAGCCAG-Synthego modified EZ scaffold (SEQ ID NO:45) and HLA-DBR3: U*G*C*AGACACAACUACGGGGU-Synthego modified EZ scaffold (SEQ ID NO:46)). SV-BR-1 cells were stored in LN2 and cultured at 37° C., 5% C02, and 95% humidity in supplemented RPMI culture medium. Cells were cryopreserved. SV-BR-1 cells were transferred back in culture and kept in culture before seeding. Cells were seeded at a concentration of 0.5×10⁶ cells per well in a 6-well plate. Next day, cells were transfected using Lipofectamine CRISPRMAX reagent. sgRNA (HLA-A) were reconstituted according to the manufactured recommendations and diluted to 30 uM. Next, the RNP complex was made by mixing 21 ul sgRNA 30 um+3.5 ul synthego provide 20 uM Cas9 nuclease+5 ul lipofectamine Cas9 plus reagent+95.5 ul Optimem, and incubating 10 minutes at room temperature. After the RNP complex was formed, it was mixed with the preincubated lipofectamine CRISPRMAX (5 ul Lipofectamine+120 ul optimen) and incubated for 5 minutes. Finally, 350 ul of mixture was added to the cell containing wells and incubated for 2-3 days. Cells were expanded for 10 days and KO was validated by flow cytometry using anti-HLA-A24 antibody on Mar. 1, 2018 (20% HLA-A24 negative cells). HLA-A null cells were sorted out via negative isolation with Dynabeads (Invitrogen) and HLA-A24 antibody. After bead sorting, 40% of cells were HLA-A24 negative. Cells were expanded and were subjected to a second round of negative isolation using HLA-A24 antibody and Dynabeads. After bead sorting 100% SV-BR-1 cells were HLA-24 negative as shown by flow cytometry. Cells were expanded and cryopreserved.

SV-BR-1 HLA-A knock out cells (KO) were transferred back in culture before seeding in 24 well plates at 0.15×10⁶ cells per well. Next day, cells were transfected using Lipofectamine CRISPRMAX reagent. sgRNA (HLA-DRB3) were reconstituted according to the manufactured recommendations and diluted to 30 uM. Next, the RNP complex was made by mixing 21 ul sgRNA 30 um+3.5 ul synthego provide 20 uM Cas9 nuclease+5 ul lipofectamine Cas9 plus reagent+95.5 ul Optimem, and incubating 10 minutes at room temperature. After the RNP complex was formed it was mixed with the preincubated lipofectamine CRISPRMAX (5 ul Lipofectamine+120 ul optimen) and incubated for 5 minutes. Finally, 350 ul of mixture was added to the cell containing wells and incubated for 2-3 days.

Single cell cloning of SV-BR-1 KO was then initiated. Cells were seeded at 1 cell/well in a 96 well plate in 30% complete medium+70% conditioned medium (20% FBS). Screening and freezing of single cell clones was subsequently performed and HLA-A/DRB3 KO clones were identified by using Guide-IT Genotyping Kit (Takara). Clone 17 was selected and used for further development. Clone 17 was further validated using Topo cloning and sequencing. Genomic DNA was isolated from the Clone 17 cell line and specific primers were used to amplify HLA-A (*11:01 and *24:02) and HLA-DRB3 (*01:01 and *02:02). Following PCR, the products were cloned according to the manufacturer's recommendations. Bacterial colonies were picked and sequenced using rolling circle amplification (RCA) prior to Sanger sequencing.

Preparation of plasmid for lentiviral production. The plasmid backbone, pCCl-c (SEQ ID NO:47) for the construction of this plasmid was originally constructed in Naldini laboratory (Dull et al., 1998) and further modified in Donald B Kohn laboratory (Department of Microbiology and immunology UCLA). For instance, the final plasmid of BC4 contains the following elements as show in FIG. 4. Specifically, Ampicillin resistance gene (AMP), is located at bases 132-992; CMV enhanced 5′ LTR, is located at bases 2358-3070; packaging signal (PSI), is located at bases 3120-3258; Rev responsive element (RRE), is located at bases 3745-3948; Central polypurine tract (cPPT), is located at bases 4461-4648; hEF1a promoter, is located at bases 4653-5842; CSF2-T2A-IFN-α, is located at bases 5843-6916; MNDU3 promoter, is located at bases 6917-7453; HLA alleles including HLA-A*24:02-T2A-HLA-A*33:03-P2A-HLA-DRB5*01:01-E2A-HLA-DRB5*01:02, are located at bases 7454-11452; U3 Sin Region 3′ LTR, is located at bases 11609-11664; R region 3′ LTR, is located at bases 11665-11761; U5 region 3′ LTR, is located at bases 11763-11845.

Cloning strategies. pCCLc-Ef1a-X-WPRE (SEQ ID NO:47) was created by removing a proprietary gene with BAMH1 and SAL1, and an MCS was cloned in. The final vector was confirmed with Sac2 with band sizes of 351 bp, 1111 bp, 6380 bp. In addition, the vector was confirmed by sequencing.

CSF2-T2A-IFN-α was synthesized by Genscript to have BamH1 sites on 5′, 3′ ends and coding sequences were codon optimized. CSF2 and IFN-α are separated by the sequence of a T2A self-cleaving peptide.

HLA-A*24:02-T2A-HLA-A*33:03-P2A-HLA-DRB5*01:01-E2A-HLA-DRB5*02:02 was synthesized by Genscript to have ECOR1 sites on 5′, 3′ ends and coding sequences were codon optimized. HLA-A*24:02 and HLA-A*33:03 are separated by the sequence of a T2A self-cleaving peptide. HLA-A*33:03 and HLA-DRB5*01:01 are separated by the sequence of a P2A self-cleaving peptide. HLA-DRB5*01:01 and HLA-DRB5*02:02 are separated by the sequence of a E2A self-cleaving peptide. This fragment was cloned into the EcoR1 site of pCCLc-EF1a-X-WPRE. Final plasmid was confirmed with NCO1 to give band sizes 221 bp, 1519 bp, 3271 bp, 3441 bp, 4439 bp, and also confirmed by sequencing.

Production of lentivirus. To produce lentiviral vectors, HEK 293T cells (Clontech Lenti-X 293 cells) were cultured in D10HG media, transfected using Mirus TRANS-IT 293 transfection Reagent with DMEM media, with the VSV-G envelope plasmid, packaging plasmid and vector (FIGS. 1-4). On the fifth day, the conditioned media was harvested, centrifuged, and the supernatant was concentrated using Millipore Centricon Plus 70 PL-100, and then filtered using Costar 0.45 Spinx centrifuge filter tubes. Viral titer was determined using the ABM LV900 Lentivirus Titer Kit with Mastermix R. Testing of the lentiviral vector was conducted, for example, by determining titer, sequence, RCL, bioburden, and/or endotoxin.

Lentiviral transduction. with the lentivirus. Cells were seeded in a 12-well plate at a density of 0.29×10⁶ cells/well and transduced at 150 MOI the next day. For transduction, the medium was 1 ml RPMI 1640+10% FBS+L-Glutamine+20 mg/mL Protamine Sulfate. Once the cells recovered and reattached, cells were lifted using 4 mL TrypLE Express for 5 minutes. Reaction was stopped with 8 mL culture media. Cells were counted and cryopreserved using CS10 at approximately 8×10⁵ cells/vial.

Lentiviral transduced cells (e.g., Clone 17 cells) were transferred into culture and expanded. HLA-DR+ cells were isolated using Dynabeads (Invitrogen) and HLA-DR antibody—L243 (BioLegend), according to the manufacturer's recommendations. Cells were further expanded and Clone 17 cells were subjected to a second round of HLA-DR+ isolation using Dynabeads. Cells were further expanded and then single cell cloning was initiated by seeding at 2 cells/well in 96 well plates. Single cell clones were transferred to 48-well plates. GM-CSF ELISA (BioLegend) was completed to identify positive clones. GM-CSF positive clones were transferred to a 12-well plate. For example, the V4-2 clone (BC4 cell line) was expanded and then cryopreserved using CS10 freezing medium.

Cell line characterization using GM-CSF and IFN-α ELISA. The BC4 cell line and Clone 17 cells were thawed and production of GM-CSF was measured using a GM-CSF ELISA kit (R&D Systems Cat. Number DGM00), according to the manufacturer's recommendations. The concentration of GM-CSF detected in BC4 was 36.9 ng/mL/24 h, but no GM-CSF was detected in Clone 17. BC4 IFN-α production was measured using an IFN-α ELISA kit (Invitrogen) and the IFN-α concentration was below the assay limit of detection for both cells.

HLAs SYBR green quantitative RT-PCR. The BC4, SV-BR-1, and Clone 17 cells were thawed and cultured for 7 days until cells reached 80% confluency. Cells were lifted using accutase dissociation reagent, then counted, and 5×10⁶ cells were stored at −80° C. for subsequent analyses.

RNA was extracted using PureLink RNA Mini kit (ThermoFisher) according to the manufacturer's recommendations. 500 ng of RNA were used for first strand cDNA synthesis. Subsequently, this cDNA (2 μL) was subjected to SYBR Green Quantitative RT-PCR (Applied Biosystems) using custom designed primers for HLA-A*24:02, HLA-A*33:03, HLA-DRB5*01:01 and HLA-DRB5*02:02 (Genescript). In BC4 but not in SV-BR-1 and Clone 17, high levels of HLA-DRB5*02:02 and HLA-A*33:03 and low levels HLA-DRB5*01:01 were confirmed. No cDNA detection of HLA-A*24:02 was observed. Thus, it was assumed that HLA-A*24:02 was not expressed, and the cell line was designated BC33.5.5 (BC4).

HLA-DR flow cytometry: The BC4, SV-BR-1, and Clone 17 cells were seeded in 6-well plates. On the following day, cells were treated with IFNγ for 48 h. The treated cells were subjected to flow cytometry using anti-HLA-DR antibody (clone L243, Biolegend). The expression levels of HLA-DR in BC4 and SV-BR-1 were comparable and higher than Clone 17.

### Example 2: Preparation of MCB for Each Cell Line

A master cell bank (MCB) may be created for each cell line and tested. As an illustration, the BC1, BC2, BC3 and BC4 cell lines will be propagated in T-25, T-75 and T-150 flasks using RPMI with 10% FBS medium. To confirm the production of GM-CSF, randomly selected flasks will be incubated in antibiotic-free RPMI+10% FBS for 72 hours. The supernatant will be collected, and the concentration of GM-CSF will be determined by ELISA. To confirm the expression of specific HLA alleles, cells will be subjected to flow cytometry using HLA-specific antibodies and RT-PCR using HLA-specific primers.

The propagated cells will be harvested at passage 8 using 0.25% porcine trypsin+1 mM EDTA (Gibco). The cells will be resuspended in freezing medium (CS10) and aliquoted into cryovials at a concentration of 1.5×10⁶ viable cells/vial. The cells will be tested as outlined below.

Testing of MCBs for each cell line. A master cell bank (MCB) may be created for each cell line and tested. Briefly, the cells may be tested for total cell recovery, viability, identity, purity, potency, mycoplasma, mycoplasma direct and indirect detection, mycoplasma culture (mycoplasmastasis), endotoxin, 14-day sterility culture, specific viruses such as AAV2, EBV, HAV, HBV, HCMV, HCV, herpesviruses (broad-range), HHV-6, HHV-7, HHV-8, HIV (HIV-1, HIV-2), HTLV-1, HTLV-2, human parvovirus, polyomavirus (broad-range), WNV, retroviral contaminants, 9 CFR detection of bovine viruses or porcine viruses, replication competent lentivirus (RCL), and/or non-endogenous/adventitious viruses. Details of the testing method and criteria are listed in Table 4 below.

TABLE 4
Release testing of MCBs for BC1 through BC4 cell lines

| Test | Method | Acceptance Criteria |
|---|---|---|
| Total cell recovery | Hemacytometer | 75%-120% |
| | (% cells recovered per vial | |
| | relative to total number of | |
| | cryopreserved cells/vial) | |
| Viability | Trypan Blue | ≥70% |
| Identity | Her2/neu IHC | Positive |
| | | (ASCO/CAP Score 3+) |
| Purity | ER/PgR IHC | Negative |
| | | (ASCO/CAP Guidelines) |
| Potency | GM-CSF ELISA | ≥5 ng per 10⁶ cells per 24 hours |
| Mycoplasma | Validated RT-PCR method* | Negative per assay-defined Ct value |
| Mycoplasma - direct and indirect | USP <63> | Not detected |
| (detection) | | |
| Mycoplasma - culture | USP <63> | Non-inhibitory (Pass) |
| (mycoplasmastasis) | | |
| Endotoxin, USP <85> | Kinetic chromogenic LAL | <5 EU/mL |
| 14-day Sterility culture | USP <71> | No growth |
| Specific viruses: | RT-PCR, PCR | Not detected |
| AAV2, EBV, HAV, HBV, | | |
| HCMV, HCV, herpesviruses | | |
| (broad-range), HHV-6, HHV-7, | | |
| HHV-8, HIV (HIV-1, HIV-2), | | |
| HTLV-1, HTLV-2, human | | |
| parvovirus, polyomavirus (broad- | | |
| range), WNV | | |
| Retroviral contaminants (Reverse | PCR-based reverse transcriptase | Not detected |
| transcriptase) | assay (PBRT) Using fluorescent | |
| | probe technology | |
| 9 CFR - Bovine viruses | 9 CFR detection of bovine | Not detected |
| | viruses in bovine serum or other | |
| | products - 21 days | |
| 9 CFR - Porcine viruses | 9 CFR detection of porcine | Not detected |
| | viruses - 21 days | |
| Replication competent lentivirus | Amplification of potential RCLs | Not detected |
| (RCL) | in a susceptible human T-cell | |
| | line with ELISA and PBRT | |
| | endpoints | |
| Non-endogenous/adventitious | In vitro (MRC-5, VERO, HeLa) | Negative (cytopathic effects, |
| viruses | | hemagglutination, hemadsorption) |
| | In vivo (hen eggs) | Negative |
| | In vivo (mice) | Negative |

*Either the validated PCR or the USP <63> method will be used for mycoplasma detection.

### Example 3: Composition of a Drug Product

This example describes one embodiment of the composition of a drug product as described herein. The drug product consists of a suspension of cells, grown from a single vial of the MCB and irradiated.

BC1 through BC4 drug product (DP) are each provided as a frozen suspension of approximately 20 million total BC cells/mL (target of 12.5 million single viable cells/mL) in CryoStor CS10 with a 1.25 mL fill in 2 mL cryovials. The DP is to be stored in 2 mL external thread, free-standing CryoElite Natural Cap vials. The vials are made from low binding, cryogenic grade virgin polypropylene.

As an illustration, the BC33.5.5 (BC4) cell line is a HER-2/neu positive, allogeneic, whole cell breast cancer (BC) cell line, which is engineered to express GM-CSF and the following HLA genes: HLA-A*33:03, HLA-DRB5*01:01, HLA-DRB5*02:02. The native HLA genes were functionally knocked out using CRISPR technology. The drug substance (DS) consists of a suspension of cells, grown from a single vial of the MCB and irradiated. The DS will be immediately resuspended in a cryopreservative solution and frozen to produce the final drug product.

The composition of the BC4 DP vaccine is provided in Table 5. The composition is the same for each of the BC1 through BC4 DP. The composition contains an active ingredient comprising approximately 20 million total BC1 cells per mL and Qs to 1 mL of CryoStor CS10 as buffer and cryoprotectant. CryoStor CS10 is an animal component-free, defined cryopreservation medium with 10% DMSO. It is manufactured under cGMPs using USP grade reagents.

TABLE 5
Composition of BC4 drug product (BC DP)

| Component | Amount per mL | Function |
|---|---|---|
| BC4 Cells | Approximately 20 | Active ingredient |
| | million total cells | |
| CryoStor CS10 | Qs to 1 mL | Buffer and |
| | | cryoprotectant |

### Example 4: Safety and Efficacy of Modified Human Cancer Cell Lines in Cancer Patients

This example illustrates one embodiment of testing the safety and efficacy of modified human cancer cell lines (e.g., Bria-OTS cell lines) in patients with advanced metastatic or locally recurrent breast cancer in a clinical trial. This study will provide preliminary data regarding the safety, tolerability, tumor response in patients with advanced (e.g., metastatic or locally recurrent) breast cancer. A brief summary of the clinical study design is provided below.

This is a Phase1/2a, open-label clinical study with patients assigned to treatment with a breast cancer immunotherapy cell line based on their HLA type. The primary objective of the study is to evaluate the safety of HLA-matched cellular immunotherapy in patients with advanced breast cancer. The secondary objective of the study is to evaluate the tumor response to HLA-matched cellular immunotherapy in patients with advanced breast cancer. Additionally, the study may include objectives to evaluate progression-free (PFS) and overall survival (OS) in advanced breast cancer patients treated with HLA-matched cellular immunotherapy, to evaluate the immune responses elicited by HLA-matched cellular immunotherapy in patients with advanced breast cancer, to evaluate patient and tumor characteristics that may be predictive of responses to HLA-matched cellular immunotherapy in patients with advanced breast cancer, and/or to evaluate Quality of Life (QOL) in advanced breast cancer patients treated with HLA-matched cellular immunotherapy. The cell lines being studied are the Bria-OTS cell lines (e.g., BC1, BC2, BC3, or BC4). Patients will be treated with the cell line that most closely matches their HLA type, with at least one match required for a patient to be treated.

Additionally, to boost the immune response, patients are pretreated with low-dose cyclophosphamide that downregulates T regulatory-cell mechanisms 48-72 hours (2-3 days) prior to each vaccine inoculation. Low-dose pegylated interferon-alpha-2a (IFN-α2a) serves as an adjuvant and is given by intradermal injection at 0.1 mcg to the inoculation site about 1-4 hours and about 24-72 hours (1-3 days) following vaccine inoculation. Biological samples are collected at regular intervals per protocol, and stored in a repository.

As an illustration, in Part 1 (Phase 1) of the study, there will be intra-patient dose escalation with each patient treated with a single cell line. Once the dose of cells has been decided in Part 1, in Part 2 (Phase 2a) patients may be treated with either 1 or 2 cell lines, with the goal to have patients match the cell lines used at least at 2 HLA types, preferably one class II (HLA-DR) type and one class I (HLA-A, B, or C) type.

Patient population. Patients will be screened to assure they fulfill the enrollment criteria. Screening must be performed within 30 days of initiating therapy, and imaging studies must be performed within 2 weeks of initiating therapy. All patients will be women with histologically confirmed breast cancer with recurrent and/or metastatic lesions via investigational site, which has failed prior therapy. Patients with any of the 4 breast cancer subtypes will be eligible, i.e., luminal A (HR+/HER2−), triple-negative (HR−/HER2−), luminal B (HR+/HER2+), and HER2-enriched (HR−/HER2+), given they meet the required specifications of failed prior treatments. Patients with new or progressive breast cancer metastatic to the brain will also be eligible if their intracranial disease is stable and not life-threatening as noted in the protocol synopsis.

Planned enrollment for both parts of the study is up to 48 patients, with 12-24 patients (at least 3 patients with each cell line) to be evaluated initially. If the cell lines are found to be safe in Part 1, then the expansion cohort of 24 patients (at least 4 patients per cell line) will be enrolled in Part 2.

Study treatment and dosage escalation. The Bria-OTS cell lines will be irradiated to render them replication incompetent prior to freezing in viable freezing media. They will be shipped to the clinical sites frozen and thawed on site for inoculation. Patients will be evaluated initially every week during the dose escalation phase, including all safety assessments.

The dosage form is formulated in suspension of irradiated cells. Patients will be administered with a therapeutically effective amount of the composition through intradermal injection in the upper back or thighs over a period of time. Table 6 illustrates dosing regimen for Phase 1 monotherapy phase.

TABLE 6
Dosing regimen for monotherapy phase

| Time | Cell count | Number of injections |
|---|---|---|
| Week 1 | 20,000,000 cells | Up to 4 intradermal injections |
| Week 3 | 20,000,000 cells | Up to 4 intradermal injections |
| Week 5 | 20,000,000 cells | Up to 4 intradermal injections |
| Week 7 | 20,000,000 cells | Up to 4 intradermal injections |

Patients will return 2±1 days following each inoculation for safety assessments and to measure the delayed-type hypersensitivity (DTH) response during the monotherapy phase. Following the monotherapy phase the patients will be treated with the top dose (i.e., maximum tolerated dose [MTD] or a pharmacologically active dose), which was the cell dose safely tolerated every 3 weeks. During this phase, the patient will also receive cyclophosphamide 300 mg/m² 2-3 days prior to each cell line inoculation, and pegylated IFN-α2a 0.1 mcg into each inoculation site, 1-4 hours and 1-3 days following each cell line inoculation. The entire cycle will be repeated every 3 weeks.

Once at least 3 patients have been safely treated with each cell line as a single cell line, Part 2 (Phase 2a) of the study will commence. During Part 2, all patients will be treated with 1 or 2 cell lines, with the goal to have patients match the cell lines used at least at two HLA types, preferably one class II (HLA-DR) type and one class I (HLA-A, B or C) type. Treatment will be preceded by cyclophosphamide, 300 mg/m², 2-3 days prior to the BC cell line inoculations. Treatment will be with 20,000,000 cells (up to 4 intradermal injections in the upper back and thighs). They will the receive pegylated IFN-α2a 0.1 mcg into each inoculation site, 1-4 hours and 1-3 days following each cell line inoculation. Patients will return 2±1 days following each inoculation for safety assessments and to measure the DTH response. Treatment cycles will be repeated every 3 weeks.

Assessments. Study participants are closely monitored for adverse events (e.g., toxicity) using the common terminology criteria for adverse events (i.e., CTCAE v 4.03) scale. Development of a new or progressive tumor, or treatment-related Grade III allergy/hypersensitivity, truncates further inoculations to any particular subject.

While the core success measure is safety and lack of toxicity, any of the following may be applied as success measures: 1) objective clinical response as defined by irRC RECIST 1.1 criteria in 25% of patients, 2) improvement in quality of life in 50% or more patients as evidenced by significant change in one or more scales in the SF-36 questionnaire (quality of life), 3) prolongation of disease-free and overall survival as compared with historical controls from reports of other salvage therapies in the published literature, 4) evidence of development or amplification of immune responses, especially if correlating with prolongation of survival. Other success measure may include induction of tumor regression.

Objective clinical response is primarily assessed by radiographic assessment of tumor burden. This may be conducted, as non-limiting examples, by computed tomography (CT), magnetic resonance imaging (MRI), and/or positron emission tomography (PET). To assess whether objective tumor regression is particularly pronounced inpatients with HLA alleles also found in SV-BR-1-GM cells, HLA types from, as non-limiting examples, buccal cells or blood cells of clinical trial subjects are determined. Several methods known to one of skill in the art are suitable to determine HLA alleles.

### Example 5: Turning Tumor Cells into Antigen-Presenting Cells for Cancer Immunotherapy

This example illustrates one embodiment of turning tumor cells into antigen-presenting cells for cancer immunotherapy. The tumor cells may comprise engineered human cancer cell lines (e.g., Bria-OTS cell lines) and may be prepared for administration to patients with advanced metastatic or locally recurrent breast cancer.

Background Therapeutic cancer vaccines are based on specific stimulation of the immune system using tumor antigens to elicit an antitumor response. Clinical trials using the breast cancer cell line SV-BR-1-GM as a therapeutic vaccine are being conducted underway. SV-BR-1-GM is a GM-CSF expressing breast cancer cell line with features of an antigen presenting cell (APC) owning to the expression of several immunomodulatory molecules, including MHC-I (HLA-A, B & C) and MHC-II (HLA-DRB3 & -DRA). Initial results in patients treated with irradiated SV-BR-1-GM cells, low dose cyclophosphamide and local IFN-α suggest that patients that match SV-BR-1-GM at least at 1 HLA allele are more likely to derive clinical benefit. This clinical observation, together with the fact that SV-BR-1-GM cells can directly activate CD4+ T-cells in an antigen-specific HLA-restricted manner, as demonstrated by an in vitro antigen presentation assay, lead us to hypothesize that SV-BR-1 (the parent cell line) can function as APC. To further enhance direct antigen presentation to T-cells, SV-BR-1 cells have been genetically modified to express co-stimulatory molecules, immunomodulatory cytokines, and an extended repertoire of HLA alleles.

Methods. To generate an off-the-shelf semi-allogeneic cell therapy covering most of the population, SV-BR-1 was genetically modified to express an extended repertoire of HLA alleles. Based on population analysis, four cell lines, each carrying two (2) HLA-A and two (2) HLA-DRB3/4/5 alleles, may produce at least a single match in 99% of the population, with a 92% match at class I HLA-A alleles and a 98% match at class II HLA-DRB3/4/5 alleles. SV-BR-1 was genetically modified using CRISPR/cas9 deletion of the endogenous HLA-A and HLA-DRB3 alleles and subsequent lentiviral mediated expression of alternative HLA-A and DRB3 alleles. To generate tumor cell lines with enhanced direct antigen presentation to T-cells, SV-BR-1 cells were genetically modified to express co-stimulatory molecules and immunomodulatory cytokines by using a lentiviral mediated expression system.

Results. Following sequential CRISPR/Cas9 editing, the SV-BR-1 cells were cloned, and one clone selected (clone 17) for further engineering. Lack of expression of HLA-A and HLA-DRB3 was confirmed using flow cytometry and DNA sequencing. Clone 17 was subsequently transduced with 6 lentiviral vectors each expressing 2 genes under the control of separate promoters: CD86-IL12, CD80-HLA-DRA, 4-1BBL-IL7, GM-CSF-IFNA2, HLA-allele-1-HLA-allele-2 and HLA-DR-allele-1-HLA-DR-allele-2. Four cell lines were generated with different combinations of HLA alleles. Following selection, cells were evaluated by ELISA, flow cytometry and RT-PCR to confirm gene expression. Cell lines that secreted GM-CSF, IFN-α2 (e.g., IFN-α2a, IFN-α2b), IL-12, IL-7 and expressed CD80, CD86, 4-1BBL, and both class I and class II HLA alleles are then transferred to GMP manufacturing. In some cases, the IFN-α such as a pegylated IFN-α2a is exogenously provided to the modified cancer cell line.

Clinical trial. These modified breast cancer cell lines are used in clinical studies designed to first evaluate the safety of intradermal inoculation with the irradiated cells and later combined with other agents to augment the immune response. Each patient is treated with the cell line(s) that match them at least at one HLA allele.

### Example 6: An Off-the-Shelf Personalized Cellular Approach to Immunotherapy for the Treatment of Advanced Solid Tumors

Background: We are developing off-the-shelf personalized cellular immunotherapies based on SV-BR-1-GM, which is in Phase I/IIa clinical trial in patients with metastatic or locally recurrent breast cancer. SV-BR-1-GM is a breast cancer cell line with features of an antigen presenting cell (APC) which has been stably transfected with the CSF2 gene encoding GM-CSF (SV-BR-1-GM). Favorable clinical outcomes have been reported in patient populations that match SV-BR-1-GM at one or more HLA alleles. This clinical observation, together with the fact that SV-BR-1-GM cells can directly activate CD4+ T-cells in an antigen-specific HLA-restricted manner, as demonstrated by an in vitro antigen presentation assay (Lacher M D et al, Front Immunol. 2018 May 15; 9:776), lead us to hypothesize that SV-BR-1-GM can function as an APC. This example describes a therapeutic approach in which a patient will be treated with a cell line expressing HLA class I and II molecules matched to their genotype. Also, to further enhance direct antigen presentation to T-cells, the parent SV-BR-1 cells were genetically modified to express co-stimulatory molecules and additional immune-modulatory cytokines.

Methods: Using CRISPR/Cas9 technology, we have inactivated several endogenous HLA-A and HLA-DRB alleles present in five cancer cell lines (SV-BR-1, PC-3, LNCaP, SK-MEL-24, and NCI-H2228). Cells with inactivated HLA-A/DRB genes were transduced with lentiviral based vectors expressing selected cytokines and costimulatory molecules (GM-CSF, IFN-α, CD80, CD86, IL-12, IL-7, HLA-DRA, and 4-1BBL). Next, unique combinations of HLA-A and HLA-DRB3/4/5 alleles were transduced into the cells using lentiviral based vectors to generate a collection of cell lines (e.g., Bria-OTS cell lines) that will match over 99% of the patient population for at least one HLA allele. Expression and functionality of the stimulatory molecules and transgenic HLA alleles was established using flow cytometry and cell-based assays.

Results: Four cell lines (for each tumor type) that secreted GM-CSF, IFN-α, IL-12, IL-7 and expressed CD80, CD86, 4-1BBL, and different combinations of both class I and class II HLA alleles were selected. Using cell-based assays, including mixed lymphocyte reaction assays, we demonstrated that the generated cells stimulate naïve T-cells.

Conclusions: We have successfully generated “off the shelf” personalized cell-based therapeutic cancer vaccines that induce potent T-cell responses. These modified cancer cell lines are used in clinical studies designed to first evaluate the safety of intradermal inoculation with the irradiated cells and later combined with other agents to augment the immune response.

### Example 7: Bria-OTS Cells with HLA-Matching Alleles for the Personalized Treatment of Advanced Solid Tumors

This example describes the development of novel, precision-based immunotherapeutic cell lines for the treatment of advanced solid tumors that can HLA match to over 99% of the cancer patient population in the US. This immunotherapy platform holds promise as a safe, potent, and efficacious treatment of solid tumors, and will be the first rapid, off-the-shelf therapy that does not require personalized manufacturing.

### SUMMARY

Cancer vaccines hold promise as immunotherapy due to strong and durable cancer-specific immune responses. Different types of cancer vaccines have been developed, including DNA/RNA-based protein/peptide-based, dendritic cell-, and whole cell-based vaccines. Tumor cell vaccines express both tumor-specific and tumor-associated antigens, but they don't elicit strong responses in the clinic and there is great variability in Human Leukocyte Antigen (HLA)-restricted cellular responses among patients that limits vaccine efficacy. Thus, there remains an urgent and unmet need for new treatment regimens with greater impact on long-term survival.

To address the needs for new immunotherapeutic approaches to treat advanced solid tumors, we are developing a novel whole-cell immunotherapeutic approach that acts through two complementary mechanisms of action: 1) cross-presentation of cancer cell antigens and 2) direct T cell activation. This dual mechanism of action is considered unique and represents a significant advance over previous attempts to develop whole-cell cancer vaccines. Enhanced clinical response via direct T cell activation is achieved when a patient's HLA molecules match those in the therapeutic cell line. Our first-generation Bria-IMT product has demonstrated substantial tumor regression in patients with metastatic breast cancer who match Bria-IMT at least one HLA alleles. Bria-IMT is a breast cancer cell line (SV-BR-1-GM) that secretes granulocyte macrophage colony-stimulating factor and functions as an antigen presenting cell (APC) and is able to directly activate CD4⁺ T cells. We completed a molecular analysis of SV-BR-1-GM cells by microarray gene expression profiling and identified a distinctive 22-gene immune signature that confers APC-like activity. Based on this unique immune signature, we have selected additional cell lines of different origins in addition to breast cancer (e.g., melanoma, prostate and lung cancers) and are engineering cell lines which express a defined set of cytokines and co-stimulatory molecules as well as a discrete collection of HLA alleles that, collectively, will have the potential to treat almost 100% of the US population at the level of one HLA class I or II allele match with 90% matching at 2 HLA alleles. These cells (termed Bria-OTS for “off-the-shelf”) will be used as a pre-manufactured and ready to use personalized immunotherapy for the treatment of advanced solid tumors.

### Specific Aims

Cellular cancer immunotherapies must be tailored to the individual patients in order to induce an effective immune response, which increases the complexity of manufacturing and elevates associated cost. To circumvent this limitation, we are developing Bria-OTS™ as an off-the-shelf, pre-manufactured and ready to use personalized immunotherapy for the treatment of advanced solid tumors.

Cancer immunotherapy relies on the activation of the patient's immune system to eliminate cancer cells based on the presence of tumor-associated antigens (TAAs). Despite great advances, current immunotherapies are effective only on a very limited number of patients.^{1,2} Cancer vaccines hold promise as they often lead to strong and durable cancer-specific immune responses. Different types of cancer vaccines have been developed, including DNA/RNA-based, protein/peptide-based, dendritic cell (DC), and whole cell-based vaccines. While autologous DC vaccines have been successful in the clinic,³ their use is limited by complex manufacturing logistics that compromise feasibility and elevate cost.⁴ Tumor cell-based vaccines express both tumor-specific and tumor-associated antigens, but they don't elicit strong responses in the clinic and there is variability in Human Leukocyte Antigen (HLA)-restricted cellular responses among patients.⁵ Thus, there remains an urgent and unmet need for new treatment regimens with greater impact on long-term survival.

We are developing off-the-shelf personalized cellular immunotherapies—Bria-OTS-based on our most advanced lead candidate—Bria-IMT™, which is in a Phase I/IIa clinical trial in patients with metastatic or locally recurrent breast cancer (NCT03328026), and preliminary data showed substantial tumor regression in heavily pre-treated patients who match Bria-IMT with two HLA alleles.⁶ Bria-IMT™ is a breast cancer cell line (SV-BR-1-GM) stably transfected with the CSF2 gene and thus secretes granulocyte macrophage colony-stimulating factor (GM-CSF). Bria-IMT™ expresses class I and II human leukocyte antigen (HLA) complex genes. SV-BR-1-GM functions as an antigen presenting cell (APC) to directly activate CD4⁺ T cells.⁷ Building on the Bria-IMT technology, which demonstrated excellent safety and tumor regression in preliminary clinical data in metastatic breast cancer patients with at least one HLA allele matched to the Bria-IMT cell line,^{6,8} we are developing engineered Bria-OTS cell lines for the treatment of solid tumors. We are engineering Bria-OTS cell lines derived from breast cancer (SV-BR-1), prostate cancer (PC-3), melanoma (SK-MEL-24), or lung cancer cells (NCI-H2228). These cell lines have been chosen based on the expression of a characteristic 22-gene immune signature that was originally characterized in SV-BR-1 cells and includes MHC class I and II components, certain cytokines and chemokines known to promote attraction of immune cells.⁷ Bria-OTS cells are engineered to express a discrete collection of HLA alleles that collectively will have the potential to treat almost 100% of the US population at the level of one HLA class I or II allele match and approximately 90% at the level of ≥1 HLA class I plus ≥1 HLA class II (double-match). Once a patient's unique HLA signature is known, they will be assigned a complementary Bria-OTS product having at least one matching HLA allele. Treatment can begin immediately, without the need for patient-specific manufacturing.

Generating Bria-OTS breast, prostate, melanoma, and lung cancer cell lines. Using CRISPR/Cas9 technology, we have inactivated several endogenous HLA-A and HLA-DRB alleles present in all four parent cell lines (SV-BR-1, PC-3, SK-MEL-24, and NCI-H2228). We used pCCLc-based lentiviral vectors to express HLA-DRA, selected cytokines (GM-CSF, IFNα, IL-12, IL-7), and costimulatory molecules (CD80, CD86, 4-1BBL), followed by overexpression of unique combinations of HLA-A and HLA-DRB3/4/5 alleles. As a final product, 4 different Bria-OTS clonal cell lines that will match over 99% of the patient population within a specific cancer type are generated. Final Bria-OTS cells are rigorously tested to confirm chromosomal integration (whole genome sequencing) and lack of major gene expression deviations from the parental cells (RNA-seq).

Demonstrating in vitro functionality of Bria-OTS cells. Using a Mixed Lymphocyte Reaction (MLR) assay, T cell proliferation, secreted interferon gamma (IFNγ), interleukin-2 (IL-2), IL-4, and IL-17 levels at baseline and after 1, 2, 5, and 7 days are measured to evaluate T cell activation (to cover CD8+ cytotoxic T cells, CD4+Th1, Th2, and Th17 T cell subsets). Eight T cell receptor (TCR) transgenic Jurkat cell lines expressing selected HLA-A- and HLA-DR-restricted TCRs are generated to evaluate transgenic HLA functionality using a T cell activation bioassay.

The clinical efficacy of Bria-OTS cells can be validated in xenograft tumor mouse models of advanced solid tumors, and safety and clinical activity can be demonstrated in first-in-human studies. The cell immunotherapy strategy described herein holds promise as a safe and efficacious treatment option for solid tumors that are in need of effective therapies, with the advantage of personalized therapy without complex manufacturing.

### Significance

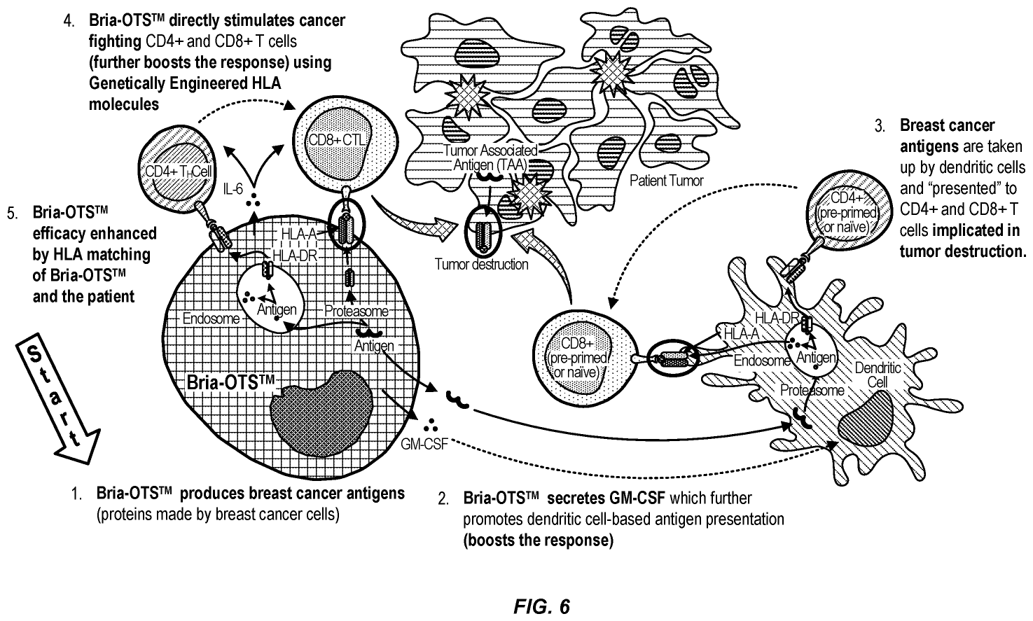

Cancer immunotherapy for advanced solid tumors. In contrast to traditional chemo- or radiation therapies that indiscriminately kill all rapidly-dividing cells, cancer immunotherapy relies on the activation of the patient's immune system to eliminate cancer cells based on the presence of tumor-associated antigens (TAAs). Despite great advances, current immunotherapies are effective only on a very limited number of patients.^{1,2} Cancer vaccines hold promise as they often lead to strong and durable immune responses. Development efforts have focused on peptide and protein tumor antigens or neoantigens,⁹⁻¹¹ dendritic cells pulsed with cancer antigens,¹²⁻²² and whole tumor cells.^{23,24} The advantage of a whole-cell vaccine is that it delivers a range of TAAs.²⁵ Sipuleucel-T (Provenge®) is currently the only US FDA approved whole-cell, vaccination-based individualized immunotherapy for advanced prostate cancer treatment. However, it does not provide the repertoire of antigens whole-cell vaccines do as it is only directed towards a fusion protein of prostatic acid phosphatase antigen (PAP) and GM-CSF through a mechanism of action resembling “cross-presentation”. In essence, sipuleucel-T is a cellular therapy comprising antigen-presenting cells (APCs) “loaded” with PAP-GM-CSF, which are recognized by a subset of patient T cells. Addition of GM-CSF to a whole-cell tumor vaccine has been proven to stimulate migration of DCs, T cells, and macrophages to the site of vaccination.²⁶ Despite great efforts and promising results in early phase studies, whole cell cancer vaccines have shown low clinical efficacy, likely due to multiple factors including but not limited to poor immunogenicity, lack of co-stimulatory molecules, downregulated expression of Human Leukocyte Antigen (HLA), and/or suppression by the tumor microenvironment (TME).²⁷ Addressing such significant obstacles, we are developing Bria-OTS cell lines that express TAAs, co-stimulatory molecules, and HLA genes, enabling direct presentation of tumor antigens to T cells, as well as immunostimulatory cytokines, leading to an enhanced immune response (FIG. 6).

Scientific rationale. Bria-IMT (also called SV-BR-1-GM) is a HER2/neu positive breast cancer cell line genetically engineered to overexpress GM-CSF. We have conducted a molecular fingerprint of SV-BR-1-GM cells by microarray gene expression profiling, quantitative reverse transcription PCR (qRT-PCR), transcript counting (nCounter), ELISA, and flow cytometry.⁷ We found that SV-BR-1-GM, despite its breast epithelial origin, expresses a gene signature associated with immunostimulatory functions. In addition to 1HLA class I molecules, Bria-IMT expresses several HLA class II genes, namely, human leukocyte antigens (HLA)-DMA, -DMB, -DRA, and -DRB3.⁷ We identified a unique 22-gene immune signature that included cell surface ligands for T cell costimulatory receptors, cytokines and other soluble factors with positive T cell-stimulatory functions, factors promoting maturation, survival, chemotaxis, and/or in vitro generation of DCs, and factors promoting antigen presentation.⁷ We also identified 31 genes expressed in SV-BR-1-GM cells that are potential TAA candidates, including Her2/neu, MIEN1 (migration and invasion enhancer 1), PGAP3 (post-GPI attachment to proteins phospholipase 3) and STARD3 (Star related lipid transfer domain containing 3) and several cancer/testis antigens (CTAs) were robustly expressed (PRAME, the kinesin-like protein KIF2C, centrosomal protein of 55 kDa—CEP55, and PDZ binding kinase—PBK).^{7,28} In vitro studies demonstrated that Bria-IMT directly stimulates T cell activation in an antigen-specific HLA-restricted manner and thus possesses features of antigen-presenting cells.⁷ Importantly, this feature of Bria-IMT requires that the endogenous HLA class II genes expressed in the Bria-IMT cell line “match” those of the co-cultured T cells. Thus, the Bria-IMT cell line possesses critical features of both breast cancer cells and dendritic cells. In the clinic, patients bearing HLA class II alleles that match the endogenous HLA alleles in Bria-IMT show substantial tumor regression and other signs of clinical activity.^{6,8} As such, our clinical data demonstrate that if the endogenous HLA bar code inherent to the Bria-IMT cell line matches that of the patient, then the therapy has the potential to induce an enhanced clinical response. These clinical data highlight direct T cell stimulation via class II HLA matching as a previously unrecognized contributor to robust immunotherapeutic activity that is either absent from or was not considered when evaluating previous whole-cell immunotherapies. Moreover, our studies collectively establish the novel “dual mechanism” of action of Bria-IMT in which the immune system is stimulated through both antigen cross-presentation and direct T cell stimulation (FIG. 6). However, despite the demonstrated clinical potential of Bria-IMT, only 47% of the US population carries one or more HLA alleles that match allele(s) in the Bria-IMT cell line and roughly 10% of the population matches at two or more HLA alleles expressed in the Bria-IMT cell line.²⁹

To expand the population of patients who may benefit from our immunotherapy, we are developing a collection of “off-the-shelf” Bria-OTS™ cell lines, four cell lines for each parent tumor cell. Each cell line expresses four exogenous HLA alleles, two HLA-A and two HLA-DRB3/4/5 alleles, for a total of 8 HLA-A and 7 HLA-DRB alleles in 4 cell lines. Together, the Bria-OTS cell line collection has the potential to treat almost 100% of the US population with ≥1 HLA allele matches and about 90% with HLA class I and II double-matches. Once a patient's unique HLA signature is known, they will be assigned one or two complementary Bria-OTS product(s) that sufficiently match his/her HLA profile.

### Innovation

The transformational technology described herein is the development of a therapeutic strategy that addresses the current gap in safe and reliable treatments for patients suffering from advanced solid tumors. Our tumor cell-based immunotherapies significantly improve upon previous attempts to develop whole-cell cancer vaccines, as our platform works through at least two discrete, but complementary mechanisms to stimulate a patient's immune system. The Bria-OTS cell line collection delivers the following features:
Dual mechanisms of action to achieve strong immune response and clinical activity. Bria-OTS cell lines have features of both cancer cells (expressing a myriad of TAAs) and dendritic cells (by presenting TAAs directly to T cells) to enhance the immune response.
Precision therapy. Bria-OTS cell lines will be matched to patients based upon HLA antigens, covering over 99% of the US population and eliciting a cellular response that will recognize the patient's cancer cells.
Improved safety. Relative to current chemotherapeutic and hormone-based therapies that are associated with severe adverse events that can be life threatening.
Rapid, cost-effective treatment. Our “off-the-shelf” cell lines will not require personalized manufacturing and can be adminstered immediately after patient HLA genotyping.

### Approach

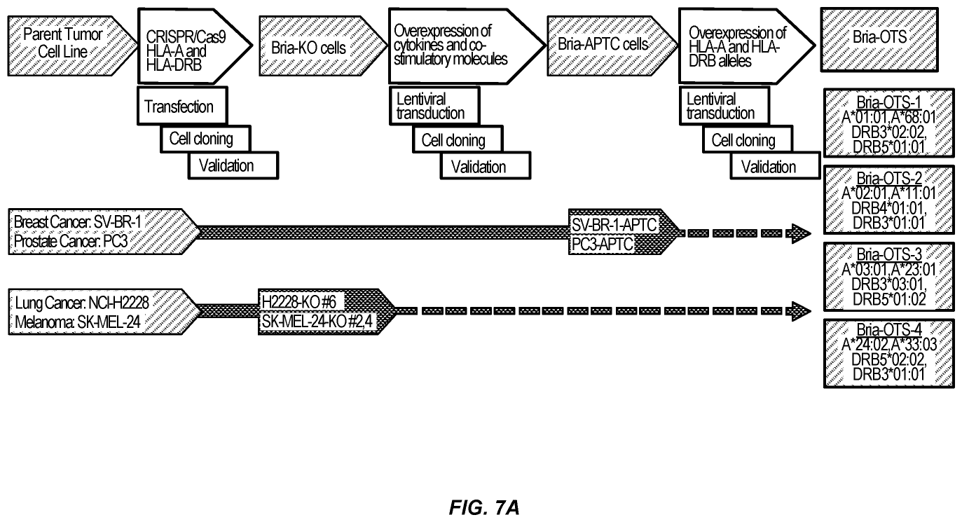

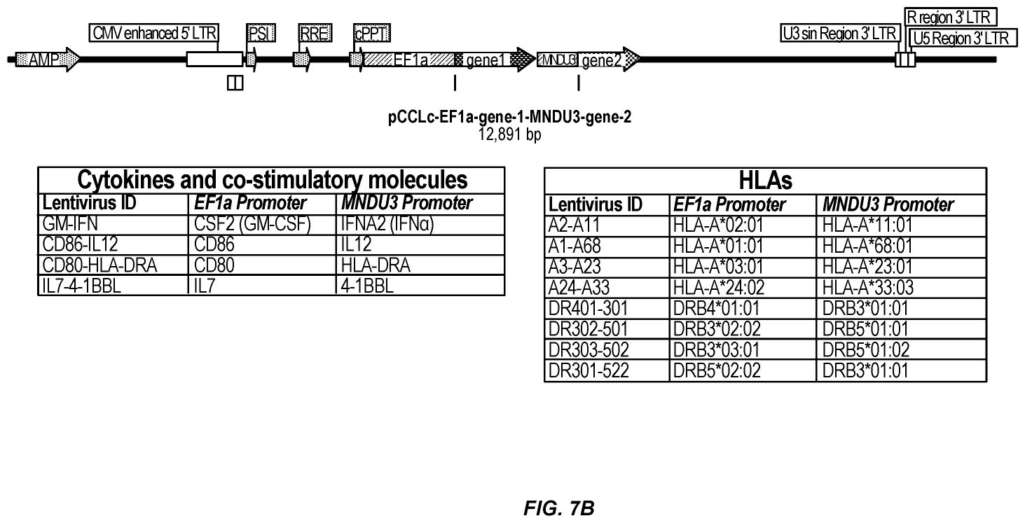

This example describes engineering Bria-OTS cell lines derived from different types of solid tumors (melanoma, breast, prostate, and lung cancers) to express GM-CSF, other cytokines and co-stimulatory molecules, as well as HLA alleles that are not present in the parent tumor cell line. FIG. 7 illustrates the general strategy to develop Bria-OTS cell lines. Briefly, parent tumor cell lines are selected by gene expression profile based on the 22-gene immune signature originally identified in SV-BR-1 breast cancer cells.⁷ Endogenous HLA-A and HLA-DR alleles are knocked out (KO) by CRISPR/Cas9 strategies, followed by single cell cloning and validation to yield Bria-KO cells. Then 8 defined cytokines and co-stimulatory molecules are overexpressed by lentiviral transduction, and their expression confirmed by qRT-PCR, flow cytometry, and/or ELISA in single cell clones, yielding Bria-APTC, which have APC properties. Lastly, unique combinations of 8 HLA-A and 7 HLA-DRB alleles (2 HLA-A and 2-HLA-DRB alleles per cell line) are overexpressed to generate the final Bria-OTS products (4 different cell lines for each cancer cell line). These cell lines deliver precision therapy for patients with solid tumors without the need for personalized manufacturing.

Generating Bria-OTS breast, prostate, melanoma, and lung cancer cell lines. We have selected SV-BR-1 (Her2-positive breast cancer cell line), PC3 (prostate cancer cell line), NCI-H2228 (lung cancer), and SK-MEL-24 cells (melanoma) as parent cancer cell lines based on the expression of a 22-gene immune signature characterized in SV-BR-1 cells, consisting of HLA molecules, cytokines, and chemokines.⁷ Following the general scheme depicted in FIG. 7A, a collection of 4 Bria-OTS cell lines expressing four unique combinations of HLA-A and HLA-DRB alleles can be generated that will match over 99% of the patient population for each specific parent cancer cell line.

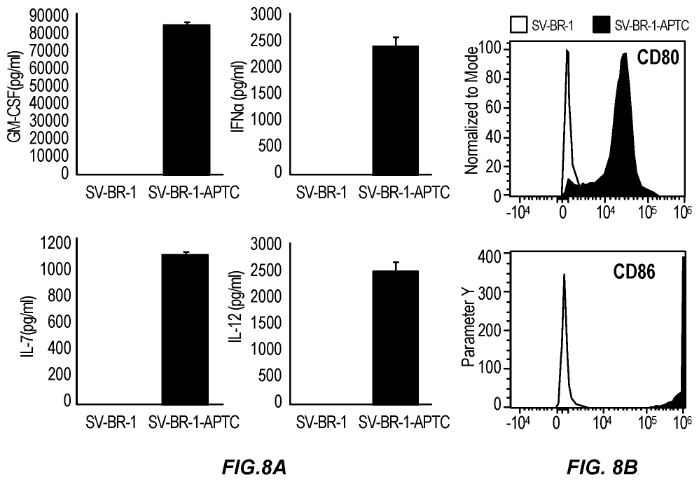

Engineering SV-BR-1-Bria-OTS cells. Following inactivation of endogenous HLA-A and HLA-DRB3 alleles present in parent SV-BR-1 cells by sequential CRISPR/Cas9, as demonstrated by flow cytometry and sequencing, we selected clone 17 for further engineering. We constructed four lentiviral vectors with a pCCLc backbone³⁰ encoding two genes each from two independent promoters (EF1a and MNDU3) to express HLA-DRA, 4 cytokines (GM-CSF, IFN-α, IL-7, IL-12), and 3 co-stimulatory molecules (CD80, CD86, and 4-1BBL). GM-CSF stimulates DC activation and antigen presentation.^{31,32} IFN-α induces a type I response diminishing the development of suppressor mechanisms.^{33,34} IL-7 is a critical cytokine for T cell homeostasis.^{35,36} IL-12 is typically expressed in DCs and provide signal 3 during the priming process.³⁷⁻³⁹ The co-stimulatory molecules are typically expressed by APCs and allow activation of naïve T cells.⁴⁰ SV-BR-1-KO cells, clone 17, were transduced sequentially with all four lentiviruses to generate SV-BR-1-APTC (APTC, for Antigen Presenting Tumor Cells). Expression of all cytokines and co-stimulatory molecules was confirmed by ELISA, flow cytometry, and quantitative real-time PCR (qRT-PCR) (FIG. 8). Enhanced expression of IL-12, IL-7, GM-CSF, and IFN-α was demonstrated by ELISA (FIG. 8A); elevated expression of the cell surface receptors CD80, and CD86 was detected by flow cytometry in clone 17 transduced with all 4 plasmids (FIG. 8B), whereas 4-1BBL was expressed at lower levels.

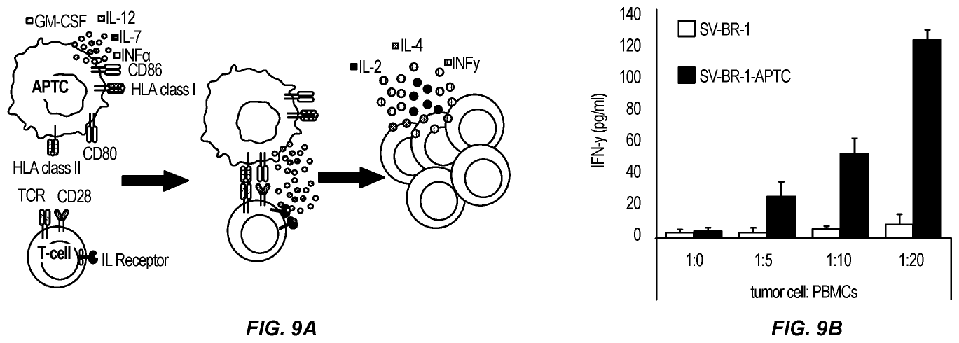

The functionality of these co-stimulatory factors was demonstrated using a modified mixed lymphocyte release (MLR) assay with pooled SV-BR-1-APTC. SV-BR-1-APTC, which express endogenous HLA class I and class II that were not knocked out by CRISPR/Cas9 (FIG. 9A), were co-cultured with peripheral blood mononuclear cells (PBMC) isolated from healthy donors at different ratios 1:5, 1:10, and 1:20 (Tumor cells:PBMCs). The supernatant was collected at days 1, 2, 3, and 7, and secreted IFNγ levels were determined by ELISA as a measure of T cell activation (FIG. 9B shows data for day 1). The levels of secreted IFNγ significantly increased in the presence of SV-BR-1-OTS-APTC in a PBMC:tumor ratio dependent manner; in contrast, reduced IFNγ was detected when PBMCs were incubated with SV-BR-1-KO cells, or when tumor cells were incubated alone.

Following selection and cellular cloning, SV-BR-1-APTC were next transduced with lentiviral vectors expressing two unique HLA-A alleles. We first constructed eight pCCLc-based lentiviral vectors, each containing two HLA-A or two HLA-DRB3/4/5 alleles. The rationale for allele selection is based on studies by Gragert et.al.,²⁹ in which the prevalence of HLA allele frequencies among 16 different US populations were reported. We ranked these frequencies based on overall prevalence, taking the different population sizes into account. The 15 HLAs selected (FIG. 7B) should produce at least a single match in 99% of the US population, with a 92% match at class I HLA-A and 98% match at class II HLA-DR3/4/5 alleles. Plasmid integrity was confirmed by restriction digests and Sanger sequencing and HLA-A expression was confirmed by qRT-PCR.

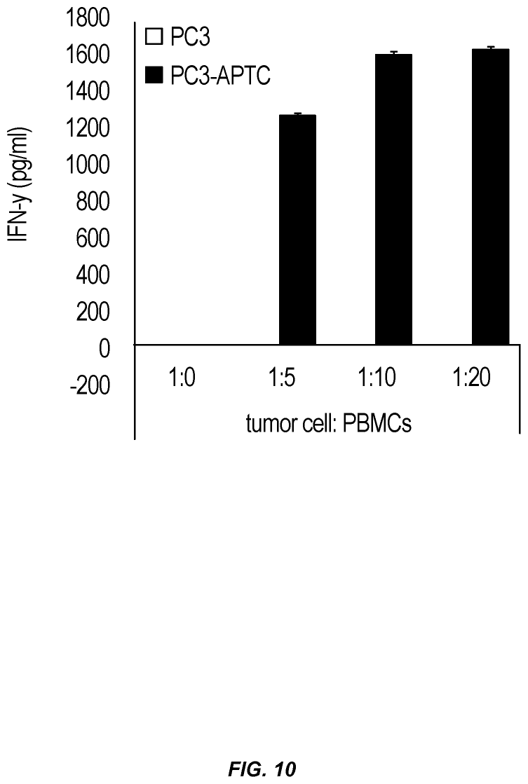

Generation of PC3-APTC cells. Following the inactivation of endogenous HLA-A and HLA-DRB3 alleles present in parental PC3 cells by sequential CRISPR/Cas9, demonstrated by flow cytometry and sequencing, we selected clone A4 for further engineering. We constructed four lentiviral vectors with a pCCLc backbone, encoding two genes each from two independent promoters (EF1a and MNDU3) to express HLA-DRA, 3 cytokines (GM-CSF, IL-7, IL-12), and 3 co-stimulatory molecules (CD80, CD86, and 4-1BBL). These genes were selected based on their immune functions. GM-CSF stimulates DC activation and antigen presentation. IL-7 is a critical cytokine for T cell homeostasis. IL-12 is typically expressed in DCs and provides signal 3 during the priming process. The co-stimulatory molecules are typically expressed by APCs and allow activation of naïve T cells. PC3-KO cells were transduced sequentially with all four lentiviruses to generate PC3-APTC (APTC, for Antigen Presenting Tumor Cells). The expression of cytokines and co-stimulatory molecules was confirmed by flow cytometry, ELISA, and qRT-PCR and their functionality was demonstrated in a MLR assay. PC3-APTC, but not PC3-KO controls, induced substantial IFNγ secretion when co-cultured with PBMCs (FIG. 10).

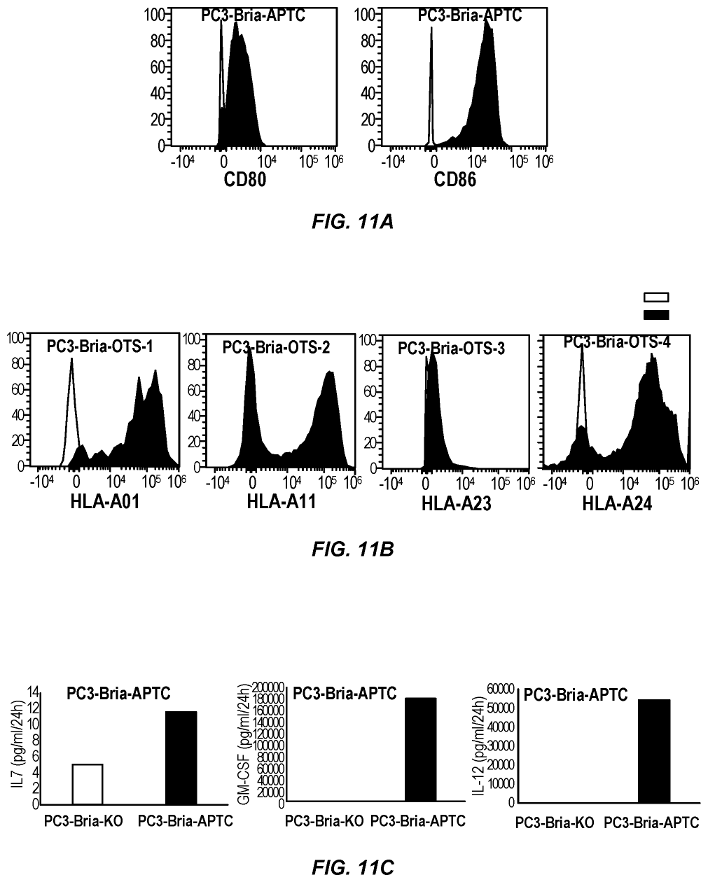

Engineering PC3-Bria-OTS cells. PC3-APTC were subsequently transduced with a pCCLc based lentiviral plasmid encoding one of the following HLA-A allele combinations: A01-A68, A02-A11, A03-A23 or A24-A33 to generate the PC3-Bria-OTS cells. Expression of cytokines and co-stimulatory molecules and HLA-A alleles was confirmed by ELISA and flow cytometry (FIG. 11). The elevated expression of the cell surface receptors CD80 and CD86 and some of the HLA-A alleles was detected by flow cytometry in clone A4 transduced with all 4 plasmids (FIGS. 11A-B). Enhanced expression of IL-7, GM-CSF and IL-12 was demonstrated by ELISA (FIG. 11C).

In summary, we have successfully generated tumor cell lines expressing cytokines, co-stimulatory molecules, and a variety of HLAs.

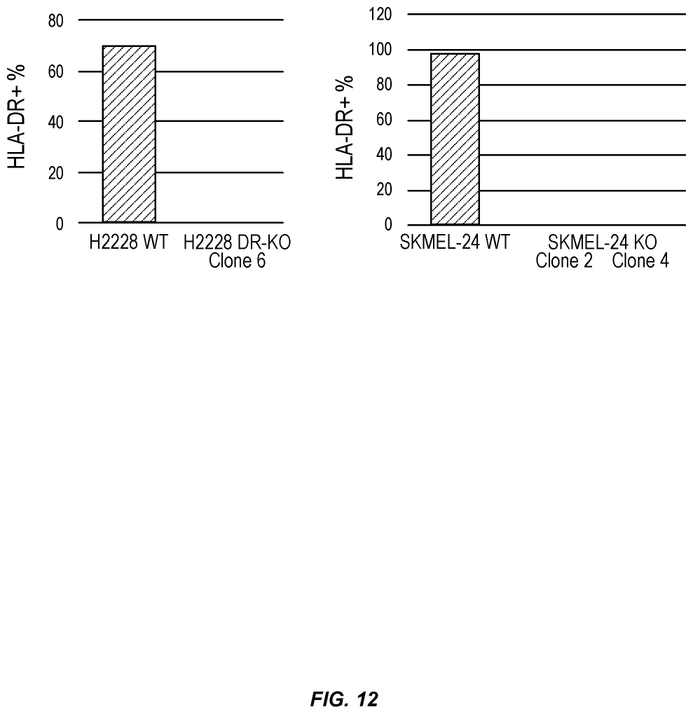

Engineering H2228-Bria-OTS (lung) and SK-MEL-24-Bria-OTS (melanoma) cells. Following the same overall strategy, we have generated H2228 and SK-MEL-24 cells lacking endogenous HLA-DR alleles (FIG. 12) and are in the process of CRISPR/Cas9 KO of endogenous HLA-A alleles. The resulting Bria-KO clonal cell lines are transduced sequentially with all four lentiviral plasmids to produce Bria-APTC by overexpression of GM-CSF, IFN-α, CD80, CD86, IL-12, IL-7, HLA-DRA, and 4-1BBL. Cytokine secretion is evaluated by ELISA, with a target secretion for GM-CSF of ≥100 ng/1×10⁶ cells/24 hours,⁶ which will be sufficient to enhance dendritic cell activity⁶⁷. Cell surface expression of co-stimulatory molecules is confirmed by flow cytometry. In addition, expression of all ectopic genes is further validated by qRT-PCR. All analysis can be performed at least in triplicate on each cell line and the average±SD can be reported. As a final step, clones can be isolated, expanded, and cryopreserved. Selected clones are sequentially transduced with two lentiviral vectors each expressing 2 HLA-A alleles and 2 HLA-DR alleles to obtain 4 final clonal cells for each H2228 and SK-MEL-24 cells.

Demonstrating in vitro functionality of Bria-OTS cells. An MLR assay is used to measure the level of T cell activation induced by Bria-OTS and control Bria-KO cell lines. T-cell proliferation, and levels of secreted IFNγ, IL-2, IL-4, and IL-17 levels are measured at baseline and after 1, 2, 3, and 7 days as a proxy for T cell activation (to cover CD8+ cytotoxic T cells, CD4+Th1, Th2, and Th17 T cell subsets). TCR transgenic Jurkat cell lines that recognize antigens in the context of HLA-A/DR alleles expressed in the Bria-OTS cells are generated to validate the HLA functionality of the final Bria-OTS products.

Modified MLR assay to demonstrate T cell activation by Bria-OTS cells. An MLR assay is performed with the final cell lines as described above. As a control, Bria-KO cells are used. Briefly, Bria-OTS cells are co-cultured in 96 well plates with PBMCs isolated from at least 2 healthy donors (one male and one female) at different tumor:PBMC ratios. T cell proliferation is measured every 24 hours by cell counts and dye dilution. Supernatants are collected at days 0, 1, 2, 3, and 7 to measure levels of IFNγ, IL-2, IL-4, and IL-17 by ELISA.

Validating HLA Functionality of Bria-OTS Cells Using a T Cell Activation Bioassay with TCR Transgenic Jurkat Cells.

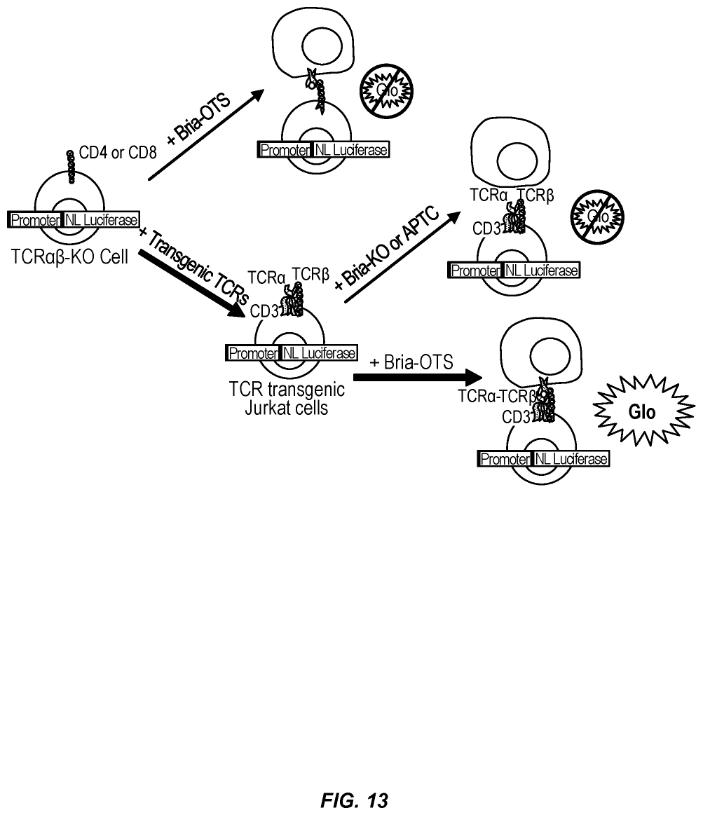

The T cell activation bioassay (Promega) consists of a genetically engineered Jurkat T cell line lacking expression of endogenous TCRα/β subunits (obtained by CRISPR/Cas9-mediated knockout of TCRα/β) that also express a NFAT-driven luciferase reporter. This cell line is useful to measure the ability of transgenic TCR constructs in activating T cells, independently of endogenous TCR expression. We have obtained 3 HLA-A-restricted TCR constructs (HLA-A*02:01, HLA-A*01:01, HLA-A*24:02) from Creative Biolabs and 4 HLA-DR-restricted TCRs (DRB4*0101, DRB3*0101, DRB3*0202, DRB5*0101) from the Benaroya Research Institute (Table 7) to generate 7 different TCR-transgenic Jurkat cell lines that are restricted by some of the HLA-A and HLA-A/DR alleles present in the Bria-OTS cells. All transgenic TCRs can be expressed using pCDTCR1 vector (HLA-As) or the lentivirus gene expression vector pLV (VectorBuilder, HLA-DRs). Co-culture of Bria-OTS cells with TCR transgenic Jurkat cells with TCRs restricted by the HLA-A or DR alleles expressed result in increased luciferase activity (FIG. 13). Negative controls include TCR-transgenic Jurkat cells incubated with Bria-KO/APTC and Bria-OTS incubated with the Jurkat TCRα/β-KO cells without TCRs. Bria-OTS cell lines, but not Bria-KO/APTC, induce a strong IFNγ response in the MLR and NFAT-driven luciferase activity in the TCR transgenic Jurkat assay in an antigen-specific HLA-restricted manner.

TABLE 7
Constructs to generate TCR transgenic Jurkat cells.
HLA-A restricted TCRs

| | | |
|---|---|---|
| TCR-C133Z | Anti-MART1 TCR | HLA-A*02:01 |
| | (AAGIGILTV; SEQ ID NO: 48) (DMF5) | |
| TCR-C124Z | Human anti-MAGE-A3 TCR | HLA-A*01:01 |
| | (EVDPIGHLY; SEQ ID NO: 49) (13-18) | |
| TCR-L360 | Human anti-WT1 TCR | HLA-A*24:02 |
| | (CMTWNQMNL; SEQ ID NO: 50) (37F8) | |

HLA-DR restricted TCRs

| | | | |
|---|---|---|---|
| DRB4*0101 | DRB3*0101 | DRB3*0202 | DRB5*0101 |
| (RSFLVNQMINEAKKQL | (ISLETVAIDRPAEV | (SASHLEQYGTNEYSIIS | (SSFERFEIFPKESS |
| LEFD; | RKV; | SMK; | WPN; |
| SEQ ID NO: 51) | SEQ ID NO: 52) | SEQ ID NO: 53) | SEQ ID NO: 54) |

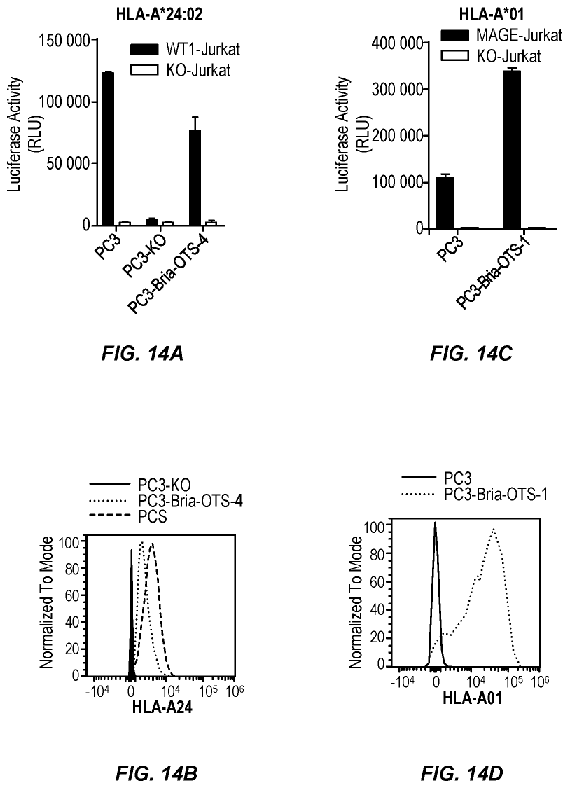

Validation of HLA functionality in PC3-Bria-OTS cells using a T Cell Activation Bioassay (NFAT). Co-culture of PC3-Bria-OTS-4 or PC3-Bria-OTS-1 cells with HLA-A*24 or HLA-A*01 TCR transgenic Jurkat cells resulted in increased luciferase activity (FIGS. 14A and 14C). Each experiment includes appropriate negative controls: TCR transgenic Jurkat cells incubated with PC3 or PC3-KO, and PC3-Bria-OTS cells incubated with the Jurkat TCRα/β-KO cells (without TCRs). Interestingly, activation of transgenic Jurkat cells correlated with HLA-A expression levels (FIGS. 14B and 14D).

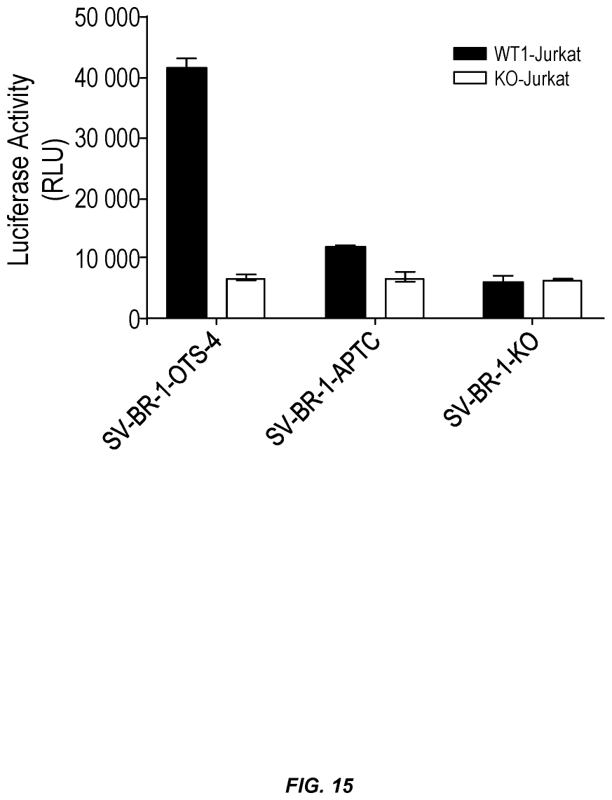

Validation of HLA functionality in SV-BR-1-Bria-OTS cells using a T Cell Activation Bioassay (NFAT). Similarly, co-culture of SV-BR-1-OTS-4 cells with HLA-A24 TCR transgenic Jurkat cells resulted in marked activation as measured by bioluminescence monitoring while the same cells lacking HLA-A24 expression (SV-BR-1-APTC and SV-BR-1-KO) failed to do so (FIG. 15).

Overall, the above results demonstrate that tumor cells expressing transgenic HLA-A alleles are able to induce T-cell activation.

### REFERENCES (EXAMPLE 7 ONLY)

(1) Waldman, A. D.; Fritz, J. M.; Lenardo, M. J. A Guide to Cancer Immunotherapy: From T Cell Basic Science to Clinical Practice. Nat. Rev. Immunol. 2020, 20 (11), 651-668.
(2) Hoteit, M.; Oneissi, Z.; Reda, R.; Wakim, F.; Zaidan, A.; Farran, M.; Abi-Khalil, E.; El-Sibai, M. Cancer Immunotherapy: A Comprehensive Appraisal of Its Modes of Application (Review). Oncol. Lett. 2021, 22 (3), 655.
(3) Anassi, E.; Ndefo, U. A. Sipuleucel-T (Provenge) Injection the First Immunotherapy Agent (Vaccine) for Hormone-Refractory Prostate Cancer. P T 2011, 36 (4), 197-202.
(4) Fu, C.; Zhou, L.; Mi, Q.-S.; Jiang, A. DC-Based Vaccines for Cancer Immunotherapy. Vaccines 2020, 8 (4), 706.
(5) Han, Q.; Wang, Y.; Pang, M.; Zhang, J. STAT3-Blocked Whole-Cell Hepatoma Vaccine Induces Cellular and Humoral Immune Response against HCC. J. Exp. Clin. Cancer Res. 2017, 36 (1), 156.
(6) Wiseman, C. L.; Kharazi, A. Objective Clinical Regression of Metastatic Breast Cancer in Disparate Sites after Use of Whole-Cell Vaccine Genetically Modified to Release Sargramostim. Breast J. 2006, 12 (5), 475-480.
(7) Lacher, M. D.; Bauer, G.; Fury, B.; Graeve, S.; Fledderman, E. L.; Petrie, T. D.; Coleal-Bergum, D. P.; Hackett, T.; Perotti, N. H.; Kong, Y. Y.; Kwok, W. W.; Wagner, J. P.; Wiseman, C. L.; Williams, W. V. SV-BR-1-GM, a Clinically Effective GM-CSF-Secreting Breast Cancer Cell Line, Expresses an Immune Signature and Directly Activates CD4+T Lymphocytes. Front. Immunol. 2018, 9 (MAY), 776.
(8) Wiseman, C. L.; Kharazi, A. Phase I Study with SV-BR-1 Breast Cancer Cell Line Vaccine and GMCSF: Clinical Experience in 14 Patients. Open Breast Cancer J. 2010, 2 (1), 4-11. (9) Wong, K. K.; Li, W. A.; Mooney, D. J.; Dranoff, G. Advances in Therapeutic Cancer Vaccines. In Advances in Immunology; 2016; Vol. 130, pp 191-249.
(10) Sheikh, N.; Cham, J.; Zhang, L.; DeVries, T.; Letarte, S.; Pufnock, J.; Hamm, D.; Trager, J.; Fong, L. Clonotypic Diversification of Intratumoral T Cells Following Sipuleucel-T Treatment in Prostate Cancer Subjects. Cancer Res. 2016, 76 (13), 3711-3718.
(11) Santegoets, S. J. A. M.; Stam, A. G. M.; Lougheed, S. M.; Gall, H.; Jooss, K.; Sacks, N.; Hege, K.; Lowy, I.; Scheper, R. J.; Gerritsen, W. R.; van den Eertwegh, A. J. M.; de Gruijl, T. D. Myeloid Derived Suppressor and Dendritic Cell Subsets Are Related to Clinical Outcome in Prostate Cancer Patients Treated with Prostate GVAX and Ipilimumab. J. Immunother. Cancer 2014, 2 (1), 31.
(12) Tsioulias, G. J. Serum TA90 Antigen-Antibody Complex as a Surrogate Marker for the Efficacy of a Polyvalent Allogeneic Whole-Cell Vaccine (CancerVax) in Melanoma. Ann. Surg. Oncol. 2001, 8 (3), 198-203.
(13) George, D. J.; Nabhan, C.; DeVries, T.; Whitmore, J. B.; Gomella, L. G. Survival Outcomes of Sipuleucel-T Phase III Studies: Impact of Control-Ann Cross-Over to Salvage Immunotherapy. Cancer Immunol. Res. 2015, 3 (9), 1063-1069.
(14) Higano, C. S.; Schellhammer, P. F.; Small, E. J.; Burch, P. A.; Nemunaitis, J.; Yuh, L.; Provost, N.; Frohlich, M. W. Integrated Data from 2 Randomized, Double-Blind, Placebo-Controlled, Phase 3 Trials of Active Cellular Immunotherapy with Sipuleucel-T in Advanced Prostate Cancer. Cancer 2009, 115 (16), 3670-3679.
(15) Small, E. J.; Schellhammer, P. F.; Higano, C. S.; Redfern, C. H.; Nemunaitis, J. J.; Valone, F. H.; Verjee, S. S.; Jones, L. A.; Hershberg, R. M. Placebo-Controlled Phase III Trial of Immunologic Therapy with Sipuleucel-T (APC8015) in Patients with Metastatic, Asymptomatic Hormone Refractory Prostate Cancer. J. Clin. Oncol. 2006, 24 (19), 3089-3094.
(16) Beinart, G.; Rini, B. I.; Weinberg, V.; Small, E. J. Antigen-Presenting Cells 8015 (Provenge®) in Patients with Androgen-Dependent, Biochemically Relapsed Prostate Cancer. Clin. Prostate Cancer 2005, 4 (1), 55-60.
(17) Burch, P. A.; Croghan, G. A.; Gastineau, D. A.; Jones, L. A.; Kaur, J. S.; Kylstra, J. W.; Richardson, R. L.; Valone, F. H.; Vuk-Pavlovid, S. Immunotherapy (APC8015, Provenge®) Targeting Prostatic Acid Phosphatase Can Induce Durable Remission of Metastatic Androgen-Independent Prostate Cancer: A Phase 2 Trial. Prostate 2004, 60 (3), 197-204.
(18) Small, E. J.; Fratesi, P.; Reese, D. M.; Strang, G.; Laus, R.; Peshwa, M. V.; Valone, F. H. Immunotherapy of Hormone-Refractory Prostate Cancer With Antigen-Loaded Dendritic Cells. J. Clin. Oncol. 2000, 18 (23), 3894-3903.
(19) Small, E. J.; Lance, R. S.; Gardner, T. A.; Karsh, L. I.; Fong, L.; McCoy, C.; DeVries, T.; Sheikh, N. A.; GuhaThakurta, D.; Chang, N.; Redfern, C. H.; Shore, N. D. A Randomized Phase II Trial of Sipuleucel-T with Concurrent versus Sequential Abiraterone Acetate plus Prednisone in Metastatic Castration-Resistant Prostate Cancer. Clin. Cancer Res. 2015, 21 (17), 3862-3869.
(20) GuhaThakurta, D.; Sheikh, N. A.; Fan, L.-Q.; Kandadi, H.; Meagher, T. C.; Hall, S. J.; Kantoff, P. W.; Higano, C. S.; Small, E. J.; Gardner, T. A.; Bailey, K.; Vu, T.; DeVries, T.; Whitmore, J. B.; Frohlich, M. W.; Trager, J. B.; Drake, C. G. Humoral Immune Response against Nontargeted Tumor Antigens after Treatment with Sipuleucel-T and Its Association with Improved Clinical Outcome. Clin. Cancer Res. 2015, 21 (16), 3619-3630.
(21) Fong, L.; Carroll, P.; Weinberg, V.; Chan, S.; Lewis, J.; Corman, J.; Amling, C. L.; Stephenson, R. A.; Simko, J.; Sheikh, N. A.; Sims, R. B.; Frohlich, M. W.; Small, E. J. Activated Lymphocyte Recruitment Into the Tumor Microenvironment Following Preoperative Sipuleucel-T for Localized Prostate Cancer. J. Natl. Cancer Inst. 2014, 106 (11).
(22) Beer, T. M.; Bernstein, G. T.; Corman, J. M.; Glode, L. M.; Hall, S. J.; Poll, W. L.; Schellhammer, P. F.; Jones, L. A.; Xu, Y.; Kylstra, J. W.; Frohlich, M. W. Randomized Trial of Autologous Cellular Immunotherapy with Sipuleucel-T in Androgen-Dependent Prostate Cancer. Clin. Cancer Res. 2011, 17 (13), 4558-4567.
(23) Dols, A.; Meijer, S. L.; Smith, J. W.; Fox, B. A.; Urba, W. J. Allogeneic Breast Cancer Cell Vaccines. Clin. Breast Cancer 2003, 3, S173-S180.
(24) Srivatsan, S.; Patel, J. M.; Bozeman, E. N.; Imasuen, I. E.; He, S.; Daniels, D.; Selvaraj, P. Allogeneic Tumor Cell Vaccines. Hum. Vaccin. Immunother. 2014, 10 (1), 52-63.
(25) Kleponis, J.; Skelton, R.; Zheng, L. Fueling the Engine and Releasing the Break: Combinational Therapy of Cancer Vaccines and Immune Checkpoint Inhibitors. Cancer Biology and Medicine. 2015, pp 201-208.
(26) Simons, J. W.; Jaffee, E. M.; Weber, C. E.; Levitsky, H. J.; Nelson, W. G.; Carducci, M. A.; Lazenby, A. J.; Cohen, L. K.; Finn, C. C.; Clift, S. M.; Hauda, K. M.; Beck, L. A.; Leiferman, K. M.; Owens, A. H.; Piantadosi, S.; Dranoff, G.; Mulligan, R. C.; Pardoll, D. M.; Marshall, F. F. Bioactivity of Autologous Irradiated Renal Cell Carcinoma Vaccines Generated by Ex Vivo Granulocyte-Macrophage Colony-Stimulating Factor Gene Transfer. J. Urol. 1998, 57 (8), 617-618.
(27) Klebanoff, C. A.; Acquavella, N.; Yu, Z.; Restifo, N. P. Therapeutic Cancer Vaccines: Are We There Yet? Immunol. Rev. 2011, 239 (1), 27-44.
(28) Li, X. F.; Ren, P.; Shen, W. Z.; Jin, X.; Zhang, J. The Expression, Modulation and Use of Cancer-Testis Antigens as Potential Biomarkers for Cancer Immunotherapy. American Journal of Translational Research. 2020, pp 7002-7019.
(29) Gragert, L.; Madbouly, A.; Freeman, J.; Maiers, M. Six-Locus High Resolution HLA Haplotype Frequencies Derived from Mixed-Resolution DNA Typing for the Entire US Donor Registry. Hum. Immunol. 2013, 74 (10), 1313-1320.
(30) Dull, T.; Zufferey, R.; Kelly, M.; Mandel, R. J.; Nguyen, M.; Trono, D.; Naldini, L. A Third-Generation Lentivirus Vector with a Conditional Packaging System. J. Virol. 1998, 72 (11), 8463-8471.
(31) Gupta, R.; Emens, L. A. GM-CSF-Secreting Vaccines for Solid Tumors: Moving Forward. Discovery medicine. 2010, pp 52-60.
(32) Van De Laar, L.; Coffer, P. J.; Woltman, A. M. Regulation of Dendritic Cell Development by GM-CSF: Molecular Control and Implications for Immune Homeostasis and Therapy. Blood. 2012, pp 3383-3393.
(33) Budhwani, M.; Mazzieri, R.; Dolcetti, R. Plasticity of Type I Interferon-Mediated Responses in Cancer Therapy: From Anti-Tumor Immunity to Resistance. Frontiers in Oncology. 2018, p 322.
(34) Yu, R.; Zhu, B.; Chen, D. Type I Interferon-Mediated Tumor Immunity and Its Role in Immunotherapy. Cell. Mol. Life Sci. 2022, 79 (3), 191.
(35) Schluns, K. S.; Kieper, W. C.; Jameson, S. C.; Lefrangois, L. Interleukin-7 Mediates the Homeostasis of Naïve and Memory CD8 T Cells in Vivo. Nat. Immunol. 2000, 1 (5), 426-432.
(36) Chen, D.; Tang, T.-X.; Deng, H.; Yang, X.-P.; Tang, Z.-H. Interleukin-7 Biology and Its Effects on Immune Cells: Mediator of Generation, Differentiation, Survival, and Homeostasis. Front. Immunol. 2021, 12, 747324.
(37) Colombo, M. P.; Trinchieri, G. Interleukin-12 in Anti-Tumor Immunity and Immunotherapy. Cytokine and Growth Factor Reviews. 2002, pp 155-168.
(38) Henry, C. J.; Ornelles, D. A.; Mitchell, L. M.; Brzoza-Lewis, K. L.; Hiltbold, E. M. IL-12 Produced by Dendritic Cells Augments CD8+ T Cell Activation through the Production of the Chemokines CCL1 and CCL17. J. Immunol. 2008, 181 (12), 8576-8584.
(39) Tugues, S.; Burkhard, S. H.; Ohs, I.; Vrohlings, M.; Nussbaum, K.; Vom Berg, J.; Kulig, P.; Becher, B. New Insights into IL-12-Mediated Tumor Suppression. Cell Death and Differentiation. 2015, pp 237-246.
(40) Jeong, S.; Park, S.-H. Co-Stimulatory Receptors in Cancers and Their Implications for Cancer Immunotherapy. Immune Netw. 2020, 20 (1).

### V. References

Al-Awadhi A, Lee Murray J, Ibrahim N K. Developing anti-HER2 vaccines: Breast cancer experience. Int J Cancer. 2018 Nov. 1; 143(9):2126-2132.
Antonarelli G, Corti C, Tarantino P, Ascione L, Cortes J, Romero P, Mittendorf E A, Disis M L, Curigliano G. Therapeutic cancer vaccines revamping: technology advancements and pitfalls. Ann Oncol. 2021 December; 32(12):1537-51.
Arico E, Belardelli F. Interferon-alpha as antiviral and antitumor vaccine adjuvants: mechanisms of action and response signature. J Interferon Cytokine Res. 2012; 32(6):235-47.
Avigan D, Vasir B, Gong J, et al. Fusion cell vaccination of patients with metastatic breast and renal cancer induces immunological and clinical responses. Clin Cancer Res. 2004; 10(14):4699-708.
Berd D, Maguire H C Jr, Mastrangelo M J. Induction of cell-mediated immunity to autologous melanoma cells and regression of metastases after treatment with a melanoma cell vaccine preceded by cyclophosphamide. Cancer Res. 1986; 46(5):2572-7.
Boyer C M, Dawson D B, Nea, SE, Winchell L F, et al. Differential induction by interferons of major histocompatibility complex-encoded and non-major histocompatibility complex-encoded antigens in human breast and ovarian carcinoma cell lines. Cancer Research 1989; 49:2928-34.
Bray F, Ferlay J, Soerjomataram I, Siegel R L, Torre L A, Jemal A. Global cancer statistics 2018: GLOBOCAN estimates of incidence and mortality worldwide for 36 cancers in 185 countries. CA Cancer J Clin. 2018 November; 68(6):394-424.
Brinkmann U, Kontermann R E. The making of bispecific antibodies. MAbs. 2017 February/March; 9(2):182-212.
Carson W E 3rd, Unger J M, Sosman J A, Flaherty L E, Tuthill R J, et al. Adjuvant vaccine immunotherapy of resected, clinically node-negative melanoma: long-term outcome and impact of HLA class I antigen expression on overall survival. Cancer Immunol Res. 2014 October; 2(10):981-7.
Chen G, Gupta R, Petrik S, Laiko M, Leatherman J M, Asquith J M, et al. A feasibility study of cyclophosphamide, trastuzumab, and an allogeneic GM-CSF-secreting breast tumor vaccine for HER2+ metastatic breast cancer. Cancer Immunol Res. 2014; 2(10):949-61.
Chornoguz, Olesya et al. “Major histocompatibility complex class II+ invariant chain negative breast cancer cells present unique peptides that activate tumor-specific T cells from breast cancer patients.” Molecular & cellular proteomics: MCP vol. 11, 11 (2012): 1457-67.
Corti C, Giachetti PPMB, Eggermont A M M, Delaloge S, Curigliano G. Therapeutic vaccines for breast cancer: Has the time finally come? Eur J Cancer. 2021 Nov. 22:S0959-8049(21)01185-0.
Creelan B C, Antonia S, Noyes D, Hunter T B, Simon G R, Bepler G, et al. Phase II trial of a GM-CSF-producing and CD40L-expressing bystander cell line combined with an allogeneic tumor cell-based vaccine for refractory lung adenocarcinoma. J Immunother. 2013; 36(8):442-50.
Disis M L, Grabstein K H, Sleath P R, Cheever M A. Generation of immunity to the HER-2/neu oncogenic protein in patients with breast and ovarian cancer using a peptide-based vaccine. Clin Cancer Res. 1999 June; 5(6):1289-97.
Dols A, Smith J W 2nd, Meijer S L, et al. Vaccination of women with metastatic breast cancer, using a costimulatory gene (CD80)-modified, HLA-A2-matched, allogeneic, breast cancer cell line: clinical and immunological results. Hum Gene Ther. 2003; 14(11):1117-23.
Dranoff G, Jaffee E, Lazenby A, Golumbek P, Levitsky H, et al. Vaccination with irradiated tumor cells engineered to secrete murine granulocyte-macrophage colony-stimulating factor stimulates potent, specific, and long-lasting anti-tumor immunity. Proc Nail Acad Sci USA. 1993 Apr. 15; 90(8):3539-43.
Dull, T et al. “A third-generation lentivirus vector with a conditional packaging system.” Journal of virology vol. 72, 11 (1998): 8463-71. doi:10.1128/JVI.72.11.8463-8471.1998.
Elliott R L, Head J F. Adjuvant breast cancer vaccine improves disease specific survival of breast cancer patients with depressed lymphocyte immunity. Surg Oncol. 2013; 22(3):172-7.
Emens L A, Armstrong D, Biedrzycki B, Davidson N, Davis-Sproul J, et al. A phase I vaccine safety and chemotherapy dose-finding trial of an allogeneic GM-CSF-secreting breast cancer vaccine given in a specifically timed sequence with immunomodulatory doses of cyclophosphamide and doxorubicin. Hum Gene Ther. 2004 March; 15(3):313-37.
Emens L A, Asquith J M, Leatherman J M, Kobrin B J, Petrik S, Laiko M, et al. Timed sequential treatment with cyclophosphamide, doxorubicin, and an allogeneic granulocyte-macrophage colony-stimulating factor-secreting breast tumor vaccine: a chemotherapy dose-ranging factorial study of safety and immune activation. J Clin Oncol. 2009; 27(35):5911-8.
Ferlay J, Colombet M, Soerjomataram I, Mathers C, Parkin D M, et al. Estimating the global cancer incidence and mortality in 2018: GLOBOCAN sources and methods. Int J Cancer. 2019 Apr. 15; 144(8):1941-53.
Garg A D, Vara Perez M, Schaaf M, Agostinis P, Zitvogel L, et al. Trial watch: Dendritic cell-based anticancer immunotherapy. Oncoimmunology. 2017 May 12; 6(7):e1328341.
Gelao L, Criscitiello C, Esposito A, De Laurentiis M, Fumagalli L, et al. Dendritic cell-based vaccines: clinical applications in breast cancer. Immunotherapy. 2014; 6(3):349-60.
Giaccone G, Bazhenova L A, Nemunaitis J, Tan M, Juhisz E, et al. A phase III study of belagenpumatucel-L, an allogeneic tumour cell vaccine, as maintenance therapy for non-small cell lung cancer. Eur J Cancer. 2015 November; 51(16):2321-9.
Groeper C, Gambazzi F, Zajac P, Bubendorf L, Adamina M, et al. Cancer/testis antigen expression and specific cytotoxic T lymphocyte responses in non small cell lung cancer. Int J Cancer. 2007; 120(2):337-43.
Gupta R, Emens L A. GM-CSF-secreting vaccines for solid tumors: moving forward. Discov Med. 2010; 10(50):52-60.
Hadden J W. The immunology and immunotherapy of breast cancer: an update. International Journal of Immunopharmacology 1999; 21:79-101.
Jacot W, Heudel P E, Fraisse J, Gourgou S, Guiu S, et al. Real-life activity of eribulin mesylate among metastatic breast cancer patients in the multicenter national observational ESME program. Int J Cancer. 2019 Dec. 15; 145(12):3359-69.
Jiang X P, Yang D C, Elliott R L, Head J F. Vaccination with a mixed vaccine of autogenous and allogeneic breast cancer cells and tumor associated antigens CA15-3, CEA and CA125—results in immune and clinical responses in breast cancer patients. Cancer Biother Radiopharm. 2000 October; 15(5):495-505.
Kazmi S, Chatterjee D, Raju D, Hauser R, Kaufman P A. Overall survival analysis in patients with metastatic breast cancer and liver or lung metastases treated with eribulin, gemcitabine, or capecitabine [published correction appears in Breast Cancer Res Treat. 2021 June; 187(2):603]. Breast Cancer Res Treat. 2020; 184(2):559-65.
Keenan B P, Jaffee E M. Whole cell vaccines—past progress and future strategies. Semin Oncol. 2012; 39(3):276-86.
Khan, S. H. (2019). Genome-editing technologies: concept, pros, and cons of various genome-editing techniques and bioethical concerns for clinical application. Molecular Therapy-Nucleic Acids, 16, 326-334.
Kharazi A, Kudinov Y, Cuevas A, Wiseman, CL. Effect of interferon-alpha on the phenotype of human monocyte-derived dendritic cells generated in the presence of GM-CSF in vitro. European Congress of Immunology, Rhodes, Greece; 2003; 87:69.
Kuroi K, Sato Y, Yamaguchi Y, Toge T. Modulation of suppressor cell activities by cyclophosphamide in breast cancer patients. Journal of Clinical Laboratory Analysis 1994; 8:123-7.
Lacher M D, Bauer G, Fury B, Graeve S, Fledderman E L, Petrie T D, Coleal-Bergum D P, Hackett T, Perotti N H, Kong Y Y, Kwok W W, Wagner J P, Wiseman C L, Williams W V. SV-BR-1-GM, a Clinically Effective GM-CSF-Secreting Breast Cancer Cell Line, Expresses an Immune Signature and Directly Activates CD4+T Lymphocytes. Front. Immunol., 15 May 2018.
Le D T, Wang-Gillam A, Picozzi V, Greten T F, Crocenzi T, Springett G, et al. Safety and survival with GVAX pancreas prime and Listeria monocytogenes-expressing mesothelin (CRS-207) boost vaccines for metastatic pancreatic cancer. J Clin Oncol. 2015; 33(12):1325-33.
Leo S, Arnoldi E, Repetto L, et al. Eribulin Mesylate as Third or Subsequent Line Chemotherapy for Elderly Patients with Locally Recurrent or Metastatic Breast Cancer: A Multicentric Observational Study of GIOGer (Italian Group of Geriatric Oncology)-ERIBE. Oncologist. 2019; 24(6):e232-e240.
Lipson E J, Sharfman W H, Chen S, McMiller T L, Pritchard T S, Salas J T, et al. Safety and immunologic correlates of melanoma GVAX, a GM-CSF secreting allogeneic melanoma cell vaccine administered in the adjuvant setting. J Transl Med. 2015; 13:214.
Logan A C, Nightingale S J, Haas D L, Cho G J, Pepper K A, Kohn D B. Factors influencing the titer and infectivity of lentiviral vectors. Hum Gene Ther. 2004 October; 15(10):976-88. doi: 10.1089/hum.2004.15.976. PMID: 15585113.
Lollini P L, Cavallo F, Nanni P, Quaglino E. The promise of preventive cancer vaccines. Vaccines (Basel). 2015; 3(2):467-89.
Machiels J P, Reilly R T, Emens L A, Ercolini A M, Lei R Y, et al. Cyclophosphamide, doxorubicin, and paclitaxel enhance the antitumor immune response of granulocyte/macrophage-colony stimulating factor-secreting whole-cell vaccines in HER-2/neu tolerized mice. Cancer Res. 2001; 61(9):3689-97.
Maeda S, Saimura M, Minami S, et al. Efficacy and safety of eribulin as first- to third-line treatment in patients with advanced or metastatic breast cancer previously treated with anthracyclines and taxanes. Breast. 2017; 32:66-72.
Manso L, Moreno Antón F, Izarzugaza Perón Y, et al. Safety of eribulin as third-line chemotherapy in HER2-negative, advanced breast cancer pre-treated with taxanes and anthracycline: OnSITE study. Breast J. 2019; 25(2):219-25.
Maude S L, Frey N, Shaw P A, Aplenc R, Barrett D M, et al. Chimeric antigen receptor T cells for sustained remissions in leukemia. N Engl J Med. 2014; 371(16):1507-17.
Maus M V, Grupp S A, Porter D L, June C H. Antibody-modified T cells: CARs take the front seat for hematologic malignancies. Blood. 2014; 123(17):2625-35.
Mittendorf E A, Clifton G T, Holmes J P, Schneble E, van Echo D, et al. Final report of the phase I/II clinical trial of the E75 (nelipepimut-S) vaccine with booster inoculations to prevent disease recurrence in highrisk breast cancer patients. Ann Oncol. 2014; 25(9):1735-42.
NCCN Clinical Practice Guidelines in Oncology: Breast Cancer. NCCN Evidence Blocks. Version 8.2021—Sep. 13, 2021. NCCN.org.
Pallerla S, Abdul AURM, Comeau J, Jois S. Cancer Vaccines, Treatment of the Future: With Emphasis on HER2-Positive Breast Cancer. Int J Mol Sci. 2021 Jan. 14; 22(2):779.
Paquette R, Hsu N, Kiertscher S, et al. Interferon-alpha and granulocyte-macrophage colony-stimulating factor differentiate peripheral blood monocytes into potent antigen-presenting cells. J Leukoc Biol. 1998; 64:358-67.
Prickett T D, Crystal J S, Cohen C J, Pasetto A, Parkhurst M R, et al. Durable complete response from metastatic melanoma after transfer of autologous T cells recognizing 10 mutated tumor antigens. Cancer Immunol Res 2016; 4(8):669-78.
Proietti E, Bracci L, Puzelli S, Di Pucchio T, Sestili P, et al. Type I IFN as a natural adjuvant for a protective immune response: lessons from the influenza vaccine model. J Immunol. 2002 Jul. 1; 169(1):375-83.
Ramamoorth, Murali, and Aparna Narvekar. “Non viral vectors in gene therapy—an overview.” Journal of clinical and diagnostic research: JCDR vol. 9, 1 (2015): GE01-6. doi:10.7860/JCDR/2015/10443.5394.
Renkvist N, Castelli C, Robbins P F, Parmiani G. A listing of human tumor antigens recognized by T cells. Cancer Immunol Immunother. 2001; 50(1):3-15.
Robbins P F, Kassim S H, Tran T L, Crystal J S, Morgan R A, et al. A pilot trial using lymphocytes genetically engineered with an NY-ESO-1-reactive T-cell receptor: long-term follow-up and correlates with response. Clin Cancer Res 2015; 21(5):1019-27.
Ross J S, Slodkowska E A, Symmans W F, Pusztai L, Ravdin P M, Hortobagyi G N. The HER-2 receptor and breast cancer: ten years of targeted anti-HER-2 therapy and personalized medicine. Oncologist. 2009 April; 14(4):320-68.
Rugo H S, Im S A, Cardoso F, Cortes J, Curigliano G, et al. and SOPHIA Study Group. Efficacy of Margetuximab vs Trastuzumab in Patients With Pretreated ERBB2-Positive Advanced Breast Cancer: A Phase 3 Randomized Clinical Trial. JAMA Oncol. 2021 Apr. 1; 7(4):573-84.
Sakai Y, Morrison B J, Burke J D, et al. Vaccination by genetically modified dendritic cells expressing a truncated neu oncogene prevents development of breast cancer in transgenic mice. Cancer Res. 2004; 64(21):8022-28.
Santegoets S J, Stain A G, Lougheed S M, Gall H, Jooss K, et al. Myeloid derived suppressor and dendritic cell subsets are related to clinical outcome in prostate cancer patients treated with prostate GVAX and ipilimumab. J Immunother Cancer. 2014; 2:31.
Santini S M, Lapenta C, Logozzi M, Parlato S, Spada M, et al. Type I interferon as a powerful adjuvant for monocyte-derived dendritic cell development and activity in vitro and in Hu-PBL-SCID mice. J Exp Med. 2000 May 15; 191(10):1777-88.
SEER Cancer Stat Facts: Female Breast Cancer. National Cancer Institute. Bethesda, MD, seer.cancer.gov/statfacts/html/breast.html. Accessed 20 Oct. 2021.
Shaimardanova et al., “Production and application of multicistronic constructs for various human disease therapies.” Pharmaceutics 2019, 11, 580.
Silvestri I, Cattarino S, Aglianó A M, Collalti G, Sciarra A. Beyond the Immune Suppression: The Immunotherapy in Prostate Cancer. Biomed Res Int. 2015; 2015:794968.
Sondak V K, Liu P Y, Tuthill R J, Kempf R A, Unger J M, et al. Adjuvant immunotherapy of resected, intermediate-thickness, node-negative melanoma with an allogeneic tumor vaccine: overall results of a randomized trial of the Southwest Oncology Group. J Clin Oncol. 2002 Apr. 15; 20(8):2058-66.
Srivatsan S, Patel J M, Bozeman E N, Imasuen I E, He S, et al. Allogeneic tumor cell vaccines: the promise and limitations in clinical trials. Hum Vaccin Immunother. 2014; 10(1):52-63. van der Bruggen Vigneron N, Stroobant V, Van den Eynde B J, van der Bruggen P. Database of T cell-defined human tumor antigens: the 2013 update. Cancer Immun. 2013; 13:15.
Varella L, Eziokwu A S, Jia X, Kruse M, Moore H C F, et al. Real-world clinical outcomes and toxicity in metastatic breast cancer patients treated with palbociclib and endocrine therapy. Breast Cancer Res Treat. 2019 July; 176(2):429-34.
Wallack M K, Sivanandham M, Balch C M, Urist M M, Bland K I, et al. Surgical adjuvant active specific immunotherapy for patients with stage III melanoma: the final analysis of data from a phase III, randomized, double-blind, multicenter vaccinia melanoma oncolysate trial. J Am Coll Surg. 1998 July; 187(1):69-77; discussion 77-9.
Wallack M K, Sivanandham M, Balch C M, Urist M M, et al. A phase III randomized, double-blind multiinstitutional trial of vaccinia melanoma oncolysate-active specific immunotherapy for patients with stage II melanoma. Cancer. 1995 Jan. 1; 75(1):34-42.
Wiseman C, Kharazi A, Cuevas A, Berliner K. Whole-cell based breast cancer vaccine/GM-CSF: Clinical experience in 13 patients with cell line SV-BR-1. SBT 2004 Annual Meeting. J Immunother. 2004a; 27:S34.
Wiseman C, Kharazi A, Cuevas A, Berliner K. Whole-cell breast cancer vaccine/GM-CSF: Clinical experience in 13 patients with cell line SV-R-1. Int. Soc. Bio. Ther. Annual Meeting. 2004b.
Wiseman C L, Kharazi A. “Regression of brain metastases in two breast cancer patients with irradiated allogeneic whole-cell vaccines (V), low-dose cyclophosphamide (CY), and GMCSF”; presented at the Annual Meeting of International Society of Biological Therapy, 2006a.
Wiseman C L, Kharazi A. Regression of brain metastases in two breast cancer patients with irradiated allogeneic whole-cell vaccines, low dose cyclophosphamide, and GM-CSF. International Society for Biological Therapy of Cancer (iSBT) Annual Meeting. J Immunother. 2006b.
Wiseman C L, Kharazi A. Objective clinical regression of metastatic breast cancer in disparate sites after use of whole-cell vaccine genetically modified to release sargramostim. Breast J. 2006c September-October; 12(5):475-80.
Wiseman C L, Kharazi A. Phase I Study with SV-BR-1 Breast Cancer Cell Line Vaccine and GMCSF: Clinical Experience in 14 Patients. The Open Breast Cancer Journal. 2010; 2:4-11.
Yin, H., Kauffman, K. & Anderson, D. Delivery technologies for genome editing. Nat Rev Drug Discov 16, 387-399 (2017). doi.org/10.1038/nrd.2016.280.
Zhang P, Yi S, Li X, et al. Preparation of triple-negative breast cancer vaccine through electrofusion with day-3 dendritic cells. PLoS One. 2014; 9(7):e102197.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for all purposes.

1. A modified human cancer cell comprising: (a) one or more recombinant polynucleotides each encoding an allele of a human leukocyte antigen (HLA) class I gene; and (b) one or more recombinant polynucleotides each encoding an allele of an HLA class II gene, wherein one or more HLA alleles endogenous to the cell have been inactivated.

2. The modified human cancer cell of claim 1, wherein (a) comprises a first recombinant polynucleotide encoding a first allele of an HLA class I gene and a second recombinant polynucleotide encoding a second allele of the HLA class I gene.

3. The modified human cancer cell of claim 1, wherein (b) comprises a first recombinant polynucleotide encoding an allele of a first HLA class II gene and a second recombinant polynucleotide encoding an allele of a second HLA class II gene.

4. (canceled)

5. The modified human cancer cell of claim 1, wherein the HLA class I gene comprises an HLA-A gene, an HLA-B gene, an HLA-C gene, an HLA-E gene, an HLA-F gene, an HLA-G gene, a beta-2-microglobulin (B2M) gene, or a combination thereof.

6. (canceled)

7. The modified human cancer cell of claim 5, wherein the allele of the HLA-A gene comprises:
(i) an HLA-A*01:01 allele and an HLA-A*68:01 allele;
(ii) an HLA-A*02:01 allele and an HLA-A*11:01 allele;
(iii) an HLA-A*03:01 allele and an HLA-A*23:01 allele; or
(iv) an HLA-A*24:02 allele and an HLA-A*33:03 allele.

8. (canceled)

9. (canceled)

10. The modified human cancer cell of claim 1, wherein the HLA class II gene comprises an HLA-DP gene, an HLA-DM gene, an HLA-DO gene, an HLA-DQ gene, an HLA-DR gene, or a combination thereof.

11. (canceled)

12. The modified human cancer cell of claim 10, wherein the HLA-DR gene comprises an HLA-DRA gene, an HLA-DRB1 gene, an HLA-DRB3 gene, an HLA-DRB4 gene, an HLA-DRB5 gene, or a combination thereof.

13-15. (canceled)

16. The modified human cancer cell of claim 10, wherein the allele of the HLA-DR gene comprises:
(i) an HLA-DRB3*02:02 allele and an HLA-DRB5*01:01 allele;
(ii) an HLA-DRB4*01:01 allele and an HLA-DRB3*01:01 allele;
(iii) an HLA-DRB3*03:01 allele and an HLA-DRB5*01:02 allele; or
(iv) an HLA-DRB5*02:02 allele and an HLA-DRB3*01:01 allele.

17-22. (canceled)

23. The modified human cancer cell of claim 1, further comprising one or more recombinant polynucleotides each encoding a cytokine.

24. The modified human cancer cell of claim 23, wherein the cytokine comprises a chemokine, an interferon, an interleukin, a tumor necrosis factor, or a combination thereof.

25. The modified human cancer cell of claim 24, wherein the the cytokine comprises an early T cell activation antigen-1 (ETA-1), a lymphocyte-activating factor (LAF), an interleukin-1 family member (IL-1α, IL-β, IL-1Ra, IL-18, IL-33, IL-36Ra, IL-36α, IL-36β), IL-36γ, IL-37, IL-38), an interleukin-2 (IL-2), an interleukin-3 (IL-3), an interleukin-4 (IL-4), an interleukin-5 (IL-5), an interleukin-6 (IL-6), an interleukin-7 (IL-7), an interleukin-8 (IL-8), an interleukin-9 (IL-9), an interleukin-10 (IL-10), an interleukin-12 (IL-12), an interleukin-13 (IL-13), an interleukin-15 (IL-15), an interleukin-17 (IL-17), an interleukin-18 (IL-18), an interleukin-21 (IL-21), an interleukin-23 (IL-23), an interleukin-25 (IL-25), an interleukin-33 (IL-33), an interferon alpha (IFN-α), an interferon lambda 1 (IFNλ1 (IL-29)), an interferon lambda 2 (IFNλ2 (IL-28A)), an interferon lambda 3 (IFNλ1 (IL-28B)), an interferon lambda 4 (IFNλ4), a granulocyte-macrophage colony-stimulating factor (GM-CSF), a macrophage CSF (CSF-1), a macrophage migration inhibitory factor (MIF), a CD40L molecule (CD40L), a RANTES molecule (RANTES), a monocyte chemoattractant protein (MCP-1), a monocyte inflammatory protein (MIP-1α, MIP-1β), a lymphotactin, a fractalkine, or a combination thereof.

26. The modified human cancer cell of claim 23, wherein the cytokine comprises a granulocyte-macrophage colony-stimulating factor (GM-CSF).

27. The modified human cancer cell of claim 23, wherein the cytokine comprises an interferon alpha (IFN-α), an interferon lambda 1 (IFNλ1 (IL-29)), an interferon lambda 2 (IFNλ2 (IL-28A)), an interferon lambda 3 (IFNλ1 (IL-28B)), or an interferon lambda 4 (IFNλ4).

28. The modified human cancer cell of claim 1, further comprising one or more recombinant polynucleotides each encoding a co-stimulatory molecule.

29. The modified human cancer cell of claim 28, wherein the co-stimulatory molecule comprises a CD86 molecule (CD86), CD80 molecule (CD80), 4-1BB ligand molecule (4-1BBL a.k.a CD137L), ICOS ligand molecule (ICOS-L), CD70 molecule (CD70 a.k.a. CD27L), CD40 molecule (CD40), OX40 ligand molecule (OX40L), GITR ligand molecule (GITRL), TIM-4 molecule (TIM-4), LIGHT molecule (LIGHT), ICAM1 molecule (ICAM1), LFA3 molecule (LFA3), a CD30 molecule (CD30), or a combination thereof.

30. (canceled)

31. The modified human cancer cell of claim 1, wherein one or more HLA-A and/or HLA-DR alleles endogenous to the cell have been inactivated.

32. The modified human cancer cell of claim 31, wherein the one or more HLA-A alleles comprises an HLA-A*24:02 allele and/or an HLA-A*11:01 allele.

33. The modified human cancer cell of claim 31, wherein the one or more HLA-DR alleles comprises one or more HLA-DRB3 alleles.

34. The modified human cancer cell of claim 33, wherein the one or more HLA-DRB3 alleles comprises an HLA-DRB3*01:01 allele and/or an HLA-DRB3*02:02 allele.

35. The modified human cancer cell of claim 1, wherein one or more of the recombinant polynucleotides is present on a vector in the cell.

36. The modified human cancer cell of claim 35, wherein the vector comprises two recombinant polynucleotides each encoding an allele of an HLA class I gene.

37. The modified human cancer cell of claim 35, wherein the vector comprises two recombinant polynucleotides each encoding an allele of an HLA class II gene.

38. (canceled)

39. (canceled)

40. The modified human cancer cell of claim 1, wherein the one or more recombinant polynucleotides each encoding an allele of an HLA class I gene and the one or more recombinant polynucleotides each encoding an allele of an HLA class II gene are present on separate vectors in the cell.

41. The modified human cancer cell of claim 1, wherein the one or more recombinant polynucleotides each encoding two alleles of HLA class I genes and the one or more recombinant polynucleotides each encoding two alleles of HLA class II genes are present on separate vectors in the cell.

42. (canceled)

43. (canceled)

44. The modified human cancer cell of claim 1, wherein the human cancer cell is a human cancer cell line.

45. The modified human cancer cell of claim 44, wherein the human cancer cell line is a breast cancer, prostate cancer, melanoma, or lung cancer cell line.

46-50. (canceled)

51. A modified human cancer cell comprising:
(a) one or more vectors each comprising a recombinant polynucleotide encoding at least one gene selected from GM-CSF, IFN-α, CD86, IL-12, CD80, HLA-DRA, IL-7, CD40, and 4-1BBL;
(b) one or more vectors each comprising a recombinant polynucleotide encoding at least one HLA-A allele selected from an HLA-A*01:01 allele, an HLA-A*68:01 allele, an HLA-A*02:01 allele, an HLA-A*11:01 allele, an HLA-A*03:01 allele, an HLA-A*23:01 allele, an HLA-A*24:02, and an HLA-A*33:03 allele; and
(c) one or more vectors each comprising a recombinant polynucleotide encoding at least one HLA-DR allele selected from an HLA-DRB3*02:02, HLA-DRB5*01:01, HLA-DRB4*01:01, HLA-DRB3*01:01, HLA-DRB3*03:01, HILA-DRB5*01:02, and/or HLA-DRB5*02:02,
wherein one or more HLA alleles endogenous to the cell have been inactivated.

52. The modified human cancer cell of claim 51, wherein:
(b) comprises one vector comprising a recombinant polynucleotide encoding an HLA-A*01:01 allele and an HLA-A*68:01 allele, or an HLA-A*02:01 allele and an HLA-A*l 1:01 allele, or an HLA-A*03:01 allele and an HLA-A*23:01 allele, or an HLA-A*24:02 allele and an HLA-A*33:03 allele; and
(c) comprises one vector comprising a recombinant polynucleotide encoding an HLA-DRB3*02:02 allele and an HLA-DRB5*01:01 allele, or an HLA-DRB4*01:01 allele and an HLA-DRB3*01:01 allele, or an HLA-DRB3*03:01 allele and an HLA-DRB5*01:02 allele, or an HLA-DRB5*02:02 allele and an HLA-DRB3*01:01 allele.

53. The modified human cancer cell of claim 51, wherein:
(a) comprises four vectors each comprising a recombinant polynucleotide encoding two different genes selected from CM-CSF, IFN-α, CD86, IL-12, CD80, HLA-DRA, IL-7, and CD40.

54. The modified human cancer cell of claim 51, wherein one or more HLA-A and/or HLA-DR alleles endogenous to the cell have been inactivated.

55. The modified human cancer cell of claim 54, wherein the one or more inactivated endogenous HLA-A alleles comprises an HLA-A*24:02 allele and/or an HLA-A*11:01 allele.

56. The modified human cancer cell of claim 54, wherein the one or more inactivated endogenous HLA-DR alleles comprises one or more HLA-DRB3 alleles.

57. The modified human cancer cell of claim 56, wherein the one or more inactivated endogenous HLA-DRB3 alleles comprises an HLA-DRB3*01:01 allele and/or an HLA-DRB3*02:02 allele.

58. The modified human cancer cell of claim 51, wherein one or more of the recombinant polynucleotides comprises a sequence having at least 85% identity to any one of SEQ ID NOS:22-44.

59. The modified human cancer cell of claim 51, wherein one or more of the recombinant polynucleotides comprises the sequence of any one of SEQ ID NOS:22-44.

60. The modified human cancer cell of claim 51, wherein the human cancer cell is a human cancer cell line.

61. The modified human cancer cell of claim 60, wherein the human cancer cell line is a breast cancer, prostate cancer, melanoma, or lung cancer cell line.

62. A replication-incompetent modified human cancer cell of claim 1.

63. (canceled)

64. A composition comprising a modified human cancer cell of claim 1.

65. (canceled)

66. A pharmaceutical composition comprising the composition of claim 64 and a pharmaceutically acceptable carrier.

67. (canceled)

68. A method for treating cancer in a subject, the method comprising administering to the subject a therapeutically effective amount of the pharmaceutical composition of claim 66.

69-73. (canceled)

74. A kit for treating a subject with cancer comprising the pharmaceutical composition of claim 66.

75-111. (canceled)


SEQUENCE LISTING (54 sequences)

SEQ ID NO: 1 - DNA, 3393 bp, Homo sapiens
  source 1..3393: mol_type = other DNA
tgagacagagcgcttcgcacaggagcagaggggtcagggcgaagtcccagggccccaggcgtggctctcagggtctcaggccccgaaggcggtgtatggattggggagtcccagccttggggattccccaactccgcagtttcttttctccctctcccaacctacgtagggtccttcatcctggatactcacgacgcggacccagttctcactcccattgggtgtcgggtttccagagaagccaatcagtgtcgtcgcggtcgctgttctaaagtccgcacgcacccaccgggactcagattctccccagacgccgaggatggccgtcatggcgccccgaaccctcctcctgctactctcgggggccctggccctgacccagacctgggcgggtgagtgcggggtcgggagggaaaccgcctctgcggggagaagcaaggggccctcctggcgggggcgcaggaccgggggagccgcgccgggaggagggtcgggcaggtctcagccactgctcgcccccaggctcccactccatgaggtatttcttcacatccgtgtcccggcccggccgcggggagccccgcttcatcgccgtgggctacgtggacgacacgcagttcgtgcggttcgacagcgacgccgcgagccagaagatggagccgcgggcgccgtggatagagcaggaggggccggagtattgggaccaggagacacggaatatgaaggcccactcacagactgaccgagcgaacctggggaccctgcgcggctactacaaccagagcgaggacggtgagtgaccccggcccggggcgcaggtcacgacccctcatcccccacggacgggccaggtcgcccacagtctccgggtccgagatccaccccgaagccgcgggactccgagacccttgtcccgggagaggcccaggcgcctttacccggtttcattttcagtttaggccaaaaatccccccgggttggtcggggcggggcggggctcgggggactgggctgaccgcggggtcggggccaggttctcacaccatccagataatgtatggctgcgacgtggggccggacgggcgcttcctccgcgggtaccggcaggacgcctacgacggcaaggattacatcgccctgaacgaggacctgcgctcttggaccgcggcggacatggcagctcagatcaccaagcgcaagtgggaggcggtccatgcggcggagcagcggagagtctacctggagggccggtgcgtggacgggctccgcagatacctggagaacgggaaggagacgctgcagcgcacgggtaccaggggccacggggcgcctccctgatcgcctatagatctcccgggctggcctcccacaaggaggggagacaattgggaccaacactagaatatcaccctccctctggtcctgagggagaggaatcctcctgggtttccagatcctgtaccagagagtgactctgaggttccgccctgctctctgacacaattaagggataaaatctctgaaggagtgacgggaagacgatccctcgaatactgatgagtggttccctttgacaccggcagcagccttgggcccgtgacttttcctctcaggccttgttctctgcttcacactcaatgtgtgtgggggtctgagtccagcacttctgagtctctcagcctccactcaggtcaggaccagaagtcgctgttcccttctcagggaatagaagattatcccaggtgcctgtgtccaggctggtgtctgggttctgtgctctcttccccatcccgggtgtcctgtccattctcaagatggccacatgcgtgctggtggagtgtcccatgacagatgcaaaatgcctgaattttctgactcttcccgtcagacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatggggtaaggagggagatgggggtgtcatgtctcttagggaaagcaggagcctctctggagacctttagcagggtcagggcccctcaccttcccctcttttcccagagctgtcttcccagcccaccatccccatcgtgggcatcattgctggcctggttctccttggagctgtgatcactggagctgtggtcgctgccgtgatgtggaggaggaagagctcaggtggagaaggggtgaagggtggggtctgagatttcttgtctcactgagggttccaagccccagctagaaatgtgccctgtctcattactgggaagcaccttccacaatcatgggccgacccagcctgggccctgtgtgccagcacttactcttttgtaaagcacctgttaaaatgaaggacagatttatcaccttgattacggcggtgatgggacctgatcccagcagtcacaagtcacaggggaaggtccctgaggacagacctcaggagggctattggtccaggacccacacctgctttcttcatgtttcctgatcccgccctgggtctgcagtcacacatttctggaaacttctctggggtccaagactaggaggttcctctaggaccttaaggccctggctcctttctggtatctcacaggacattttcttcccacagatagaaaaggagggagttacactcaggctgcaagtaagtatgaaggaggctgatgcctgaggtccttgggatattgtgtttgggagcccatgggggagctcacccaccccacaattcctcctctagccacatcttctgtgggatctgaccaggttctgtttttgttctaccccaggcagtgacagtgcccagggctctgatgtgtctctcacagcttgtaaaggtgagagcttggagggcctgatgtgtgttgggtgttgggtggaacagtggacacagctgtgctatggggtttctttgcgttggatgtattgagcatgcgatgggctgtttaaggtgtgacccctcactgtgatggatatgaatttgttcatgaatatttttttctatagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagagcaccccacccccatgtccaccatgaccct

SEQ ID NO: 2 - DNA, 3407 bp, Homo sapiens
  source 1..3407: mol_type = other DNA
tgagacagagcgcttggcacaggagcagaggggtcagggcgaagtcccagggccccaggcgtggctctcagggtctcaggccccgaaggcggtgtatggattggggagtcccagccttggggattccccaactccgcagtttcttttctccctctcccaacctatgtagggtccttcttcctggatactcacgacgcggacccagttctcactcccattgggtgtcgggtttccagagaagccaatcagtgtcgtcgcggtcgcggttctaaagtccgcacgcacccaccgggactcagattctccccagacgccgaggatggccgtcatggcgccccgaaccctcgtcctgctactctcgggggccctggccctgacccagacctgggcgggtgagtgcggggtcgggagggaaacggcctctgtggggagaagcaaggggcccgcccggcgggggcgcaggacccgggaagccgcgcctggaggagggtcgggcgggtctcagccactcctcgcccccaggctcccactccatgaggtatttctacacctccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccggaacacacggaatgtgaaggcccagtcacagactgaccgagtggacctggggaccctgcgcggctactacaaccagagcgaggccggtgagtgaccccggcccggggcgcaggtcacgacccctcatcccccacggacgggccaggtcgcccacagtctccgggtccgagatccgccccgaagccgcgggaccccgagacccttgccccgggagaggcccaggcgcctttacccggtttcattttcagtttaggccaaaaatccccccgggttggtcggggcggggcggggctcgggggaccgggctgacctcggggtccgggccaggttctcacaccatccagatgatgtatggctgcgacgtggggtcggacgggcgcttcctccgcgggtaccggcaggacgcctacgacggcaaggattacatcgccctgaaagaggacctgcgctcttggaccgcggcggacatggcagctcagaccaccaagcacaagtgggaggcggcccatgtggcggagcagtggagagcctacctggagggcacgtgcgtggagtggctccgcagatacctggagaacgggaaggagacgctgcagcgcacgggtaccgggggccacggggcgcctccctgatcgcctgtagatctcccgggctggcctcccacaaggaggggagacaattgggaccaacactagaatatcgccctccctctggtcctgagggagaggaatcctcctgggtttccagatcctgtaccagagagtgactctgaggttccgccctgctctctgacacaattaagggataaaatctctgaaggaatgacgggaagacgatccctcgaatactgatgagtggttccctttgacacacacaggcagcagccttgggcccgtgacttttcctctcaggccttgttctctgcttcacactcaatgtgtgtgggggtctgagtccagcacttctgagtccctcagcctccactcaggtcaggaccagaagtcgctgttccctcttcagggactagaattttccacggaataggagattatcccaggtgcctgtgtccaggctggtgtctgggttctgtgctcccttccccatcccaggtgtcctgtccattctcaagatagccacatgtgtgctggaggagtgtcccatgacagatgcaaaatgcctgaatgatctgactcttcctgacagacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggtggctgtggtggtgccttctggacaggagcagagatacacctgccatgtgcagcatgagggtttgcccaagcccctcaccctgagatggggtaaggagggagacgggggtgtcatgtcttttagggaaagcaggagcctctctgacctttagcagggtcagggcccctcaccttcccctcttttcccagagccgtcttcccagcccaccatccccatcgtgggcatcattgctggcctggttctctttggagctgtgatcactggagctgtggtcgctgctgtgatgtggaggaggaagagctcaggtggggaaggggtgaagggtgggtctgagatttcttgtctcactgagggttccaagacccaggtagaagtgtgccctgcctcgttactgggaagcaccacccacaattatgggcctacccagcctgggccctgtgtgccagcacttactcttttgtaaagcacctgttaaaatgaaggacagatttatcaccttgattacagcggtgatgggacctgatcccagcagtcacaagtcacaggggaaggtccctgaggaccttcaggagggcggttggtccaggacccacacctgctttcttcatgtttcctgatcccgccctgggtctgcagtcacacatttctggaaacttctctgaggtccaagacttggaggttcctctaggaccttaaggccctgactcctttctggtatctcacaggacattttcttcccacagatagaaaaggagggagctactctcaggctgcaagtaagtatgaaggaggctgatgcctgaggtccttgggatattgtgtttgggagcccatgggggagctcacccaccccacaattcctcctctagccacatcttctgtgggatctgaccaggttctgtttttgttctaccccaggcagtgacagtgcccagggctctgatgtgtctctcacagcttgtaaaggtgagagcctggagggcctgatgtgtgttgggtgttgggcggaacagtggacacagctgtgctatggggtttctttccattggatgtattgagcatgcgatgggctgtttaaagtgtgacccctcactgtgacagatacgaatttgttcatgaatatttttttctatagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagaccaccccacccccatgtccaccatgaccct

SEQ ID NO: 3 - DNA, 3237 bp, Homo sapiens
  source 1..3237: mol_type = other DNA
gggccccaggcgtggctctcagggtctcaggccccgaaggcggtgtatggattggggagtcccagccttggggattccccaactccgcagtttcttttctccctctcccaacctatgtagggtccttcttcctggatactcacgacgcggacccagttctcactcccattgggtgtcgggtttccagagaagccaatcagtgtcgtcgcggtcgcggttctaaagtccgcacgcacccaccgggactcagattctccccagacgccgaggatggccgtcatggcgccccgaaccctcgtcctgctactctcgggggctctggccctgacccagacctgggcgggtgagtgcggggtcgggagggaaacggcctctgtggggagaagcaacgggcccgcctggcgggggcgcaggacccgggaagccgcgccgggaggagggtcgggcgggtctcagccactcctcgtccccaagctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggtgagtgaccccggcccggggcgcaggtcacgacctctcatcccccacggacgggccaggtcgcccacagtctccgggtccgagatccgccccgaagccgcgggaccccgagacccttgccccgggagaggcccaggcgcctttacccggtttcattttcagtttaggccaaaaatccccccaggttggtcggggcggggcggggctcgggggaccgggctgaccgcggggtccgggccaggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacgggtaccaggggccacggggcgcctccctgatcgcctgtagatctcccgggctggcctcccacaaggaggggagacaattgggaccaacactagaatatcgccctccctctggtcctgagggagaggaatcctcctgggtttccagatcctgtaccagagagtgactctgaggttccgccctgctctctgacacaattaagggataaaatctctgaaggaatgacgggaagacgatccctcgaatactgatgagtggttccctttgacacacacaggcagcagccttgggcccgtgacttttcctctcaggccttgttctctgcttcacactcaatgtgtgtgggggtctgagtccagcacttctgagtccttcagcctccactcaggtcaggaccagaagtcgctgttccctcttcagggactagaattttccacggaataggagattatcccaggtgcctgtgtccaggctggtgtctgggttctgtgctcccttccccatcccaggtgtcctgtccattctcaagatagccacatgtgtgctggaggagtgtcccatgacagatgcaaaatgcctgaatgatctgactcttcctgacagacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatggggtaaggagggagacgggggtgtcatgtcttttagggaaagcaggagcctctctgacctttagcagggtcagggcccctcaccttcccctcttttcccagagccgtcttcccagcccaccatccccatcgtgggcatcattgctggcctggttctctttggagctgtgatcactggagctgtggtcgctgctgtgatgtggaggaggaagagctcaggtggggaaggggtgaagggtgggtctgagatttcttgtctcactgagggttccaagacccaggtagaagtgtgccctgcctcgttactgggaagcaccacccacaattatgggcctacccagcctgggccctgtgtgccagcacttactcttttgtaaagcacctgttaaaatgaaggacagatttatcaccttgattacagcggtgatgggacctgatcccagcagtcacaagtcacaggggaaggtccctgaggaccttcaggagggcggttggtccaggacccacacctgctttcttcatgtttcctgatcccgccctgggtctgcagtcacacatttctggaaacttctctgaggtccaagacttggaggttcctctaggaccttaaggccctgactcctttctggtatctcacaggacattttcttcccacagatagaaaaggagggagctactctcaggctgcaagtaagtatgaaggaggctgatgcctgaggtccttgggatattgtgtttgggagcccatgggggagctcacccaccccacaattcctcctctagccacatcttctgtgggatctgaccaggttctgtttttgttctaccccaggcagtgacagtgcccagggctctgatgtgtctctcacagcttgtaaaggtgagagcctggagggcctgatgtgtgttgggtgttgggcggaacagtggacacagctgtgctatggggtttctttccattggatgtattgagcatgcgatgggctgtttaaagtgtgacccctcactgtgacagatacgaatttgttcatgaatatttttttctatagtgtgagacagctgccttgtgtgggactgagaggcaagagttgttcctgcccttcc

SEQ ID NO: 4 - DNA, 3300 bp, Homo sapiens
  source 1..3300: mol_type = other DNA
caagagcagaggggtcagggcgaagtcccagggccccaggcgtggctctcagggtctcaggccccgaaggcggtgtatggattggggagtcccagccttggggattccccaactccgcagtttcttttctccctctcccaacctacgtagggtccttcatcctggatactcacgacgcggacccagttctcactcccattgggtgtcgggtttccagagaagccaatcagtgtcttcgcggtcgctcttctaaagtccgcacgcacccaccgggactcagattctccccagaccccgaggatggccgtcatggcgccccgaaccctcctcctgctactctcgggggccctggccctgacccagacctgggcgggtgagtgcggggtcgggagggaaaccgcctctgcggggagaagcaaggggccctcctggcgggggcgcaggaccgggggagccgcgccgggaggagggtcgggcaggtctcagccactgctcgcccccaggctcccactccgtgaggtatttctacacctccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccaggagacacggaatgtgaaggcccagtcacagactgaccgagtggacctggggaccctgcgcggctactacaaccagagcgaggacggtgagtgaccccggcccggggcgcaggtcacgacccctcatcccccacggacgggccaggtggcccacagtctccgggtccgagatccaccccgaagccgcgggaccccgagacccttgccccgggagaggcccaggcgcctttacccggtttcattttcagtttaggccaaaaatccccccgggttggtcggggccgggcagggcttgggggactgggctgaccgcggggtcggggccaggttctcacaccatccagataatgtatggctgcgacgtggggccggacgggcgcttcctccgcgggtaccggcaggacgcctacgacggcaaggattacatcgccctgaacgaggacctgcgctcttggaccgcggcggacatggcagctcagatcaccaagcgcaagtgggaggcggcccatgcggcggagcagcagagagcctacctggagggccggtgcgtggagtggctccgcagatacctggagaacgggaaggagacgctgcagcgcacgggtaccaggggccacggggcgcctccctgatcgcctatagatctcccgggctggcctcccacaaggaggggagacaattgggaccaacactagaatatcaccctccctctggtcctgagggagaggaatcctcctgggtttccagatcctgtaccagagagtgactctgaggttccgccctgctctctgacacaattaagggataaaatctctgaaggagtgacgggaagacgatccctcgaatactgatgagtggttccctttgacaccggcagcagccttgggcccgtgacttttcctctcaggccttgttctctgcttcacactcaatgtgtgtgggggtctgagtccagcacttctgagtctctcagcctccactcaggtcaggaccagaagtcgctgttcccttctcagggaatagaagattatcccaggtgcctgtgtccaggctggtgtctgggttctgtgctctcttccccatcccgggtgtcctgtccattctcaagatggccacatgcgtgctggtggagtgtcccatgacagatgcaaaatgcctgaattttctgactcttcccgtcagacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatggggtaaggagggagatgggggtgtcatgtctcttagggaaagcaggagcctctctggagacctttagcagggtcagggcccctcaccttcccctcttttcccagagctgtcttcccagcccaccatccccatcgtgggcatcattgctggcctggttctccttggagctgtgatcactggagctgtggtcgctgccgtgatgtggaggaggaagagctcaggtggagaaggggtgaaaggtggggtctgagatttcttgtctcactgagggttccaagccccagctagaaatgtgccctgtctcattactgggaagcaccttccacaatcatgggccgacccagcctgggccctgtgtgccagcacttactcttttgtaaagcacctgttaaaatgaaggacagatttatcaccttgattacggcggtgatgggacctgataccagcagtcacaagtcacaggggaaggtccctgaggacagacctcaggagggctattggtccaggacccacacctgctttcttcatgtttcctgatcccgccctgggtctgcagtcacacatttctggaaacttctctggggtccaagactaggaggttcctctaggaccttaaggccctggctcctttctggtatctcacaggacattttcttcccacagatagaaaaggagggagttacactcaggctgcaagtaagtatgaaggaggctgatgcctgaggtccttgggatattgtgtttgggagcccatgggggagctcacccaccccacaattcctcctctagccacatcttctgtgggatctgaccaggttctgtttttgttctaccccaggcagtgacagtgcccagggctctgatgtgtctctcacagcttgtaaaggtgagagcttggagggcctgatgtgtgttgggtgttgggcggaacagtggacacagctgtgctatggggtttctttgcattggatgtattgagcatgcgatgggctgtttaaggtgtgacccctcactgtgatggatatgaatttgttcatgaatatttttttctatagtgtgagacagctgccttgtgtgggactgagaggcaagagttgttcctgcccttccctttgtgacttgaagaaccctgactttgtttctgcaaaggcacctgc

SEQ ID NO: 5 - DNA, 3291 bp, Homo sapiens
  source 1..3291: mol_type = other DNA
caggagcagaggggtcagggcgaagtcccagggccccaggcgtggctctcagagtctcaggccccgaaggcggtgtatggattggggagtcccagccttggggattccccaactccgcagtttcttttctccctctcccaacctacgtagggtccttcatcctggatactcacgacgcggacccagttctcactcccattgggtgtcgggtttccagagaagccaatcagtgtcgtcgcggtcgctgttctaaagcccgcacgcacccaccgggactcagattctccccagacgccgaggatggccgtcatggcgccccgaaccctcctcctgctactctcgggggccctggccctgacccagacctgggcgggtgagtgcggggtcgggagggaaaccgcctctgcggggagaagcaaggggccctcctggcgggggcgcaggaccgggggagccgcgagggtcgggcaggtctcagccactgctcgcccccaggctcccactccatgaggtatttcttcacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccaggagacacggaatgtgaaggcccagtcacagactgaccgagtggacctggggaccctgcgcggctactacaaccagagcgaggccggtgagtgaccccggccgggggcgcaggtcaggacccctcatcccccacggacgggccaggtcgcccacagtctccgggtccgagatccaccccgaagccgcgggaccccgagacccttgccccgggagaggcccaggcgcctttacccggtttcattttcagtttaggccaaaaatccccccgggttggtcggggctgggcggggctcgggggactgggctgaccgcggggtcggggccaggttctcacaccatccagataatgtatggctgcgacgtggggtcggacgggcgcttcctccgcgggtaccggcaggacgcctacgacggcaaggattacatcgccctgaacgaggacctgcgctcttggaccgcggcggacatggcggctcagatcaccaagcgcaagtgggaggcggcccatgaggcggagcagttgagagcctacctggatggcacgtgcgtggagtggctccgcagatacctggagaacgggaaggagacgctgcagcgcacgggtaccaggggccacggggcgcctccctgatcgcctgtagatctcccgggctggcctcccacaaggaggggagacaattgggaccaacactagaatatcaccctccctctggtcctgagggagaggaatcctcctgggttccagatcctgtaccagagagtgactctgaggttccgccctgctctctgacacaattaagggataaaatctctgaaggagtgacgggaagacgatccctcgaatactgatgagtggttccctttgacaccggcagcagccttgggcccgtgacttttcctctcaggccttgttctctgcttcacactcaatgtgtgtgggggtctgagtccagcacttctgagtccctcagcctccactcaggtcaggaccagaagtcgctgttcccttctcagggaatagaagattatcccaggtgcctgtgtccaggctggtgtctgggttctgtgctctcttccccatcccgggtgtcctgtccattctcaagatggccacatgcgtgctggtggagtgtcccatgacagatgcaaaatgcctgaattttctgactcttcccgtcagacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatggggtaaggagggagatgggggtgtcatgtctcttagggaaagcaggagcctctctggagacctttagcagggtcagggcccctcaccttcccctcttttcccagagctgtcttcccagcccaccatccccatcgtgggcatcattgctggcctggttctccttggagctgtgatcactggagctgtggtcgctgccgtgatgtggaggaggaagagctcaggtggagaaggggtgaagggtggggtctgagatttcttgtctcactgagggttccaagccccagctagaaatgtgccctgtctcattactgggaagcaccgtccacaatcatgggcctacccagtctgggccctgtgtgccagcacttactcttttgtaaagcacctgttaaaatgaaggacagatttatcaccttgattacggcggtgatgggacctgatcccagcagtcacaagtcacaggggaaggtccctgaggacagacctcaggagggctattggtccaggacccacacctgctttcttcatgtttcctgatcccgccctgggtctgcagtcacacatttctggaaacttctctggggtccaagactaggaggttcctctaggaccttaaggccctggctcctttctggtatctcacaggacattttcttctcacagatagaaaaggagggagttacactcaggctgcaagtaagtatgaaggaggctgatgcctgaggtccttgggatattgtgtttgggagcccatgggggagcccacccacctcacaattcctcctctagccacatcttctgtgggatctgaccaggttctgtttttgttctaccccaggcagtgacagtgcccagggctctgatgtgtccctcacagcttgtaaaggtgagagcttggaggacctaatgtgtgttgggtgttgggcggaacagtggacacagctgtgctatggggtttctttgcattggatgtattgagcatgcgatgggctgtttaaggtgtgacccctcactgtgatggatatgaatttgttcatgaatatttttttctatagtgtgagacagctgccttgtgtgggactgagaggcaagagttgttcctgcccttccctttgtgacttgaagaaccctgactttgtttctgcaaaggcacctgc

SEQ ID NO: 6 - DNA, 3373 bp, Homo sapiens
  source 1..3373: mol_type = other DNA
cagaagcagaggggtcagggcgaagtcccagggccccaggcgtggctctcagggtctcaggccccgaaggcggtgtatggattggggagtcccagccttggggattccccaactccgcagtttcttttctccctctcccaacctatgtagggtccttcttcctggatactcacgacgcggacccagttctcactcccattgggtgtcgggtttccagagaagccaatcagtgtcgtcgcggtcgctgttctaaagtccgcacgcacccaccgggactcagattctccccagacgccgaggatggccgtcatggcgccccgaaccctcgtcctgctactctcgggggccctggccctgacccagacctgggcaggtgagtgcggggtcgggagggaaacggcctctgcggggagaagcaaggggcccgcctggcgggggcgcaagacccgggaagccgcgccgggaggagggtcgggcgggtctcagccactcctcgtccccaggctcccactccatgaggtatttctccacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggacgaggagacagggaaagtgaaggcccactcacagactgaccgagagaacctgcggatcgcgctccgctactacaaccagagcgaggccggtgagtgaccccggcccggggcgcaggtcacgacccctcatcccccacggacgggccgggtcgcccacagtctccgggtccgagatccaccccgaagccgcgggaccccgagacccttgccccgggagaggcccaggcgccttaacccggtttcattttcagtttaggccaaaaatccccccgggttggtcggggccgggcggggctcgggggactgggctgaccgcggggtcggggccaggttctcacaccctccagatgatgtttggctgcgacgtggggtcggacgggcgcttcctccgcgggtaccaccagtacgcctacgacggcaaggattacatcgccctgaaagaggacctgcgctcttggaccgcggcggacatggcggctcagatcacccagcgcaagtgggaggcggcccgtgtggcggagcagttgagagcctacctggagggcacgtgcgtggacgggctccgcagatacctggagaacgggaaggagacgctgcagcgcacgggtaccaggggccacggggcgcctacctgatcgcctgtaggtctcccgggctggcctcccacaaggaggggagacaattgggaccaacactagaatatcgccctccctctggtcctgagggagaggaatcctcctgggtttccagatcctgtaccagagagtgactctgaggttccgccctgctctctgacacaattaagggataaaatctctgacggaatgacggaaagacgatccctcgaatactgatgactggttccctttgacaccggcagcagccttgggaccgtgacttttcctctcaggccttgttctctgcttcacactcaatgtgtgtgggggtctgagtccagcacttctgagtccctcagcctccactcaggtcaggaccagaagtcgctgttccctcctcagggaatagaagattatcccaggtgcctgtgtccaggctggtgtctgggttctgtgctctcttccccatcccgggtgtcctgtccattctcaagatggccacatgcatgctggtggagtgtcccatgacagatgcaaaatgcctgaattttctgactcttcccgtcagacccccccaagacacatatgacccaccaccccatctctgaccatgaggccactctgagatgctgggccctgggcttctaccctgcggagatcacactgacctggcagcgggatggggaggaccagacccaggacacggagcttgtggagaccaggcctgcaggggatggaaccttccagaagtgggcagctgtggtggtaccttctggagaggagcagagatacacctgccatgtgcagcatgagggtctgcccaagcccctcaccctgagatggggtaaggagggagatgggggtgtcatgtctcttagggaaagcaggagcctctctggagacctttagcagggtcagggcccctcaccttcccctcttttcccagagccatcttcccagcccaccgtccacatcgtgggcatcattgctggcctggttctccttggagctgtgatcactggagctgtggtcgctgctgtgatgtggaggaggaacagctcaggtggagaaggggtgaagggtggggtctgagatttcttgtctcactgagggttccaagccccagctagaaatgtgccctgtctcattactgggaagcaccatccacaatcatgggccgacccagcctgggccctgtgtgccagcacttactcttttgtaaagcacctgtgacaatgaaggacagatttatcaccttgattatggcggtgatgggacctgatcccagcagtcacaagtcacaggggaaggtccctgacgacagatctcaggagggcgattggtccagggcccacatctgctttcttcatgtttcctgatcctgccctgggtctgcagtcacacatttctggaaacttctctggggtccaagactaggaggttcctctaggaccttaaggccctggctcctttctggtatctcacaggacattttcttcccacagatagaaaaggagggagctactctcaggctgcaagtaagtatgaaggaggctgatgcctgaggtccttgggatattgtgtttgggagcccatgggggagctcaaccaccccacaattcctcctctagccacatcttctgtgggatctgaccaggttctgtttttgttctaccccaggcagtgacagtgcccagggctctgatgtgtctctcacagcttgtaaaggtgagagcttggagggcctgatgtgtgttgggtgttgggcggaacagtggacacagctgtgctatggggtttctttgcattggatgtattgagcatgcgatgggctgtttaaagtgtgacccctcactgtgacagatatgaagttgttcatgaattttttttctatagtgtgagacagctgccttgtgtgggactgagaggcaagagttgttcctgcccttccctttgtgacttgaagaaccctgactttgtttctgcaaaggcacctgcatgtgtctgtgttcatgtaggcataatgtgaggaggtggggagaccaccccacccccatgtccaccatgaccct

SEQ ID NO: 7 - DNA, 3315 bp, Homo sapiens
  source 1..3315: mol_type = other DNA
caggagcagaggggtcagggcgaagtaccagggccccaggcgtggctctcagggtctcaggccccgaaggcggtgtatggattggggagtcccagccttggggattccccaactccgcagtttcttttctccctctcccaacctatgtagggtccttcttcctggatactcacgacgcggacccagttctcactcccattgggtgtcgggtttccagagaagccaatcagtgtcgtcgcggtcgcggttctaaagtccgcacgcacccaccgggactcagattctccccagacgccgaggatggccgtcatggcgccccgaaccctcctcctgctactcttgggggccctggccctgacccagacctgggcgggtgagtgcggggtcgtggggaaaccgcctctgcggggagaagcaaggggcccgcccggcgggggcgcaggacccgggtagccgcgccgggaggagggtcgggcggatctcagccactcctcgcccccaggctcccactccatgaggtatttcaccacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccggaacacacggaatgtgaaggcccactcacagattgaccgagtggacctggggaccctgcgcggctactacaaccagagcgaggccggtgagtgaccccagcccggggcgcaggtcacgacctctcatcccccacggacgggccaggtcacccacagtctccgggtccgagatccaccccgaagccgcgggaccccgagacccttgccccgggagaggcccaggcgcctttacccggtttcattttcagtttaggccaaaaatccccccgggttggtcggggccggacggggctcgggggactgggctgaccgtggggtcggggccaggttctcacaccatccagatgatgtatggctgcgacgtggggtcggacgggcgcttcctccgcgggtaccagcaggacgcctacgacggcaaggattacatcgccttgaacgaggacctgcgctcttggaccgcggcggacatggcggctcagatcacccagcgcaagtgggaggcggcccgtgtggcggagcagttgagagcctacctggagggcacgtgcgtggagtggctccgcagatacctggagaacgggaaggagacgctgcagcgcacgggtaccaggggccacggggcgcctccctgatcgcctgtagatctcccgggctggcctcccacaaggaggggagacaattgggaccaacactagaatatcaccctccctctggtcctgagggagaggaatcctcctgggtttccagatcctgtaccagagagtgactctgaggttccgccctgctctgtgacacaattaagggataaaatctctgaaggaatgacgggaagacgatccctcgaatactgatgagtggttccctttgacacacaccggcagcagccttgggcccgtgacttttcctctcaggccttgttctctgcttcacactcaatgtgtgtgggggtctgagtccagcacttctgagtccctcagcctccactcaggtcaggaccagaagtcgctgttccctcttcagggactagaattttccacggaataggagattatcccaggtgcctgtgtccaggctggtgtctgggttctgcgctcccttccccatcccaggtgtcctgtccattctcaagatagccacatgtgtgctggaggagtgtcccattacagatgcaaaatgcctgaatgttctgactcttcctgacagacccccccaagacgcatatgactcaccacgctgtctctgaccatgaggccaccctgaggtgctgggccctgagcttctaccctgcggagatcacactgacctggcagcgggatggggaggaccagacccaggacacggagctcgtggagaccaggcctgcaggggatggaaccttccagaagtgggcgtctgtggtggtgccttctggacaggagcagagatacacctgccatgtgcagcatgagggtctccccaagcccctcaccctgagatggggtaaggagggagatgggggtgtcatgtcttttagggaaagcaggagcctctctgacctttagcagggtcagggcccctcaccttcccctcttttcccagagccgtcttcccagcccaccatccccatcgtgggcatcattgctggcctagttctctttggagctgtgttcgctggagctgtggtcgctgctgtgaggtggaggaggaagagctcaggtggggtgaaggggtgaagggtgggtctgagatttcttgtctcactgagggttccaagacccaggtagaagtgtgccctgcctcgttactgggaagcaccatccacaattatgggcctacccagcctgggccctgtgtgccagcacttactcttttgtaaagcacctgttaaaatgaaggacagatttatcaccttgattatggcggtgatgggacctgatcccagcagtcacaagtcacaggggaaggtccctgaggaccttcaggagggcggttggtccaggacccacacctgctttcttcatgtttcctgatcccgccctgggtctgcagtcacacatttctggaaacttctctgaggtccaagacttggaggttcctctaggaccttaaggccctggctcctttctggtatctcacaggacattttcttcccacagatagaaaaggagggagctactctcaggctgcaagtaagtatgaaggaggatgatccaagaaatcactgggatattgtgtttgggagcccgtgggggagctcacccaccccacaattcctcctctagccacatcttctgtgggatctgaccaggttctgtttttgtcctaccccaggcagtgacagtgcccagggctctgatatgtctctcacagcttgtaaaggtgagagcctggagggcctgatgtgtgttgggtgttgggcggaacagtggacgcagctgtgctatggggtttctttgcattggatgtattgagcatgcgatgggctgtttaaagtgtgactcctcactgtgacagatacgaatttgttcatgaatatttttttctatagtgtgagacagctgccttgtgtgggactgagaggcaagatttgttcctgcccttccctttgtgacttgaagtaccctgactttgtttctgcaaaggcacctgc

SEQ ID NO: 8 - DNA, 3392 bp, Homo sapiens
  source 1..3392: mol_type = other DNA
tgagacagagcgcttggcacagaagcagaggggtcagggcgaagtcccagggccccaggcgtggctctcagggtctcaggccccgaaggcggtgtatggattggggagtcccagccttggggattccccaactccgcagtttcttttctccctctcccaacctatgtagggtccttcttcctggatactcacgacgcggacccagttctcactcccattgggtgtcgggtttccagagaagccaatcagtgtcgtcgcggtcgctgttctaaagtccgcacgcacccaccgggactcagattctccccagacgccgaggatggccgtcatggcgccccgaaccctcgtcctgctactctcgggggccctggccctgacccagacctgggcaggtgagtgcggggtcgggagggaaacggcctctgcggggagaagcaaggggcccgcctggcgggggcgcaagacccgggaagccgcgccgggaggagggtcgggcgggtctcagccactcctcgtccccaggctcccactccatgaggtatttctccacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggacgaggagacagggaaagtgaaggcccactcacagactgaccgagagaacctgcggatcgcgctccgctactacaaccagagcgaggccggtgagtgaccccggcccggggcgcaggtcacgacccctcatcccccacggacgggccgggtcgcccacagtctccgggtccgagatccaccccgaagccgcgggaccccgagacccttgccccgggagaggcccaggcgccttaacccggtttcattttcagtttaggccaaaaatccccccgggttggtcggggccgggcggggctcgggggactgggctgaccgcggggtcggggccaggttctcacaccctccagatgatgtttggctgcgacgtggggtcggacgggcgcttcctccgcgggtaccaccagtacgcctacgacggcaaggattacatcgccctgaaagaggacctgcgctcttggaccgcggcggacatggcggctcagatcaccaagcgcaagtgggaggcggcccatgtggcggagcagcagagagcctacctggagggcacgtgcgtggacgggctccgcagatacctggagaacgggaaggagacgctgcagcgcacgggtaccaggggccacggggcgcctacctgatcgcctgtaggtctcccgggctggcctcccacaaggaggggagacaattgggaccaacactagaatatcgccctccctctggtcctgagggagaggaatcctcctgggtttccagatcctgtaccagagagtgactctgaggttccgccctgctctctgacacaattaagggataaaatctctgacggaatgacggaaagacgatccctcgaatactgatgactggttccctttgacaccggcagcagccttgggaccgtgacttttcctctcaggccttgttctctgcttcacactcaatgtgtgtgggggtctgagtccagcacttctgagtccctcagcctccactcaggtcaggaccagaagtcgctgttccctcctcagggaatagaagattatcccaggtgcctgtgtccaggctggtgtctgggttctgtgctctcttccccatcccgggtgtcctgtccattctcaagatggccacatgcatgctggtggagtgtcccatgacagatgcaaaatgcctgaattttctgactcttcccgtcagacccccccaagacacatatgacccaccaccccatctctgaccatgaggccactctgagatgctgggccctgggcttctaccctgcggagatcacactgacctggcagcgggatggggaggaccagacccaggacacggagcttgtggagaccaggcctgcaggggatggaaccttccagaagtgggcagctgtggtggtaccttctggagaggagcagagatacacctgccatgtgcagcatgagggtctgcccaagcccctcaccctgagatggggtaaggagggagatgggggtgtcatgtctcttagggaaagcaggagcctctctggagacctttagcagggtcagggcccctcaccttcccctcttttcccagagccatcttcccagcccaccgtccccatcgtgggcatcattgctggcctggttctccttggagctgtgatcactggagctgtggtcgctgctgtgatgtggaggaggaacagctcaggtggagaaggggtgaagggtggggtctgagatttcttgtctcactgagggttccaagccccagctagaaatgtgccctgtctcattactgggaagcaccatccacaatcatgggccgacccagcctgggccctgtgtgccagcacttactcttttgtaaagcacctgtgacaatgaaggacagatttatcaccttgattatgggggtgatgggacctgatcccagcagtcacaagtcacaggggaaggtccctgacgacagatctcaggagggcgattggtccagggcccacatctgctttcttcatgtttcctgatcctgccctgggtctgcagtcacacatttctggaaacttctctggggtccaagactaggaggttcctctaggaccttaaggccctggctcctttctggtatctcacaggacattttcttcccacagatagaaaaggagggagctactctcaggctgcaagtaagtatgaaggaggctgatgcctgaggtccttgggatattgtgtttgggagcccatgggggagctcaaccaccccacaattcctcctctagccacatcttctgtgggatctgaccaggttctgtttttgttctaccccaggcagtgacagtgcccagggctctgatgtgtctctcacagcttgtaaaggtgagagcttggagggcctgatgtgtgttgggtgttgggcggaacagtggacacagctgtgctatggggtttctttgcattggatgtattgagcatgcgatgggctgtttaaagtgtgacccctcactgtgacagatatgaagttgttcatgaattttttttctatagtgtgagacagctgccttgtgtgggactgagaggcaagagttgttcctgcccttccctttgtgacttgaagaaccctgactttgtttctgcaaaggcacctgcatgtgtctgtgttcatgtaggcataatgtgaggaggtggggagaccaccccacccccatgtccaccatgaccct

SEQ ID NO: 9 - DNA, 3314 bp, Homo sapiens
  source 1..3314: mol_type = other DNA
cagaagcagaggggtcagggcgaagtcccagggccccaggcgtggctctcagggtctcaggccccgaaggcggtgtatggattggggagtcccagccttggggattccccaactccgcagtttcttttctccctctcccaacctatgtagggtccttcttcctggatactcacgacgcggacccagttctcactcccattgggtgtcgggtttccagagaagccaatcagtgtcgtcgcggtcgcggttctaaagtccgcacgcacccaccgggactcagattctccccagacgccgaggatggctgtcatggcgccccgaaccctcgtcctgctactctcgggggctctggccctgacccagacctgggcgggtgagtgcggggtcgggagggaaacggcctctgtggggagaagcaacgggcccgcctggcgggggcgcaggacccgggaagccgcgccgggaggagggtcgggcgggtctcagccactcctcgtccccaggctctcactccatgaggtatttctacacctccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggtgagtgaccccggcccggggcgcaggtcacgacctctcatcccccacggacgggccaggtcgcccacagtctccgggtccgagatccgccccgaagccgcgggaccccgagacccttgccccgggagaggcccaggcgcctttacccggtttcattttcagtttaggccaaaaatccccccaggttggtcggggcggggcggggctcgggggaccgggctgaccgcggggtccgggccaggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacgggtaccaggggccacggggcgcctccctgatcgcctgtagatctcccgggctggcctcccacaaggaggggagacaattgggaccaacactagaatatcgccctccctctggtcctgagggagaggaatcctcctgggtttccagatcctgtaccagagagtgactctgaggttccgccctgctctctgacacaattaagggataaaatctctgaaggaatgacgggaagacgatccctcgaatactgatgagtggttccctttgacacacacaggcagcagccttgggcccgtgacttttcctctcaggccttgttctctgcttcacactcaatgtgtgtgggggtctgagtccagcacttctgagtccttcagcctccactcaggtcaggaccagaagtcgctgttccctcttcagggactagaattttccacggaataggagattatcccaggtgcctgtgtccaggctggtgtctgggttctgtgctcccttccccatcccaggtgtcctgtccattctcaagatagccacatgtgtgctggaggagtgtcccatgacagatgcaaaatgcctgaatgatctgactcttcctgacagacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatggggtaaggagggagacgggggtgtcatgtcttttagggaaagcaggagcctctctgacctttagcagggtcagggcccctcaccttcccctcttttcccagagccgtcttcccagcccaccatccccatcgtgggcatcattgctggcctggttctctttggagctgtgatcactggagctgtggtcgctgctgtgatgtggaggaggaagagctcaggtggggaaggggtgaagggtgggtctgagatttcttgtctcactgagggttccaagacccaggtagaagtgtgccctgcctcgttactgggaagcaccacccacaattatgggcctacccagcctgggccctgtgtgccagcacttactcttttgtaaagcacctgttaaaatgaaggacagatttatcaccttgattacagcggtgatgggacctgatcccagcagtcacaagtcacaggggaaggtccctgaggaccttcaggagggcggttggtccaggacccacacctgctttcttcatgtttcctgatcccgccctgggtctgcagtcacacatttctggaaacttctctgaggtccaagacttggaggttcctctaggaccttaaggccctgactcctttctggtatctcacaggacattttcttcccacagatagaaaaggagggagctactctcaggctgcaagtaagtatgaaggaggctgatgcctgaggtccttgggatattgtgtttgggagcccatgggggagctcacccaccccacaattcctcctctagccacatcttctgtgggatctgaccaggttctgtttttgttctaccccaggcagtgacagtgcccagggctctgatgtgtctctcacagcttgtaaaggtgagagcctggagggcctgatgtgtgttgggtgttgggcggaacagtggacacagctgtgctatggggtttctttccattggatgtattgagcatgcgatgggctgtttaaagtgtgacccctcactgtgacagatacgaatttgttcatgaatatttttttctatagtgtgagacagctgccttgtgtgggactgagaggcaagagttgttcctgcccttccctttgtgacttgaagaaccctgactttgtttctgcaaaggcacctgc

SEQ ID NO: 10 - DNA, 3314 bp, Homo sapiens
  source 1..3314: mol_type = other DNA
caggagcagaggggtcagggcgaagtcccagggccccaggcgtggctctcagggtctcaggccccgaaggcggtatatggattggggagtcccagccttggggattccccaactccgcagtttcttttctccctctcccaacctatgtagggtccttcttcctggatactcacgacgcggacccagttctcactcccattgggtgtcgggtttccagagaagccaatcagtgtcgtcgcggtcgcggttctaaagtccgcacgcacccaccgggactcagattctccccagacgccgaggatggccgtcatggcgccccgaaccctcgtcctgctactctcgggggccctggccctgacccagacctgggcgggtgagtgcggggtcgggagggaaacggcctctgtggggagaagcaaggggcccgcccggcgggggcgcaggacccgggaagccgcgcctggaggagggtcgggcgggtctcagccactcctcgcccccaggctcccactccatgaggtatttctacacctccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccggaacacacggaatgtgaaggcccactcacagactgaccgagcgaacctggggaccctgcgcggctactacaaccagagcgaggacggtgagtgaccccggcccggggcgcaggtcacgacccctcatcccccacggacgggccaggtcgcccacagtctccgggtccgagatccgccccgaagccgcgggaccccgagacccttgccccgggagaggcccaggcgcctttacccggtttcattttcagtttaggccaaaaatccccccgggttggtcggggcggggcggggctcgggggaccgggctgacctcggggtccgggccaggttctcacaccatccagaggatgtatggctgcgacgtggggccggacgggcgcttcctccgcgggtaccagcaggacgcttacgacggcaaggattacatcgccctgaacgaggacctgcgctcttggaccgcggcggacatggcggctcagatcacccagcgcaagtgggagacggcccatgaggcggagcagtggagagcctacctggagggccggtgcgtggagtggctccgcagatacctggagaacgggaaggagacgctgcagcgcacgggtaccaggggccacggggcgcctccctgatcgcctgtagatctcccgggctggcctcccacaaggaggggagacaattgggaccaacactagaatatcgccctccctctggtcctgagggagaggaatcctcctgggtttccagatcctgtaccagagagtgactctgaggttccgccctgctctctgacacaattaagggataaaatctctgaaggaatgacgggaagacgatccctcgaatactgatgagtggttccctttgacacacaccggcagcagccttgggcccgtgacttttcctctcaggccttgttctctgcttcacactcaatgtgtgtgggggtctgagtccagcacttctgagtccctcagcctccactcaggtcaggaccagaagtcgctgttccctcttcagggactagaattttccacggaataggagattatcccaggtgcctgtgtccaggctggtgtctgggttctgtgctcccttccccatcccaggtgtcctgtccattctcaagatagccacatgtgtgctggaggagtgtcccatgacagatgcaaaatgcctgaatgttctgactcttcctgacagacgcccccaagacgcatatgactcaccacgctgtctctgaccatgaggccaccctgaggtgctgggccctgagcttctaccctgcggagatcacactgacctggcagcgggatggggaggaccagacccaggacacggagctcgtggagaccaggcctgcaggggatgggaccttccagaagtgggcgtctgtggtggtgccttctggacaggagcagagatacacctgccatgtgcagcatgagggtctgcccaagcccctcaccctgagatggggtaaggagggagacgggggtgtcatgtcttttagggaaagcaggagcctctctgacctttagcagggtcagggcccctcaccttcccctcttttcccagagccgtcttcccagcccaccatccccatcgtgggcatcattgctggcctggttctctttggagctgtgatcgctggagctgtggtcgctgctgtgatgtggaggaggaagagctcaggtggggaagggatgaagggtgggtctgagatttcttgtctcactgagggttccaagacccaggaagaagtgtgccctgcctcgttactgggaagcaccatccacaattatgagcctacccagcctgggccctgtgtgccagcacttactcttttgtaaagcacctgttaaaatgaaggacagatttatcaccttgattacggcggtgatgggacctgatcccagcagtcacaagtcacaggggaaggtccctgaggaccttcaggagggcggttggtccaggacccacacctgctttcttcatgtttcctgatcccgccctgggtctgcagtcacacatttctggaaacttctctgaggtccaagacttggaggttcctctaggaccttaaggccctggctcctttctggtatctcacaggacattttcttcccacagatagaaaaggagggagctactctcaggctgcaagtaagtatgaaggaggctgatgcctgaggtccttgggatattgtgtttgggagcccgtgggggagctcacccaccccacaattcctcctctagccacatgttctgtgggatctgaccaggttctgtttttgtcctaccccaggcagtgacagtgcccagggctctgatatgtctctcacagcttgtaaaggtgagagcctggagggcctgatgtgtgttgggtgttgggcggaacagtggacgcagctgtgctatggggtttctttgcattggatgtattgagcatgcgatgggctgtttaaagtgtgactcctcactgtgacagatacgaatttgttcatgaatatttttttctatagtgtgagacagctgccttgtgtgggactgagaggcaagatttgttcctgcccttccctttgtgacttgaagaaccctgactttgtttctgcaaaggcacctgc

SEQ ID NO: 11 - DNA, 3082 bp, Homo sapiens
  source 1..3082: mol_type = other DNA
ggtccttcttcctggatactcacgacgcggacccagttctcactcccattgggtgtcgggtttccagagaagccaatcagtgtcgtcgcggtcgcggttctaaagtccgcacgcacccaccgggactcagattctccccagacgccgaggatggccgtcatggcgccccgaaccctcctcatgctactcttgggggccctggccctgacccagacctgggcgggtgagtgcggggtcgtggggaaaccgcctctgcggggagaagcaaggggcccgcccggcggggacgcaggacccgggtagccgcgccgggaggagggtcgggtgggtctcagccactcctcgcccccaggctcccactccatgaggtatttcaccacatccgtgtcccggcccggccgcggggagccccgcttcatcgccgtgggctacgtggacgacacgcagttcgtgcggtttgacagcgacgccgcgagccagaggatggagccgcgggcaccgtggatagagcaggaggggccggagtattgggacctgcagacacggaatgtgaaggcccagtcacagactgaccgagcgaacctggggaccctgcgcggctactacaaccagagcgaggccggtgagtgaccccggcccggggcgcaggtcacgacctctcatcccccacggacgggccgggtcgcccacagtctccgggtccgagatccaccccgaagccgcgggaccccgagacccttgccccgggagaggcccaggcgcctttacccggtttcattttcagtttaggccaaaaatccccccgggttggtcggggccggacggggctcgggggactgggctgaccgtggggtcggggccaggttctcacaccatccagatgatgtatggctgcgacgtggggtcggacgggcgcttcctccgcgggtaccggcaggacgcctacgacggcaaggattacatcgccttgaacgaggacctgcgctcttggaccgcggcggacatggcggctcagatcacccagcgcaagtgggaggcggcccgtgtggcggagcagttgagagcctacctggagggcacgtgcgtggagtggctccgcagatacctggagaacgggaaggagacgctgcagcgcacgggtaccgggggccacggggcgcctccctgatcgcctgtagatctcccgggctggcctcccacaaggaggggagacaattgggaccaacactagaatatcgccctccctctggtcctgagggagaggaatcctcctgggtttccagatcctgtaccagagagtgactctgaggttccgccctgctctctgacacaattaagggataaaatctctgaaggaatgacgggaagacgatccctcgaatactgatgagtggttccctttgacacacaccggcagcagccttgggcccgtgacttttcctctcaggccttgttctctgcttcacactcaatgtgtgtgggggtctgagtccagcacttctgagtccctcagcctccactcaggtcaggaccagaagtcgctgttccctcttcagggactagaattttccacggaataggagattatcccaggtgcctgtgtccaggctggtgtctgggttctgtgctcccttccccatcccaggtgtcctgtccattctcaagatagccacatgtgtgctggaggagtgtcccatgacagatgcaaaatgcctgaatgttctgactcttcctgacagacgcccccaagacgcatatgactcaccacgctgtctctgaccatgaggccaccctgaggtgctgggccctgagcttctaccctgcggagatcacactgacctggcagcgggatggggaggaccagacccaggacacggagcttgtggagaccaggcctgcaggggatggaaccttccagaagtgggcgtctgtggtggtgccttctggacaggagcagagatacacctgccatgtgcagcatgagggtctgcccaagcccctcaccctgagatggggtaaggagggagatgggggtgtcatgtcttttagggaaagcaggagcctctctgacctttagcagggtcagggcccctcaccttcccctcttttcccagagccgtcttcccagcccaccatccccatcgtgggcatcattgctggcctagttctctttggagctgtgttcgctggagctgtggtcgctgctgtgaggtggaggaggaagagctcaggtggggtgaagggatgaagggtgggtctgagatttcttgtctcactgagggttccaagacccaggtagaagtgtgccctgcctcgttactgggaagcaccatccacaattatgggcctacccagcctgggccctgtgtgccagcacttactcttttgtaaagcacctgttaaaatgaaggacagatttatcaccttgattacggcggtgatgggacctgatcccagcagtcacaagtcacaggggaaggtccctgaggaccttcaggagggcggttggtccaggacccacacctgctttcttcatgtttcctgatcccgccctgggtctgcagtcacacatttctggaaacttctctgaggtccaagacttggaggttcctctaggaccttaaggccctggctcctttctggtatctcacaggacattttcttcccacagatagaaaaggagggagctactctcaggctgcaagtaagtatgaaggaggctgatgcctgaggtccttgggatattgtgtttgggagcccgtgggggagctcacccaccccacaattcctcctctagccacatcttctgtgggatctgaccaggttctgtttttgtcctaccccaggcagtgacagtgcccagggctctgatatgtctctcacagcttgtaaaggtgagagcctggagggcctgatgtgtgttgggtgttgggcggaacagtggacgcagctgtgctatggggtttctttgcattggatgtattgagcatgcgatgggctgtttaaagtgtgactcctcactgtgacagatacgaatttgttcatgaatatttttttctatagtgtgagacagctgccttgt

SEQ ID NO: 12 - DNA, 3315 bp, Homo sapiens
  source 1..3315: mol_type = other DNA
caggagcagaggggtcagggcgaagtaccagggccccaggcgtggctctcagggtctcaggccccgaaggcggtgtatggattggggagtcccagccttggggattccccaactccgcagtttcttttctccctctcccaacctatgtagggtccttcttcctggatactcacgacgcggacccagttctcactcccattgggtgtcgggtttccagagaagccaatcagtgtcgtcgcggtcgcggttctaaagtccgcacgcacccaccgggactcagattctccccagacgccgaggatggccgtcatggcgccccgaaccctcctcctgctactcttgggggccctggccctgacccagacctgggcgggtgagtgcggggtcgtggggaaaccgcctctgcggggagaagcaaggggcccgcccggcgggggcgcaggacccgggtagccgcgccgggaggagggtcgggcggatctcagccactcctcgcccccaggctcccactccatgaggtatttcaccacatccgtgtcccggcccggccgcggggagccccgcttcatcgccgtgggctacgtggacgacacgcagttcgtgcggttcgacagcgacgccgcgagccagaggatggagccgcgggcgccgtggatagagcaggagaggcctgagtattgggaccaggagacacggaatgtgaaggcccactcacagattgaccgagtggacctggggaccctgcgcggctactacaaccagagcgaggccggtgagtgaccccagcccggggcgcaggtcacgacctctcatcccccacggacgggccaggtcacccacagtctccgggtccgagatccaccccgaagccgcgggaccccgagacccttgccccgggagaggcccaggcgcctttacccggtttcattttcagtttaggccaaaaatccccccgggttggtcggggccggacggggctcgggggactgggctgaccgtggggtcggggccaggttctcacaccatccagatgatgtatggctgcgacgtggggtcggacgggcgcttcctccgcgggtaccagcaggacgcctacgacggcaaggattacatcgccttgaacgaggacctgcgctcttggaccgcggcggacatggcggctcagatcacccagcgcaagtgggaggcggcccgtgtggcggagcagttgagagcctacctggagggcacgtgcgtggagtggctccgcagatacctggagaacgggaaggagacgctgcagcgcacgggtaccaggggccacggggcgcctccctgatcgcctgtagatctcccgggctggcctcccacaaggaggggagacaattgggaccaacactagaatatcaccctccctctggtcctgagggagaggaatcctcctgggtttccagatcctgtaccagagagtgactctgaggttccgccctgctctgtgacacaattaagggataaaatctctgaaggaatgacgggaagacgatccctcgaatactgatgagtggttccctttgacacacaccggcagcagccttgggcccgtgacttttcctctcaggccttgttctctgcttcacactcaatgtgtgtgggggtctgagtccagcacttctgagtccctcagcctccactcaggtcaggaccagaagtcgctgttccctcttcagggactagaattttccacggaataggagattatcccaggtgcctgtgtccaggctggtgtctgggttctgtgctcccttccccatcccaggtgtcctgtccattctcaagatagccacatgtgtgctggaggagtgtcccattacagatgcaaaatgcctgaatgttctgactcttcctgacagacccccccaagacgcatatgactcaccacgctgtctctgaccatgaggccaccctgaggtgctgggccctgagcttctaccctgcggagatcacactgacctggcagcgggatggggaggaccagacccaggacacggagctcgtggagaccaggcctgcaggggatggaaccttccagaagtgggcgtctgtggtggtgccttctggacaggagcagagatacacctgccatgtgcagcatgagggtctccccaagcccctcaccctgagatggggtaaggagggagatgggggtgtcatgtcttttagggaaagcaggagcctctctgacctttagcagggtcagggcccctcaccttcccctcttttcccagagccgtcttcccagcccaccatccccatcgtgggcatcattgctggcctagttctctttggagctgtgttcgctggagctgtggtcgctgctgtgaggtggaggaggaagagctcaggtggggtgaaggggtgaagggtgggtctgagatttcttgtctcactgagggttccaagacccaggtagaagtgtgccctgcctcgttactgggaagcaccatccacaattatgggcctacccagcctgggccctgtgtgccagcacttactcttttgtaaagcacctgttaaaatgaaggacagatttatcaccttgattatggcggtgatgggacctgatcccagcagtcacaagtcacaggggaaggtccctgaggaccttcaggagggcggttggtccaggacccacacctgctttcttcatgtttcctgatcccgccctgggtctgcagtcacacatttctggaaacttctctgaggtccaagacttggaggttcctctaggaccttaaggccctggctcctttctggtatctcacaggacattttcttcccacagatagaaaaggagggagctactctcaggctgcaagtaagtatgaaggaggatgatccaagaaatcactgggatattgtgtttgggagcccgtgggggagctcacccaccccacaattcctcctctagccacatcttctgtgggatctgaccaggttctgtttttgtcctaccccaggcagtgacagtgcccagggctctgatgtgtctctcacagcttgtaaaggtgagagcctggagggcctgatgtgtgttgggtgttgggcggaacagtggacgcagctgtgctatggggtttctttgcattggatgtattgagcatgcgatgggctgtttaaagtgtgactcctcactgtgacagatacgaatttgttcatgaatatttttttctatagtgtgagacagctgccttgtgtgggactgagaggcaagatttgttcctgcccttccctttgtgacttgaagtaccctgactttgtttctgcaaaggcacctgc

SEQ ID NO: 13 - DNA, 3315 bp, Homo sapiens
  source 1..3315: mol_type = other DNA
caggagcagaggggtcagggcgaagtcccagggccccaggcgtggctctcagggtctcaggccccgaaggcggtgtatggattggggagtcccagccttggggattccccaactccgcagtttcttttctccctctgccaacctatgtagggtccttcttcctggatactcacgacgcggacccagttctcactcccattgggtgtcgggtttccagagaagccaatcagtgtcgtcgcggtcgcggttctaaagtccgcacgcacccaccgggactcagattctccccagacgccgaggatggccgtcatggcgccccgaaccctcctcctgctactcttgggggccctggccctgacccagacctgggcgggtgagtgcggggtcgtggggaaaccgcctctgcggggagaagcaaggggcccgcccggcgggggcgcaggacccgggtagccgcgccgggaggagggtcgggcgggtctcagccactcctcgcccccaggctcccactccatgaggtatttcttcacatccgtgtcccggcccggccgcggggagccccgcttcatcgccgtgggctacgtggacgacacgcagttcgtgcggtttgacagcgacgccgcgagccagaggatggagccgcgggcgccgtggatagagcaggaggggccggagtattgggaccaggagacacggaatgtgaaggcccactcacagactgaccgagagagcctgcggatcgcgctccgctactacaaccagagcgaggccggtgagtgaccccggcccggggcgcaggtcacgacctctcatcccccacggacgggccaggtcgcccacagtctccgggtccgagatccaccccgaagccgcgggaccccgagacccttgccccgggagaggcccaggcgcctttacccggtttcattttcagtttaggccaaaaatccccccgggttggtcggggccggacggggctcgggggactgggctgaccgtggggtcggggccaggttctcacaccatccagatgatgtatggctgcgacgtggggccggacgggcgcctcctccgcgggtaccagcaggacgcctacgacggcaaggattacatcgccttgaacgaggacctgcgctcttggaccgcggcggacatggcggctcagatcacccagcgcaagtgggaggcggcccgtgtggcggagcagttgagagcctacctggagggcacgtgcgtggagtggctccgcagatacctggagaacgggaaggagacgctgcagcgcacgggtaccaggggccacggggcgcctccctgatcgcctgtagatctcccgggctggcctcccacaaggaggggagacaattgggaccaacactagaatatcgccctccctctggtcctgagggagaggaatcctcctgggtttccagatcctgtaccagagagtgactctgaggttccgccctgctctctgacacaattaagggataaaatctctgaaggaatgacgggaagacgatccctcgaatactgatgagtggttccctttgacacacaccggcagcagccttgggcccgtgacttttcctctcaggccttgttctctgcttcacactcaatgtgtgtgggggtctgagtccagcacttctgagtccctcagcctccactcaggtcaggaccagaagtcgctgttccctcttcagggactagaattttccacggaataggagattatcccaggtgcctgtgtccaggctggtgtctgggttctgtgctcccttccccatcccaggtgtcctgtccattctcaagatagccacatgtgtgctggaggagtgtcccatgacagatgcaaaatgcctgaatgttctgactcttcctgacagacgcccccaagacgcatatgactcaccacgctgtctctgaccatgaggccaccctgaggtgctgggccctgagcttctaccctgcggagatcacactgacctggcagcgggatggggaggaccagacccaggacacggagcttgtggagaccaggcctgcaggggatggaaccttccagaagtgggcgtctgtggtggtgccttctggacaggagcagagatacacctgccatgtgcagcatgagggtctgcccgagcccctcaccctgagatggggtaaggagggagatgggggtgtcatgtcttttacggaaagcaggagcctctctgacctttagcagggtcagggcccctcaccttcccctcttttcccagagccgtcttcccagcccaccatccccatcgtgggcatcattgctggcctagttctctttggagctatgttcgctggagctgtggtcgctgctgtgaggtggaggaggaagagctcaggtggggtgaagggatgaagggtgggtctgagatttcttgtctcactgagggttccaagacccaggtagaagtgtgccctgcctcgttactgggaagcaccatccacaattatgggcctacccagcctgggccctgtgtgccagcacttactcttttgtaaagcacctgttaaaatgaaggacagatttatcaccttgattacggcggtgatgggacctgatcccagcagtcacaagtcacaggggaaggtccctgaggaccttcaggagggcggttggtccaggacccacacctgctttcttcatgtttcctgatcccgccctgggtctgcagtcacacatttctggaaacttctctgaggtccaagacttggaggttcctctaggaccttaaggccctggctcctttctggtatctcacaggacattttcttcccacagatagaaaaggagggagctactctcaggctgcaagtaagtatgaaggaggctgatgcctgaggtccttgggatattgtgtttgggagcccgtgggggagctcacccaccccacaattcctcctctagccacatcttctgtgggatctgaccaggttctgtttttgtcctaccccaggcagtgacagtgcccagggctctgatatgtctctcacagcttgtaaaggtgagagcctggagggcctgatgtgtgttgggtgttgggcggaacagtggacgcagctgtgctatggggtttctttgcattggatgtattgagcatgcgatgggctgtttaaagtgtgactcctcactgtgacagatacgaatttgttcatgaatatttttttctatagtgtgagacagctgccttgtgtgggactgagaggcaagatttgttcctgcccttccctttgtgacttgaagaaccctgactttgtttctgcaaaggcacctgc

SEQ ID NO: 14 - DNA, 12835 bp, Homo sapiens
  source 1..12835: mol_type = other DNA
ctctggcccctggtcctgtcctcttctccagcatggtgtgtctgaagctccctggaggctccagcttggcagcgttgacagtgacactgatggtgctgagctcccgactggctttcgctggggacacccgacgtaagagcacattgtgggtgctgagctactatggggtgggggaatatagggagttgtgttaacattgtgcccaggccatgtcccttaagaaattgtgaggttttcttcagagattgctcatctttatcaagggatcgcaaattatttcctccacaaaaggaacttggctacttgccctctccatgagacttgtgtaaggggcctttgtacaggccatttcttctcaaatctccaccaataaaacctttgcatcacatgtcctcagggtctttagaggatttggaaataaggatgctaaaataaattccccaaacagcaattccctttattatgttgacttatgtcagacaaaaggaggtttttactgaaaattttgtgggagtcaagggaattcaatgggtctctcctagacgatcctgggttatgtcctccacaggacctgtggtgttggcccctcttcctcatatgtgaggatggacccagtggcctccctagtatctcctttcttttctttctgaactccaatgtttataaagcctgtatccctgtagtgtatgtaggttgtctgacagaagttatacttagtgctctttctttcttatggggaaaaatccctggaactgaagctgagatcattagtacttggaatcaccttacagatacagagcatttatgaggtattctttggtgcctaaagaacttaaggcatcctctgaaaaactggcccaggttagtgtttattatgaatctttttaacctttctatacttgtttctcctacatctcctacatgctctaactagacatgacagaagagatttaactaatgtagtataaattatatgaaattctatttttgtaagtcaaaaataatcaaatatcagaaatttaataatgttcaaactatatactgtgtggggttaccgagacaatgtggacattgttcacatctcatagggctgaaagtcaatgggcaagtcctggaaactcattgtcttactgcggtcttgtcctcagtttcataggttcacccatcatgccctcagctttccttaattagccatgtctgcttacctcttcctcccatttctctctatttttccccaactatgttgtcatcatttccagaaatctctaaagcttgcacagatccttagcactatgagatccattgaaagagataatatttttctctttgagatagggcctgcctctgtcacccaggctgtagctcagtggtgtgatcgaggctcactgcaacctctgcctcccatgctcaagcaatcctccctcctcaggctccagagtagctgagaatacaggcaggcaaccacgcccagctaatttttgcaattttggtagagaggagattttgccatattgcccaggctggtcttaaactgctgaactcaagcaatcctcctgccttagcctcccaacatgctaggattatagatgtgagccactgtgcccaggcaaaagagatgactcttaataaaaagatttcctttttcttaaatcactgtttctttatccgtgaattcttcttccaactagaaggaggagaaagaagtttgcctgtatttatcaccgggaggagaaggggtctagtgtgacatcaaaatgaaagagtgctggaacttgagccccttcttgctttccaggatccccacagtgatcagttcccataccctggtttattcatgtaaaccacacttatttttctcagcagctactgtgtactgggctccattctaggttcaaatcattctatttgattaagatagagagggtcccgactctcaaggaagttacaagagtagaggagacagacactaacccaataagcatttaacaaagaagataatgttagagagtcatagtgcactgaagaaaagacatcagatgtgtgttgaaagagagaaatggattcacctactttagtttgtatgtttagggagctctacctgagaaagtgatattcagctgagacaacaaaataagtagacagtcataaagatctaacggacgaaagattcagggagaccgaatcgagggaaagcactggtgtgggaaattatgtggagggagagaaagaaggctagaagggctgaggtatagaaagcaaggaaatggagaggcagaagatgaggtaggacaaggagaggaaatcaggagcctcatcattataggctctgatgtccacggtaaaaaatttgaattttattttatttttatttatttttaaattttatttatttatttattttgagatggagtttcattcttgttgcccaggctggcatgccatggcgcaatctctgctcactgcaacctccacctcctgggttcaagtgattctcctgcctcagcttcccaagtaactgggattacaggcactcaccaccatacctggctaatttttttgtatttttagtatagatggggtatcaccatgttgaccaggttgatcttgaactcctgacctcagataatctgtctgccttggcctcccaaagtgctgggattacaggcgtgagccaccacgcccggactgaattttatttaaatagatatgagaagctgctgtatggttacaaggagagtcaatttatattcaacttttgtgtgtgtgtgtgatggagtctcactctgttgcccaggctcgattgcagtggcacaatctcggctcactgcaacctccgcctcccgggttcaagaaattatcctgcctcagcctcctgagtagctgggaccacaggtgcatgccaccacagtcggctactttttgtatttttagtagggatggggtttcaccatgttagccaagatgggctcgatctcctgaccttgtgatccacccgcctcggcctcccaaagtgctgggattacaggcatgagccactgcgcccagcttatattcaattattacaattaattctagctactttgtggggattggattgttggggttcacaagtggttaggaagactatttaggagcacagcagggaattctccagggaaaacaggcttgtggcttcatggagtgcattagtgataaagacggtgaaaaagataaagtggacagacttcgcatgtatttttgcttagcttgttaatgaattactgtaaagggggtggaacaatcaagtttattcctgaggattttgttttgacaaataagtgggtggtagttttgtttattgagataggaaaaactatgggaggaaatgatttgaagtgggtggttggaaataaaagttttgtttaaatttgagatgatttattgacatttatgtagagcaatcagaaggtcagtggcatttaagagactcatggtgaggctagggcttcagatatttatgttggcagcatcaatacgtgtagtgtgttaaattctagggagtggaagaggatacatagggagatggattgtgtggagaaaaaagaagagggcacaggccagcaaaggcagctgagacagagcccagggatgttggagaaaaaccaagagaacataatgagtgtaagtcaaggaaaatagatttttttcaaggagaagggagaggccaattgtggtgagtaccactaagcggagggggaagtgagaacttgacagagaagcaagtgctgggtttgctggagttgatatttgcagtcaatggagtatccagggaggaaactggattggaccatttgaagagcgagtagaagtgaggacgaggttaaggttgactgttttgagtagaacttccggaaggactgtgctctgggttcaggaagcccgctgaatctaaaggaaaaggctaaagaagctaaagagacagaggaggacctgtgaacaagagatgctcagtcattagcaaggaaatactggagatcccctgtgtgcagtggtgactactcatgcaaaaggtcacacagccaatatttattagtgacatagaatataccagttattactctaggtcatgagaatggtgataaataaaatgaatccgctcgccatcagtatatgccatgtaacagtttgcagtgactgtgtaccaggcctgtgaatttctgtatgcaatttcaataatgatcctgctgtatctgtggtatttaaaaacatatacatctctggaatctaaaattgagggattataagtaaaacccagtattagaaatttagtgctggaaatcagattgcagtttaaatctgagcatatagaaagtccctttcttctatgtcagcagatgccttttatgtgaggcttaggtatactacattattagacataaaccagtgtttctgccctatgttttcagaatgacaattctttatgaaactcatagaagaacagaagacaattgcaaaatcatgatgaagatactaattgctttagaattaaggaatacaaaaaataatgtgagttacagttatagggatcataaaagtgaaaatgggaatgtatttgagtgtttattatgtgatcagtgctaagaagagtcatcatttaatttgacacttaacagtaatcctgtgaggattatgctattattaaatgcatttgatagattacaaaaaggcttactgttggcaaaaattgacccaagtagaagaggtcacctttttattcagattttctgattgtagagtttgagagtttttccatcattagggagtagtgactatattgtgtctgaattattgacagaatttctgatattcatatataccaggttgtttctttgagtggggacagagatgcaagggctgctagttccaatgtacaggagaaacttttaatcattttgcatttatcattttaaaagttcagtatgtctataatggtcatgtgttgacaaacacaaggaagtattaaatcactcctccttctgaggtttgactagcaaattgggcaagagttaccaaataaaatacatgttcctagttaaatctgaatttcagatacacgaccataatttattggaaatccaaatttaattgggcaccctctggttttatttgccaaatctgtcaactctaagtgagacacatggacatggattacagtgctacccatgcaagccacagtgacagcaacttcacacatgtttacattttaactttctctctgtaagaaagtgcttagataatttggtgataaaaagatagccattgtttgatccagggtggacacctctctgccatcgtttctaaagggcaaacggagatttctgcaggtcttgctcacagtctggggacctgctcatgttttgaaactgtctgtatcagaatgtcattttcttggtttctccctttctgaggggacttgactacaaaaccaagagttctgcctctggccaaggctggaaatgtgatgcctgctagtattgttgggagtgggagactgagagaaatgagttagttggggcatttaacgggaataaaatagctgtggttgtgattcattactatagataattagtggaccagtggcggagaaattaagaaaaaagatgatgtgaacgataaatgatatgattagtgactgcttggtaaggcaaggaaatcattaaatcttggttctcatcaagttcattttctgaaaagatagcactgcattgggaccagaattctacaaaacattctttttacataggaccaagattttcaagaaatatttttcaatacaattctcagctgctccataactaatagtagcttgttcaacacagatttttttagatggttcacacctgtggttcttacccagggatagttcaccacccctcccttccctcccatcatccttggggaacagtgacaatgtttggaaaaatttttggttgtcacaacaggggtttcttctgatatttaatgagtagaagccaggaacactgctagagaacctacaatgttcagaacagcctccgccatcaacaaagaattatctggtccaaaatgtcaatagtgctgaggctgagagcactgcttcacgctgtgctctttctgaaaattctacactcacatctgttatacactcaccacacagttcagtttttcatagtttatttttgcttcttccattataaaaaattagacagttgcataaatttaaccactttcttgttgaatccatttagtcaatgcaagctcagtattttcatatttagtttttgccttatgcaatatttttcaacattttcatgagttgttggtcatcaccatctctattaactttcaacaacttgctcttgtaagtcacaaatagtgatgctgctgaaattatttctcactaacatgcctcagatttctgtagtgattctacattttatattattcacaatgtaaaatgcttctatgtactcatttcacttttacccaaggattatttttaagttatttttgtcatttacacacttcaaacataaagacaaaaacatcaaaaatatatactgtgttttacataggtgtctattttcacacatatatgtatgtatgtttatatgtatggaaactacagaagcacatgtcgccaataagagctctgagacacctttgaccacttacccttatcagatgagatttgccaaatgagttgtggaaacaagttttttaaactgaatttctgagctttgtgaatttagaaatgcaaaggaaagtttgtggacatttacagggatcatggttttattgtcctttaaactcttcgatactttaccattgtcttactataaatccaaaatcctaaccccacccacgaggctttaaatacctggcttcttgtgatttctccaggctaatcttttaccctccttcccctcagcctctctgctttagtgaactttctcctagttttttgaagaagttcatcaattcaagcttttgtagatgggatttcctaaacctgaaatgtgcctcccgttttgtccaaacagacacgggctccactctgccccctggctcacacctgcttaacccgtcaagtcacatctgaactgtcactctttagagggtccttctctggcaccctaatgtaattgagatcatcctattattctctgctctagaacttcccacttccgagatttctcattcctgtctaacctcttgtgtgtttggtgtttggccatcgctttcactgctctttaagctcccccagcggagtggagaggtctgttttcccttgtttggattcctagaggcagcgcaggccagacacaaggtcatcactaagaaagtgttcacaggatgaacgcggtgggtgctgtttaaggaaccggtaaacatgtgggatgagagaaggagcagagagtgtttttcgggtggaggctcccaggaggaggaggggcgggctgcggtgctgggcggatcctcctccagctcctgcttggaggtctccagaacaagctggaggcagggatggggggtcccaaaagcctggggatgagaaggggtttccccgcatagtcccccaggcccccgttcgcctcaggaaaacggaggatgagctcctgggctgctggtggtgggcgttgcggggggggccggttaaggttcccagtgcccgcacccggcccagggggccccggatggcggcgtcgctgtcagtgtcttctcaggaggccgcctgtgtgaccggagcattcgtgtccccacagcacgtttcttggagctgcttaagtctgagtgtcatttcttcaatgggacggagcgggtgcggttcctggagagacacttccataaccaggaggagtacgcgcgcttcgacagcgacgtgggggagtaccgggcggtgagggagctggggcggcctgatgccgagtactggaacagccagaaggacctcctggagcagaagcggggccaggtggacaattactgcagacacaactacggggttggtgagagcttcacagtgcagcggcgaggtgagcatgtcggggggcggggcctgggtccctgtgagctggaaatctgagagtgtgtgtgtgtgtgtgtgtgtgtgtgtgtgtgtgtgtgtgtgtgtgtgagagagagagagagagagagagagaggaagagagagacagagagagggagcgcgccatctgtgagcatttagaatcctctcaatcccgagcaagcagttctgaaggcacaggtgtgtgtgtagagtgtggatttgtctgtgtctgtgtgcctgttgtgggagggaaagcaggagggggctgcttcttatccttggagacttctgtggagaggtgacaagggaggtcggtgcgggcggctggagagagaggagaccttgattgtcctgggtccttagagatgcagggaaggaaagtgtaaggtgtgtgtgggtggggtgaaggtttaggggaggagagctgaggggtaaggaaggtttgggataatgtgaggaggccggttccagactgtccctggcacacacccttcatgtaatctctgaaataaaagtttgtgctgtttgtaaaagcattagattaagttctaggggaattgagtagacctctaaggcacctctgaaacttctttaggtataaatttcttgctagttttttgtttttctagtgtgtatatttttacatagtagaaatgacagtgaaactaacttttgaattaaagttttaacacaattactgtattattataatgctaatagttttctagtagttacatattattcttttatatataatagttgtgacacaacttagggatcactttcccctttgttgacctttattatgacattcaccaaaagttgaaaatgtatgtttctggttaatttttaatttatattttttcatttgaaaaaatgtggagaggtctgtttttcattcaaaagagttcttttgaattattttgaccgatttattggtcaattttaataactgctctaagaattccctattttatttggtaggtaatggacaatgatctactgcctaatatctctagggcttagtatttttctcagtgactttgtgggttctttgtactgtaagattattaacactttattgatatttgattcagcatttgctccagtttgtggtttgtatgttgattttgaaaattcttttccatgttaagaatttgaacatttttatttaataaaatgtattgcaaaaattttattaatgatttacaatccatcttaaatctgccattttgtggtatttttgtctctaggtttctccttacttctaaaaacattgtatttattgagagtatgctaatgtcggggattttcctgggcataagcaccacaagtaatgagttccagaccctgccttaatccaaatgtgattctggaaagaaaaatcattttacaactggcctgataataattctgcttgtgttgcacgggggatacattgagcagctgaatgaaaatataagaactttcaaaactaaaatgacgctcctaaatccttctgtctgctttaggactcatgcttttctaggaacgtaaaaatttggagaatcatttctgtctgtcccaccttcccaggggcagaaccatttctgtggtgttctaaggtgtgagtgcatggcagtagtattcctaaaaattcatactcggttttctcatgtacccaactctgtccctttgtctatgtacattgctttaaatcatatttttctgtcacggagtacaaggatgataaataggtgccaaatggagcacccaagtgtgatgagcccccttacggtggaatggagtgagaagctttctgatctcataaattgaaggctatcttcagtcattatgtattttacataaattaatcctcatgtaaccccaagaggtaaattagtataattatccttcattgggggtgacaaagttgagacacagaagaatcaactcttccaggatcaaccagtaaaaggcagaccttggatttgaaccaggcaacctggctcagatatcagttttaattactacactctgtactttcaaaaatttgtaaacactttgacaatgcatgccaatttcaagttatgaagaagcaaacgcaatttttcacatctctcaaatctaatgggtcctcactatcaagattaaattccaggctgatgacactgtaaggccacatggccagctgtgctggaggcctggtcaaggtcagagcctgggtttgcagagaagcagacaaacagccaaaccagcagaattactctgtcttcctgactcattccctctacctttttttctcctagtccatcctcaggtgactgtgtatcctgcaaagacccagcccctgcagcaccacaacctcctggtctgctctgtgagtggtttctatccaggcagcattgaagtcaggtggttccggaacggccaggaagagaaggctggggtggtgtccacgggcctgatccagaatggagactggaccttccagaccctggtgatgctagaaacagttcctcggagtggagaggtttacacctgccaagtggagcacccaagcgtaacgagccctctcacagtggaatggagtgagcagctttctgatttcatacatttctcacccaccaagaaggggactgtgctaatccctgagtgtcaggtttctcctctcccacgtcctattttcatttgctccatgttctcatctccatcagcacaggtcactggggatagccctgtaacagtgtgtagaaacacctgtactccctgtggaatcagtcatgcttttttttttttgaacctccccatgatgtcacaggtcagggtcacccgatctccccaagctccaggccctgcttctgggtctgagactgagtttctggtgctgctgctctgagttacttgttttgatctgggaagaggggaagcataggggcctacctgacatgaggggagtccaatctcagctctaccttttattagctctgtcactctagacaaactacttagcttcattgagtctcaggctttctgtggatcagatgttgaactcttgccttacatcaaggctgtaatatttgaatgagtttgatgtcggaaccttgtaactgttcagtgtgatttgaaatcctttttttctcccgaaatggctagttattttagttcttgtggggcagccttcttccccattttcaaagctctgaatcttagagtctcaagtaaagaggttcaatctggaataagcatcactaaacctggcttcctctctcaggtgcacggtctgaatctgcacagagcaagatgctgagtggagtcgggggctttgtgctgggcctgctcttccttggggccgggctgttcatctacttcaggaatcagaaaggtaaggagtctttggtagctggctctctccataggcttttctggaggaggaactatggctttgctgaggttagttctcagtatatgagtggccctgaataaagcctttctttccccaaacggctctaatgtcctgctaatccagaaatcatcagtgcatggttactatgtgaaagcataatagcttgtggcctgcagagacaagaggaaggttaacaagtaggggtcctttggtttgagatcttggagcaaattaaggaagagccactaaagttaatggaattacactggatcctgtgacagacacttcatgcttcatgggtcacatggtctgtttctgctcctctctgccctggttggtgtgggttttggtgttagaactctccggtgggagatctgggactgggatattgtgttggaggacagatttgcttcaatatcttttaagtgtatatcttttcctctttttcccaggacactctggacttcagccaacaggtaataccttttaatcctcttttagacacagattcagtttctccagtgagaggtgaagccagctggacttctgggttgggtggggacttggagaacttttctgtcttacaagaggtttctaaatgcaccaatgagtgctctgtaaaaacacaccaatgagtgctctgtaaaaattgacactctgtggctcgctagatgtttgtaagatggaccaatcagcactctgtaaaatggaccaatccacactctgtaaaatggaccaatcagcactctttaaaatggaccaatcagcaggatacgggcagggacaaataagggaataaaagctggtcaccctagccagcaccggcaacctgcttaggtccttttctatgctgtggaaggtctgttctttcactcttcacaataaatcttgtgctcactctttgggtccgtgccacctttaagagctataacactcactgcaagggtctgtggcttcactcttgaagtcagccagaccctgaacctaccggaaggaacaaactcaggacacactagaatgatggtagaggtgataaggcatgagacagaaataataggaaagactttggatccaaatttctgatcaggcaatttacaccaaaactcctcctctccacttaaaaaaggcctgtgctctgcaggactattggctctgggagactcaggaacttgtttttcttcttcctgcagtgttctcatctgagtccttcaaagagggggaaagaaacgttagtagagccaggttgaaaacaacactctcctctgtcttttgcaggattcctgagctgaagtgcagatgacaatttaaggaagaatcttcttccccagctttgcaggatgaaaagctttcccgcctggctgttattcttccacgagagaggg

SEQ ID NO: 15 - DNA, 14883 bp, Homo sapiens
  source 1..14883: mol_type = other DNA
aaccttttgagtactaaattaaaacaatcctttaaagaagacaattgtttcagaggaggaccttcatacagcatctctgaccagcaactgatgatgctagtgaactcagatgctgactggttctccaacacgagattacccaatcaaggagcaaggaaatcagtaacttccttcctataatttgaaatgtgggtggaggggggtcatagttctccctgagtgagacttgcctgctgctctgaccactggtcttgtcctcttctccagcatggtgtgtctgaagctccctggaggctcctgtatggcagcgctgacagtgacattgacggtgctgagctccccactggctttggctggggacacccaacgtaagtgcacattgtgactgcagagctactatggggtgggggcaaaaataggaagttttgttaacattgtgcccaggccatgtctcttatgaaattgtgacactttcttcagagattgcccatctttatcagatggatcccaaattatttccaccacaaaaggcgcttggctacttgtcctgtccatgagatttgtgtaaagggcctacacacaggtcatttcttctcaaatctccaccaataaaacctatgcatcacatgtcctcagggtctttagaggatttagaaataagtatgctaaaataatttccccatacagcacttccctttattatgttgacttatgtcagacaaaaggaggtttttactgaaaattttgtgggagtcaagggaattcaaagggtctctcctagacgatccagtgttatgtcctccacaggacctttggtgttggcccctcttcctcatatgtgaggatggatccagtggcctccccattatctcctttcttttctttctgaactccaatgtttataaagcctgtacccctgttgcttatgtaggttctctgacaaaagttatacttagtgctctttctttcttatggggaaaaatccgtggaactgaagctgagatctttagtacgtggagtcaccttacagatacagagcatttatgcggtattcattggtgcctaaagaactttaggcatcctctggaaaaccggcccaggttagtgtttattatgaatcttttttaaccgttctatacttgtttctcctacatctcttacatgctctaactagacatgacagaagagatttaactaatgtagtataaattgtatgaaattctatttttgtaagtcaaaaataatcaaatatcagaaatgtaataatgttcaaactatatactctgtgtggggtttttgagacaatgtggacattgttcatgtctcataaggctgaaagtcaatgagcaagtcctggaaactcattgtcttactggggtcttgtcctaaatttcataggttcacccatcatgccctcagctttccttaattagccatgtctgcttacctcttcctccactttctctcttttccccagctatgttgtcatcatttccaaaaatctctaaagcttgcacagatccttagcactatgagatccattgaaagacataatttttttctttttgagatagggcctagctctgtcacccgggctgtagctcagtggtgtgatcgaggctcactgcaaccactgcctcccatgctcaagcgatcttccctcctcaagctccggaatagctgggaatagaggcaggcaaccacgcccagctaattttttaattttggtagagatgagattttgccatgttgcccaggctggtcttaaactgctggactcaagcaatcctcctgccttggcctcccatcgtgctaggattatagatgtgagccactgtgcccaggcaaaaagaaatgactcttaataaaaaaaatttcctttttcttaaatcactgtttctttatctgtgaattcttcttccaagtagaaggaggagaaagaagaagtttgcctgtatttctcaccggggggagaaggggtctagtgtgacatcaaaatgaaagggtgctggagcttgagccccttcttgctttccgggatccccacagtgatcagttctcataccctggtttattcgtgtaaaccacacttatttttctcagcagctactgtgtactaggctccattctaggttcaaatcattctatttgagtaagatagagagggtcccgactctcaaggaagttacacaacaatagaggagacagacactaacccaataagcatttaacaaaaaagaaaatgttagagagtcatagtgcactgaagaaaagacaacaggtttgtcggaacgatagaaatggattcacctactttagttcatatgtttagggagccctacctgagaaagtgacattcagctgagacaacaaagtaagtagacagtcgtgaagatctaacggacgaaagttccagggagactgaatcgagggaaagccctggtgtgggaaattatgtggagggagagaaagaaggctagaggggctgatgtataggaagcaaggaaataggggggcagaagatgaggtagaacacagagaggaagtcaggagcctcatcatattaggctctgaaatccacggtaaaaaatttgaatttcatttatttatttattttgagacggagtttcattcttgtggcccagactggagtgcaatggcacaatctcggctcactgcaacctccgcctcctgggttcaagtgattctcctgcctcagcttcccaagtagctgggattacaggcacccaccaccatacctggctatttttttgtatttttagtggagatggggtatcaccatgttgaccaggctggtctcgaactcctgacctcagataatctgtctgccttggcctcccaaagtgctgggattacaagagtgggccactgtgcccagactgaattttatttaaatagatatgagaaactactgtatggttacacggagagtcaatttatattcaattttgtgtgtgtgtgtttgtgagatcgagtcttgctctgttgcccaggctagattgcagtggcacaatctcggtttactgcaacctctgcctcccaggttcaagcgattctattgcctcagcctcccgagtaactgggaccacaggcgcatgccactacacccggctaattttttgtatttttaatagggatggggtttcaccgtgttagccaggttggtctcgatctcctgacctcgtgatctgcccaccttggcctcccaaagtgctgggattacaggcgtgagccactgcacccggcctatattcaatttttaaaactaactctagctactctgtaggaattggattgttggggttcaaaagtggtcaggaagactatttaggagcacagcagggaattatccagggaaaacaggcttgtggcttcatggagtgcattagtgataaagagagtgaaaaagataaagtggacagacttggcatgtatttttgcttagcttgctaatgaattactgtaaaggggttagaacaatcaagcttattcctaaggattttgttttgacaaataagtgggtggtagtgttgtttattgagataggaaaaactatgggaggaaatgatttgaagtgggtggttggaaataaaagttttgtttaaatttgagatgatttattgacatttatgtggagcaatccgaaggtcaatggcatttaagagactcatggtgaggtgaagctagggcttcaggtatttatgttggcggcatcaataagtgtagtgtgttaaattccagggagtggaagatgatacatagggagatggattgtgtggagaaaaaagaagagggtacaggccagcaaagggggctgagaaagagcccagggaggctggagaaaacccaagataacataatgcgtgtaagtgaagaaaaacagattattttcaaggagaagggagaggccaattgtggtgagtaccactaagaggagagggaagtgagaacgtgacagagaagcaagtgctgggttgctggtttgctggagttgatatttgcagtcaatggagtatccagggaggaaactggattggaccatttgaagagcaagtagaagtgaggacgaagttaagattgactgttttgagtagagagcttcagggaaggactgtgctctgggttcagggagccagctgattctaaaggaaaaggctaaagaagctgaagaaccagctttcccagcaccatttattaaatagggaatcctttccccatttctggtttttgtcagatttatcaaacatcagatggttgtagatgtgtggtattatttctgagggctctgttctgttccattggtctatatctctgttttggtaccagtaccatgctgttttggttactgtaggcttgtagtacagtttgaagtcaggcagcatgatgcctccagctttgttcttttggcttaggattgtcttggcaatgcgggctcttttttggttccatatgaattttaaagtagttttttgcaattctgtgaagaaagtcattggtagcttgatggggatggcactgaatctataaattaccttgggcagtatggccattttcacgatatttattcttcttatccatgagcatggaatgttcttccatttgtttgtgtcctcttttatttagttgagcagtggtttgtagttctccttgaagaggtccttcacatgccttgtaagttggattcctaggtattctgttctctttgaagcaactgtgaatgggagttcactcatgatttggctctctgtttgtctgttattggtgtataagaatgcttgtgattttttgcacatatgtagaaagctaaaactggatcctttctttgcacctcatacaaaaattaattcaagatggattaaagacttaaatgttagacttaaaaccataaaaaccctagaagaaaacctaggcaataccattcgggacataggcatgggcaaggacttcatgtctaaaataccaaaagcaatggcaacaaaagtcaaaattgacaaatgggatctgattaaactaaagagcttctgcacagcaaaagaaactaccatcagagtcaacaggcaacctacagaatgggagaaaatttttgcaatctactcatctgacaaagggctaatatccagaatctacaaagaactcaaacaaatttacaagaaaaaaacaaacaaccccatccaaaagtgggcaaaggatatgaacagacacttctcaaaagaagacatttatgcagccaacagacacatgaaaaaatgctcatcatcactggccatcagagaaatgcaaatcaaaaccacaatgagataccatctcacaccagttagaatggcaatcattaaaaagtcaggaaacaacaggtgctagagaggatgtggagaaataggaacacttttacactgttggtgggactgtaaacgagttcaaccattgtggaagacagtgtggtgattccttaaggatctagaactagaaatactgtttgacccagccatcccattactgggtatatacccaaaggattataaatcatgctgctataaagacacatgcacacgtatgtttattgtggcactattcacaatagcaaagacttggaaccaacccaaatgtccatcaatgatagactggatgaagaaaatgtggcacatacacaccatggaatactatgcagccataaaaaaggatgagttcatgtcctttgtagggacatggatgaagctggaaaccataattctcagcaaactatcacaagcagaaaaaaaccaaacaccacatgttctcactcataggtgggaattgaacaatgagaacacttggacacaggaagaggaacatcacacaccggggggaggggggagggatagcattaggagatatgcataatgtaaatgacgagttaatgggtgcagcacaccaacacggcacatgtatacgtgtgtaacaaacctgcacattgtgcacatgtaccctagaacttaaagtataataaaaaataaataaataaataaataaataaaaagaagctgaagagaaggaggaggacttgtgaaccagagatgctcagtcattattagcgaggaaatgctagagagcccctgtgtgcagtgctgactacttatgcaaaaggtcacacagccaatatttaacacagccagtatttcacacagccaatatttattaatgacatagcatacatataccagttattactctaggtcataagaatggagtgataaataaaatgaatctggtcgccatcagtatatgccaggtaacattttgcagtgactgtgtaccaggcctatgaatttcagtatgcaatttcaataattatcctgttgtatctgtggtatttaaaaaacatatacatctctggaatctaaaattgagaggatatgagtaaaacccagtgttacaaatttagtgctggaaatcagattgcagtttaaatctgagcatgtagaaagtccctttcttctatgtcagcagatgccttttgtgtgaggtttaggtatactgctttattagacataaaccagtgtttctgccctatgttttcagaatgacaattccttatgaaactaatagaagaacagaagacaattgcaaaatcatgatgaagatactaattgctctagaattcaggaatacataaaatgatgtgagctgcagttatagggatcataaaagttaaaatgggaatgcatttgagtgtttattatgtgatcagtgctaataagagtcatcatttaattttacacttaacagtaatcctgtgaggattacgctattattaaatgcatttgatagattacaaaaaggattatggttggtaaaaattgccccaagtagaagagatcatgttttttattcaggttatctgattctagagtttgagagtttgaccatcattagtgagtagtgactatattgtgtctgaattattgacagaatttctgatattcaaatgtaccaggttgcttcttagagtgcagacagagatgcaagggctgctagttccgatgtataggagaaactttcattcatttcacatttatcattttaaaagttctgtatgtctataatggtcatgtgttgaagaacacaaggaagtattaaatcactccttcttctgaggtttgactagcaagttgggctagagttaccaaataaaatacatgttcctagttaaatctgaatttcagatacacaaccatgatttcttggaaattcaaatttacctgggcatcctctggttttatttgccaaatctgtcaaccctaagtgagacacatggacatggattacagtgctagtcatgcaaaccacagtgacagcaacttcacacatgtttattttttaactttctgtctgtaaagaaagtacttagataatttagggataaaaagatagacattgtttgatccagggtgcacacctctctgccatcatttctaaagggcaaagggacatttctgcaggtcttgctcacagtctggggacctgctcgtttttgcaaactgtctgtatgagtatgtcattttcttggtttctccctttctaaggggacttgactgcaaaaccaggagttctgcctctggccaaggctggaaatttgatgcctgctggtattgttgggagtgggagactgaaagaaatgagttagttggggcatcaaatgggaaataaaatagctgtggttgtgattcattattacagaaaattagtggaccagtggcagagaaattaaaaaaaaagatgatgtgaacgataaacgatatgattagtggctacttggtaaggcaaggaaatcattaaatcttggttctcatcaagttcattttctgaaaagatagcactgtattgggaccagaattctacaaaacattctttttatgtaggaccaagattttcaacaaatatttttcaatgcaattctcagcgctccataactaatagtagcttgttcaacacagattttttcggatggttcacacctgtagttcttacccagggatagttcaccacccctcccttccctcccatcatcgttggtgaacggtggcaatgtttggaacaatttttggttgtcacaacaggggtttcttctgatattcaatgagtagaagccagggacactgctaaagaacctacaatattcagaacagcctctgctatcaacaaggaattatctggtccaaaatgtcaatagtgctgaggctaagagcactggttcacactgtgctctttctgaaaattctaggctcacatcttgtatacactcaccacacagttcagttttttatggtttatttttggttgtttcattataaaaaattagacagttacataaattcaaccattttcttgttgaatccatttagtcaatgcaagcttaatattttcatatttattttttgccttatgcaatatttttcaacattttcatgagttgttggtcatcactatctctattaactttcaacaacttgctcttgtaagtcacaaatagtgatgctgctgaaactgtttctcactaacaggcctcagatttgtgtagtgattctacattttatattattcacattgtaaaatgctactatttattcatttcacttttacccacaggattattttaagttattttttgtcattttcacacttcaaacataaagacaaaaacatcaaaaatatatacagtgttttacatatgtgtatattttcacatatatctgtatgtatgtttatatgtattgaaactacagaagcacatgtcaccaataagagctctgagacacctttgaccacttacccttatcagatgatatttgccaaatgagttgtggaaacaagttttttttaactgaatttcagagctttgtggatttagaaatgcaaaggaaagtttgtggacatttaccgggatcatggttttattctccttaaaactcttcaatactttaccattgtctttagttaaatccaaaatcctaacaccacccacgaggcttttcaatacctgccttcttgtgatttctccagcctaatcttttaccttccttcccctcagcctctctgctttagtgaactttgttctagtttcttgaagaagttcatcaattcaaccttttgtacatgggatttcctaaacctgaaaatgtgcctcccgttttgtccagacagacacacaggctccatccactctgccccctggctctcacctgcttaacctgtcaagtcacatctgaactgtcactcttcagagggtccttctctggcaccctaatgtaattgagatcatccttttattctctgttccagaactccacacttctgacgtttctcattcctgtctaagctcttgtgtgtttggtttttggccatcacgttcactgctctttaagctcccccagcggagtggagaggtctgttttcctttgcttggattcctagaggcagcgcagggcaggcacaaggtcagcactaaggaagggttcacaggatgaacacggtgggtgctgttgaaggaaccggtaaagcgtgtgggatgagaggagcagagagtgtctttcaggtgaagactcccagaaggaggccgcgtgggccgcggtgctgggcggatcctcctccagctcctgcttggagttctccagaacaggctggaggcagggagggggtccccaaaagcctgaggatcagaaggggttttcccgcctggtcccccagaccccggttcgcctcaggaagactgaggacgagcccctgggctgctggtggtgggcgttgcgggtggggccggttaaggttcccagcgcccgcaccccgcccagggagcccgggttggtggcgtcgctgtcagtgtcttctcaggaggccgcccgtgtgaccggatcgttcgtgtccccacagcacgtttcttggagcaggctaagtgtgagtgtcatttcctcaatgggacggagcgagtgtggaacctgatcagatacatctataaccaagaggagtacgcgcgctacaacagtgacctgggggagtaccaggcggtgacggagctggggcggcctgacgctgagtactggaacagccagaaggacctcctggagcggaggcgggccgaggtggacacctactgcagatacaactacggggttgtggagagcttcacagtgcagcggcgaggtgagcatggtggagggcggggcctgggtccctgtgagcggggaatctgtgtatgtgtgtgtgtgtgtgtgtgtgagagagagagagagagagagagacagacagaggaagagagagagagcgtgtgccatctgtgagcatttagaatcctctctatcctttcttatggctgcgtagtattccctggtgtataggtgccacattgtcttaatccagtctatcattgatggacattagggttaattccaagtctttgctattgtgaatagtgtcagttcatgtcctttgtagggacatggatgcagctggaaaccatcattctcagcaaactatcacaaggacagaaaaccaaacaccacttgttctcactcataggtgggaattgaacaatgagaacacatggacacaggaaggggaacatcacacacaggggcctgtcgtggggtggggggagcagggagtgatagcattaggagatatacctaatgtaaatgactagttaatgggtgcagcataccaacatgacacatgtgtacatatgtaacaaacctgcacgttgtgcacatgtatcctagaacttaaagtataataataataattatatatatatatatatatattagaaaaacaaacaaaaagaatcttctgtatcctgagcaaggagttctgaaggcacaggtgtgtgtagagtgtggatttgtctgtgtggctgttgtgggaggggaaacaggagggggctgcttcttattcttggaggcctctgtgcagaggtgacatgggaggtgggtgtagggggctggagagagaggagaccttgattgtcctgggtccttagagatgcagggaagggaagaataaggtgtgtgtggttggggtgaaggtttaggggaggagagctgaggagtgtggaaggtttgggataatgtgaggaggccagttccagactgtccctggcacacacccttcatgtaatctctgaaataaaagtgtgtgctgtttgtttgtaaaagcattagattaatttctaggggaattgaggagacctctgaggcacctctgaagcttctttaggtctaaatttcttgctagtttttgtttttttagggtgtatatttttacatagcagaaatgactgtgaaactaactttttgaattaaagttttaacacagttactattttattataatgctaatagttttctagtagttacatcttattcttttgtatataatagttgtgacacaacttacctcactttcccctttttgttgacctttattatgactttcaccaaaagttgaaaatgtatgtttctggttaatttttactttatatttttcatttgtaattcttttgaattattttgacctatttattggccaattataattactgctgtaagaattccctatttcatttggtagggaatgtacaatgatctactgtctaatatcttaagggcttagtatttttctcagtgactttgtgggttctttgtactgtaagattattaacactttattgatatttgattcagcattttctccagtttgtggtttgtatgtggattttgaaaagtattttccatgttaagaatttgaacatttttatttaataaaatatattgcgaaatattaattaatgatttacaatcatcttaaatccaccattttgtggtattattgtctccaggtttctccttacttctaaaaaaaaactgtatttattgagtatgctagtgtcagggattttcctgggcataagcaccccaagtgacgagtcccagacactgccttaatccaaatgtcattctggaaagaaaaatcattttacaatgataggcctaataataattaagcttgtgttgcacaggagatacattgatcagctaaatgtaaacgtaagaactttcaaaactaaaatgacattccttaatcattctctctgcgttaggactcatgcttttctaggaacgtaaaaatttgagaatcatttctgtctgtcccaccttcccaggggcagaaccatttctgtggtgttctaaggtgtgagttcatggtggtagtattcccaaaaattcatactcggtttcctcatgtacccaactctgtccctttatctacccacattgctctaagtcatatttttctgtcacggtgtacaaggatgataaataggtgccaagtggagcacccaagtgtgatgagccccctcacagtggaatggagtgagaagctttctaacctcataaattgaaggctatcttcagtcattgttttatatattttacgtgcattaatcctcatataaccccaagaggtaaattagtataattatccttcattgtaggtgacaaagttgagacacagaagaatcaaaaaactcttccagcatcaaccagtaaaaggcagaccttggatttcaaccaggcaacatggctcagatatcagttttaattactacactctactgtcaaagatttgtaaacactttgacaatgcatgccaatttcaatctatgaagaaacaaacacaatttttcacaacatctctcaaatctaatgggtcctcactatcaagattaaattccaggctgatgacactgtaaggccacatggccagctgtgctggaggcctggtcaaggtcagagcctgggtttgcagagaagcagacaaacagccaaacaaggagacttactctgtcttcatgactcattcccactaccttttttttctcctagtccaacctaaggtgactgtgtatccttcaaagacccagcccctgcagcaccacaacctcctggtctgctctgtgaatggtttctatccaggcagcattgaagtcaggtggttccggaacagccaggaagagaaggctggggtggtgtccacaggcctgatccagaatggagactggaccttccagaccctggtgatgctggaaacagttcctcggagtggagaggtttacacctgccaagtggagcatccaagcatgatgagccctctcacggtgcaatggagttagcagctttctgacttcctaaatttctcacccaccaagaaggggactgtgctaatccctgagtgtcaggtttctcctctcccacatcctattttcatttgctccatgttctcatctccatcagcacaggtcactgggctactgggggtagccctgtaggtgtttctagaaacacctgtaccccctggggaatcagtcacgcctgccaggcaggagaggctgtccctcttttgaacctccccatgatgtcccagttcagggtcacccactctccccaggctccaggccctgcctctgggtctgagactgcgtttctggtgctgttgatctgagttgtttgttgtgagaagaggagaactctaggggccttcctgacatgaggggagtccaatctcagctctgccttttattagctctgtcactctagacaaactacttaacctcactgagactcaggctttctgttgatcagattttgaagttgcgccttacatcaaggctgtaatatttgaaatgagtttgatgcctggaccttgtaactgttacagtgtgatttgaaaaccgttttttcccccagaaatggctagttattttagttcttacagggcagccttctttcccattttgaaagctctgaatcttagggtctcaattacagaggttcaatttggaataaacactaaacctggcttcctctctcaggtgcacggtctgaatctgcacagagcaagatgctgagtggagtcgggggctttgtgctgggcctgctcttccttgggacagggctgttcatctacttcaggaatcagaaaggtgaggagcttttgggagctgaatctccataggcttttctggaggaggaactatgactttgctagggttagttctcagtatatcagtggccctggataaagcctttctttccccaaatgacctccaatgtcctgataatccagaaatcatcagtgcatggttactatgtcaaagcataatagcttgtggcctgcagagataagagaaaggttaacaagtagggattctttggttggagatcctggagcaaattaaggaagagccactaaggctaatgcaattacgctggatcctgtgacagacacctcatgcttcatgggtcacatggtctgtttctgttcctctctgccctggttggtgtgggttgtggtgttagagaaatctcaggtgggagatctggggctgggacattgtgttagaagacagatttgcttccatatcttttaagtgtatatcttttcctctttttcccaggacactctggacttcagccaacaggtaataccttttaatcctctttcagaaagagattcagtttccctagaatgatggcagaggtcataaggcatgagacagaaataataggaaagactttggatccagatttctgatcaggcaatttaccccagaactcctcctctccacttagaaaaggcctgtgctttgcaggatcattggctcagggagacttaggaacttgtttttcttctccctgcagtgctctcatctgagtcgttgaaagcggggaaaagaagttttagtaaagccaggtctgaaaacaatttcctctgtctctgcaggactcttgagctgaagtgcagatgaccacattcaaggaagaaccttctgcc

SEQ ID NO: 16 - DNA, 12866 bp, Homo sapiens
  source 1..12866: mol_type = other DNA
ctctggcccctggtcctgtcctcttctccagcatggtgtgtctgaagctccctggaggctccagcttggcagcgttgacagtgacactgatggtgctgagctcccgactggctttcgctggggacacccgacgtaagagcacattgagggtgctgagctactatggggtgggggaatatagggagttgtgttaacattgtgcccaggccatgtcccttaagaaattgtgaggttttcttcagagattgctcatctttatcaagggatcgcaaattatttcctccacaaaaggaacttggctacttgccctctccatgagacttgtgtaaggggcctttgtacaggccatttcttctcaaatctccaccaataaaacctttgcatcacatgtactcagggtctttagaggatttggaaataaggatgctaaaataaattccccatacagcaattccctttattatgttgacttatgtcagacaaaaggaggtttttactgaaaattttgtgggagtcaagggaattcaaagggtctctcctagacgatcctgggttatgtcctccacaggacctgtggtgttggcccctcttcctcatatgtgaggatggacccagtggcctccctagtatctcctttcttttctttctgaactccaatgtttataaagcctgtatccctgtagtgtatgtaggttgtctgacagaagttatacttagtgctctttctttcttttggggaaaaatccctggaactgaagctgagatcattagtacttggaatcaccttacagatacagagcacttatgaggtattctttggtgcctaaagaacttaaggcatcctctgaaaaactggcccaggttagtgtttattatgaatctttttaacctttctatacttgtttctcctacatctcctacatgctctaactagacatgacagaagagatttaactaatgtagtataaattatatgaaattctatttttgtaagtcaaaaataatcaaatatcagaaatttaataatgttcaaactatatactgtgtggggttaccgagacaatgtggacattgttcacatctcatagggctgaaagtcaatgggcaagtcctggaaactcattgtcttactgcggtcttgtcctcagtttcataggttcacccatcatgccctcagctttccttaattagccatgtctgcttacctcttcctcccatttctctctatttttccccaactatgttgtcatcatttccagaaatctctaaagcttgcacagatccttagcactatgagatccattgaaagagataatatttttctctttgagatagggcctggctctgtcacccaggctgtagctcagtggtgcgatcgaggctcactgcaacctctgcctcccacactcaagcaatcctccctcctcaggctccagagtagctgggaatacaggcaggcaaccacgcccagctaatttttgcaattttggtagagaggagattttgccatattgcccaggctggtcttaaactgctgaactcaagcaatcctcctgccttagcctcccaacatgctaggattatagatgtgagccagtgtgcccaggcaaaagagatgactcttaataaaaagatttcctttttcttaaatcactgttcctttatccgtgaattcttcttccaactagaagaaggagaaagaagtttgcctgtatttctcaccaggaggagaaggggtctagtgtgacatcaaaatgaaagagtgctggaacttgagccccttcttgctttccaggatccccacagtgatcagttcccataccctggtttatttgtgtaaaccacacttatttttctcagcagctactgtgtactgggttccattctaggttcaaatcattctatttgattaagatagagagggtcccgactctcaaggaagttacaagagtggaggagacagacactaacccaataagcatttaacaaagaagataatgttagagagtcatagtgcactgaagaaaagacatcagatgtgtgttgaaagagagaaatggattcatctactttagtttgtatgtttagggagctctacctgagaaagtgatattcagctgagacaacaaaataagtagacagtcatgaagatctaacggacgaaagtttcagggagaccgaatcgagggaaagcactggtgtgggaaattatgtggagggagagaaagaaggctagaaaggctgacgtatagaaagcaaggaaatggagaggcagaagatgaggtaggacacagagaggaagtcaggagcctcatcattataggctctgatgtccacggtaaaaaatttgaattttattttatttttatttatttttaaattttatttatttatttattttgagatggagtttcattcttgttgcccaggctggagtgccatggcgcaatctccactcactgcaacctccacctcctgggttcaagtgattttcctgcctcagcttcccaagtaactgggattacaggcacccaccaccatacctggctaatttttttgtatttttagtatacatggggtatcaccatgttgaccaggctggtcttgaactcctgacctcagataatctgtctgccttggcctcccaaagtgctgggattacaggcgtgagccaccacccccggactgaattttatttaaatagatatgagaagctgctgtatggttacaaggagagtcaatttatattcaacttttgtgtgtgtgtgtgatggagtctcactctgttgcccaggctagattgcagtggcacaatctcggctcactgcaacctccgcctcccgggttcaagaaattatccttcctcagcctcctgagtagctgggaccacaggtgcatgccaccacagtcggctactttttgtatttttagtagggatggggtttcaccgtgttagccaagatggtctcgatctcctgaccttgtgatccaccagcctcggcctcccaaagtgctgggattacaggcatgagccactgcgcccagcttatattcaattattacaattaattctagctactttgtggggattggattgttggggttcacaagtggttaggaggactatttaggagcacagcagggaattctccagggaaaacaggcttgtggcttcatggagtgcattagtgataaagacggtgaaaaagataaagtggacagacttggcatgtatttttgcttagcttgttaatgaattactgtaaagggggtggaacaatcaagtttattcctgaggattttgttttgacaaataagtgggtggtagttttgtttattgagataggaaaaactgtgggaggaaatgatttgaagtgggtggttggaaataaaagttttgtttaaattagagatgatttattgacatttatgtagagcaatcagaaggtcagtggcatttaagagactcatggtgaggtgaggctagggcttcagatatttatgttggcggcatcaatacgtgtagtgtgttaaattctagggagtggaagaggatacatagggagatggattgtgtggagaaaaaagaacagggcacaggccagcaaagtgggctgagacagagcccagggatgttggagaaaaaccaagagaacataatgcgtgtaagtcaaggaaaatatatttttttcagggagaagggagaggccaattgtggtgagtaccactaagcagagggggaagtgagaacgtgacagagaagcaagtgctgggtttggtggagttgatatttgtagtcaatggagtatccagggaggaaactggattggactatttgaagagtgagtagaagtgaggacgaggttaaggttgactgttttgagtagaacttcgggaaggactgtgctctgggttcagggagcccgctgaatctaaaggaaaaggctaaagaagctaaagagacggaggaagacctgtgaacaagagatgctcagtcattagcaaggaaatactggagagcccctgtgtgcagtggtgactactcatgcaaaaggtcacacagccaatatttaacacagccagtatttcacagagccaatatttattagtgacatagaatataccagttattactctaggtcatgagagtggtgataaataaaatgaatccgctcgccatcagtatatgccatgtaacagtttgcagtgactgtgtaccaggcctgtgaatttctgtatgcaatttcaataatgatcctgctgtatctgtggtatttaaaaacatatacatctctggaatctaaaattgagaggttataagtaaaacccagtattagaaatttagtgctggaaatcagattgcagtttaaatctgagcatatagaaagtccctttcttctatgtcagcagatgccttttatgtgaggtttaggtatactacattattagacataaaccagtgtttctgccctatgttttcagaatgacaattctttatgaaactcatagaagaacagaagacaattgcaaaatcatgaagatactaattgctttagaattaaggaatacaaaaaataatgtgagctacagttatagggatcataaaagttaaaatgggaatgtatttgagtgtttattatgtgatcagtgctaagaagagtcatcatttaattttacacttaacagtaatcctgtgaggattatgctattattaaatgcatttgatatattacaaaaaggcttacggttggtaaaaattgacccaagtagaagaggtcacctttttattcagattttctgattgtagggtttgagagtttttccatcattagtgagtagtgactatattgtgtctgaattattgacagaatttctgatattcatatataccaggttgtttctttgagtggggacagagatgcaagggctgctaattccaatgtataggagaaactttcaatgattttgcatttatcattttaaaagttcagtatgtctataatggtcatgcgttgacaaacacaaggaagtattaaatcactccttcttctgaggtttgactagcaaattgggcaagagttaccaaataaaacacatgttcctagttaaatctaaatttcagatacacgaccataatttattggaaatccaaatttaattgggtaccctctggttttatttgccaaatctgtcaaccctaagtgggacacatggacatggattacagtgctacccatgcaagccacagtgacagcaactttacacatgtttacattttaactttctctctgtaagaaagtgcttagataatctagggataaaaagatagacattgtttgatccagggtggacacctctctgccatcgtttctaaagggcaaagggagatttctgcaggtcttgctcacagtctggggacctgctcatgttttgaaactgtctgtatcagaatgtcattttcttggtttctccctttctgaggggacttgactacaaaaccaagagttctgcctctggccaaggctggaaatgtgatgcctgctagcattgttgggagtgggagactgagagaaatgagttagttggggcatttaatgggaataaaatagctgtggttgtgattcattactatagataattagtggaccagtggcagagaaattaagaaaaaagatgatgtgaatgataaatgatatgattagtgactgcttggtaaggcaagtaaatcattaaatcttggttctcatcaagttcattttctgaaaagatagcactgtattgggaccagaattctacaaaacattctttttacctaagaccaagattttcaagaaatatttttcaatacaattctcagctgctccataactaatagtagcttgttcaacacagattttttcagatggttcacacctgtagttcttacccagggatagttcaccacccctcccttccctcccatcatccttggggaacagtgacaatgtttggaacaatttttggttgtcacaacaggggtttcttctgatatttaatgagtagaagccaggaacactgctagagaacctacaatgttcagaacagcctccgccatcaacaaagaattatctggtccaaaatgtcaatagtgctgaggctgagagcactggttcacgctgtgctctttttgaaaattctacactcacatctgttatacactcaccacacagttcagttttttatagtttatttttgcttgttccattataaaaaaattagacagttgcataaattcaaccactttcttgttgaatccacttagtcaatgcaagcttaatattttcatatttagtttttgccttatgcaatatttttcaatattttcatgagttgttggtcatcaccatctctattaacttccaacaacttgctcttgtaagtcacaaatagtgatgctgctgaaattatttctcagtaacatgcctcagatttctgtagtgattctacattttatattattcacaatgtaaaatgcttctatgtactcatttcacttttacccaaggattatttttaagttatttttgtcgttttcacacttcaaacataaagacaaaaacatcaaaaatatatattgtgttttacatatgtgtctattttcacacatatatgtatgtatgtttatatgtatggaaactacagaagcacatgtcgccaataagagctctgagacacctttgaccacttacccttatcagatgagatttgccaaatgagttgtggaaacaagttttttaaactgaatttctgagctttgtgaatttagaaatgcaaaggaaagtttgtggacatttacagggatcatggttttattgtcctttaaactcttcgatactttaccattgtcttactataaatccaaaatcctaacgccacccacgaggctttcaatacctggcttcttgtgatttctccaggctaaccttttaccctccttcccctcagcctctctgctttagtgaactttctcctagttttttgaagaagttcatcaattcaagcttttgtacatgggatttcctaaacctgaaatgtgcctcccgttttgtccaaacagacacaggctccactctgccccctggctcacacctgcttaacctgtcaagtcacatctgaactgtcactcttcagagggtccttctctggcaccctaatgtaattgagatcatcctattattctctgctctagaacttcccacttccgagatttctcattcctgtctaacctcttgtgtgtctggtttttggccatcgctttcactgctctttaagctcccccagcggagtggagaggtctgttttccctcgtttggattcctagaggcagcgcagaccaggcacaaggtcagcactaaggaagtgttcacaggatgaacgcggtgggtgctgtttaaggaaccggtaaacgtgtgggatgagagaaggagcagagagtgtctttggggtggaggctcccaggaggaggcggggcgggctgcggtgctgggcggatcctcctccagctcctgcttggaggtctccagaacaagctggaggcagggatggggggtcccaaaagcctggggatgagaaggggttttcccgcatagtcccccaggcccccgttcgcctcaggaaaacggaggatgagctcctgggctgctggtggtgggcgttgcgggtggggcccgttaaggttcccagtgcccgcaccccacccagggagccccggatggtggcgtcactgtcagtgtcttctcaggaggccgcctgtgtgaccggagcattcgtgtccccacagcacgtttcttggagctgcttaagtctgagtgtcatttcttcaatgggacggagcgggtgcggttcctggagagatacttccataaccaggaggagttcgtgcgcttcgacagcgacgtgggggagtaccgggcggtgacggagctggggcggcctgtcgccgagtcctggaacagccagaaggacctcctggagcagaagcggggccaggtggacaattactgcagacacaactacggggttgtggagagcttcacagtgcagcggcgaggtgagcatgtcagggggcggggcctgggtccctgtgagctggaaatctgagagtgtgtgtgtgtgtatgtgtgtatgtgtgtgtgtgagagagagagacagagaggaagagagagacagagagagggagcgcgccatctgtgagcatttagaatcctctcaatcccgagcaagcagttctgaaggcacaggtgtgtgtgtagagtgtggatttgtctgtgtctgtgtgcctgttgtgggaggggaagcagaagggggctgcttcttatccttggagacttctgtggagaggtgaaaagggaggtgggtgcgggcggctggagagagaggagaccttgattgtcctgggtccttagagatgcagggaaggaaagtgtaaggtgtgtgtgggtggggtgaaggtttaggggaggagagctgaggggtaaggaaggtttgggataatgtgaggaggccggttccagactgtccctggcacacacccttcatgtaatctctgaaataaaagtgcgtgctgtttgtaaaagcattagattaagttctaggggaattgagtagacctctaaggcacctctgaaacttctttaggtataaatttcttgctagttttttgtttttttagtgtgtatatttttacatagtagaaatgacagtgaaactaacttttgaattaaagttttaacacagttactgtattattataatgctaatagttttctagtagttacatattattcttttatatataatagttgtgacacaacttagggatcactttcccctttgttgacctttattatgacattcaccaaaagttgaaaatgtatgtttctggttaatttttaatttatattttttcatttgaaaaaatgtggagaggtctgtttttcattcaaaagagttcttttgaattattttgaccgatttattggtcaattttaataactgctctaagaattccctattttatttggtaggtaatggacaatgatctactgcctaatatctctagggcttagtatttttctcagtgactttgtgggttctttgtactgtaagattattaacactttattgatatttgattcagcatttgctccagtttgtggtttttatgttgattttgaaaattcttttccatgttaagaatttgaacatttttatttaataaaatgtattgcaaaaattttattaatgatttacaatccatcttaaatctgccattttgtggtatttttgtctctaggtttctccttacttctaaaaacattgtatttattgagagtatgctaatgtcggggattttcctgggcataagcatcacaagtaatgagttccagaccctgccttaatccaaatgtgattctggaaagaaaaatcattttacaattggcctgataataattctgcttgtgttgcacgggggatacattgagcagctgaatgaaaatataagaactttcaaaactaaaatgacgctcctaaatccttctgtctgctttaggactcatgcttttctaggaacgtaaaaatttggagaatcatttctgtctgtcccaccttcccaggggcagaaccatttctgtggtgttctaaggtgtgagtgcatggcagcagtattcctaaaaattcatactctgttttctcatgtacccaactctgtccctttgtctatgtacattgctttaaatcatatttttctgtcacggtgtacaaggatgataaataggtgccaagtggagcacccaagtgtgatgagcccccttatggtggaatggagcgagaagctttctgacctcataaattgaaggctatcttcactcattatgtattttacgtaaattaatcctcatataaccccaagaggtaaattagtataattatccttcattgggggtgacaaagttgagacacagaagaatcaactcttccaggatcaaccagtaaaaggcagaccttggatttgaaccatgcaacctggctcagatatcagttttaattactacactctgtactttcaaaaatttgtaaacactttgacaatgcatgccaatttcaagctatgaagaagcaaacgcaatttttcacatctctcaaatctagtgggtcctcactatcaagattaaattccaggctgatgacactgtaaggccacatggccagctgtgctggaggcctgttcaaggtcagagcctgggtttgcagagaagcagacaaacaccaaaaccaggagaattactctgtcttcctgactcattccctctacctttttttctactagtccatcctcaggtgactgtgtatcctgcaaagacccagcccctgcagcaccacaacctcctggtctgttctgtgagtggtttctatccaggcagcattgaagtcaggtggttccggaatggccaggaagagaagactggggtggtgtccacaggcctgatccataatggagactggaccttccagaccctggtgatgctagaaacagttcctcggagtggagaggtttacacttgccaagtggagcacccaagcgtaacaagccctctcacagtggaatggagtgagcagctttctgatttcatacatttctcacccaccaagaaggggactgtgctaatccctgagtgtcaggtttctcctctcccacgtcctattttcatttgttccatgttctcatctccatcagcacaggtcactggggatagccctgtaacagtgtgtagaaacacctgtaccccctgtggaatcagtcatgcttttttttttttgaacctccccatgatgtcacaggtcagggtcacccgctctccccaagctccaggccctgcttttgggtctgagactgagtttctggtgctgctgctctgagttacttgttttgatccgggaagaggggaagcataggggcctacctgacatgaggggagtctaatctcagctctaccttttattagctctgtcactctagacaaactacttagcttcattgagtctcaggctttctgttgatcagatgttgaacgcttgccttacatcaatgctgtaatatttgaatgagtttgatgtcggaaccttgtaactgttcagtgtgatttgaaatcctttttttctcccgaaatggctagttattttagttcttgtggggcagccttcttccccattttcaaagctctgaatcttagagtctcaattaaagaggttcaatttggaataaacgtcactaaacctggcttcctctctcaggagcacggtctgaatctgcacagagcaagatgctgagtggagtcgggggctttgtgctgggcctgctcttccttggggccgggctgttcatctacttcaggaatcagaaaggtaaggagtctttggtagctggctctctccgtaggcttttctggaggaggaactatagctttgctgaggttagttctcagtatatgagtggccctgaataaagcctttctttccccaaacggctctaatgtcctgctaatccagaaatcatcagtgcatggttactatgtgaaagcataatagcttgtggcctgcagagacaagaggaaggttaacaagtaggggtcctttggtttgagatcttggagcaaattaaggaagagccactaaagttaatggaattacactggatcctgtgacagacacttcatgcttcatgggtcacatggtctgtttctgctcctctctgccctggttggtgtgggttttggtgttagagaactctccggtgggagatctgggactgggatattgtgttggaggacagatttgcttcaatatcttttaagtgtatatcttttcctctttttcccaggacactctggacttcagccaacaggtaataccttttaatcctcttttagacacagattcagtttctctagtgagaggtgaagccagctggacttctgggttgggtggggacttggagaacttttctgtcttacaagaggtttctaaatgcaccaatgagtgctctgtaaaaacacaccaatgagtgctctgtaaaaattgacactctgtggcttgctagatgtttgtaagatggaccaatcagcactctgtaaaatggaccaatccacactctgtaaaatggaccaatcagcactctttaaaatggaccaatcagcaggatacgggtggggacaaataagggaataaaagctggtcaccctagccagcaccggcaacctgcttaggtccttttctatgctgtggaaggtctgttctttcactcttcacaataaatcttgtgctcactctttgggtccgtgccacctttaagagctataacactcactgcaagggtctgtggcttcactcttgaagtcagccagaccctgaacccaccggaaggaacaaactcaggacacactagaatggtggtagaggtgataaggcatgagacagaaataataggaaagactttggatccaaatttctgatcaggcaatttacaccaaaactcctcctctccacttagaaaaggcctgtgctctgcaggactattggctctgggagactcaggaacttgtttttcttcttcctgcagtgttctcatctgagtccttcaaagagggggaaagaaacgttagtagagccaggttgaaaacaacactctcctctgtcttttgcaggattcctgagctgaagtgcagatgacaatttaaggaagaatcttctgccccagctttgcaggatgaaaagctttcccgcctggctgttattcttccacgagagagggctttctcaggacctgg

SEQ ID NO: 17 - DNA, 12906 bp, Homo sapiens
  source 1..12906: mol_type = other DNA
ctctggcccctggtcctgtcctcttctccagcatggtgtgtctgaagctccctggaggctccagcttggcagcgttgacagtgacactgatggtgctgagctcccgactggctttcgctggggacacccgacgtaagagcacattgagggtgctgagctactatggggtgggggaatatagggagttgtgttaacattgtgcccaggccatgtcccttaagaaattgtgaggttttcttcagagattgctcatctttatcaagggatcgcaaattatttcctccacaaaaggaacttggctacttgccctctccatgagacttgtgtaaggggcctttgtacaggccatttcttctcaaatctccaccaataaaacctttgcatcacatgtactcagggtctttagaggatttggaaataaggatgctaaaataaattccccatacagcaattccctttattatgttgacttatgtcagacaaaaggaggtttttactgaaaattttgtgggagtcaagggaattcaaagggtctctcctagacgatcctgggttatgtcctccacaggacttgtggtgttggcccctcttcctcatatgtgaggatggacccagtggcctccctagtatctcctttcttttctttctgaactccaatgtttataaagcctgtatccctgtagtgtatgtaggttgtctgacagaagttatacttagtgctctttctttcttttggggaaaaatccctggaactgaagctgagatcattagtacttggaatcaccttacagatacagagcacttatgaggtattctttggtgcctaaagaacttaaggcatcctctgaaaaactggcccaggttagtgtttattatgaatctttttaacctttctatacttgtttctcctacatctcctacatgctctaactagacatgacagaagagatttaactaatgtagtataaattatatgaaattctatttttgtaagtcaaaaataatcaaatatcagaaatttaataatgttcaaactatatactgtgtggggttaccgagacaatgtggacattgttcacatctcatagggctgaaagtcaatgggcaagtcctggaaactcattgtcttactgcggtcttgtcctcagtttcataggttcacccatcatgccctcagctttccttaattagccatgtctgcttacctcttcctcccatttctctctatttttccccaactatgttgtcatcatttccagaaatctctaaagcttgcacagatccttagcactatgagatccattgaaagagataatatttttctctttgagatagggcctggctctgtcacccaggctgtagctcagtggtgcgatcgaggctcactgcaacctctgcctcccacactcaagcaatcctccctcctcaggctccagagtagctgggaatacaggcaggcaaccacgcccagctaatttttgcaattttggtagagaggagattttgccatattgcccaggctggtcttaaactgctgaactcaagcaatcctcctgccttagcctcccaacatgctaggattatagatgtgagccagtgtgcccaggcaaaagagatgactcttaataaaaagatttcctttttcttaaatcactgttcctttatccgtgaattcttcttccaactagaagaaggagaaagaagtttgcctgtatttctcaccaggaggagaaggggtctagtgtgacatcaaaatgaaagagtgctggaacttgagccccttcttgctttccaggatccccacagtgatcagttcccataccctggtttatttgtgtaaaccacacttatttttctcagcagctactgtgtactgggttccattctaggttcaaatcattctatttgattaagatagagagggtcccgactctcaaggaagttacaagagtggaggagacagacactaacccaataagcatttaacaaagaagataatgttagagagtcatagtgcactgaagaaaagacatcagatgtgtgttgaaagagagaaatggattcatctactttagtttgtatgtttagggagctctacctgagaaagtgatattcagctgagacaacaaaataagtagacagtcatgaagatctaacggacgaaagtttcagggagaccgaatcgagggaaagcactggtgtgggaaattatgtggagggagagaaagaaggctagaaaggctgacgtatagaaagcaaggaaatggagaggcagaagatgaggtaggacacagagaggaagtcaggagcctcatcattataggctctgatgtccacggtaaaaaatttgaattttattttatttttatttatttttaaattttatttatttatttattttgagatggagtttcattcttgttgcccaggctggagtgccatggcgcaatctccactcactgcaacctccacctcctgggttcaagtgattttcctgcctcagcttcccaagtaactgggattacaggcacccaccaccatacctggctaatttttttgtatttttagtatacatggggtatcaccatgttgaccaggctggtcttgaactcctgacctcagataatctgtctgccttggcctcccaaagtgctgggattacaggcgtgagccaccacccccggactgaattttatttaaatagatatgagaagctgctgtatggttacaaggagagtcaatttatattcaacttttgtgtgtgtgtgtgatggagtctcactctgttgcccaggctagattgcagtggcacaatctcggctcactgcaacctccgcctcccgggttcaagaaattatccttcctcagcctcctgagtagctgggaccacaggtgcatgccaccacagtcggctactttttgtatttttagtagggatggggtttcaccgtgttagccaagatggtctcgatctcctgaccttgtgatccaccagcctcggcctcccaaagtgctgggattacaggcatgagccactgcgcccagcttatattcaattattacaattaattctagctactttgtggggattggattgttggggttcacaagtggttaggaggactatttaggagcacagcagggaattctccagggaaaacaggcttgtggcttcatggagtgcattagtgataaagacggtgaaaaagataaagtggacagacttggcatgtatttttgcttagcttgttaatgaattactgtaaagggggtggaacaatcaagtttattcctgaggattttgttttgacaaataagtgggtggtagttttgtttattgagataggaaaaactgtgggaggaaatgatttgaagtgggtggttggaaataaaagttttgtttaaattagagatgatttattgacatttatgtagagcaatcagaaggtcagtggcatttaagagactcatggtgaggtgaggctagggcttcagatatttatgttggcggcatcaatacgtgtagtgtgttaaattctagggagtggaagaggatacatagggagatggattgtgtggagaaaaaagaacagggcacaggccagcaaagtgggctgagacagagcccagggatgttggagaaaaaccaagagaacataatgcgtgtaagtcaaggaaaatatatttttttcagggagaagggagaggccaattgtggtgagtaccactaagcagagggggaagtgagaacgtgacagagaagcaagtgctgggtttggtggagttgatatttgtagtcaatggagtatccagggaggaaactggattggactatttgaagagtgagtagaagtgaggacgaggttaaggttgactgttttgagtagaacttcgggaaggactgtgctctgggttcagggagcccgctgaatctaaaggaaaaggctaaagaagctaaagagacggaggaagacctgtgaacaagagatgctcagtcattagcaaggaaatactggagagcccctgtgtgcagtggtgactactcatgcaaaaggtcacacagccaatatttaacacagccagtatttcacagagccaatatttattagtgacatagaatataccagttattactctaggtcatgagagtggtgataaataaaatgaatccgctcgccatcagtatatgccatgtaacagtttgcagtgactgtgtaccaggcctgtgaatttctgtatgcaatttcaataatgatcctgctgtatctgtggtatttaaaaacatatacatctctggaatctaaaattgagaggttataagtaaaacccagtattagaaatttagtgctggaaatcagattgcagtttaaatctgagcatatagaaagtccctttcttctatgtcagcagatgccttttatgtgaggtttaggtatactacattattagacataaaccagtgtttctgccctatgttttcagaatgacaattctttatgaaactcatagaagaacagaagacaattgcaaaatcatgaagatactaattgctttagaattaaggaatacaaaaaataatgtgagctacagttatagggatcataaaagttaaaatgggaatgtatttgagtgtttattatgtgatcagtgctaagaagagtcatcatttaattttacacttaacagtaatcctgtgaggattatgctattattaaatgcatttgatatattacaaaaaggcttacggttggtaaaaattgacccaagtagaagaggtcacctttttattcagattttctgattgtagggtttgagagtttttccatcattagtgagtagtgactatattgtgtctgaattattgacagaatttctgatattcatatataccaggttgtttctttgagtggggacagagatgcaagggctgctaattccaatgtataggagaaactttcaatgattttgcatttatcattttaaaagttcagtatgtctataatggtcatgcgttgacaaacacaaggaagtattaaatcactccttcttctgaggtttgactagcaaattgggcaagagttaccaaataaaacacatgttcctagttaaatctaaatttcagatacacgaccataatttattggaaatccaaatttaattgggtaccctctggttttatttgccaaatctgtcaaccctaagtgggacacatggacatggattacagtgctacccatgcaagccacagtgacagcaactttacacatgtttacattttaactttctctctgtaagaaagtgcttagataatctagggataaaaagatagacattgtttgatccagggtggacacctctctgccatcgtttctaaagggcaaagggagatttctgcaggtcttgctcacagtctggggacctgctcatgttttgaaactgtctgtatcagaatgtcattttcttggtttctccctttctgaggggacttgactacaaaaccaagagttctgcctctggccaaggctggaaatgtgatgcctgctagcattgttgggagtgggagactgagagaaatgagttagttggggcatttaatgggaataaaatagctgtggttgtgattcattactatagataattagtggaccagtggcagagaaattaagaaaaaagatgatgtgaatgataaatgatatgattagtgactgcttggtaaggcaagtaaatcattaaatcttggttctcatcaagttcattttctgaaaagatagcactgtattgggaccagaattctacaaaacattctttttacctaggaccaagattttcaagaaatatttttcaatacaattctcagctgctccataactaatagtagcttgttcaacacagattttttcagatggttcacacctgtagttcttacccagggatagttcaccacccctcccttccctcccatcatccttggggaacagtgacaatgtttggaacaatttttggttgtcacaacaggggtttcttctgatatttaatgagtagaagccaggaacactgctagagaacctacaatgttcagaacagcctccgccatcaacaaagaattatctggtccaaaatgtcaatagtgctgaggctgagagcactggttcacgctgtgctctttttgaaaattctacactcacatctgttatacactcaccacacagttcagttttttatagtttatttttgcttgttccattataaaaaaattagacagttgcataaattcaaccactttcttgttgaatccacttagtcaatgcaagcttaatattttcatatttagtttttgccttatgcaatatttttcaacattttcatgagttgttggtcatcaccatctctattaacttccaacaacttgctcttgtaagtcacaaatagtgatgctgctgaaattatttctcagtaacatgcctcagatttctgtagtgattctacattttatattattcacaatgtaaaatgcttctatgtactcatttcacttttacccaaggattatttttaagttatttttgtcgttttcacacttcaaacataaagacaaaaacatcaaaaatatatattgtgttttacatatgtgtctattttcacacatatatgtatgtatgtttatatgtatggaaactacagaagcacatgtcgccaataagagctctgagacacctttgaccacttacccttatcagatgagatttgccaaatgagttgtggaaacaagttttttaaactgaatttctgagctttgtgaatttagaaatgcaaaggaaagtttgtggacatttacagggatcatggttttattgtcctttaaactcttcgatactttaccattgtcttactataaatccaaaatcctaacgccacccacgaggctttcaatacctggcttcttgtgatttctccaggctaaccttttaccctccttcccctcagcctctctgctttagtgaactttctcctagttttttgaagaagttcatcaattcaagcttttgtacatgggatttcctaaacctgaaatgtgcctcccgttttgtccaaacagacacaggctccactctgccccctggctcacacctgcttaacctgtcaagtcacatctgaactgtcactcttcagagggtccttctctggcaccctaatgtaattgagatcatcctattattctctgctctagaacttcccacttccgagatttctcattcctgtctaacctcttgtgtgtctggtttttggccatcgctttcactgctctttaagctcccccagcggagtggagaggtctgttttcccttgtttggattcctagaggcagcgcaggcctggcacaaggtcatcactaagaaagtgttcacaggatgaacgcggtgggtgctgtttaaggaaccggtaaacatgtgggatgagagaaggagcagagtgtctttggggtggaggctcctaggaggaggcggggcgggctgcggtgcggggcggatcctcctccagctcctgcttggaggtctccagaacaagctggaggcagggagggggtcccaaaagcctggggatgagaaggggttttcccgcatagtcccctaggcccccgttcgcctgaggaaaacggaggatgagctcctgggctgcaggtggtgggcgttgcgggtggggccggttaaggttcccagtgcccgcaccccacccagggagccccggatggcggcgtcgctgtcagtgtcttctcaggaggccgcccgtgtgaccggatccttcgtgtacccgcagcacgtttcttggagctgcgtaagtctgagtgtcatttcttcaatgggacggagcgggtgcggtacctggacagatacttccataaccaggaggagttcctgcgcttcgacagcgacgtgggggagtaccgggcggtgacggagctggggcggcctgtcgccgagtcctggaacagccagaaggacctcctggagcagaagcggggccgggtggacaattactgcagacacaactacggggttggtgagagcttcacagtgcagcggcgaggtgagcatgtcggggggcggggcctgggtccctgtgagctggaaatctgagagagagtgtgtgtgtgtgtgtgtgtgtgtgtgagagagagagagagagagaggaagagacagagagagggagcgcgccatctgtgagcatttagaatcctctcaatcccgagcaagcagttatgaaggcacaggtgtgtgtgtagagtgtggattggtctgtgtctgtgtgcctgttgtgggaggggaagcaggagggggctgcttcttatccttggagacttctgtggagaggtgacaagggaggtgggtgcgggcggctggagagagaggagaccttgattgtcctgggtccttagagatgcagggaaggaaagtgtaaggtgtgtgtgggtggggtgaaggtttaggggaggagagctgaggggtaaggaaggtttgggataatgtgaggaggccggttccagactgtccctggcacacacccttcatgtaatctctgaaataaaagtgcgtgctgtttgtaaaagcattagattaagttctaggggaattgagtagacctctaaggcacctctgaaacttctttaggtataaatttcttgctagttttttgtttttctagtgtgtatatttttacatagtagaaatgacagtgaaactaacttttgaattaaagttttaacacagttactgtattattataatgctaatagttttctagtagttacatattattcttttatatataatagttgtgacacaacttagggatcactttcccctttgttgacctttattatgacattcaccaaaagttgaaaatgtatgtttctggttaatttttaatttatattttttcatttgaaaaaatgtggagaggtctgtttttcattcaaaagagttcttttgaattattttgaccgatttattggtcaattttaataactgctctaagaattccctattttatttggtaggtaatggacaatgatctactgcctaatatctctagggcttagtatttttctcagtgactttgtgggttctttgtactgtaagattattaacactttattgatatttgattcagcatttgctccagtttgtggtttgtatgttgattttgaaaattcttttccatgttaagaatttgaacatttttatttaataaaatgtattgcaaaaattttattaatgatttacaatccatcttaaatctgccattttgtggtatttttgtctctaggtttctccttacttctaaaaacattgtatttattgagagtatgctaatgtcggggattttcctgggcataagcaccaccagtaatgagttccagaccctgccttaatccaaatgtgattctggaaagaaaaatcattttacaattggcctgataataattctgcttgtgttgcacgggggatacattgagcagctgaatgaaaatataagaactttcaaaactaaaatgacgctcctaaatccttctgtctgctttaggactcatgcttttctaggaacgtaaaaatttggagaatcatttctgtctgtcccaccttcccaggggcagaaccatttctgtggtgttctaaggtgtgagtgcatggcagtagtattcctaaaaattcatactcggttttctcatgtacccaactctgtccctttgtctatgtacattgctttaaatcatatttttctgtcacggtgtacaaggatgataaataggtgccaagtggagcacccaagtgtgatgagcccccttacggtggaatggagtgagaagctttctgatctcataaattgaaggctatcttcagtcattatgtattttacataaattaatcctcatataaccccaagaggtaaattagtataattatccttcattgggggtgacaaagttgagacacagaagaatcaactcttccaggatcaaccagtaaaaggcagaccttggatttgaaccatgcaacctggctcaggtatcagttttaattactacactctgtactttcaaaaatttgtaaacactttgacaatgcatgccaatttcaagttatgaagaagcaaacgcaatttttcacatctctcaaatctaatgggtcctcactatcaagattaaattccaggctgatgacactgtaaggccacatggccagctgtgctggaggcctggtcaaggtcagagcctgggtttgcagagaagcagacaaacagccaaaccagcagaattactctgtcttcctgactcattccctctacctttttttctcctagtccatcctcaggtgactgtgtatcctgcaaagacccagcccctgcagcaccacaacctcctggtctgctctgtgagtggtttctatccaggcagcattgaagtcaggtggttccggaacggccaggaagagaaggctggggtggtgtccacgggcctgatccagaatggagactggaccttccagaccctggtgatgctagaaacagttcctcggagtggagaggtttacacttgccaagtggagcacccaagcgtaacgagcgctctcacagtggaatggagtgagcagctttctgatttcatacatttctcacccaccaagaaggggactgtgctaatccctgagtgtcaggtttctcctctcccacgtcctattttcatttgctccatgttctcatctccatcagcacaggtcactggggatagccctgtaacagtgtgtagaaacacctgtactccctgtggaatcagtcatgcttttttttttttgaacctccccatgatgtcacaggtcagggtcacccgatctccccaagctccaggccctgcttctgggtctgagactgagtttctggtgctgctgctctgagttacttgttttgatctgggaagaggggaagcataggggcctacctgacatgaggggagtccaatctcagctctgccttttattagctctgtcactctagacaaactacttagcctcactgagtctcaggctttctgtggatcagatgttgaacgcttgccttacatcaaggctgtaatatttgaatgagtttgatgtcggaaccttgtaactgttcagtgtgatttgaaatcctttttttctcccaaaatggctagttattttagttcttgtggggcagccttcttccccattttcaaagctctgaatcttagagtctcaattaaagaggttcaatttggaataagcatcactaaacctggcttcctctctcaggagcacggtctgaatctgcacagagcaagatgctgagtggagtcgggggctttgtgctgggcctgctcttccttggggccgggctgttcatctacttcaggaatcagaaaggtgaggagcctttggtagctgggcctttccataggcttttctgcaggaggaaatagagctttgctgaggttagttctcagtatatgagtggctctgaataaagcctttctttccccaaacggctctaatgtcctgctaatccagaaatcatcagtgcatggttactatgtgaaagcataatagcttgtggcctgcagagacaagaggaaggttaacaagtaggggtcctttggtttgagatcttggagcaaattaaggaagagccactaaagttaatggaattacactggatcctgtgacagacacttcatgcttcatgggtcacatggtctgtttctgctcctctctgccctggttggtgtgggttttggtgttagaactctccggtgggagatctgggactgtgatattgtgttggaggacagatttgcttcaatatcttttaagtgtatatcttttcctctttttcccaggacactctggacttcagccaacaggtaataccttttaatcctcttttagacacagattcagtttctccagtgagaggtgaagccagctggacttctgggttgggtggggacttggagaacttttctgtcttacaagaggtttctaaatgcaccaatgagtgctctgtaaaaacacaccaatgagtgctctgtaaaaattgacactctgtggctcgctagatgtttgtaagatggaccaatcagcactctgtaaaatggaccaatccacactctgtaaaatggaccaatcagcactctttaaaatggaccaatcagcaggatacgggcggggacaaataagggaataaaagctggtcaccctagccagcacctgcaacctgcttaggtccttttctatgctgtggaagttttgttcttttgatcttcacaataaatcttgtgctcactctttgggtccgtgccacctttaagagctataacactcactgcaagggtctgtggcttcactcttgaagtcagccagaccctgaacctaccggaaggaacaaactcaggacacactagaatgatggtagaggtgataaggcatgagacagaaataataggaaagactttggatccaaatttctgatcaggcaatttacaccaaaactcctcctctccacttagaaaaggcctgtgctctgcaggactattggctctgggagactcaggaacttgtttttcttcttcctgcagtgttctcatctgagtccttcaaagagggggaaagaaacgttagtagagccaggttgaaaacaacactctcctctgtcttttgcaggattcctgagctgaagtgcagatgacaatttaaggaagaatcttctgccccagctttgcaggatgaaaagctttcccgcctggctgttattcttccacgagagagggctttctcaggacctagttgctactggttcagcaactgcagaaaatgtcctcccttgtggcttcctcag

SEQ ID NO: 18 - DNA, 270 bp, Homo sapiens
  source 1..270: mol_type = other DNA
catgtttcttgcagcaggataagtatgagtgtcatttcttcaacgggacggagcgggtgcggttcctgcacagaggcatctataaccaagaggagaacgtgcgcttcgacagcgacgtgggggagtaccgggcggtgacggagctggggcggcctgacgctgagtactggaacagccagaaggacttcctggaagacaggcgcgccgcggtggacacctactgcagacacaactacggggctgtggagagcttcacagtgcagcggcgag

SEQ ID NO: 19 - DNA, 12599 bp, Homo sapiens
  source 1..12599: mol_type = other DNA
atggtgtgtctgaagctccctggaggttcctacatggcaaagctgacagtgacactgatggtgctgagctccccactggctttggctggggacacccgacgtaagtgcacattgtgggtgctgagctactatggggtggggaaaatagggagttttgttaacattgtgcccaggccaggtgccttaagaaattgtgacattttcttcagagattgcccatctttatcatgggatcccaaattatttcctccacaaaaggagcttgactacttgccctctccatgagactgtgtaaggggcctccatacaggtcatttcttctcaaatctccaccaatgaaaccttttcatcacatgtcctcagggtctttagaggatttagaaataaggatgctaaaataaattccccatacagcacttccctttattatgttgacctatgtcagacaaaacgagtttttttctgaaaattttgtgggagtcaagggaattcaaagggtctctcctagacaatcctgtgttatgcccttgacagaacctgtgatattggctcctcttcctcatatgtgagaatggacccagtggcctccccattacctcctttcttttctttctgaactccaatgtttaccctgtaatgtatgcaaggtctctgacagaagttatgcttagtgctttttcttatggggaaaaatccttggagctgaagctgagatctttagtatgtggagtcaccctacagttaaagggcatctatgaggtattctttggtgcctaaaggacttaaggcatcctctaaaaacctggcccaggttagagtttattacaaatcttttttaacctttctatactatttgctcctatatctcctacatgctctaactagacatgacaggaagagattcaactaacataggacaaattatatgaaattctatttttgtaagtcaaaaatagtcaaatatcagaaatttgataaggttaaaactatatactctgtgtggggttacagagagaatgtggacattgttcacatctcatagggctgaaagttaatgatcaagtcctgggaactcattgtcttactggggtcttgtcctaaatttcataggttcacccgtcatgccctcagctttccttaattagccatgtctgcttacctcttcctccagtttctctctatttttccccagctatgttgtcatcatatccagaaatccctaaagcttgcacagatccttagcactatgagatccattgaaagagataatttttttctttttgagatagggcctggctctgtcacccaggctgtaatgcagtggtccgatctaggctcactgcaacctctgcctccaagactcaagctatccttcctcctcagcctccagagcagcggagactacaagcccaactaattttgttggagatgagatttctgccatgttgcccagggtggtcttaaactcctggactcaaacaatcctcctgccttggcctcccagcatgctaggattgtaggtgtaagccaccgcacccaggcacggtgaattcattgcccaggcaatgaatctcaatgaaaaatttcttttttcttaaatcactgtttctttatctgtgaattcttcttccaattagaaggaggagaaagaagaaatttgcctgtatttctcaccgggaggagaaggattctagtgtgacatcaaattgaaagagtgctggagctcgagccccttcttgctttccaggatccttgcagtgatcagttcccagaaccatggtttattaatataaaccacagttatttttatcagcagctactctgtactgggctcccttctaggttcaaatcattctatttgagtaagatagagagggtcccgactctcaaggttacacaagagtggaggagacagacactaacccaataagcatttaacaaagaagaaaatttcagagagtcatagtgcactgaagaaaagacatcaggtttgtggaaaagagagaaatggattcacctactttagttcatatgtctagagagccctacttgagaaagtgacatccagctgagaaaacaaaataagtagacagtcgtgaagatctaagggacgaaagttccagggagaccgaattgggggggaagccctggtgtgggaaattatgtgcagggacagaaagaaggctagagggactgaagtgagcaagcaaggaaatgcagtggcagaagatgaggtaggatgcagagaggaagtcgggagcctcatatcaggctctgatgtccatggtaagaaacttgaattttattttattttattaatttatttatttatcttatttatttttgagagggagtttcattcttgttgcccaggctggagtgcaatggcatggtctcagctcactgcaacctctgcctcccgggttcaagtgattctcttgcctcagtctcccaagtagctgggattacaggtacctgccaccatgctcagctattttttctgtgtttttactagagacagactttcaccatgttggccaggctggtcttcaactcctgacctcaagtaatctgcccgcctgagactcccaaagtgctgggattacaggcgtgagccactgcgcatggcctgaattttatttaaatagatatgggaagctactggatggttacaaggaaagtcaatttatattctttttttttaattttttaattttttttttttttgagatatagtcttgctctgtcaccaggctggagtgcagtggcactatctcggctcactgcagcctctgccttctgggttcaagagattctcctgcctcagcctcccaagtagctgggattacaggagtgcaccaccacgcccagctaattttggtatttttagtaaagacagggtttcaccatgttggccaggatggtcttgatctcttgacctcgtgatccacctgcctcagcctcccagtgtgctgggattacaggcgtgagccaccgtgcccggcctatattgaatttttaaaactaattctagctactctgtggggattggattgttggggttgacaagtggttaggaagactatttaggagcacagcagggaatcctcagggaaaataggcttgagttcactagtgataaagacagtgaaaaagataaagtggattttgcttaccttgttaatggattactgtaaagggggtagaaatatcaagcttattcctaaggattttgttttgacaaattagtgaaaggtggtggtgtttattgagatagggaaaactgtgggaggaaatgatttgaagtgggtggttggaaataaaagttttgtttaagtttgagatgatttattgacatttgtgtggagcaatcagaaggtcaatggcatttaaaagactcatggtgggccaggcgcagtgactcatgcctgtaatcccagcactttgggaggcctagacaggcggatcaccaggtcaggagatcaagaccttcctggctaacacggtgaaaccccgtctctactaaaaatacaaaaaattagctgggcatggtggtgggtgcctgtagtcccagctacttgggaggctgaagcacgagaatggcgtgaaccagggaggtggagcttgcagtgagctgagatcacaccacttcactccaggctgggtgacagagcaagactccgtctcaaaaaaaaaaaaaaaaaaaaagagactcatggtgaggccagggctggaggtatttatgttggtggcatcaataggtgtactgtgttaaattccagggagtggaagaggttacatagggagattgtctggagaaaaaagaaaagggcacaggccagcaaaggggactaagaaagagcccagggatgttgggaaaaaaccaagattacatcatatgtgtaagtcaagaaaaatagatttttttttttcaaggagaagggagaggccaattgtggtgagtaccactaagaggagggtgaagtgagaatgtgacagagaagcaacggctgggattggtggagctgatatttgcagtcagtagagtatccagggaggaaactggattggaccatttgaagagcgagtagaagagaggacaaggttaaggttgactgttttgagtagcgagcttcagggaaggactgtgctctgggttcagggagccagctggatcaaaaggaaaaggctaaagaggctgaagagaagcaggaggacctgtgaaccagagatgctcggtcattattatcaaggaaatattagagggcccctgtgtgcagtggtgactgctcatgaagaggtcacacagccagtatttcacacagcccatatttattagtgacttagaatataccagttactactctaggtcatgagaatggagtgatgaataaaatgaatctggtctccatcagtatatgccatgtaacattttgcagtgactgtgtaccaggcctatgaatttcagtatgcaatttcaataatgatcctgctgtatctgtggtatataaaaacatgtacatctctggaatctaaaattgaaaggttataagtaaaacccagtatcacaaatttggtgctagaaatcagattgcagtttaaatctgagcatgtagaaagtccatttcttctatgtcagcaggtgccttttgtgtgaggtttaggtatactgcattattagacataaaccagtgtttctgccctctgttttcagaatgacaattctttatgaaactaatagaagaacagaagacaattgcaaaatcatgatgaagatactaattgctttagaatcatgtaatagaaaaaataatgtgagctccagttatagggatcaaaaaattaaaatgggaatacatttgagtgtttattatgtgatcactgctaagaagactctttattaaatttcacacttacaaaaatcctgtgaggattattataaaatgcatttgatagattacaaataggcttatggttggcaaaaagtgacccatttaaaagaggtcatatttcaattcatattttctgattctagaatatgagagtttttccatcattagtgagtagtgacttgattgtgtctgaattattgacaaaatttctaatattcatatgtaccaggttgtttcttagagtgggggcagagatgcaagggcttctagttccaatgtataaaagaaactttcattcattttgcatttatcattttaaaagttctataagtctatcataggcatgtgttgaagaacataaagaagtattaaatcattccttcttctgaggtttgactagcaagtctggctagggttgtcagataacaaacaggttcctagttaaatctgaatttcagacacacgaccacaatttattggaaatccagatttacctgggcatcctctggttttatttgtcaaatctgtcaaccctaagtgggacacatgagcatggattacagtgctagccatgcaagccacagtgacagtgactttacaaatgtttattttttaacattctgtctgtaaagaaagtgcttacataatttaggaatagaaagatagacattgtttgatccagggtgcacacctctctgccatcatttctaaagggcaaacggagctttgtgaaggtctctacacaaagtctggggacctgctcatgttttgtaaactgtctgtatgagaacgtcattttcttggtttctgactttctgaggggacttgactacaaaaccaagagttctatctctggccaaggctggtaatttgatgcctgctagtattgttgggagtgggagactgaaagacgtgagttagttggggcattaaatgggaataaaatagctgtggttgtgattcactactacagataattagtggacaagtggcagagaaattaagaaagaagatgatatgaaagacaaatgatatgatttggtgactggttggtaatgcaaggaaatcagtaaatcttagttctcaacaagttcattttctggaaagatagcactgtattgggaccagaattctataaagcattcgttttatgtaggaccaagatattcacagatatttttcaatacaattctcagctgctccataaccactagtggcttgttcaacacaggttttttcagatggttcacacctgtggttcttacccagggatcgttcaccaccgctcctttccctcccatcacccttggggaacttgtggcaatgtttgagcaatttttggttgtcaccacaggggtttcttctgatatttaatgagtagaagccagggacactgctagagaacctacaatgttcagaacagcctcctttaccaacaaagaattatctggaccaaaatgtcaatagtgctgaggctgagagcactgtttcacactgtgctctttctgaaaattctagactcacatcttttatacactcagcacccaattcagttctttatggtttatttttgcttgcttcattatgcaaaagtagacagttgcataaattcaaccactttcttgttgaatccatttagtcaatgcaagtttaatagcttcatagttagtttttgccttatgcaatatttttcaacattttcatgagttgttggtcagcactacctctattaactttcaacaacttgcccttataagtcacaaatagtgatgctctgcaattatttttcactaacatgcctgagatttctgtagtgattctacatttggtatgattcataatgtaaaatgcttttatttattcatttcacttttaaccagaggattatttttcagttatttttgtcatttttacacttcaatcaaacataaagacaaaaacatctaaaatatatagtattttacatatgtgtatatttacacacatatatgtatgtatgtttacatgtattgaaactacagaagctcatgtcaccaataagagctctgagacacttttgaccccttactcttatcaggtgaaatttgccaaataagttttatgaacaaatttcttttaactgaatatctgagctttgtggatttagaaatgcaaaggaaggtttgtggacattcatgggatcatggttttattctccttaaatctctttgatactttcccactgtctttagcataaatctcagatcctaacaccacccacaacgcttttcaatacctggcttcttgtgatgtctccagtctaatcttttaccctccttcccctcagcctctctgctttagtgaactttgttctagtttcttgaagaagttcatcaattcaagcttttgtgcatggtatttcctaaacctgaaatgtgcttcccccgttttgcccaaacagacacagagtctccactccgccccctggttcacacctgcttaacctgccaagtcacatctgaactgtcacccttcagaaggtcctcctctggcaccctaatgtaattgagatcatcctattactctgtgttctggaactccacacttcagacatttctcattcctgtctaagctcttgtgtgtttggtttttggcctcacttttactgctctttaagctcccccagcggagtggagaggtctgttttcccgtgtttggattcccagaggcagtgcaagcctggcacaaggtcatcactaaaggagtgttcacaggatgaacgcggtgggagttagggaacgggtaaagcctgtgggatgagagaaggagcagagattgtctttggggtgcaggctcccaggaggaggcggcgcgggctccggtgctgggcggatcctcctccagctcctgcttggaggtctccagaataggctggaggcggggagggggtcccaaaagccttgggatcagaaggggttttcctgcctggtcccccagacaccccgttcgcctcaggaagacggaggatgagcctctgggctgcgcgtggtcggggttatggttgggatcagttaaggttccagtgcccgcaccccgcccagggagccccggatggcggcgtctctgtcagtatcttccccggaggccgcccctgtgaccggatccttcgtgtccccacagcacgtttcttgcagcaggataagtatgagtgtcatttcttcaacgggacggagcgggtgcggttcctgcacagagacatctataaccaagaggaggacttgcgcttcgacagcgacgtgggggagtaccgggcggtgacggagctggggcggcctgacgctgagtactggaacagccagaaggacttcctggaagacaggcgcgccgcggtggacacctactgcagacacaactacggggttggtgagagcttcacagtgcagcggcgaggtgagcatggtggggggcggggcctgggtccttgtgagctgggaatctgagtgtgtgtgtgtgtgtgtgtgtgtgtgtgtgtgtgtgtgtgtgagagagagagagagaggaagagagagacagagagggagcgcgccatctgtgagcatttagaatcctctcaatcttgagcaaggacttctgaggacacaggtgtgtgtgtagagtgtggatttgtctttgtggcctttgtgggaggggaagcaggagggggcttcttcttatccttggaggcctctgtggggaggtgacatgggaggtgggtgcagggggctggagagagaggagaccttggttgtctcgtgtccttagagatgcagggaaggaaagtgtaaggtgtgtgtggttggggtgaaggtttaggggaggagagctgaggggtgtggaagatttgtgataatatgaggaggccagttccagactgtccctggcacccacccttcacgcagtctctgaaataaaagtgtgtggtgtttgtttgcaaaagcattagattaatttctaggggaattgaggagacctctgaggcacttctgaagcttctttaggtctaaatttcttgctattttttctgtttgttttcttagtgtgtatatttttacatagttgaaatgactgtgaaactaactttttgattaaaattttaatacactgttactattttattataatgctaatagttttctactacttacctattattcttttatatataatagttgtgacacaacttatctcactttcccctttgttgacctttattatgacattcaccaaaagttgaaaatatatgtttctggttattatttaatttatttttttatttggaattcttttgaattatcttgacctatttattgcccaattataattactgctctaagaattccctattgtatttggtaggtaatggacaatgatctattttctgttatctccagggcttagtatttttctcagtgattttgtgggttctttgtactgtaagattattaacactttattgatatttgattccacattttctccagtttttgatttgtatgttgattttgaaaattctatgttaagaatttgaacatttctgtttaataaaatatattccaaaacttttattaatggtttacaaaccatcttaaatctatcattttgtggtatttttgtctccaggtttctccttccttcttaaaaaaaatgtatttattgagagtctgctagtgttagggattttcctaggcataagcaccccaagtaatgagtcccagaccctgcctgccttgatccaaatgtcattctggaaagaaaaattattttacaatgatagtataataatagttatgcttgtttgcatgggagatgcattgatcagctaaatgtaaatgtaagaattttcaaaactaaaataactttccttaatccttctctctgctttatgactcatgctttgctgggaacttaaagattggagaaccatttctgtctgtcctaccttcccaggggcacaaccatttctgtgttgttctaaggtgtgagtgcatggcagtagtattcctaaacattcatactcagtctccttttgtaccctactcggtccctttatctatccacattactttaaatcatatttttctctcaagctgtacaaggatgataaataggtggcaagtggaacacccaagtgtgatgagccctctcacagtggaatggagtgagaagctttatgacctcataaattaaaggctatcttcagtcattgttttatatattttatgtgcattaatcctcatataaccccaagaggtaaattactataattatcctctattatgggtgagaaagttgagacacagaagaatcgaaaaactcttccagcatcaaccagtaaaaggcagaccttggatttgagccaggcaacctggctcaggtatcagttttaattactacactctgtactttcaaagacttgtaaacactttgacaatgcatcccaatttcaagtgatgaagaaacaaacacaatttttcacatctctcaaatctgatgagcccccactataaagactaaattccaggctgatgacactgtgaggcctcatggccagctgtgctggaggcctggtcaaggccagagcctgggtttacagagaagcagacaaacagccaaaaagggagattcactctgtcttcctgagtcattccctctacatttcctttctcctagttgagcctaaggtgactgtgtatcctgcaaggacccagaccctgcagcaccacaacctcctggtctgctctgtgaatggtttctatccaggcagcattgaagtcaggtggttccggaacagccaggaagagaaggctggggtggtgtccacaggcctgattcagaatggagactggaccttccagaccctggtgatgctggaaacagttcctcgaagtggagaggtttacacctgccaagtggagcacccaagcgtgacgagccctctcacagtggaatggagtgagcagctttctgacttcataaatttctcaaccaccaagaagcaaactttactaatccctgagtgtcaggtttctcatctcccacatcctattttcatttgctccatgttctcatctccattagcacaggtcactgggggtagccctgtatagtttctagaaacacctgtatcctctggggaagcagtcattcctggcaggaaggagaggctgtccctgttttgaacctccccatgatgtcacaggtcagggtcaccccctctcccagggctccagaccctgcctctgggtctgagactgtgtttctggtgctgttgatctgagttatttgttgtgatctgggaagaggagaagtgtaggggccttcctgacatgaggggagtccaatatcagctctgccttttattagctctgtcactctagacaaactatttaacctcattgagtctcaggctttctgtttatcagatgttgaagccgtgccttacatcagggctgtaatattagaatgaatttgatccctgaaacttgtaactgttcagtgtgatttgaaaaccttttttttctccagaaatggctagttattttagttcttacagagcagccttcttccccattttcaaagctatgaatattgcagggtctcaattaaaaaggttcaatttgggataaaaatcactaaacctggtttcctctcccaggagcacagtctgaatctgcacagagcaagatgctgagtggagtcgggggctttgtgctgggcctgctcttccttggggccgggctattcatctacttcaagaatcagaaaggtgaggagcctttggtagcggctctctccatagacttttccagaggaggaaatagggctttgctgaggttagttctcagtatatgagtggccctggataaagcctttctttccacaaatgacctccaatgccctgctaatccagaaatcattaatgcatggttactatgtcaaaagcataatagcttgtggcctgcagagataagagaaaggttaactagttaagggtcctttggtttgaaatcctggagcaaattaaggaagagccactaaggctaatgcaattacactggatcctgtgacagacatgtcatgcttcatgggtcacatggtctgtttctgctcctctctgccctggttggtgtgggttgtggtgttagagaaatctcaggtgggagatctgggactaggacattgtgttgggagaatagatttgcttccataccttttaagtgtatatcttttcctctttttcccagggcactctggacttcacccaacaggtaataccttttaatcctcttttagaaacagattcagttttcctagaatgatggcagaggtgataaggcatgagacagaaatagcaggaaagactttggatccaaattcctgatcaggcaatttataccaaaactcctcctctccacttaggcctgtgctctgcaggagtattggttcagggagacttaggaacttgtttttcttcttcctgcagtgctctcatctgagtccttgaaagagaggaaaagaagctgttagtggaaccaggtctgaaaacaacactttcctctctctctgcaggactcgtgagctgaagtgcagatgaccacattcaagggggaaccttctgccccagctttgcatgatgaaaagctttcctgcttggctcttattcttccacaagagaggactttctcaggc

SEQ ID NO: 20 - DNA, 12657 bp, Homo sapiens
  source 1..12657: mol_type = other DNA
ctctggcccctggtcctgtcctgttctccagcatggtgtgtctgaagctccctggaggttcctacatggcagtgctgacagtgacactgatggtgctgagctccccactggctttggctggggacacccgacgtaagtgcacattgtgggtgctgagctactatggggtggggaaaatagggagttttgttaacattgtgcccaggccaggtgccttaagaaattgtgacattttcttcagagattgcccatctttatcatgggatcccaaattatttcctccacaaaaggagcttgactacttgccctctccaagagactgtgtaaggggcctccatacaggtcatttcttctcaaatctccaccaatgaaaccttttcatcacatgtcctcagggtctttagaggatttagaaataaggatgctaaaataaattccccatacagcacttccctttattatgttgacctatgtcagacaaaacgagtttttttctgaaaattttgtgggagtcaagggaattcaaagggtctctcctagacaatcctgtgttatgcccttgacagaacctgtgatattggcccctcttcctcatatgtgagaatggacccagtggcctccccattacctcctttcttttctttctgaactccaatgtttaccctgtaatgtatgcaaggtctctgacagaagttatgcttagtgctttttcttatggggaaaaatccttggagctgaagctgagatctttagtatgtggagtcaccctacagttaaagggcatctatgaggtattctttggtgcctaaaggacttaaggcatcctctaaaaacctggcccaggttagagtttattacaaatcttttttaacctttctatactatttgctcctatatctcctacatgctctaactagacatgacaggaagagattcaactaacataggacaaattatatgaaattctatttttgtaagtcaaaaatagtcaaatatcagaaatttgataaggttaaaactatatactctgtgtggggttacagagagaatgtggacattgttcacatctcatagggctgaaagttaatgatcaagtcctgggaactcattgtcttactggggtcttgtcctaaatttcataggttcatccgtcatgccctcagctttccttaattagccatgtctgcttacctcttcctccagtttctctctatttttccccagctatgttgtcatcatatccagaaatccctaaagcttgcacagatccttagcactatgagatccattgaaagagataatttttttctttttgagatagggcctggctctgtcacccaggctgtaatgcagtggtccgatctaggctcactgcaacctctgcctccaagactcaagctatccttcctcctcagcctccagagcagcggagactacaagcccaactaattttgttggagatgagatttctgccatgttgcccagggtggtcttaaactcctggactcaaacaatcctcctgccttggcctcccaacatgctaggattgtaggtgtaagccaccacgcccaggcacggtgaattcattgcccaggcaatgaatctcaatgaaaaatttcttttttcttaaatcactgtttctttatctgtgaattcttcttccaattagaaggaggagaaagaagaaatttgcctgtatttctcaccgggaagagaaggaatctagtgtgacatcaaattgaaagagtgctggagctcaagccccttcttgctttccagggtccttgcagtgatcagttcccagaaccatggtttattaatataaaccacagttatttttatcagcagctactctgtactgggctcccttctaggttcaaatcattctatttgagtaagatagagagggtcccgactctcaaggaggttacacaagagtggaggagacagacactaacccaataagcatttaacaaagaagaaaatttcagagagtcatagtgcactgaagaaaagacatcaggtttgtggaaaagagagaaatggattcacctactttagtttgtatgtctagagagccctacttgagaaagtgacatccagctgagaaaacaaaataagtagacagtcgtgaagatctaagggacgaaagttccagggagaccgaattgggggggaagccctggtgtgggaaattatgtgcagggacagaaagaaggctagagggactgaagtgagcaagcaaggaaatgcagtggcagaagatgaggtaggatgcagagaggaagtcaggagcctcatatcaggctctgatgtccctggtaagaaatttgaattttattttattttattaatttatttatttatcttatttatttttgagagggagtttcattcttgttgcccaggctggagtgcaatggcatggtctcagctcactgcaacctctgcctcccgggttcaagtgattctcttgcctcagtctcccaagtagctgggattacaggtacctgccaccatgctcagctattttttctgtgttttcactagagacagactttcaccatgttggccaggctggtcttcaactcctgacctcaagtaatctgcccgcctgagactcccaaagtgctgggattacaggcgtgagccactgcgcatggcctgaattttatttaaatagatatgggaagctactggatggttacaaggaaagtcaatttatattctttttttttaattttttaattttttttttttgagatatagtcttgctctgtcaccaggctggagtgcagtggcactatctcggctcactgcaacctctgccttctgggttcaagagattctcctgcctcagcctcccaagtagctgggattacaggagtgcaccaccacgcccagctaattttggtatttttagtaaagacagggtttcaccatgttggccaggatggtcttgatctcttgacctcgtgatccacctgcctcagcctcccagtgtgctgggattacaggcgtgagccaccgtgcccggcctatattgaatttttaaaactaattctagctactctgtggggattggattgttggggttgacaagtggttaggaagactatttaggagcacagcagggaatcctcagggaaaacaggcttgagttcactagtgataaagacagtgaaaaagataaagtggattttgcttaccttgttaatggattactgtaaagggggtagaaatatcaagcttattcctaaggattttgttttgacaaattagtgaaaggtggtggtgtttattgagatagggaaaactgtgggaggaaatgatttgaagtgggtggttggaaataaaagttttgtttaagtttgagatgatttattgacatttgtgtggagcaatcagaaggtcaatggcatttaaaagactcatggtgggccgggcgcagtgactcatgcctgtaatcccagcactttgggaggcctagacaggcggatcacgaggtcaggagatcaagaccttcctggctaacacggtgaaaccccgtctctactaaaaatacaaaaaattagctgggcatggtggtgggtgcctgtagtcccagctacttgggaggctgaagcaggagaatggcgtgaaccagggaggtggagcttgcagtgagctgagatcgcaccacttcactccaggctgggtgacagagcaagactccgtctcaaaaaaaaaaaaaaagagactcatggtgaggccagggctggaggtatttatgttggtggcatcaataggtgtactgtgttaaattccagggagtggaagaggttacatagggagattgtctggagaaaaaagaaaagggcacaggccagcaaaggggactaagaaagagcccagggatgttgggaaaaaaccaagattacatcatatgtgtaagtcaagaaaaatagatttttttttttccaaggagaagggagaggccaattgtggtgagtaccactaagaggagggtgaagtgagaatgtgacagagaagcaacggctgggattggtggagctgatatttgcagtcagtagagtatccagggaggaaactggattggaccatttgaagagcgagtagaagagaggacaaggttaaggttgactgttttgagtagcgagcttcagggaaggactgtgctctgggttcagggagccagctggatcaaaaggaaaaggctaaagaggctgaagagaagcaggaggacctgtgaaccagagatgctcggtcattattatcaaggaaatattagagggcccctgtgtgcagtggtgactgctcatgaagaggtcacacagccaatatttcacacagcccatatttattagtgacttagaatataccagttactactctaggtcatgagaatggagtgatgaataaaatgaatctggtctccatcagtatatgccatgtaacattttgcagtgactgtgtaccaggcctatgaatttcagtatgcaatttcaataatgatcctgctgtatctgtggtatataaaaacatgtacatctctggaatctaaaattgaaaggttataagtaaaacccagtatcacaaatttggtgctagaaatcagattgcagtttaaatctgagcatgtagaaagtccatttcttctatgtcagcaggtgccttttgtgtgaggtttaggtatactgcattattagacataaaccagtgtttctgccctctgttttcagaatgacaattctttatgaaactaatagaagaacagaagacaattgcaaaatcatgatgaagatactaattgctttagaatcatgtaatagaaaaaataatgtgagctccagttatagggatcaaaaaattaaaatgggaatacatttgagtgtttattatgtgatcactgctaagaagactctttattaaatttcacacttacaaataatcctgtgaggattattataaaatgcatttgatagattacaaataggcttatggttggcaaaaattgacccatttaaaagaggtcatatttcaattcatattttctgattctagaatatgagagtttttccatcattagtgagtagtgacttgattgtgtctgaattattgacaaaatttctaatattcatatgtaccaggttgtttcttagagtgggggcagagatgcaagggcttctagttccaatgtataaaagaaactttcattcattttgcatttatcattttaaaagttctataagtctatcataggcatgtgttgaagaacataaagaagtattaaatcattccttcttctgaggtttgactagcaagtctggctagggttgtcagataacaaacaggttcctagttaaatctgaatttcagacacacgaccacaatttattggaaatccagatttacctgggcatcctctggttttatttgtcaaatctgtcaaccctaagtgggacacatgagcatggattacagtgctagccatgcaagccacagtgacagtgactttacaaatgtttattttttaacattctgtctgtaaagaaagtgcttacataatttaggaatagaaagatagacattgtttgatccagggtgcacacctctctgccatcatttctaaagggcaaacggagctttgtgaaggtctctacacaaagtctggggacctgctcatgttttgtaaactgtatgagaacgtcattttcttggtttctgactttctgaggggacttgactacaaaaccaagagttttatctctggccaaggctggtaatttgatgcctgctagtattgttgggagtgggagactgaaagacgtgagttagttggggcattaaatgggaataaaatagctgtggttgtgattcactactacagataattagtggacaagtggcagagaaattaagaaagaagatgatatgaaagacaaatgatatgatttggtgactggttggtaatgcaaggaaatcagtaaatcttagttctcaacaagttcattttctggaaagatagcactgtattggggccagaattctataaaacattcgttttatgtaggaccaagatattcacagatatttttcaatacaattctcagctgctccataaccactagtggcttgttcaacacaggttttttcagatggttcacacctgtggttcttacccagggatcgttcaccaccgctcctttccctcccatcacccttggggaacttgtggcaatgtttgagcaatttttggttgtcaccacaggggtttcttctgatatttaatgagtagaagccagggacactgctagagaacctacaatattcagaacagcctcctttaccaacaaagaattatctggaccaaaatgtcaatagtgctgaggctgagagcactgcttcacactgtgctctttctgaaaattctagactcacatcttttatacactcagcgcccaattcagttctttatggtttatttttgcttgcttcattatgcaaaagtagacagttgcataaattcaaccactttcttgttgaatccatttagtcaatgcaagtttaatagcttcatagttagtttttgccttatgcaatatttttcaacattttcatgagttgttggtcagcactacctctattaactttcaacaacttgcccttataagtcacaaatagtgatgctctgcaattatttttcactaacatgcctgagatttctgtagtgattctacatttggtatgattcataatgtaaaatgcttttatttattcatttcacttttaaccagaggattatttttcagttatttttgtcatttttacacttcaatcaaacataaagacaaaaacatctaaaatatatagtattttacatatgtgtatatttacacacatatatgtatgtatgtttacatgtattgaaactacagaagctcatgtcaccaataagagctctgagacacttttgaccccttactcttatcaggtgaaatttgccaaataagttttatgaacaaatttcttttaactgaatatctgagctttgtggatttagaaatgcaaaggaaggtttgtggacattcatgggatcatggttttattctccttaaatctctttgatactttcccactgtctttagcataaatctcagatcctaacaccacccacgacgcttttcaatacctggcttcttgtgatgtctccagtctaatcttttaccctccttcccctcagcctctctgctttagtgaactttgttctagtttcttgaagaagttcatcaattcaagcttttgtgcatggtatttcctaaacctgaaatgtgcttcccccgttttgcccaaacagacacagtctccactccgccccctggttcacacctgcttaacctgccaagtcacatctgaactgtcacccttcagagggtccttctctggcaccctaatgtaattgagatcatcctattactctgtgttctggaactccacacttcagacatttctcatttctgtctaagctcttgtgtgtttggtttttggccatcacttttactgctctttaagctcccccagcggagtggagaggtctgttttcccgtgtttggattcccagaggcagtgcaagcctggcacaaggtcatcactaagggagtgttcacaggatgaacgcggtgggagttagggaacgggtaaagcctgtgggatgagagaaggagcagagattgtctttggggtggaggctcccaggaggaggcggcgcgggctccggtgctgggcggatcctcctccagctcctgcttggaggtctccagaataggctggaggcggggagggggtcccaaaagccttgggatcagaaggggttttcccgcctggtcccccagacagcccgttcgcctcaggaagacggaggatgagcctctgggctgcgcgtggtcggggttatggttgggatcagttaaggttccagtgcccgcaccccgcccagggagccccggatggcggcgtctctgtcagtgtcttccccggaggccgcccctgtgaccggatccttcgtgtccccacagcatgtttcttgcagcaggataagtatgagtgtcatttcttcaacgggacggagcgggtgcggttcctgcacagaggcatctataaccaagaggagaacgtgcgcttcgacagcgacgtgggggagtaccgggcggtgacggagctggggcggcctgacgctgagtactggaacagccagaaggacatcctggagcaggcgcgggccgcggtggacacctactgcagacacaactacggggctgtggagagcttcacagtgcagcggcgaggtgagcatggtggggggcggggcctgggtccttgtgagctgggaatctgagtgtgtgtgtgtgtgtgtgtgtgtgtgtgtgagagagagagagagagagagaggaagagagagacagagagggagcgcgccatctgtgagcatttagaatcctctcaatcttgagcaaggacttctgaggacacaggtgtgtgtgtagagtgtggatttgtctttgtggcctttgtgggaggggaagcaggagggggcttcttcttatccttggaggcctctgtggggaggtgacatgggaggtgggtgcagggggctggagagagaggagaccttggttgtctcgtgtccttagagatgcagggaaggaaagtgtaaggtgtgtgtggttggggtgaaggtttaggggaggagagctgaggggtgtggaagatttgtgataatatgaggaggccagttccagactgtccctggcacccacccttcacgcagtctctgaaataaaagggtgtggtgtttgtttgcaaaagcattagattaatttctaggggaattgaagagacctctgaggcacctctgaagcttctttaggtctaaatttcttgctattttttctgtttgttttcttagtgtgtatatttttacatagttgaaatgactgtgaaactaactttttgattaaaattttaatacactgttactattttattataatgctaatagttttctactacttacctattattcttttatatataatagttgtgacacaacttatctcactttcccctttgttgacctttattatgacattcaccaaaagttgaaaatatatgtttctggttaatatttaatttatttttttatttggaattcttttgaattatcttgacctatttattgcccaattataattactgctctaagaattccctattgtatttggtaggtaatggacaatgatctattttctgttatctccagggcttagtatttttctcagtgattttgtgggttctttgtactgtaagattattaacactttattgatatttgattccacattttctccagtttttgatttgtatgttgattttgaaaattctatgttaagaatttgaacatttctgtttaataaaatatattccaaaacttttattaatggtttacaaaccatcttaaatctatcattttgtggtatttttgtctccaggtttctccttccttcttaaaaaaaatgtatttattgagagtctgctagtgttagggattttcctaggcataagcaccccaagtaatgagtcccagaccctgcctgccttgatccaaatgtcattctggaaagaaaaattattttacaatgatagtataataatagttatgcttgtttgcatgggagatgcattgatcagctaaatgtaaatgtaagaattttcaaaactaaaataactttccttaatccttctctctgctttatgactcatgctttcctgggaacttaaagattggagaaccatttctgtctgtcctaccttcccaggggcacaaccatttctgtggtgttctaaggtgtgagtgcatggcagtagtattcctaaacattcatactcagtctccttttgtaccctactcggtccctttatctatccacattactttaaatcatatttttctctcaagctgtacaaggatgataaataggtggcaagtggaacacccaagtgtgatgagccctctcacagtggaatggagtgagaagctttatgacctcataaattaaaggctatcttcagtcattgttttatatattttacgtgcattaatcctcatataaccccaagaggtaaattactataattatcctctattatgggtgagaaagttgagacacagaagaattgaaaaactcttccagcatcaaccagtaaaaggcagaccttggatttgagccaggcaacctggctcaggtatcagttttaattactacactctgtactttcaaagacttgtaaacactttgacaatgcatcccaatttcaagtgatgaagaaacaaacacaatttttcacatctctcaaatctgatgagcccccactataaagactaaattccaggctgatgacactgtgaggcctcatggccagctgtgctggaggcctggtcaaggccagagcctgggtttacagagaagcagacaaacagccaaaaagggagattcactctgtcttcctgagtcattccctctacatttcctttctcctagttgagcctaaggtgactgtgtatcctgcaaggacccagaccctgcagcaccacaacctcctggtctgctctgtgaatggtttctatccaggcagcattgaagtcaggtggttccggaacggccaggaagagaaggctggggtggtgtccacaggcctgattcagaatggagactggaccttccagattctggtgatgctggaaacagttcctcggagtggagaggtttacacctgccaagtggagcacccaagcgtgacgagccctctcacagtggaatggagtgagcagctttctgacttcataaatttctcaaccaccaagaagcaaactttactaatccctgagtgtcaggtttctcatctcccacatcctattttcatttgctccatgttctcatctccgttagcacaggtcactgggggtagccctgtatagtttctagaaacacctgtatcctctggggaagcagtcattcctggcaggaaggagaggctgtccctgttttgaacctccccatgatgtcacaggtcagggtcaccccctctcccagggctccagaccctgcctctgggtctgagactgtgtttctggtgctgttgatctgagttatttgttgtgatctgggaagaggagaagtgtaggggccttcctgacatgaggggagtccaatctcagctctgccttttattagttctgtcactctagacaaactatttaacctcattgagtctcaggctttctgtttatcagatgttgaagccgtgccttacatcagggctgtaatattagaatgaatttgatccctgaaacttgtaactgttcagtgtgatttgaaaaccttttttttctccagaaatggctagttattttagttcttacagagcagccttcttccccattttcaaagctatgaatattgcagggtctcaattaaaaaggttcaatttgggataaaaatcactaaacctggcttcctctcccaggagcacagtctgaatctgcacagagcaagatgctgagtggaatcgggggctttgtgctgggcctgctcttccttggggccgggctattcatctacttcaagaatcagaaaggtgaggagcctttggtagcggctctctccatagacttttccagaggaggaaatagggctttgctgaggttagttctcagtatatgagtggccctggataaagcctttctttccacaaatgacctccaatgccctgctaatccagaaatcattaatgcatggttactatgtcaaaagcataatagcttgtggcctgcagagataagagaaaggttaactagttaagggtcctttggtttgaaatcctggagcaaattaaggaagagccactaaggctaatgcaattacactggatcctgtgacagacatgtcatgcttcatgggtcacatggtctgtttctgctcctctctgccctggttggtgtgggttgtggtgttagagaaatctcaggtgggagatctgggactaggacattgtgttgggagaatagatttgcttccataccttttaagtgtatatcttttcctctttttcccagggcactctggacttcacccaacaggtaataccttttaatcctcttttagaaacagattcagttttcctagaatgatggcagaggtgataaggcatgagacagaaatagcaggaaagactttggatccaaattcctgatcaggcaatttataccaaaactcctcctctccacttaggcctgtgctctgcaggagtattggttcagggagacttaggaacttgtttttcttcttcctgcagtgctctcatctgagtccttgaaagagaggaaaagaagctgttagtggaaccaggtctgaaaacaacactttcctctctctctgcaggactcgtgagctgaagtgcagatgaccacattcaagggggaaccttctgccccagctttgcatgatgaaaagctttcctgcttggctcttattcttccacaagagaggactttctcaggccctggttgctaccggttcagcaactctgcagaaaatgtcc

SEQ ID NO: 21 - DNA, 14942 bp, Homo sapiens
  source 1..14942: mol_type = other DNA
aaccttttgagtactaaattaaaacaatcctttaaagaagacaattgtttcagaggaggaccttcatacagcatctctgaccagcaactgatgatgctagtgaactcaggtgctgactggttctccaacacgagattacccaatcaaggagcaaggaaatcagtaacttccttcctataatttggaatgtgggtggaggggggccatagttctccctgattgagacttgcctgctgctgtgaccactggtcttgtcctcttctccagcatggtgtgtctgaagctccctggaggctcctgtatggcagcgctgacagtgacattgacggtgctgagctccccactggctttggctggggacacccaacgtaagtgcacattgtgactgcagagctactatggggtgggggcaaaaataggaagttttgttaacattgtgcccaggctatgtctcttatgaaattgtgacactttcttcagagattgcccatctttatcagatggatcccaaattatttccaccacaaaaggcgcttggctacttgtcctgtccatgagatttgtgtaaagggcctacacacaggtcatttcttctcaaatctccaccaataaaacctatgcatcacatgtcctcagggtctttagaggatttagaaataagtatgctaaaataatttccccatacagcacttccctttattatgttgacttatgtcagacaaaaggaggtttttactgaaaattttgtgggagtcaagggaattcaaagggtctctcctagacgatccagtgttatgtcctccacaggacctttggtgttggcccctcttcctcatatgtgaggatggatccagtggcctccccattatctcctttcttttctttctgaactccaatgtttataaagcctgtacccctgttgcctatgtaggttctctgacaaaagttatacttagtgctctttctttcttatggggaaaaatccgtggaactgaagctgagatctttagtacgtggagtcaccttacagatatagagcatttatgcggtattcattggtgcctaaagaactttaggcatcctctggaaaaccggcccaggttagtgtttattatgaatcttttttaaccgttctatacttgtttctcctacatctcttacgtgctctaactagacatgacagaagagatttaactaatgtagtataaattgtatgaaattctatttttgtaagtcaaaaataatcaaatatcagaaatttaataatgttcaaactatatactctgtgtggggtttttgagacaatgtggacattgttcatgtctcataaggctgaaagtcaatgagcaagtcctggaaactcattgtcttactggggtcttgtcctaaatttcataggttcacccatcatgccctcagctttccttaattagccatgtctgcttacctcttcctccactttctctcttttccccagctatgttgtcatcatttccaaaaatctctaaagcttgcacagatccttagcactatgagatccattgaaagacataatttttttctttttgagatagggcctagctctgtcacccaggctgtagctcagtggtgtgatcgaggctcactgcaaccactgcctcccatgctcaagcgatcttccctcctcaagctccggaatagctgggaatagaggcaggcaaccacgcccagctaattttttaattttggtagagatgagattttgccatgttgcccaggctggtcttaaactgctggactcaagcaatcctcctgccttggcctcccatcgtgctaggattatagatgtgagccactgtgcccaggcaaaaagaaatgactcttaataaaaaaatttcctttttcttaaatcactgtttctttatctgtgaattcttcttccaagtagaaggaggagaaagaagaagtttgcctgtatttctcaccgggaggagaaggggtctagtgtgacatcaaaatgaaagggtgctggagcttgagccccttcttgctttccgggatccccacagtgatcagttctcataccctggtttattcgtgtaaaccacacttatttttctcagcagctactgtgtactgggctccattctaggttcaaatcattctatttgagtaagatagagagggtcccgactctcaaggaaattacacaacaatagaggagacagacactaacccaataagcatttaacaaaaaagaaaatgttagagagtcatagtgcactgaagaaaagacaacaggtttgtcggaaagatagaaatggattcacctactttagttcatatgtttagggagccctacctgagaaagtgacattcagctgagacaacaaagtaagtagacagttgtgaagatctaatggacaaaagttccagggagactgaatcgagggaaagccctggtgtgggaaattatgtggagggagagaaagaaggctagaggggctgatgtataggaagcaaggaaataggggggcagaagatgaggtagaacacagagaggaagtcaggagcctcctcatattaggctctgaaatccacggtaaaaaatttgaatttcatttatttatttattttgagacagagtttcattcttgtggcccagactggagtgcaatggcacaatctcggctcactgcaacctccgcctcctgggttcaagtgattctcctgcctcagcttcccaagtagctgggattacaggcacccaccaccatacctggctatttttttgtatttttagtggagatggggtatcaccatgttgaccaggctggtctcgaactcctgacctcagataatctgtctgccttggcctcccaaagtgctgggattacaagagtgggccactgtgcccagactgaattttatttaaatagatatgagaaactactgtacggttacacggagagtcaatttatattcaattttgtgtgtgtgtgtttgtgagatcgagtcttgctctgttgcccaggctagattgcagtggcacaatctcggtttactgcaacctccgcctcccgggttcaagcgattctattgcctcagcctcccgagtaactgggaccacaggcgcatgccactacacctggctaattttttgtatttttaatagggatgaggtttcaccgtgttagccaggttggtctcgatctcctgacctcgtgatctgcccaccttggcctcccaaagtgctgggattacaggcgtgagccactgcacccggcctatattcaatttttaaaactaactctagctactctgtaggaattggattgttggggttcaaaagtggtcaggaagactatttaggagcacagcagagaattatccagggaaaacaggcttgtggcttcatggagtgcattagtgataaagagagtgaaaaagataaagtggacagacttggcatgtatttttgcttagcttgctaatgaattactgtaaaggggttagaacaatcaagcttattcctaaggattttgttttgacaaataagtgggtggtagtgttgtttattgagataggaaaaactatgggaggaaatgatttgaagtgggtggttggaaataaaagttttgtttaaatttgagatgatttattgacatttatgtggagcaatcagaaggtcaatggcatttaagagactcatggtgaggtgaagctagggcttcaggtatttatgttggcggcatcaataagtgtagtgtgttaaattccagggagtgtaagatgatacatagggagatggattgtgtggagaaaaaagaagagggtacaggccagcaaagggggctgagaaagagcccagggaggctggagaaaacccaagataacataatgcgtgtaagtgaagaaaaacagattattttcaaggagaagggagaggccaattgtggtgagtaccactaagaggagagggaagtgagaacgtgacagagaagcaagtgctgggttgctggtttgctggagttgatatttgcagtcaatggagtatccagggaggaaactggattggaccatttgaagagcaagtagaagtgaggacgaagttaagattgactgttttgagtagagagcttcagggaaggactgtgctctgggttcagggagccagctgattctaaaggaaaaggctaaagaagctgaagagccagctttcccagcaccatttattaaatagggaatcctttccccatttctgttttttgtcagatttatcaaacatcagatggttgtagatgtgtggtattatttctgagggctctgttctgttccattggtctatatctctgttttggtaccagtaccatgctgttttggttactgtaggcttgtagtacagtttgaagtcaggcagcatgatgcctccagctttgttcttttggcttaggattgtcttggcaatgcgggctcttttttggttccatatgaattttaaagtagttttttgcaattctgtgaagaaagtcattggtagcttgatgtggatggcactgaatctataaattaccttgggcagtatggccattttcacgatatttattcttcttatccatgagcatggaatgttcttccatttgtttgtgtcctcttttatttagttgagcagtggtttgtagttctccttgaagaggcccttcacgtcccttgtaagttggattcctaggtattctatactctttgaagcaactgtgaatgggagttcactcatgatttggctctctgtttgtctgttattggtgtataagaatgcttgtgattttttgcacatatgtagaaagctaaaactggatcctttctttacacctcatacaaaaattaattcaagatggattaaagacttaaatgttagacttaaaaccataaaaaccctagaagaaaacctaggcaataccattcgggacataggcatgggcaaggacttcatgtctaaaacaccaaaagcaatggcaacaaaagccaaaatggacaaatgggatctgattaaactaaagagcttctgcacagcaaaagaaaataccatcagagtcaacaggcaacctacagaatgggagaaaatttttgcaatctactcatctgacaaagggctaatatccagaatctacaaagaactcaaacaaatttacaagaaaaaaacaaacaaccccatccaaaagtgggcaaaggatatgaacagacacttctcaaaagaagacatttatgcagccaacagacacatgaaaaaatgctcatcatcactggccatcagagaaatgcaaatcaaaaccacaatgagataccatctcacaccagttagaatggcattcattaaaaagtcaggaaacaacaggtgctagagaggatgtggagaaataggaacacttttacactgttggtgggactgtaaacgagttcaaccattgtggaagacagtgtggtgattccttaaggatctagaactagaaatactgtttgacccagccatcccattactgggtatatacccaaaggattataaatcatgctgctataaagacacatgcacacgtatgtttattgtggcactattcacaatagcaaagacttggaaccaacccaaatgtccatcaatgatagactggatgaagaaaatgtggcacatacacaccatggaatactatgcagccataaaaaaggatgagttcatgtcctttgtagggacatggatgaagctggaaaccataattctcagcaaactatcacaagcagaaaaaaaccaaacaccacatgttctcactcataggtgggaattgaacaatgagaacacttggacacaggaagaggaacatcacacaccggggggaggggggagggatagcattaggagatatgcataatgtaaatgacgagttaatgggtgcagcacaccaacacggcacatgtatatgtgtgtaacaaacctgcacattgtgcacatgtaccctagaacttaaagtataataaaaaataaataaataaataaataaataaataaaaagaagctgaagagaaggaggaggacttgtgaaccagagatgctcagtcattattagcgaggaaatgctagagagcccctgtgtgcagtgctgactacttatgcaaaaggtcacacagccaatatttaacgcagccagtatttcacacagccaatatttattaatgacatagcatacatataccagttattactctaggtcatgagaatggagtgataaataaaatgaatctggtcgccatcagtatatgccaggtaacattttgcagtgactgtgtaccaggcctatgattttcagtatgcaatttcaataattatcctgttgtatctgtggtatttaaaaaacatatacatctctggaatctaaaattgagaggatataagtaaaacccagtgttacaaatttagtgctggaaatcagattgcagtttaaatctgagcatgtagaaagtccctttcttctatgtcagcagatgccttttgtgtgaggtttaggtatactgctttattagacataaaccagtgtttctgccctatgttttcagaatgacaattccttatgaaactaatagaagaacagaagacaattgcaaaatcatgatgaagatactaattgctctagaattcaggaatacataaaatgatgtgagctgcagttatagggatcataaaagttaaaatgggaatgcatttgagtgtttattatgtgatcagtgctaataagagtcatcatttaattttacacttaacagtaatcctgtgaggattacgctattattaaatgcatttgatagattacaaaaaggattatggttggtaaaaattgccccaagtagaagagatcatgttttttattcaggttatctgattctagagtttgagagtttgactgaccatcattagtgagtagtgactatattgtgtctgaattattgacagaatttctgatattcaaatgtaccaggttgcttcttagagtgcagacagagatgcaagggctgctagttccgatgtataggagaaactttcattcatttcacatttatcattttaaaagttctgtatgtctataatggtcatgtgttgaagaacacaaggaagtattaaatcactccttcttctgaggtttgactagcaagttgggctagagttaccaaataaaatacatgttcctagttaaatctgaatttcagatacacaaccatgatttcttggaaattcaaatttacctgggcatcctctggttttatttgccaaatctgtcaaccctaagtgagacacatggacatggattacagtgctagtcatgcaaaccacagtgacagcaacttcacacatgtttattttttaactttctgtctgtaaagaaagtacttagataatttagggataaaaagatagacattgttttatccagggtgcacacctctctgccatcatttctaaagggcaaagggacatttctgcaggtcttgctcacagtctggggacctgctcgtttttgcaaactgtctgtatgagtatgtcattttcttggtttctccctttctaaggggacttgactgcaaaaccaggagttctgcctctggccaaggctggaaatttgatgcctgctggtattgttgggagtgggagactgaaagaaatgagttagttggggcatcaaatgggaaataaaatagctgtggttgtgattcattattacagaaaattagtggaccagtggcagagaaattaagaaaaaagatgatgtgaatgataaacgatatgattagtggctacttggtaaggcaaggaaatcattaaatcttggttctcatcaagttcattttctgaaaagatagcactgtattgggaccagaattctacaaaacattctttttatgtaggaccaagattttcaacaaatatttttcaatgcaattctcagcgctccataactaatagtagcttgttcaacacagattttttcggatggttcacacctgtagttcttacccagggatagttcaccacccctcccttccctcccatcatcgttggtgaacggtggcaatgtttggaacaatttttggttgtcacaacaggggtttcttctgatattcaatgagtagaagccagggacactgctaaagaacctacaatattcagaacagcctctgctatcaacaaggaattatctggtccaaaatgtcaatagtgctgaggctaagagcactggttcacactgtgctctttctgaaaattctaggctcacatctggtatacactcaccacacagttcagttttttatggtttatttttggttgtttcattataaaaaattagacagttacataaattcaaccattttcttgttgaatccatttagtcaatgcaagcttaatattttcatatttattttttgccttatgcaatatttttcaacattttcatgagttgttggtcatcactatctctattaactttcaacaacttgctcttgtaagtcacaaatagtgatgctgctgaaactgtttctcactaacaggcctcagatttgtgtagtgattctacattttatattattcacaatgtaaaatgctactatttattcatttcacttttacccacaggattattttaagttattttttgtcattttcacacttcaaacataaagacaaaaacatcaaaaatatatacagtgttttacatatgtgtatattttcacatatatctgtatgtgtgtttatatgtattgaaactacagaagcacatgtcaccaataagagctctgagacacctttgaccacttacccttatcagatgatatttgccaaatgagttgtggaaacaagttttttttaactgaatttcagagctttgtggatttagaaattcaaaggaaagtttgtggacatttaccgggatcatggttttattctccttaaaactcttcaatactttaccattgtctttagttaaatccaaaatcctaacaccacccacgaggcttttcaatacctgccttcttgtgatttctccagcctaatcttttaccttccttcccctcagcctctctgctttagtgaactttgttctagttttttgaagaagctcatcaattcaaccttttgtacatgggatttcctaaacctgaaaatgtgcctcccgttttgtccagacagacacacaggctccatccactctgccccctggctctcacctgcttaacctgtcaagtcacatctgaactgtcactcttcagagggtccttctctggcaccctaatgtaattgagatcatccttttattctctgttccagaactccacacttctgacgtttctcattcctgtctaagctcttgtgtgtttggtttttggccatcacgttcactgctctttaagctcccccagcggagtggagaggtctgttttcctttgtttggattcctagaggcagcgcagaccaggcacaaggtcagcactaaggaagggttcacgggatgaacacggtgggtgctgttgaaggaaccggtaaagcgtgtgggatgagaggagcagagagtgtctttcaggtgaagactcccagaaggaggccgcgtgggccgcggtgctgggcggatcctcctccagctcctgcttggagttctccagaacaggctggaggcagggagggggtccccaaaagcctgaggatcagaaggggttttcccgcctggtcccccagaccccggttcgcctcaggaagactgaggacgagcccctgggctgctggtggtgggcgttgcgggtggggccggttaaggttcccagcgcccgcaccccgcccagggagtccgggttggtggcgtcgctgtcagtgtcttctcaggaggccgcccgtgtgaccggatcgttcgtgtccccacagcacgtttcttggagcaggctaagtgtgagtgtcatttcctcaatgggacggagcgagtgtggaacctgatcagatacatctataaccaagaggagtacgcgcgctacaacagtgacctgggggagtaccaggcggtgacggagctggggcggcctgacgctgagtactggaacagccagaaggacctcctggagcggaggcgggccgaggtggacacctactgcagatacaactacggggttgtggagagcttcacagtgcagcggcgaggtgagcatggtggagggcggggcctgggtccctgtgagcggggaatctgtgtatgtgtgtgtgtgtgtgtgtgtgagagagagagagagagagagagacagaggaagagagagagagcgtgcgccatctgtgagcatttagaatcctctctatcctttcttatggctgcgtagtattccctggtgtataggtgccacattgtcttaatccagtctatcattgatggacattagggttaattccaagtctttgctattgtgaatagtgtcagttcatgtcctttgtagggacatggatgcagctggaaaccatcattctcagcaaactatcacaaggacagaaaaccaaacaccacttgttctcactcataggtgggaattgaacaatgagaacacatggacacaggaaggggaacatcacacacaggggcctgtcgtggggtggggggagcagggagtgatagcattaggagatatacctaatgtaaatgactagttaatgggtgcagcataccaacatgacacatgtgtacatatgtaacaaacctgcacgttgtgcacatgtatcctagaacttaaagtataataataataattatatatatatatatattagaaaaacaaacaaaaagaatcttctgtatcctgagcaaggagttctgaaggcacaggtgtgtgtagagtgtggatttgtctgtgtggctgttgtgggaggggaaacaggagggggctgcttcttattcttggaggcctctgtgcagaggtgacatgggaggtgggtgtagggggctggagagagaggagaccttgattgtcctgggtccttagagatgcagggaagggaagaataaggtgtgtgtggttggggtgaaggtttaggggaggagagctgaggagtgtggaaggtttgggataatgtgaggaggccagttccagactgtccctggcacacacccttcatgtaatctctgaaataaaagtgtgtgctgtttgtttgtaaaagcattagattaatttctaggggaattgaggagacctctgaggcacctctgaagcttctttaggtctaaatttcttgctagtttttgtttttttagggtgtatatttttacatagcagaagtgactgtgaaactaactttttgaattaaagttttaacacagttactattttattataatgctaatagttttctagtagttacatcttattcttttgtatataatagttgtgacacaacttacctcactttcccctttttgttgacctttattatgactttcaccaaaagttgaaaatgtatgtttctggttaatttttactttatatttttcatttgtaattcttttgaattattttgacctatttattggccaattataattactgctgtaagaattccctatttcatttggtagggaatgtacaatgatctactgtctaatatcttaagggcttagtatttttctcagtgactttgtgggttctttgtactgtaagattattaacactttattgatatttgattcagcattttctccagtttgtggtttgtatgtggattttgaaaagtattttccatgttaagaatttgaacatttttatttaataaaatatattgcgaaatttgtattaatgatttacaatcatcttaaatccaccattttgtggtattattgtctccaggtttctccttacttctaaaaaaaaactgtatttattgagagtatgctagtgtcagggattttcctgggcataagcaccccaagtgacgagtcccagacactgccttaatccaaatgtcattctggaaagaaaaatcattttacaatgataggcctaataataattaagcttgtgttgcacaggagatacattgatcagctaaatgtaaacgtaagaactttcaaaactaaaatgacattccttaatcattctctctgcgttaggactcatgcttttctaggaacgtaaaaatttgagaatcatttctgtctgtcccaccttcccaggggcagaaccatttctgtggtgttctaaggtgtgagtgcatggtggtagtattcccaaaaattcatactcggtttcctcatgtacccaactctgtccctttatctacccacattgctttaagtcatatttttctgtcacggtgtacaaggatgataaataggtgccaagtggagcacccaagtgtgatgagccccctcacagtggaatggagtgacaagctttctaacctcataaattgaaggctatcttcagtcattgttttatatattttacgtgcattaatcctcatataaccccaagaggtaaattagtataattatccttcattgtaggtgacaaagttgagacacagaagaatcaaaaaactcttccagcatcaaccagtaaaaggcagaccttggatttcaaccaggcaacatggctcagatatcagttttaattactacactctactgtcaaagatttgtaaacactttgacaatgcatgccaatttcaagctatgaagaaacaaacacaatttttcacaacatctctcaaatctaatgggtcctcactatcaagattaaattccaggctgatgacactgtaaggccacatggccagctgtgctggaggcctggtcaaggtcagagcctgggtttgcagagaagcagacaaacagccaaacaaggagacttactctgtcttcatgactcattcccactaccttttttttctcctagtccaacctaaggtgactgtgtatccttcaaagacccagcccctgcagcaccacaacctcctggtctgctctgtgaatggtttctatccaggcagcattgaagtcaggtggttccggaacggccaggaagagaaggctggggtggtgtccacaggcctgatccagaatggagactggaccttccagaccctggtgatgctggaaacagttcctcggagtggagaggtttacacctgccaagtggagcatccaagcatgatgagccctctcacggtgcaatggagttagcagctttctgacttcctaaatttctcacccaccaagaaggggactgtgctaatccctgagtgtcaggtttctcctctcccacatcctattttcatttgctccatgttctcatctccatcagcacaggtcactgggctactgggggtagccctgtaggtgtttctagaaacacctgtaccccctggggaatcagtcacgcctgccaggcgggagaggctgtccctcttttgaacctccccatgatgtcccagttcagggtcacccactctccccaggctccaggccctgcctctgggtctgagactgcgtttctggtgctgttgatctgagttgtttgttgtgagaagaggagaactctaggggccttcctgacatgaggggagtccaatctcaactctgccttttattagctctgtcactctagacaaactacttaacctcactgagactcaggctttctgttgatcagattttgaagttgcgccttacatcaaggctgtaatatttgaaatgagtttgatgcctggaccttgtaactgttacagtgtgatttgaaaaccgttttttcccccagaaatggctagttattttagttcttacagggcagccttctttcccattttgaaagctctgaatcttagggtctcaattacagaggttcaatttggaataaacactaaacctggcttcctctctcaggtgcacggtctgaatctgcacagagcaagatgctgagtggagtcgggggctttgtgctgggcctgctcttccttgggacagggctgttcatctacttcaggaatcagaaaggtgaggagcttttgggagctgaatctccataggcttttctggaggaggaactatgactttgctagggttagttctcagtatatcagtggccctggataaagcctttctttccccaaatgacctccaatgtcctgataatccagaaatcatcagtgcatggttactatgtcaaagcataatagcttgtggcctgcagagataagagaaaggttaacaagtagggattctttggttggagatcctggagcaaattaaggaagagccactaaggctaatgcaattacgctggatcctgtgacagacacctcatgcttcatgggtcacatggtctgtttctgttcctctctgccctggttggtgtgggttgtggtgttagagaaatctcaggtgggagatctggggctgggacattgtgttagaagacagatttgcttccatatcttttaagtgtatatcttttcctctttttcccaggacactctggacttcagccaacaggtaataccttttaatcctctttcagaaagagattcagtttccctagaatgatggcagaggtgataaggcatgagacagaaataataggaaagactttggatccagatttctgatcaggcaatttaccccagaactcctcctctccacttagaaaaggcctgtgctttgcaggatcattggctcagggagacttaggaacttgtttttcttctccctgcagtgctctcatctgagtcgttgaaagcggggaaaagaagttttagtaaagccaggtctgaaaacaatttcctctgtctctgcaggactcttgagctgaagtgcagatgaccacattcaaggaagaaccttctgccccagctttgcaagatgaaaagctttcccacttggctcttattcttccacaagagcttt

SEQ ID NO: 22 - DNA, 1098 bp, synthetic construct
  source 1..1098: mol_type = other DNA
atggccgtgatggccccaagaacactgctgctgctgctgtccggcgcactggccctgacccagacctgggccggcagccacagcatgcggtacttcttcacaagcgtgtcaagacccggcagaggcgagcctagattcatcgcggttggatacgtggacgacacccaatttgtgcggttcgactctgatgccgccagccagaaaatggaacctagagctccttggatcgagcaagagggtcctgagtactgggaccaggagacaagaaacatgaaggcccattctcaaacagatagggccaatctgggaaccctgcggggctactacaaccagagcgaggatggcagccacaccattcagatcatgtacggctgcgacgtgggccccgacggcagatttctgagaggctatagacaggatgcttacgacgggaaggactacatcgccctgaacgaggacctgcgatcttggaccgccgctgatatggctgctcagatcaccaagcggaagtgggaagccgtgcacgccgccgagcagcggagagtgtacctggaaggcagatgcgtggacggactgagaagatatctggaaaacggcaaggaaaccctgcagcggacagatcctccaaagacccacatgacccaccaccccatcagcgaccacgaggccaccctgcgctgctgggctctgggcttctaccctgccgaaatcaccctgacatggcagagagatggcgaggaccagacacaggacaccgagctggtggaaaccagacctgccggcgacggaacattccagaagtgggccgccgtggtggtccccagcggcgaggaacagagatacacctgccacgtgcaacacgagggactgcctaagcccctgaccctgagatgggagctgagctctcagcctaccatccctatcgtgggcatcatcgccggactcgtgctgctgggcgccgtgatcaccggcgccgtcgtggccgccgtgatgtggcggcggaaaagcagcgacagaaagggcggctcctacactcaggccgcttctagcgatagcgcccagggcagcgacgtgtccctgacagcctgtaaagtgtga

SEQ ID NO: 23 - DNA, 1098 bp, synthetic construct
  source 1..1098: mol_type = other DNA
atggccgtgatggccccaagaacactggtcctgctgctgagcggcgccctggccctgacacagacctgggccggatctcacagcatgcggtacttctacaccagcgtgtctagacctggaagaggcgagccaagattcatcgccgtgggctatgtggacgacacccagttcgtgcggttcgacagcgacgccgcttctcagagaatggaacccagagccccttggatcgagcaagagggccctgagtactgggacagaaataccagaaacgtgaaggcccaaagccaaacagacagagtggacctgggcaccctcagaggctactacaaccagagcgaggccggcagccacaccattcagatgatgtacggctgcgacgtgggctccgatggcagatttctgcggggatatagacaggatgcctacgacggaaaggactacatcgccctgaaggaagatctgaggtcctggaccgccgctgatatggccgctcagacgaccaagcacaagtgggaagccgcccatgtggctgaacagtggcgggcctacctggaaggcacatgcgtggaatggctccggcggtacctggaaaacggcaaagagacactgcagcggacagatgcccctaagacccacatgacccaccacgccgtgtccgaccacgaggccaccctgcggtgctgggccctgtctttttaccccgccgagatcacactgacctggcagagggacggcgaggaccagacccaggacaccgagctggtggaaacacggcctgctggcgacggcaccttccagaagtgggtggccgttgtggttcctagcggccaggagcagagatacacctgccacgtgcagcacgagggactgcctaagcctctgaccctgagatgggagcccagcagccaacctaccatccccatcgtgggcatcatcgccggcctggtgctgttcggggctgtcatcaccggcgctgtggtggccgccgtgatgtggcgcagaaaaagctctgatagaaagggcggaagctacagccaggccgccagcagcgatagcgcccagggctctgatgtgtccctgacagcttgtaaagtgtga

SEQ ID NO: 24 - DNA, 1098 bp, synthetic construct
  source 1..1098: mol_type = other DNA
atggccgttatggccccaagaacactggtgctgctgctgtcaggcgctctggccctgacccagacctgggctggcagccacagcatgagatactttttcaccagcgtgtctagacccggcagaggagaacccagattcatcgccgtcggatatgtggacgacacccagttcgttagattcgacagcgatgccgcctctcagagaatggaacctagagctccctggatcgagcaagagggacctgagtactgggacggcgaaaccagaaaagtgaaggcccatagccagactcatcgggtggacctgggaacactgagaggctactacaaccagagcgaggccggttctcacaccgtgcagcgcatgtacggctgcgacgtgggcagcgattggcggtttctgcggggctaccaccagtacgcctacgacggcaaagactacatcgccctgaaggaggacctgcgatcttggaccgctgcagatatggccgctcagaccaccaagcacaagtgggaggccgcccacgtggccgagcaactgcgggcctacctggaaggcacctgtgtggaatggctgagaagatacctggaaaacggcaaggaaacactgcagcggacagacgcccctaagacccacatgacccaccacgccgtgtccgaccacgaggcaaccctccggtgctgggccctcagcttctaccccgctgagatcaccctgacctggcagagagatggcgaagatcagacacaggacaccgagctggtcgagacaagacctgccggcgacggcaccttccagaagtgggccgccgtggtggtgccatctggccaggagcagagatatacatgccacgtgcagcacgagggcctgcctaagcctctgacactgaggtgggaacctagcagccagcctaccatccccattgtgggcatcatcgccggactggtgctgttcggcgccgtgatcacaggcgctgtggtcgccgctgtgatgtggcggcggaaaagctccgatagaaagggcggcagctacagccaagccgccagcagcgactccgcccagggcagcgatgtgtccctgaccgcctgcaaggtgtga

SEQ ID NO: 25 - DNA, 1098 bp, synthetic construct
  source 1..1098: mol_type = other DNA
atggccgtgatggcccctagaaccctgctgctgctgctcagcggcgccctggctctgacccaaacatgggccggcagccacagcatgagatacttctacacctccgtgtctagacccggcagaggcgagcccaggttcatcgccgtgggctacgtggacgacacccagttcgtccggttcgacagcgacgccgccagccagagaatggaacctagagccccttggatcgagcaggagggccctgagtactgggaccaggagacaagaaatgtgaaggcccaatctcagaccgatagagtggacctgggcaccctgcgcggctactacaaccagagcgaggatggctcccacacaattcagatcatgtacggctgcgacgtgggaccagatggaagatttctgagaggctatagacaggatgcctacgacggaaaggactacatcgccctgaacgaggacctgcggagctggacggccgcagatatggctgcccagatcaccaagcggaaatgggaagccgcccacgccgccgagcagcagagagcttatctggaaggcagatgcgtggaatggctgaggagatacctggaaaacggcaaggaaaccctgcagcggaccgaccctccaaagacccacatgacccaccatcctatcagcgatcacgaggccacactgcgctgctgggctctgggcttctaccccgctgaaatcacactgacctggcagcgggacggggaggaccagacccaggacaccgagctggtggaaacaagacctgctggcgacggcacatttcagaagtgggccgctgttgtggtccccagcggcgaggaacagcggtacacctgtcacgtgcagcacgagggcctgcctaagcctctgacactcagatgggagctgagcagccaacctaccatccccatcgtgggcatcatcgccggtctggtcctgcttggagccgtgatcaccggcgccgtggtggccgccgtgatgtggcggagaaaaagcagcgaccggaagggcggaagctacacacaggccgcttcttctgatagcgcccagggctctgatgtgtccctgaccgcctgcaaggtgtga

SEQ ID NO: 26 - DNA, 1098 bp, synthetic construct
  source 1..1098: mol_type = other DNA
atggccgtgatggcccctagaaccctgctgctgctgctgtctggcgctctggccctgacccaaacctgggccggatctcacagcatgcggtatttcttcaccagcgtgtctagacccggcagaggcgagcccaggttcatcgccgtgggctacgtggacgacacccagttcgtgcggttcgacagcgacgccgctagccagagaatggaacctagagccccttggatcgagcaggagggccctgagtactgggaccaggagacaagaaacgtgaaggcccaaagccagacagacagagtggacctgggcaccctgagaggctactacaaccagagcgaggccggcagccacaccatccagattatgtacggctgcgacgtgggttctgatggcagatttctgcgcggatatagacaggatgcctacgacggaaaggactacatcgccctgaatgaggatctgcggtcctggaccgccgcagatatggcagctcagatcaccaagcggaaatgggaggccgcccacgaggccgaacagctgcgggcctacctggacggcacatgcgtggaatggctgagacggtacctggaaaacggcaaggaaaccctccagagaacagatcctccaaagacccatatgacccaccaccccatcagcgatcacgaggctacactgagatgctgggctctgggcttctaccccgccgagatcacactcacatggcagcgggacggagaggaccagacccaggacacagagctggtggaaaccagacctgccggcgacggcacctttcagaaatgggccgccgtggtggtgcccagcggcgaggaacagagatacacttgtcacgtgcagcacgaaggcctgcctaagcctctgaccctgagatgggaactgagcagccaacctacaatcccaatcgtgggcatcatcgccggcctggtcctgctgggagccgttatcaccggcgccgtggtcgccgctgtgatgtggcggcggaagagctccgatagaaagggcggaagctacacccaggccgccagcagcgactccgcccagggcagcgacgtttctctgacagcctgcaaggtgtga

SEQ ID NO: 27 - DNA, 1098 bp, synthetic construct
  source 1..1098: mol_type = other DNA
atggctgtgatggcccctagaacactggtcctgctgctgtcaggcgccctcgccctgacccagacttgggctggcagccatagcatgagatacttcagcaccagcgtgtctagaccaggaagaggcgagccccggttcatcgccgttggctacgtggacgacacacagttcgtgcggttcgactccgatgccgcatctcagcggatggaacccagagccccttggatcgagcaggagggacctgagtactgggacgaggaaaccggcaaggtcaaggcccacagccaaaccgacagagagaacctgcggatcgccctgagatactacaaccagagcgaggccggcagccacacactgcagatgatgttcggctgcgacgtgggctccgatggcagattcctgcggggataccaccagtacgcctacgacggaaaggactacatcgccctgaaggaagacctgcggtcctggaccgccgccgacatggcagctcaaatcacacagagaaagtgggaggccgccagagtggccgagcagctgagggcttacctggaaggcacctgcgtggatggccttagacgctacctggaaaatggcaaagaaaccctgcagaggaccgatcctcctaagacccacatgacacaccaccccatcagcgaccacgaggccaccctgagatgctgggccctgggtttttatcctgctgaaatcaccctgacctggcagagagatggcgaggaccagacccaagacaccgagctggtggaaacaagacctgccggagatggcacctttcagaaatgggccgctgtcgtggtgcccagcggcgaagagcagcggtatacatgtcacgtgcagcacgagggcctgcccaagcctctgacactgcggtgggaaccatctagccagcctaccgtgcacatcgtgggcatcatcgccggcctggtgctgctgggcgctgtgattaccggcgccgtggtggccgccgtgatgtggcggagaaacagcagcgatagaaagggaggctcttacagccaggccgcttctagcgacagcgcccagggcagcgacgtgtccctgacagcctgcaaggtgtga

SEQ ID NO: 28 - DNA, 1098 bp, synthetic construct
  source 1..1098: mol_type = other DNA
atggccgtgatggctcctagaaccctggtgctgctgctgagcggcgccctggccctgacacagacctgggccggctcccactccatgagatacttctcaacaagcgtgtctagaccaggcagaggcgaacctagattcatcgccgtgggctatgtggacgacacccagttcgtgcggtttgattctgatgccgcttctcaacggatggaaccccgggccccttggatcgagcaggagggacctgagtactgggacgaggaaacaggaaaagtgaaggcccacagccaaaccgatagggagaacctgagaatcgccctgagatactacaaccagagcgaggccggcagccacaccctgcagatgatgttcggctgcgacgtgggcagcgacggaagattcctgcggggctaccaccagtacgcctacgacggcaaggactacatcgccctgaaggaagatctgagaagctggaccgccgccgacatggccgcccaaatcacaaagcggaagtgggaagctgcacatgtggccgagcagcagagagcctacctggaaggcacctgcgtggacggcctgagaagatatctggaaaatggcaaagagacactgcagaggaccgacccccccaagacccacatgacccaccaccccatcagcgaccacgaggctaccctgcggtgctgggccctcggattttaccccgccgagatcaccttgacatggcagcgggacggcgaggatcagacacaggacaccgagctggtggaaaccagacctgccggcgatggaacattccaaaagtgggctgccgtcgtggtgccttctggcgaggaacagagatacacctgtcacgtgcagcacgagggcctgcctaaacctctgacactgcgctgggagcctagctctcagcctaccgtgccaattgtgggaatcatcgccggactggtcctgctgggcgctgtgatcaccggcgccgttgtggccgctgtgatgtggcggagaaacagcagcgacagaaagggcggctcctacagccaggccgccagcagcgatagcgcccagggcagcgacgtgagcctgactgcctgcaaggtgtga

SEQ ID NO: 29 - DNA, 1098 bp, synthetic construct
  source 1..1098: mol_type = other DNA
atggccgtgatggctcctagaacactgctgctgctcctgctgggagccctggccctgacacagacatgggctggctcccacagcatgcggtacttcaccaccagcgtgtctagacccggcagaggcgagcccagattcatcgctgtgggatacgtggacgacacccaatttgtgcggttcgactctgatgccgctagccagcgcatggaacctagagccccttggatcgagcaagagggacctgagtactgggaccggaacaccagaaatgtgaaggcccactctcaaatcgacagagtcgacctgggcaccctgagaggatattacaaccagagcgaggccggcagccacaccatccagatgatgtacggctgcgacgtgggcagcgatggcagattcctgaggggctaccagcaggacgcctacgacggaaaggactacatcgccctgaacgaggatctgagaagctggaccgccgcagatatggccgctcagatcacacagcggaagtgggaggccgccagagtggccgaacagctgagagcctatctggaaggcacatgcgtggaatggctgcggcggtacctggaaaacggcaaagagacactgcagagaaccgacccccccaagacccacatgacacaccacgccgtgtccgaccacgaggccaccctgcggtgctgggccctttctttctaccctgctgagatcaccctgacctggcagagggacggcgaggaccagacccaggataccgagctggtggaaaccagaccagccggcgacggcacttttcagaagtgggccagcgtggtggtgcctagcggccaggaacagagatacacctgccacgtgcagcatgagggcctgcctaagccactgaccctgagatgggaaccttccagccaacctacaatccccatcgtgggcattatcgccggactggtgctgttcggcgctgttttcgccggcgccgtcgtggccgccgtgcggtggcggagaaagggctctgatagaaagggcggcagctacagccaggccgcatccagcgacagcgcccagggttcagatatgagcctgacagcctgtaaagtgtga

SEQ ID NO: 30 - DNA, 801 bp, synthetic construct
  source 1..801: mol_type = other DNA
atggtctgcctgaagctgcctggcggcagcagcctggctgctctgacagtgaccctgatggtgctgagctctagactggccttcgccggcgacaccagacctagattcctggagctgctgaagagcgagtgccacttcttcaacggcaccgagagagtgcgcttcctggaaagacatttccacaatcaagaggaatacgcccggttcgacagcgatgtgggcgagtatagagccgtgagggaactgggcagacccgacgccgagtactggaacagccagaaagacctgctggagcagaagcggggccaggtggacaactactgcagacacaactacggcgtgggagagagcttcaccgtgcagcggagagtgcacccccaggtcaccgtttaccctgccaagacacagcctctgcaacaccacaacctgctcgtgtgcagcgtgtccggcttctaccccggctctatcgaggtccggtggttccggaacggacaggaggaaaaggccggcgtggtgtcaacaggcctgatccagaatggcgattggaccttccagaccctggtgatgctggaaaccgtgcctcggagcggcgaggtgtacacctgtcaggtggaacacccttctgtgacaagccctctgaccgtggaatggtccgccagaagcgagtccgctcagagcaagatgctgtctggagtgggcggatttgtgctgggactgctgtttctgggcgccggtctttttatctacttcagaaaccagaaaggccacagcggcctccagccaacaggctttctgagctga

SEQ ID NO: 31 - DNA, 801 bp, synthetic construct
  source 1..801: mol_type = other DNA
atggtgtgcctgaagctgcctggaggaagctacatggccaaactgaccgtgaccctgatggtcctgtctagcccacttgctctggctggcgacacccggcctagatttctgcagcaggacaagtacgagtgccacttcttcaacggtacagagagagtgcggttcctgcatagagatatctacaaccaggaggaagatctgagattcgacagcgacgtgggcgagtatagagccgtgacagagctgggcagacctgacgccgagtactggaacagccagaaggacttcctggaagatagaagggccgctgtggacacatactgcagacacaattacggcgtgggcgaaagcttcaccgtgcagcggagagtggagcctaaggtgaccgtgtaccccgccagaacacagaccctgcaacaccacaatctgctggtctgcagcgtgaacggcttctaccctggctctatcgaggtgcggtggttccgcaactcccaggaggaaaaggccggcgttgtgtccaccggactgatccagaacggcgattggaccttccagacactggtgatgctggaaaccgtgccaagaagcggagaagtgtacacctgtcaggtggaacaccccagcgtgaccagccccctgaccgtcgagtggcgggcccagagcgagagcgcccaaagcaagatgctgtccggcgtgggcggctttgtgctgggcctcctgttcctgggcgccggactgtttatctacttcaagaaccagaaaggccactctggcctgcaccctacaggcctggtgtcttga

SEQ ID NO: 32 - DNA, 801 bp, synthetic construct
  source 1..801: mol_type = other DNA
atggtctgtctgaagctgcctggcggctccagcctggccgctctgaccgtgacactgatggtgctgagctctagactggccttcgccggcgacaccagacccagattcctggaactgctgaaatctgagtgccattttttcaacggaacagagagagtgagatttctggaaagatacttccacaaccaggaggaattcgtgcggttcgacagcgatgtgggagagtacagggccgtgaccgagctgggccggcctgtggccgagagctggaacagccagaaggacctgctggagcagaaaagaggccaggtcgacaactactgcagacacaactacggcgtggtggaaagcttcaccgtgcagcgccgggtgcaccctcaggtgaccgtgtacccagccaagacacagcctctgcagcaccacaacctcctggtgtgcagcgtgtccggcttctaccccggcagcatcgaggtgcggtggttccggaacggccaggaggaaaagaccggcgtggtctccaccggactgatccacaatggcgattggaccttccagaccctggtgatgctggaaaccgttcctagaagcggcgaggtgtatacatgccaggtggagcaccccagcgtgacatctccactgacagtggaatggcgggccagaagcgagagcgcccagagcaagatgctgagcggcgtgggcggctttgtgctgggactcctgttcctgggtgctggccttttcatctactttagaaatcaaaagggccacagcggcctgcaacctaccggattcctgtcttga

SEQ ID NO: 33 - DNA, 801 bp, synthetic construct
  source 1..801: mol_type = other DNA
atggtctgcctgaagctgcctggcggcagctacatggccaagctgaccgtgaccctgatggtgctgagcagccccctggctctggccggcgataccagaccaagattcctgcagcaggacaagtacgagtgccacttcttcaacggcaccgagcgggtgcggttcctgcacagaggcatctacaaccaggaggaaaacgtgagattcgactctgatgtgggagaatatagagccgtgaccgagctgggcagacctgacgccgagtactggaatagccagaaggacttcctggaagatagacgggctgctgtggacacatactgcagacacaactacggcgtcggcgaatcctttacagtgcagagaagggtggaacctaaagtgaccgtgtaccccgccagaacccagaccctgcaacaccacaatctgctggtttgttctgtgaacggcttttacccaggcagcatcgaggtgcggtggttccgcaactctcaggaggaaaaggccggagtggtgagtacaggtctgatccagaacggcgactggaccttccagacactggtgatgctcgagacagtgcccagaagcggcgaggtgtacacatgccaggtggagcaccctagcgtcaccagccctctgaccgttgaatggcgggcccagagcgagagcgcccaaagcaagatgctgagcggagtgggaggcttcgtgcttggactgctgttcctgggcgccggcctgtttatctacttcaagaaccagaaaggccactctggcctgcatcctaccggcctggtgtcctga

SEQ ID NO: 34 - DNA, 801 bp, synthetic construct
  source 1..801: mol_type = other DNA
atggtgtgcctgaagctgcctggaggctcttgtatggccgctctgaccgtgacactgaccgttctgtctagccctctggctctggccggagatacacagcctagattcctggaacaggccaagtgcgagtgccacttcctgaacggcacagagagagtgtggaatctgatcagatatatctacaaccaggaggaatacgccagatacaacagcgacctgggcgagtaccaggctgtgaccgagctgggacgccccgacgccgagtactggaacagccagaaagatctgctggaacggagacgggccgaggtggacacctactgcagatataattacggcgtggtggaaagcttcaccgtgcaaagaagggtgcagcctaaggtgaccgtgtaccccagcaagacccagccactgcaacaccacaacctgctggtgtgtagcgtgaacggattttaccctggcagcatcgaggtgcggtggttccggaactctcaggaggaaaaggccggcgtcgtgtccaccggcctgatccagaacggcgactggaccttccagaccctggtgatgctcgagacagtccccagatccggcgaagtgtacacctgccaggtggaacatcctagcatgatgagccccctgacagttcaatggtccgcccggagcgagagcgcccagtctaaaatgctgagcggcgtgggcggcttcgtgctgggactgctctttctgggcaccggcctgttcatctacttcagaaaccagaagggccacagcggcctgcagcctacaggccttctgagctga

SEQ ID NO: 35 - DNA, 801 bp, synthetic construct
  source 1..801: mol_type = other DNA
atggtgtgtctgaagctgcctggcggaagcagcctggctgctctgaccgtgacactgatggtgctgagctctagactggccttcgccggagataccagacctagattcctggaactgagaaagtccgagtgccatttcttcaacggcaccgagcgggtgcggtacctggatagatatttccacaatcaagaggaattcctcagattcgacagcgacgtgggagagtaccgcgccgtgaccgagctgggcagaccagtggccgagtcttggaacagccagaaggacctgctggaacagaaacgtggaagagtggacaactactgcagacacaattacggcgtgggcgagagcttcaccgtgcagaggcgggtgcacccccaggttacagtgtaccctgccaagacccagcctctgcaacaccacaacctgctggtgtgcagcgtgtccggcttctaccccggcagcatcgaggtgcggtggttccggaacggccaggaggaaaaggccggcgtggtgtcaacaggcctgatccagaacggcgactggaccttccagacactggtcatgctggagacagtccccagatccggcgaagtgtacacctgtcaggtggaacacccttctgtgaccagcgctctgaccgtcgagtggcgggccagaagcgaaagcgcccagagcaagatgctgtctggcgttggaggctttgtgctgggcctgctgttcctcggtgccggcttgtttatctactttagaaaccagaaaggccacagcggcctgcagcctaccggcttcctgagctga

SEQ ID NO: 36 - DNA, 765 bp, synthetic construct
  source 1..765: mol_type = other DNA
atggctatctctggcgtgccagtgctgggcttcttcatcatcgccgtgctgatgagcgcccaggagtcttgggccatcaaagaggaacatgtgatcattcaggccgaattctatctgaaccccgatcagagcggcgagtttatgttcgacttcgatggcgacgagatcttccacgtggacatggccaagaaggaaaccgtgtggcggctggaagagtttggcagattcgccagcttcgaggctcaaggagctctggccaacatcgccgtggataaggccaatctggaaatcatgaccaagagaagcaactacacccctatcacaaacgtgccccccgaggtgaccgtgctcaccaacagccccgtcgagctgcgggaacctaacgtgctgatctgcttcatcgacaagttcacccctccagtggtgaacgtgacatggctgagaaacggcaagcctgtgaccacaggcgtgtccgagacagtgttcctgcctagagaggaccacctgttccggaagtttcactacctgcctttcctgcctagcaccgaggatgtgtacgactgcagagtggaacactggggcctggacgagcctctgctgaagcactgggagttcgacgccccttcccctctgcccgaaaccacagagaacgtggtttgtgccctgggactgaccgtcggactggtgggcattatcatcggcactatctttatcatcaagggcgttagaaaaagcaatgccgcagaaagacgcggccctctttga

SEQ ID NO: 37 - DNA, 771 bp, synthetic construct
  source 1..771: mol_type = other DNA
atggaatatgccagcgacgccagcctggaccctgaagccccttggcctcctgcccctagagcccgggcctgcagagtgctgccttgggccctggttgctggcctgctgctgctgctgctgctcgccgccgcttgcgccgtgtttctggcttgtccttgggctgtgtccggcgcccgggccagccccggcagcgctgcctctccaagactgagggaaggccctgagctgagccctgatgatccagctggactgctggacctgcgccagggcatgttcgcccagctggtcgcccagaacgtgctgctgatcgacggccccctgagctggtacagcgaccctggcctggccggagtgtccctcaccggcggcctgtcctacaaagaagacaccaaggaactggtggtggcaaaggccggcgtgtactacgtgttcttccagcttgagctgagacgggtcgtggccggcgagggatctggctctgtgtctctggccctgcacctgcaacctctgcggagcgccgccggagccgctgccctggccctgacagtggacctgccccccgcttctagcgaggctagaaacagcgccttcggctttcagggaagactgcttcatctgagcgccggccagagactgggcgtgcacctgcacaccgaggccagagccagacacgcctggcagctgacacagggcgccaccgtgctgggcctgttccgggtgacccctgagatccccgccggcctgcccagcccaagaagcgagtaagctagc

SEQ ID NO: 38 - DNA, 978 bp, synthetic construct
  source 1..978: mol_type = other DNA
atgggactgagtaacattctcttcgtgatggccttcctgctctctggtgctgctccgctgaagattcaagcttatttcaatgagactgcagacctgccttgccaatttgcaaactctcagaaccaaagcctgagtgagctagtagtattttggcaggaccaagaaaacttggttctgaatgaggtgtacttaggcaaagagaaatttgacagtgttcattccaagtatatgggacgcacaagttttgattcggacagttggaccctgagacttcataatcttcagatcaaggacaagggcttgtatcagtgtatcatccatcacaaaaagcccacaggaatgattcgcatccaccagatgaattcagaactgtcagtgcttgctaatttcagtcaacctgaaatagtaccaatctctaatataacagaaaatgtgtacataaatttgacctgctcatcgatacacggttacccagaacctaagaagatgagtgttttgttaagaaccaagaattcaactatcgagtatgatggtattatgcagaagtctcaagataatgtcacagaactgtacgacgtttccatcagcttatctgtttcattccctgatgttacgagcaatatgaccatcttctgtatcctggaaactgacaagacgcggcttttatcttcacctttctctatagagctcgaggaccctcagcctcccccagaccacattccttggatcacagctgtacttccaacagttattatatgtgtgatggttttctgtctaatactatggaaatggaagaagaagaagcggcctcgcaactcttataaatgtggaaccaatacaatggagagggaagagagtgaacagaccaagaaaagggaaaaaatccatatacctgaaagatctgatgaagctcagcgtgtttttaaaagttcgaagacatcttcatgtgacaaaagtgatacatgtttttaaggatcc

SEQ ID NO: 39 - DNA, 453 bp, synthetic construct
  source 1..453: mol_type = other DNA
atgtggctgcagagcctgctgctgctgggcaccgtcgcctgcagcatctccgcccctgctagatccccatctccaagcacccagccttgggaacacgtgaacgccatccaggaggccagacggctgctgaatctgagccgggacaccgccgctgaaatgaacgagacagtggaagtgattagcgagatgttcgatctgcaggagcctacctgtctgcaaacaagactggaactgtacaagcagggcctgagaggaagcctgaccaagctgaagggccctctgaccatgatggccagccactacaaacagcactgcccccccacacctgagacaagctgcgccacccagatcatcaccttcgagtcttttaaggaaaacctgaaggacttcctgctcgtgatccccttcgactgctgggagcctgtgcaggagtgactcgaggaattcggatcc

SEQ ID NO: 40 - DNA, 579 bp, synthetic construct
  source 1..579: mol_type = other DNA
atggccctcaccttcgccctgctggtcgccctgctggtgctgtcctgcaagtctagctgcagcgtgggctgtgacctgcctcaaacacactccctgggctctagaagaaccctgatgctgctggcccagatgcggagaatcagcctgttcagctgtctgaaagatagacacgacttcggcttccctcaggaggaattcggcaaccagtttcagaaggccgaaaccatccctgtgctgcacgagatgattcagcagatcttcaatcttttttctacaaaggatagcagcgccgcttgggacgagaccctgctggataagttctacacagagctgtatcagcagctgaacgacctggaagcctgcgtgatccagggcgtgggagtgaccgagaccccactgatgaaagaggacagcatcctggctgtgcggaagtacttccaaagaatcaccctgtacctgaaggaaaagaagtacagcccctgcgcctgggaggtggtgcgggccgagatcatgaggtccttctctctgagcacaaacctgcaggagagcctgagaagcaaggaatgactcgaggctagc

SEQ ID NO: 41 - DNA, 546 bp, synthetic construct
  source 1..546: mol_type = other DNA
atgtttcacgtgtccttccggtacatcttcggcctgcctccactgatcctggtgctgctccccgtggcctctagcgactgcgatatcgagggcaaggacggcaaacagtacgagagcgtgctgatggtctccatcgaccagctgctggacagcatgaaggaaatcggcagcaactgcctgaacaacgagttcaacttcttcaagcggcacatctgtgatgccaacaaggaaggcatgttcttgtttagagccgctagaaagctgaggcagttcctgaagatgaacagcaccggcgacttcgacctgcacctgctgaaggtgtctgagggaacaaccatcctgcttaattgcaccggacaagtgaagggcagaaagcctgctgccctgggcgaggcccagcctacaaagagcctggaagagaacaaaagcctgaaagaacagaaaaagctgaatgatctgtgcttcctgaagagactgctgcaggagattaagacctgttggaacaagatcctgatgggcaccaaggaacattgactcgagggatcc

SEQ ID NO: 42 - DNA, 1619 bp, synthetic construct
  source 1..1619: mol_type = other DNA
atgtgccatcagcagctggtcattagctggttctccctggtgttcctggccagccctctggtggccatctgggaactgaagaaggacgtgtacgtagtggaactggactggtaccctgatgcccctggcgagatggtggtgctgacatgcgacacccctgaggaagatggcatcacatggaccctggatcagtcttctgaggtgctgggatctggcaaaaccctcacaattcaggtgaaggaatttggcgatgccggacagtacacatgccacaaaggcggagaggtgctgagccactcgcttctgctgctgcacaagaaagaggacggcatctggtccaccgacatcctgaaggaccagaaggaacccaagaacaaaaccttcctgcgctgcgaggccaagaactacagcggcagattcacctgttggtggctgaccaccatcagcacagatctgaccttttctgtgaagagcagcagaggcagcagcgacccccagggcgtgacatgcggcgccgccaccctgtctgccgagagagtgcggggcgacaacaaggaatatgagtacagcgtggagtgccaggaggacagcgcctgtcctgctgctgaggaaagcctgcctatcgaggtgatggtcgacgctgtgcacaagctgaagtacgagaactacaccagcagttttttcatccgggacatcatcaagcctgatccacctaagaacctgcaactgaaacctctgaaaaatagcagacaggtggaagtcagctgggagtaccccgatacatggagcaccccacacagctacttcagcctgaccttctgcgtgcaggttcagggcaagtccaagcgggaaaagaaagaccgggtgttcaccgataagacatctgctacagtgatctgcagaaagaacgccagcatctccgtcagagcccaggacagatactacagctctagctggtcggagtgggccagcgtgccttgcagcgtgcccggcgtgggcgtgcccggagtgggagctagaaacctgccagtggccacccctgaccctggcatgttcccctgtctgcaccacagccagaacctcctgcgggccgtgtccaacatgctgcagaaagcccggcagaccctggaattctacccctgtacctccgaagagatcgaccacgaggatatcaccaaggacaagacctccaccgtggaagcctgcctgcctcttgaactgaccaagaatgagagctgtctgaatagcagagaaaccagcttcatcacgaacggcagctgcctggccagccggaagacatcttttatgatggccctgtgtctgagcagcatctacgaggacctgaagatgtaccaggtggagttcaagaccatgaacgctaagctgctgatggaccccaagagacaaatcttcctggaccaaaatatgctggctgttatcgacgagctgatgcaggccctgaacttcaacagcgagacagtgcctcagaaaagcagcctggaagagcctgacttctataagacaaagatcaagctgtgcatcctgctgcacgccttcagaatcagagccgtgaccatcgatagggtgatgtcttacctgaacgcctcataagtttaaac

SEQ ID NO: 43 - DNA, 867 bp, synthetic construct
  source 1..867: mol_type = other DNA
atgggccacacaagaaggcagggcaccagccctagcaagtgtccttacctgaatttcttccagctgctggtgctggctggcctgagccacttctgcagcggcgtgatccacgtgacaaaggaagtcaaggaggttgctaccctgagctgcggccacaacgtgtccgtggaagagctggcccagacaagaatctactggcaaaaggaaaagaaaatggtgctgacaatgatgagcggagatatgaacatctggcctgagtacaagaaccggacaatcttcgacatcaccaacaacctgtctatcgtgatcctggccctgagaccttctgatgagggcacctatgagtgcgtggtgctgaagtacgagaaggacgccttcaagcgcgaacatctcgccgaggtgaccctgagcgtgaaggccgacttccctacccctagcatcagcgacttcgagatccccacctccaacatcagaagaatcatctgtagcaccagcggcggatttccagaaccccacctgagctggctggaaaatggcgaggaactgaacgccattaacaccaccgtgtcccaggaccccgagacagagctgtacgccgtgtcaagcaaactggactttaacatgacaaccaatcacagctttatgtgcctgatcaagtacggtcacctcagagtgaaccagaccttcaactggaacaccacaaaacaggagcacttccccgataacctgctgccatcttgggccatcaccctgatcagcgtcaatggcatcttcgtgatctgctgcctgacctactgcttcgcccctagatgtagagagagaagacggaacgagcggctgcggcgggaatctgtgcggcctgtgtga

SEQ ID NO: 44 - DNA, 1611 bp, synthetic construct
  source 1..1611: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 45 - RNA, 20 nt, synthetic construct
  source 1..20: mol_type = other RNA
  ncRNA 1..20: ncRNA_class = sgRNA
acagcgacgccgcgagccag

SEQ ID NO: 46 - RNA, 20 nt, synthetic construct
  source 1..20: mol_type = other RNA
  ncRNA 1..20: ncRNA_class = sgRNA
tgcagacacaactacggggt

SEQ ID NO: 47 - DNA, 8399 bp, synthetic construct
  source 1..8399: mol_type = other DNA
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattaatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagagagatccagtttgggtcgaggatcgatcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgagccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgccgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtggatccccgggtaccgagctcgaattcatcgatcgattagtccaatttgttaaagacaggatatcagtggtccaggctctagttttgactcaacaatatcaccagctgaagcctatagagtacgagccatagatagaataaaagattttatttgtctccagaaaaaggggggaatgaaagaccccacctgtaggtttggcaagctaggatcaaggttaggacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatctagatccgctagccacgtggtttaaacgtcgacaatcaacctctggattacaaaatttgtgaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgatatccccgggtgatcaggtacctttaagaccaatgacttacaaggcagctgtagtcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtgttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 48 - protein, 9 aa, synthetic construct
AAGIGILTV

SEQ ID NO: 49 - protein, 9 aa, synthetic construct
EVDPIGHLY

SEQ ID NO: 50 - protein, 9 aa, synthetic construct
CMTWNQMNL

SEQ ID NO: 51 - protein, 20 aa, synthetic construct
RSFLVNQMINEAKKQLLEFD

SEQ ID NO: 52 - protein, 17 aa, synthetic construct
ISLETVAIDRPAEVRKV

SEQ ID NO: 53 - protein, 20 aa, synthetic construct
SASHLEQYGTNEYSIISSMK

SEQ ID NO: 54 - protein, 17 aa, synthetic construct
SSFERFEIFPKESSWPN